(19) United States
(12) Patent Application Publication
(10) Pub. No.: US 2009/0156594 A1
(43) Pub. Date: Jun. 18, 2009

(54) HETEROCYCLIC KINASE MODULATORS

(75) Inventors: Pierre-Yves Bounaud, San Diego, CA (US); Christopher Ronald Smith, San Diego, CA (US); Elizabeth A. Jefferson, San Diego, CA (US); Jorg Hendle, San Diego, CA (US); Patrick S. Lee, San Diego, CA (US); Angelina Marie Thayer, San Diego, CA (US); Gavin Charles Hirst, San Diego, CA (US)

Correspondence Address: WILSON SONSINI GOODRICH & ROSATI, 650 PAGE MILL ROAD, PALO ALTO, CA, 94304-1050, US

(73) Assignee: SGX Pharmaceuticals, Inc., San Diego, CA (US)

(21) Appl. No.: 12/124,988

(22) Filed: May 21, 2008

Related U.S. Application Data
(60) Provisional application No. 60/939,313, filed on May 21, 2007.

(51) Int. Cl. A61K 31/5377; C07D 215/00; A61K 31/47; C07D 253/10; A61K 31/53; A61P 35/00; C12N 9/00; C07D 487/02; A61K 31/519; C07D 295/00

(52) U.S. Cl. 514/233.2; 546/152; 514/314; 544/184; 514/243; 544/263; 514/259.1; 544/128; 435/183

(57) ABSTRACT

The present disclosure provides heterocyclic protein kinase modulators and methods of using these compounds to treat diseases mediated by kinase activity.

### CROSS-REFERENCE TO RELATED APPLICATIONS

This application claims the benefit of U.S. Provisional Application No. 60/939,313, entitled “Heterocyclic Kinase Modulators” filed on May 21, 2007, the disclosure of which is hereby incorporated by reference in its entirety.

### BACKGROUND OF THE INVENTION

Mammalian protein kinases are important regulators of cellular functions. Because dysfunctions in protein kinase activity have been associated with several diseases and disorders, protein kinases are targets for drug development.

The tyrosine kinase receptor, FMS-like tyrosine kinase 3 (FLT3), is implicated in cancers, including leukemia, such as acute myeloid leukemia (AML), acute lymphoblastic leukemia (ALL), and myelodysplasia. About one-quarter to one-third of AML patients have FLT3 mutations that lead to constitutive activation of the kinase and downstream signaling pathways. Although in normal humans, FLT3 is expressed mainly by normal myeloid and lymphoid progenitor cells, FLT3 is expressed in the leukemic cells of 70-80% of patients with AML and ALL. Inhibitors that target FLT3 have been reported to be toxic to leukemic cells expressing mutated and/or constitutively-active FLT3.

The Abelson non-receptor tyrosine kinase (c-Abl) is involved in signal transduction, via phosphorylation of its substrate proteins. In the cell, c-Abl shuttles between the cytoplasm and nucleus, and its activity is normally tightly regulated through a number of diverse mechanisms. Abl has been implicated in the control of growth-factor and integrin signaling, cell cycle, cell differentiation and neurogenesis, apoptosis, cell adhesion, cytoskeletal structure, and response to DNA damage and oxidative stress.

The c-Abl protein contains approximately 1150 amino-acid residues, organized into a N-terminal cap region, an SH3 and an SH2 domain, a tyrosine kinase domain, a nuclear localization sequence, a DNA-binding domain, and an actin-binding domain.

Chronic myelogenous leukemia (CML) is associated with the Philadelphia chromosomal translocation, between chromosomes 9 and 22. This translocation generates an aberrant fusion between the ber gene and the gene encoding c-Abl. The resultant Bcr-Abl fusion protein has constitutively active tyrosine-kinase activity. The elevated kinase activity is reported to be the primary causative factor of CML, and is responsible for cellular transformation, loss of growth-factor dependence, and cell proliferation.

The 2-phenylaminopyrimidine compound imatinib (also referred to as STI-571, CGP 57148, or Gleevec) has been identified as a specific and potent inhibitor of Bcr-Abl, as well as two other tyrosine kinases, c-kit and platelet-derived growth factor receptor. Imatinib blocks the tyrosine-kinase activity of these proteins. Imatinib has been reported to be an effective therapeutic agent for the treatment of all stages of CML. However, the majority of patients with advanced-stage or blast crisis CML suffer a relapse despite continued imatinib therapy, due to the development of resistance to the drug. Frequently, the molecular basis for this resistance is the emergence of imatinib-resistant variants of the kinase domain of Bcr-Abl. The most commonly observed underlying amino-acid substitutions include Glu255Lys, Thr315Ile, Tyr293Phe, and Met351Thr.

MET was first identified as a transforming DNA rearrangement (TPR-MET) in a human osteosarcoma cell line that had been treated with N-methyl-N′-nitro-nitrosoguanidine. The MET receptor tyrosine kinase (also known as hepatocyte growth factor receptor, HGFR, MET or c-Met) and its ligand hepatocyte growth factor (“HGF”) have numerous biological activities including the stimulation of proliferation, survival, differentiation and morphogenesis, branching tubulogenesis, cell motility and invasive growth. Pathologically, MET has been implicated in the growth, invasion and metastasis of many different forms of cancer including kidney cancer, lung cancer, ovarian cancer, liver cancer and breast cancer. Somatic, activating mutations in MET have been found in human carcinoma metastases and in sporadic cancers such as papillary renal cell carcinoma. In addition to cancer there is evidence that MET inhibition may have value in the treatment of various indications including: Listeria invasion, Osteolysis associated with multiple myeloma, Malaria infection, diabetic retinopathies, psoriasis, and arthritis.

The tyrosine kinase RON is the receptor for the macrophage stimulating protein and belongs to the MET family of receptor tyrosine kinases. Like MET, RON is implicated in growth, invasion and metastasis of several different forms of cancer including gastric cancer and bladder cancer.

The present disclosure is directed to potent protein kinase inhibitors that are used, among other things, to treat numerous diseases and conditions which kinases have been implicated, such as cancer. Although certain protein kinases are specifically named herein, the present disclosure is not limited to inhibitors of these kinases, and, includes, within its scope, inhibitors of related protein kinases, and inhibitors of homologous proteins.

### BRIEF SUMMARY OF THE INVENTION

The present disclosure provides heterocyclic compounds used to modulate kinase activity and to treat diseases mediated by kinase activity. These heterocyclic kinase modulators are described in detail below. In addition, inhibitory activities of selected compounds are disclosed herein.

In one aspect is a compound having the structure of Formulas (I1), (I2), (I3) or (I4):

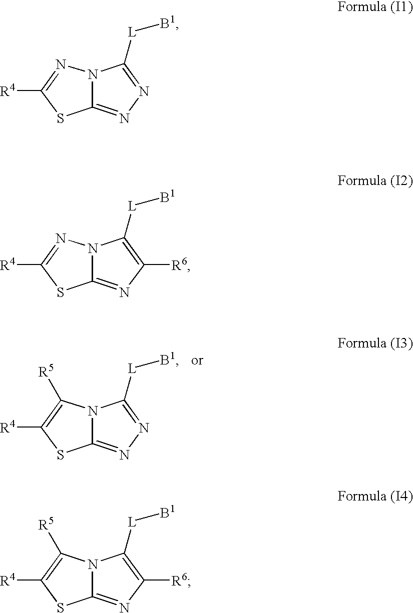

wherein:

L is

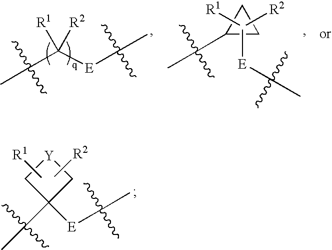

E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;

R⁴ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹;

R⁵ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)NR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹;

R⁴ and R⁵ optionally form substituted or unsubstituted cycloalkyl, substituted or unsubstituted heterocycloalkyl, substituted or unsubstituted aryl, or substituted or unsubstituted heteroaryl,

R⁶ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, (CH₂)ⱼS(O)ₘR²¹, —(CH₂)ⱼNR²⁰S(O)₂R²¹, —(CH₂)ⱼS(O)₂NR¹⁸R¹⁹;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

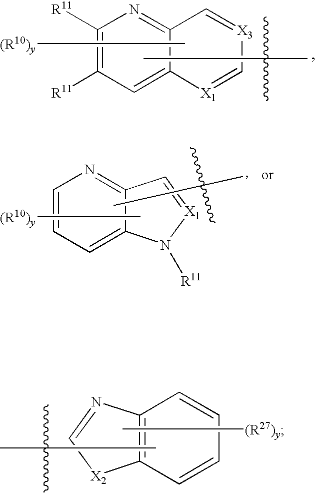

B¹ is

wherein:
X₁ is independently N or CR¹¹;
X₂ is NR¹¹, O, or S; and
X₃ is CR¹⁰ or N;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 4;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴; wherein each j is independently an integer from 0 to 6, and m is independently an integer from 0 to 2;

with the proviso that when R¹¹ is independently a direct bond, then R¹⁰ or R²⁷ cannot all be H;

R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

In one embodiment is a compound having the structure of Formulas (I1a), (I1b), (I2a), (I2b), (I3a), (I3b), (I4a), and (I4b):

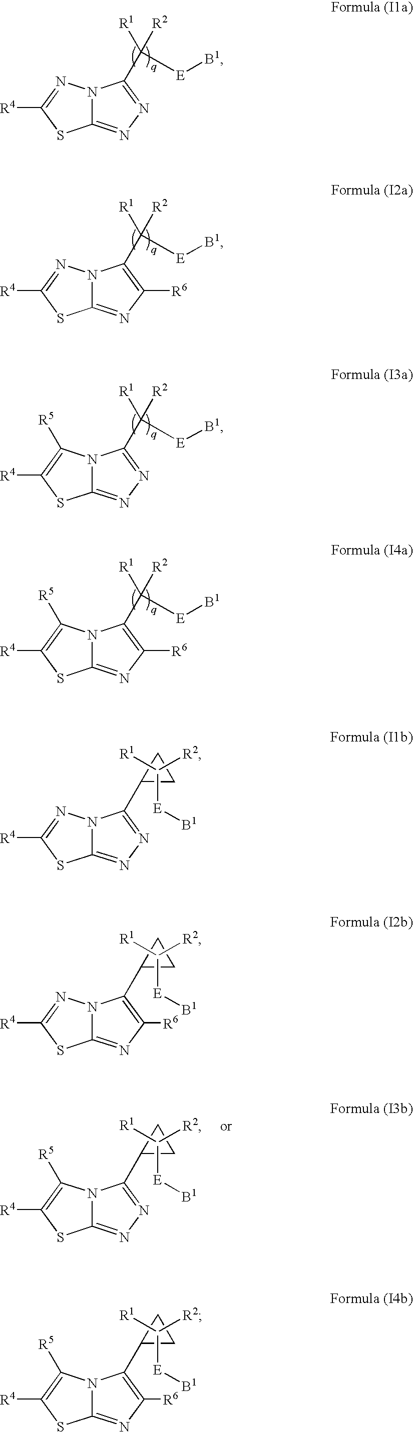

or an enantiomer, diastereomer, racemate, or pharmaceutically acceptable salt, or solvate thereof.

In another embodiment B¹ is

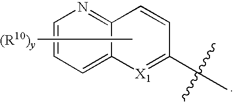

In a further embodiment, X₁ is CR¹¹; and wherein R¹¹ and each R¹⁰ are independently hydrogen, halogen, nitro, cyano, hydroxyl, substituted or unsubstituted alkyl, substituted or unsubstituted aryl, substituted or unsubstituted heteroaryl, and y is an integer from 0 to 5.

Also presented herein are compounds having the structure of Formulas (I1a), (I2a), (I3a), and (I4a):

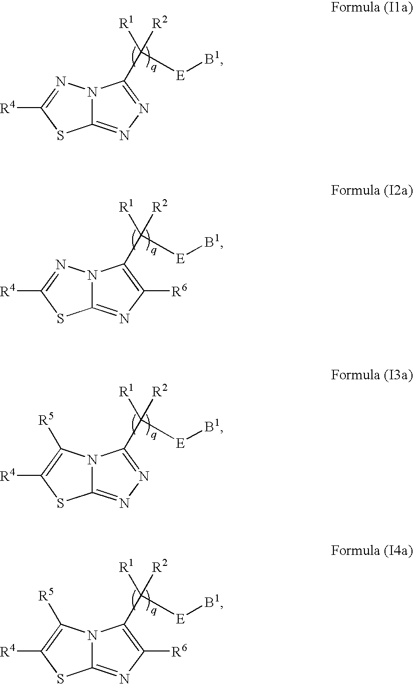

wherein y is 1 or 2; q is 0-2; and E is a direct bond or S. In one embodiment, R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, (CH₂)ⱼNR²⁵C(O)²², —(CH₂)ⱼNR²⁵C(O)OR²², —(CH₂)ⱼNR²⁵C(O)NR²³R²⁴, —(CH₂)ⱼS(O)⁶, —(CH₂)ⱼNR²⁵S(O)₂R²⁶, —(CH₂)ⱼS(O)₂NR²³R²⁴, and y is independently an integer from 0 to 3.

In another embodiment, R¹⁰ is independently hydrogen, halogen or substituted or unsubstituted heteroaryl, wherein the optional heteroaryl substituents are selected from halogen, C₁-C₃ alkyl, and C₁-C₃ haloalkyl. In a further embodiment, R⁴ is selected from a group consisting of a substituted or unsubstituted alkyl, perfluoroalkyl, substituted or unsubstituted alkylaminoalkyl, substituted or unsubstituted aminocycloalkyl, substituted or unsubstituted aminoalkylenecycloalkyl, substituted or unsubstituted heterocycloalkyl, substituted or unsubstituted alkylheterocycloalkyl, substituted or unsubstituted heteroalkyl, substituted or unsubstituted heteroaryl, substituted or unsubstituted aryl, and (CH₂)ⱼNR¹⁸R¹⁹.

In yet a further embodiment, R¹⁰ is independently a substituted or unsubstituted 2H-pyrrolyl, substituted or unsubstituted 2-pyrrolinyl, substituted or unsubstituted 3-pyrrolinyl, substituted or unsubstituted pyrrolidinyl, substituted or unsubstituted dioxolanyl, substituted or unsubstituted 2-imidazolinyl, substituted or unsubstituted imidazolidinyl, substituted or unsubstituted 2-pyrazolinyl, substituted or unsubstituted pyrazolidinyl, substituted or unsubstituted piperidinyl, substituted or unsubstituted morpholinyl, substituted or unsubstituted thiomorpholinyl, substituted or unsubstituted piperazinyl, substituted or unsubstituted phenyl, substituted or unsubstituted phenoxy, substituted or unsubstituted naphthyl, substituted or unsubstituted biphenyl, substituted or unsubstituted thiophenyl, substituted or unsubstituted pyrrolyl, substituted or unsubstituted pyrazolyl, substituted or unsubstituted imidazolyl, substituted or unsubstituted pyrazinyl, substituted or unsubstituted oxazolyl, substituted or unsubstituted isoxazolyl, substituted or unsubstituted thiazolyl, substituted or unsubstituted furyl, substituted or unsubstituted thienyl, substituted or unsubstituted pyridinyl, substituted or unsubstituted O-pyridinyl, substituted or unsubstituted pyrimidyl, substituted or unsubstituted benzothiazolyl, substituted or unsubstituted purinyl, substituted or unsubstituted benzimidazolyl, substituted or unsubstituted indolyl, substituted or unsubstituted isoquinolinyl, substituted or unsubstituted quinoxalinyl, substituted or unsubstituted quinolinyl, substituted or unsubstituted benzooxazolyl, substituted or unsubstituted[1,5]naphthyridinyl, substituted or unsubstituted pyrido[3,2-d]pyrimidinyl, substituted or unsubstituted[1,7]naphthyridinyl, substituted or unsubstituted 1H-pyrrolo[2,3-b]pyridinyl, substituted or unsubstituted pyrazolo[4,3-b]pyridinyl, substituted or unsubstituted pyrrolo[2,3-b]pyridinyl, substituted or unsubstituted thieno[2,3-b]pyridinyl, substituted or unsubstituted thiazolo[5,4-b]pyridinyl, substituted or unsubstituted pyridinyl-2-one, substituted or unsubstituted imidazo[1,2-b]pyridazinyl, substituted or unsubstituted pyrazolo[1,5-a]pyrimidinyl, substituted or unsubstituted pyridazinyl-3-one, substituted or unsubstituted imidazo[2,1-b][1,3,4]thiaciazolyl, substituted or unsubstituted imidazo[2,1-b]thiazolyl, substituted or unsubstituted isothiazolyl, substituted or unsubstituted triazolyl, or substituted or unsubstituted imidazo[4,5-b]pyridinyl.

In one embodiment, R¹⁰ is substituted with 1 to 3 R²⁹ groups, wherein:

R²⁹ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR³⁰, —(CH₂)ⱼC(O)R³⁰, —(CH₂)ⱼC(O)OR³⁰, —(CH₂)ⱼNR³¹R³², —(CH₂)ⱼC(O)NR³¹R³², —(CH₂)ⱼOC(O)NR³¹R³², —(CH₂)ⱼNR³³C(O)R³⁰, —(CH₂)ⱼNR³³C(O)OR³⁰, —(CH₂)ⱼNR³³C(O)NR³¹R³², —(CH₂)ⱼS(O)ₘR³⁴, —(CH₂)ⱼNR³³S(O)₂R³⁴, —(CH₂)ⱼS(O)₂NR³¹R³², wherein each j is independently an integer from 0 to 6; and m is independently an integer from 0 to 2;

R³⁰ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;

R³¹, R³², R³³, and R³⁴ are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or

R³¹ and R³² together with the N atom to which they are attached, independently form substituted or unsubstituted heterocycloalkyl, or substituted or unsubstituted heteroaryl, or

R³⁰ and R³³ together with the N atom to which they are attached, independently form substituted or unsubstituted heterocycloalkyl, or substituted or unsubstituted heteroaryl, or

R³³ and R³¹ or R³³ and R³² together with the N atom to which they are attached, each independently form substituted or unsubstituted heterocycloalkyl, or substituted or unsubstituted heteroaryl, or

R³³ and R³⁴ together with the N atom to which they are attached, independently form substituted or unsubstituted heterocycloalkyl, or substituted or unsubstituted heteroaryl;

wherein any of the R³⁰, R³¹, R³², R³³, and R³⁴ groups are each optionally independently substituted with 1 to 3 groups, each group independently selected from hydrogen, halogen, hydroxyl, amino, aminomonoalkyl, aminodialkyl, cyano, nitro, difluoromethyl, trifluoromethyl, oxo, alkyl, —O-alkyl, and —S-alkyl.

In another embodiment, R¹⁰ is independently a substituted or unsubstituted pyrazolyl group. In yet another embodiment is a compound of Formulas (I1a), (I2a), (I3a), and (I4a) wherein R⁴ is selected from a group consisting of a substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted heterocycloalkyl, substituted or unsubstituted alkylheterocycloalkyl, substituted or unsubstituted heteroalkyl, substituted or unsubstituted heteroaryl, substituted or unsubstituted aryl, substituted or unsubstituted alkylaryl, and —(CH₂)ⱼNR¹⁸R¹⁹.

Presented herein are compounds having the structure of Formulas (I5), (I6), (I7) or (I8):

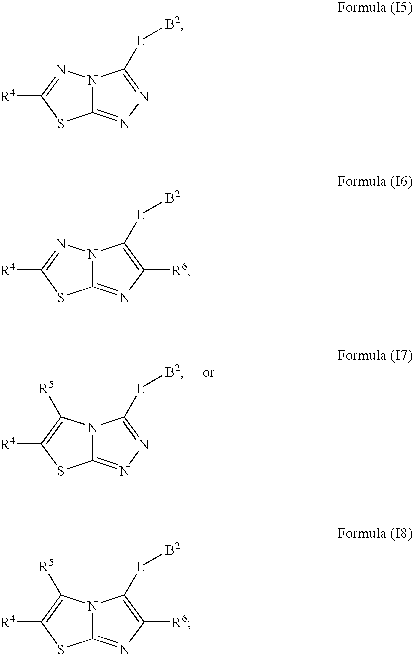

wherein:

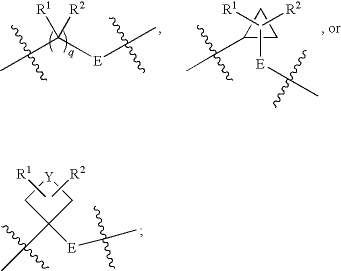

L is
E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;

R⁴, R⁵, and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, (CH₂)ⱼNR¹⁸R¹⁹, (CH₂)C(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹;

wherein each j is independently an integer from 0 to 6; and m is independently an integer from 0 to 2;

R⁴ and R⁵ optionally form substituted or unsubstituted cycloalkyl, substituted or unsubstituted heterocycloalkyl, substituted or unsubstituted aryl, or substituted or unsubstituted heteroaryl;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼ NR¹⁵S(O)₂R¹⁶, wherein each j is independently an integer from 0 to 6, and m is independently an integer from 0 to 2;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B² is:

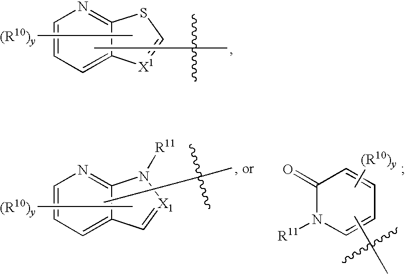

wherein:

X₁ is independently N or CR¹¹;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²¹C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

y is independently an integer from 0 to 4;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

In one embodiment is a compound having the structure of Formulas (I5a), (I5b), (I6a), (I6b), (I7a), (I7b), (I8a), or (I8b):

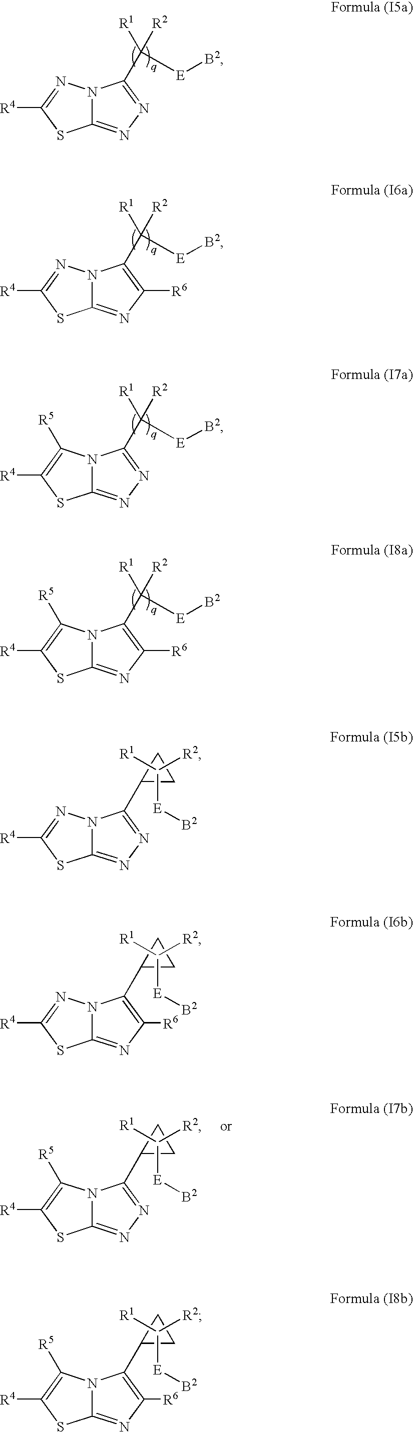

or an enantiomer, diastereomer, racemate, or pharmaceutically acceptable salt, or solvate thereof.

In another embodiment is a compound having the structure of Formulas (I9), (I10), (I11), (I12), (I13), (I14), or (I15):

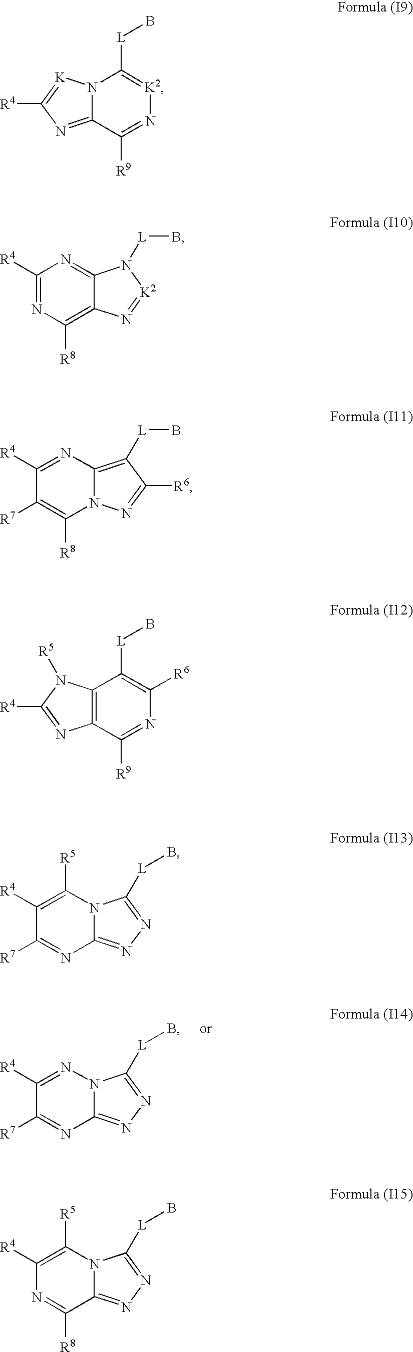

wherein:

K is N or CR⁵;

K² is N or CR⁶;

L is

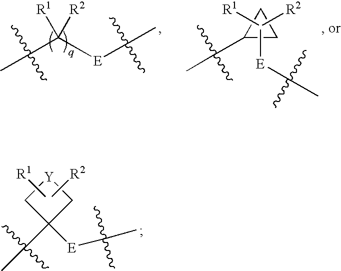

wherein:

E is independently a direct bond, O, C═O, S(O)ᵤ, or NR³;

Y is CH₂, CF₂, O, C(O)—, OC(O)—, NR³, or S(O)ᵤ;

q is an integer from 0 to 4;

u is an integer from 0 to 2;

R⁴, R⁵, R⁶, R⁷, R⁸, and R⁹ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2;

R⁴ and R⁵ optionally form substituted or unsubstituted cycloalkyl, substituted or unsubstituted heterocycloalkyl, substituted or unsubstituted aryl, or substituted or unsubstituted heteroaryl, or

R⁴ and R⁷ optionally form substituted or unsubstituted cycloalkyl, substituted or unsubstituted heterocycloalkyl, substituted or unsubstituted aryl, or substituted or unsubstituted heteroaryl, or

R⁷ and R⁸ optionally form substituted or unsubstituted cycloalkyl, substituted or unsubstituted heterocycloalkyl, substituted or unsubstituted aryl, or substituted or unsubstituted heteroaryl;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼ NR¹⁵S(O)₂R¹⁶, wherein each j is independently an integer from 0 to 6, and m is independently an integer from 0 to 2;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B is a substituted or unsubstituted heteroaryl selected from:

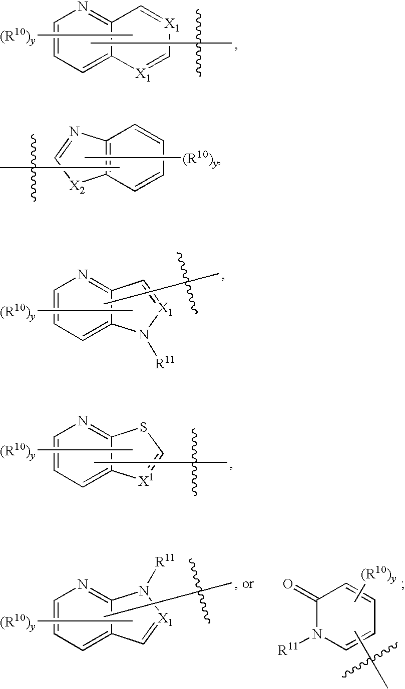

wherein:

X₁ is independently N or C; and

X₂ is N(R¹¹), O, or S;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, (CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², (CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²¹R²⁴, wherein each j is independently an integer from 0 to 6; and m is independently an integer from 0 to 2;

R¹², R¹⁷ and R²² are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;

R¹³, R¹⁴, R¹⁵, R¹⁶, R¹⁸, R¹⁹, R²⁰, R²¹, R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or

R¹³ and R¹⁴, R¹⁸ and R¹⁹, and R²³ and R²⁴ together with the N atom to which they are attached, each independently form substituted or unsubstituted heterocycloalkyl, or substituted or unsubstituted heteroaryl, or

R¹² and R¹⁵, R¹⁷ and R²⁰, and R²² and R²⁵ together with the N atom to which they are attached, each independently form substituted or unsubstituted heterocycloalkyl, or substituted or unsubstituted heteroaryl, or

R¹³ and R¹⁵ or R¹⁴ and R¹⁵, R¹⁸ and R²⁰ or R¹⁹ and R²⁰, and R²³ and R²⁵ or R²⁴ and R²⁵ together with the N atom to which they are attached, each independently form substituted or unsubstituted heterocycloalkyl, or substituted or unsubstituted heteroaryl, or

R¹⁵ and R¹⁶, R²⁰ and R²¹, and R²⁵ and R²⁶ together with the N atom to which they are attached, each independently form substituted or unsubstituted heterocycloalkyl, or substituted or unsubstituted heteroaryl;

wherein any of the R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, and R²⁴ groups are each optionally independently substituted with 1 to 3 groups, each group independently selected from hydrogen, halogen, hydroxyl, amino, aminomonoalkyl, aminodialkyl, cyano, morpholine, nitro, difluoromethyl, trifluoromethyl, oxo, alkyl, —O-alkyl, and —S-alkyl;

with the proviso that when the core structure of the compound having a structure of Formula (I14) is [1,2,4]triazolo-[4,3-b][1,2,4]triazine, then R¹⁰ is not hydrogen, halogen, nitro, cyano, hydroxyl, substituted or unsubstituted alkyl, perfluoroalkyl, —(CH₂)ⱼOR²², —(CH₂)ⱼC(O)R²², —(CH₂)ⱼC(O)OR²², —(CH₂)ⱼNR²³R²⁴, —(CH₂)ⱼS(O)ₘR²⁶ (CH₂)ⱼC(O)NR²³R²⁴, —(CH₂)ⱼS(O)₂NR²³R²⁴; or when the core structure of the compound having a structure of Formula (I13) is [1,2,4]triazolo[4,3-a]pyrimidine then R¹⁰ is not H;

or an enantiomer, diastereomer, racemate, or pharmaceutically acceptable salt, or solvate thereof.

In a further embodiment, is a compound having the structure of Formulas (I9a), (I9b), (I10a), (I10b), (I11a), (I11b), (I12a), (I12b), (I13a), (I13b), (I14a), (I14b), (I15a) or (I15b):

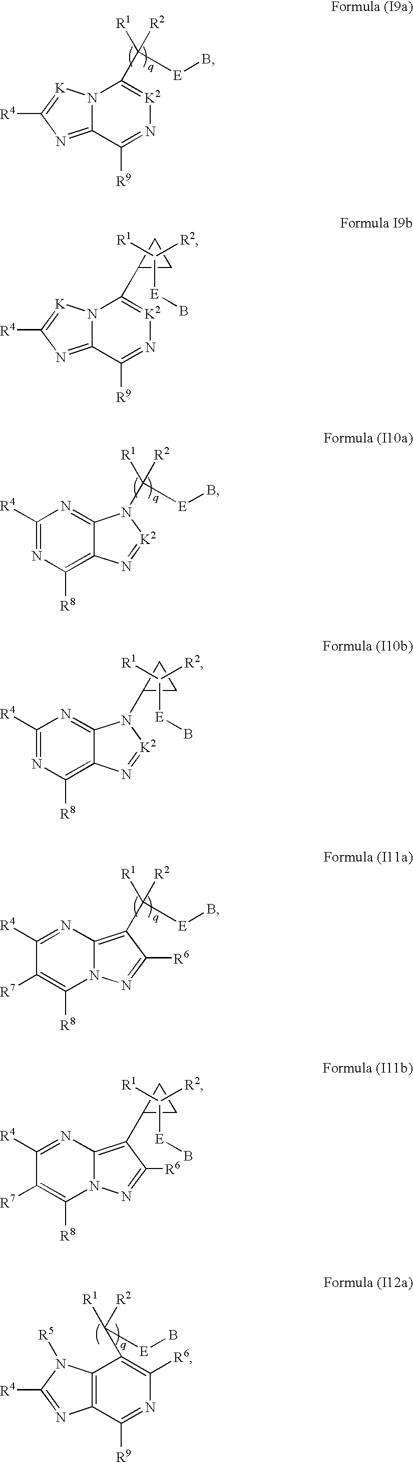

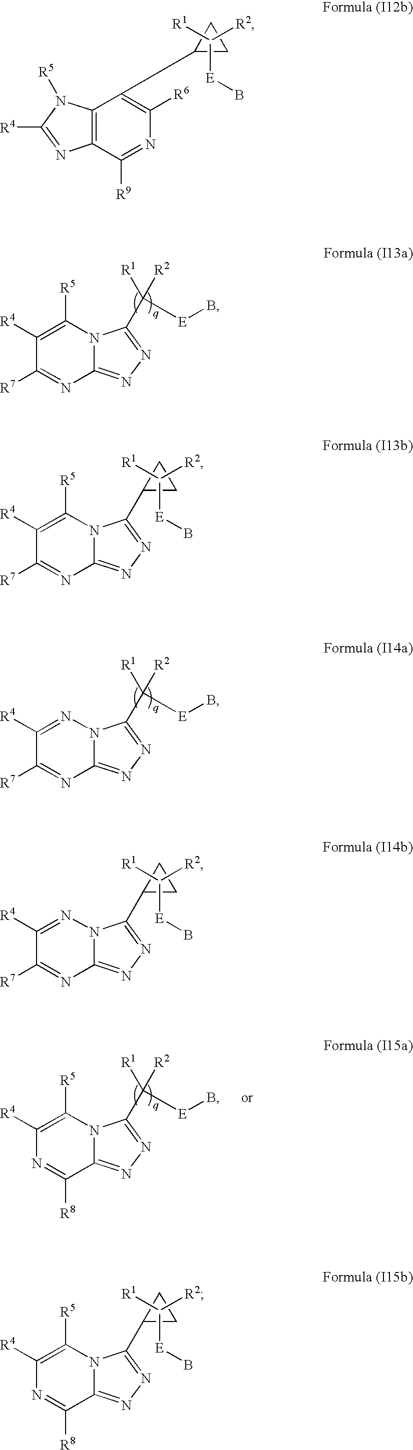

or an enantiomer, diastereomer, racemate, or pharmaceutically acceptable salt, or solvate thereof

In one embodiment, B is

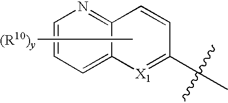

and R¹⁰ is independently a substituted or unsubstituted pyrazolyl.

In one aspect is a pharmaceutical composition comprising a compound described herein and a pharmaceutically acceptable carrier, excipient, binder or diluent.

In one aspect is a method of modulating the activity of a protein tyrosine kinase comprising contacting the protein tyrosine kinase with a compound described herein.

In one embodiment is a method of modulating the activity of a protein kinase comprising contacting the protein kinase with a compound described herein, wherein the protein kinase is Abelson tyrosine kinase, Ron receptor tyrosine kinase, Met receptor tyrosine kinase, Fms-like tyrosine kinase-3, or p21-activated kinase-4.

In another embodiment, the protein tyrosine kinase is Met tyrosine kinase.

In another aspect is a method for treating cancer in a human patient in need of such treatment, the method comprising administering to the patient a therapeutically effective amount of a compound described herein.

In one embodiment the cancer is bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

In one aspect is a method for treating cancer in a subject in need of treatment, comprising administering to a subject in need of treatment a therapeutically effective amount of a compound described herein in combination with ionizing radiation and/or one or more chemotherapeutic agents.

In one embodiment is a method for treating cancer wherein the compound described herein is administered simultaneously with ionizing radiation and/or one or more chemotherapeutic agents.

In another embodiment is a method for treating cancer wherein the compound described herein is administered sequentially with ionizing radiation and/or one or more chemotherapeutic agents.

In one aspect is the use of a compound described herein for the formulation of a medicament for the treatment of a kinase mediated disease or condition.

In another aspect is an article of manufacture, comprising packaging material, a compound described herein which is effective for modulating kinase activity, or for the treatment, prevention or amelioration of one or more symptoms of kinase mediated disease or condition, within the packaging material, and a label that indicates that the compound or composition, or pharmaceutically acceptable salt, pharmaceutically acceptable N-oxide, pharmaceutically active metabolite, pharmaceutically acceptable prodrug, or pharmaceutically acceptable solvate thereof, is used for modulating kinase activity, or for treatment, prevention or amelioration of one or more symptoms of kinase mediated disease or condition.

In other aspects, the present disclosure provides methods for modulating the activity of protein kinases; methods for treating cancer and pharmaceutical compositions using a compound described herein.

### DETAILED DESCRIPTION OF THE INVENTION

### Definitions

Abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or cyclic hydrocarbon radical, or combinations thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, N-propyl, isopropyl, N-butyl, sec-butyl, tert-butyl, isobutyl, cyclobutyl, pentyl, cyclopentyl, hexyl, cyclohexyl, (cyclohexyl)methyl, cyclopropylmethyl, homologs and isomers of, for example, N-pentyl, N-hexyl, N-heptyl, N-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C≡CCH₂—, —CH₂CH₂CH(CH₂CH₂CH₃)CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

As used herein, the terms “alkyl” and “alkylene” are interchangeable depending on the placement of the “alkyl” or “alkylene” group within the molecule.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. As used herein, the terms “heteroalkyl” and “heteroalkylene” are interchangeable depending on the placement of the “heteroalkyl” or “heteroalkylene” group within the molecule.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, when the heteroatom is nitrogen, it can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively. As used herein, the terms “cycloalkyl” and “cycloalkylene” are interchangeable depending on the placement of the “cycloalkyl” or “cycloalkylene” group within the molecule. As used herein, the terms “heterocycloalkyl” and “heterocycloalkylene” are interchangeable depending on the placement of the “heterocycloalkyl” or “heterocycloalkylene” group within the molecule.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. As used herein, the terms “haloalkyl” and “haloalkylene” are interchangeable depending on the placement of the “haloalkyl” or “haloalkylene” group within the molecule.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. For example, pyridine N-oxide moieties are included within the description of “heteroaryl.” A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively. As used herein, the terms “aryl” and “arylene” are interchangeable depending on the placement of the “aryl” and “arylene” group within the molecule. As used herein, the terms “heteroaryl” and “heteroarylene” are interchangeable depending on the placement of the “heteroaryl” and “heteroarylene” group within the molecule.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g., “3 to 7 membered”), the term “member” referrers to a carbon or heteroatom.

The term “oxo” as used herein means an oxygen that is double bonded to a carbon atom.

Each of the above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Preferred substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR″—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′- or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from at least the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

As presented herein L can be a linker having the following structures:

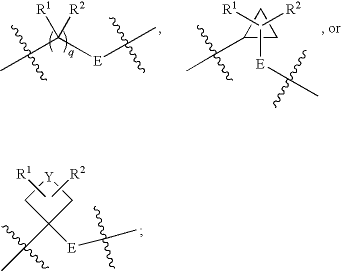

wherein E can form a spiro moiety with the cycloalkyl to which it is attached. For example, E can form a cyclopentyl ring attached to, for example, a cyclobutyl ring such that the spiro linker is a spiro[3.4]octane. Other spiro moieties known in the art are also contemplated herein.

The compounds of the present invention may exist as salts. The present invention includes such salts. Non-limiting examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogen-phosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science, 66:1-19 (1977)). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol [symbol] denotes the point of attachment of a moiety to the remainder of the molecule.

### Heterocyclic Compounds

In one aspect, are compounds having the structure of Formula (I):

A-L-B; Formula (I)

or an enantiomer, diastereomer, racemate or pharmaceutically acceptable salt or solvate thereof, wherein:

### L is

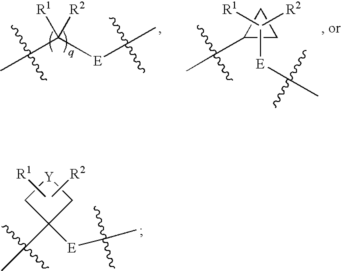

E is independently a direct bond, O, C═O, S(O)ᵤ, or NR³;

### Y is CH₂, CF₂, O, C(O)—, OC(O)—, NR³, or S(O)ᵤ;

q is an integer from 0 to 4;
u is an integer from 0 to 2;
A is independently substituted or unsubstituted heteroaryl selected from:

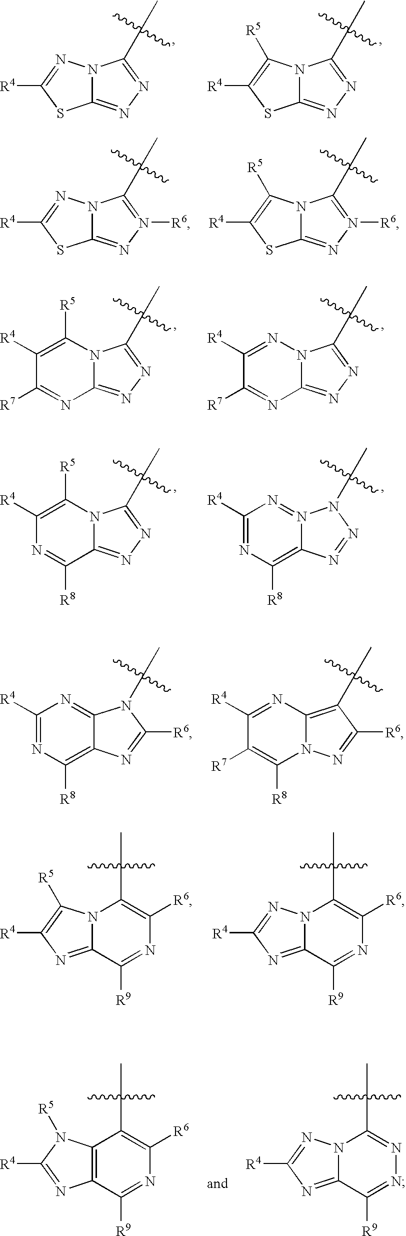

B is independently substituted or unsubstituted heteroaryl selected from:

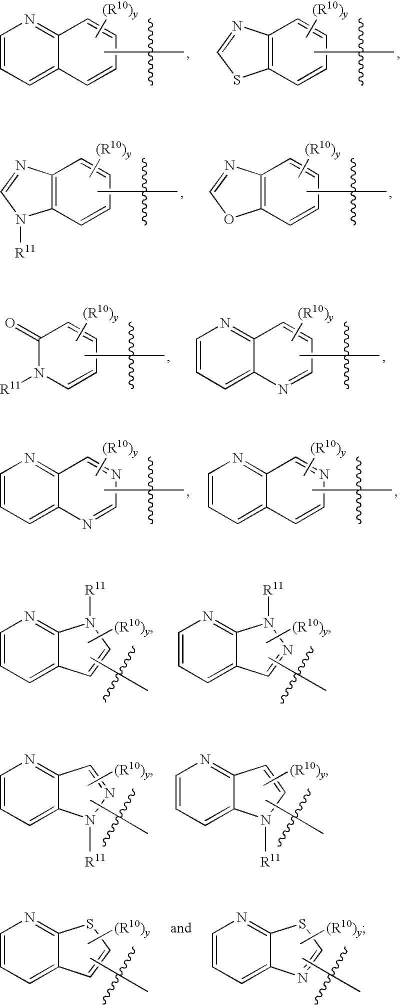

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)C(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼ NR¹⁵S(O)₂R¹⁶, wherein each j is independently an integer from 0 to 6, and m is independently an integer from 0 to 2;
R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;
R⁴, R⁵, R⁶, R⁷, R⁸, and R⁹ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, (CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2; wherein:
R⁴ and R⁵ optionally form substituted or unsubstituted cycloalkyl, substituted or unsubstituted heterocycloalkyl, substituted or unsubstituted aryl, or substituted or unsubstituted heteroaryl, or
R⁴ and R⁷ optionally form substituted or unsubstituted cycloalkyl, substituted or unsubstituted heterocycloalkyl, substituted or unsubstituted aryl, or substituted or unsubstituted heteroaryl, or
R⁷ and R⁸ optionally form substituted or unsubstituted cycloalkyl, substituted or unsubstituted heterocycloalkyl, substituted or unsubstituted aryl, or substituted or unsubstituted heteroaryl,
R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)NR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²¹R¹⁴, wherein each j is independently an integer from 0 to 6; and m is independently an integer from 0 to 2;
y is independently an integer from 0 to 6;
R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁰C(O)NR²³R²⁴, —(CH₂)ⱼS(O)ₘR², —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;
R¹², R¹⁷ and R²² are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;
R¹³, R¹⁴, R¹⁵, R¹⁶, R¹⁸, R¹⁹, R²⁰, R²¹, R²³, R²⁴, R²⁵, and R²⁶ are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or
R¹³ and R¹⁴, R¹⁸ and R¹⁹, and R²³ and R²⁴ together with the N atom to which they are attached, each independently form substituted or unsubstituted heterocycloalkyl, or substituted or unsubstituted heteroaryl, or
R¹² and R¹⁵, R¹⁷ and R²⁰, and R²² and R²⁵ together with the N atom to which they are attached, each independently form substituted or unsubstituted heterocycloalkyl, or substituted or unsubstituted heteroaryl, or

R¹³ and R¹⁵ or R¹⁴ and R¹⁵, R¹⁸ and R²⁰ or R¹⁹ and R²⁰, and R²³ and R²⁵ or R²⁴ and R²⁵ together with the N atom to which they are attached, each independently form substituted or unsubstituted heterocycloalkyl, or substituted or unsubstituted heteroaryl, or

R¹⁵ and R¹⁶, R²⁰ and R²¹, and R²⁵ and R²⁶ together with the N atom to which they are attached, each independently form substituted or unsubstituted heterocycloalkyl, or substituted or unsubstituted heteroaryl,

wherein any of the R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, and R²⁴ groups are each optionally independently substituted with 1 to 3 groups, each group independently selected from hydrogen, halogen, hydroxyl, amino, aminomonoalkyl, aminodialkyl, cyano, nitro, difluoromethyl, trifluoromethyl, oxo, alkyl, —O-alkyl, and —S-alkyl; and
with the proviso that when A is

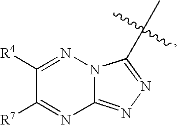

then R¹⁰ is not hydrogen, halogen, nitro, cyano, hydroxyl, substituted or unsubstituted alkyl, perfluoroalkyl, —(CH₂)ⱼOR²², —(CH₂)ⱼC(O)R²², —(CH₂)ⱼC(O)OR²², —(CH₂)ⱼNR²³R²⁴, —(CH₂)ⱼS(O)ₘR²⁶ (CH₂)ⱼC(C)NR²³R²⁴, —(CH₂)ⱼS(O)₂NR²³R²⁴.

In one embodiment, the disclosure provides compounds of Formula (I), wherein:

R⁴, R⁵, R⁶, R⁷, R⁸, and R⁹ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, and substituted or unsubstituted heteroarylalkyl.

In another embodiment, the present disclosure provides compounds of Formula (I), wherein:

R⁴, R⁵, R⁶, R⁷, R⁸, and R⁹ are each independently substituted with 1 to 3 R²⁷ groups, wherein:
R²⁷ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹⁷, —(CH₂)ⱼC(O)R¹⁷, —(CH₂)ⱼC(O)OR¹⁷ (CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2.

In a further embodiment, are compounds having the structure of Formula (I), wherein:

R¹⁰ is independently substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted Or unsubstituted —O-aryl, substituted or unsubstituted arylalkyl, substituted or unsubstituted heteroaryl, substituted or unsubstituted —O-heteroaryl, or substituted or unsubstituted heteroarylalkyl;
R¹¹ is independently substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl.

In yet another embodiment, the disclosure provides compounds of Formula (I), wherein:

R¹⁰ and R¹¹ are each independently substituted with 1 to 3 R²⁸ groups, wherein:
R²⁸ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁰C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2.

In one embodiment, the present disclosure provides compounds of Formula (I), wherein R¹⁰ is a substituted or unsubstituted alkyl, substituted or unsubstituted aryl, or substituted or unsubstituted heteroaryl. In another embodiment, R¹⁰ is a substituted or unsubstituted heteroaryl. In a further embodiment, R¹⁰ is a substituted or unsubstituted heteroaryl having at least one N, O, or S atom. In yet a further embodiment, R¹⁰ is a substituted or unsubstituted pyrazole. In one embodiment, R¹⁰ is a substituted pyrazole, substituted with C₁-C₆ alkyl, hydroxy, halogen, cyano, or SH. In another embodiment, the pyrazole is substituted with methyl, ethyl, n-propyl, isopropyl, n-butyl, iso-butyl, t-butyl, pentyl, or hexyl. In a further embodiment, the pyrazole is substituted with methyl. In another embodiment, R¹⁰ is halogen. In a further embodiment, R¹⁰ is selected from fluorine, bromine, and chlorine and y is an integer from 1-6. In another embodiment, y is 2 or 3. In a further embodiment are compounds having the structure of Formula (I), wherein:

### L is

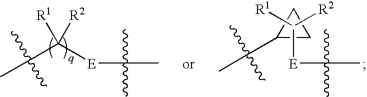

E is independently a direct bond, O, C═O, S, or NH;
q is 0 or 1;

### A is:

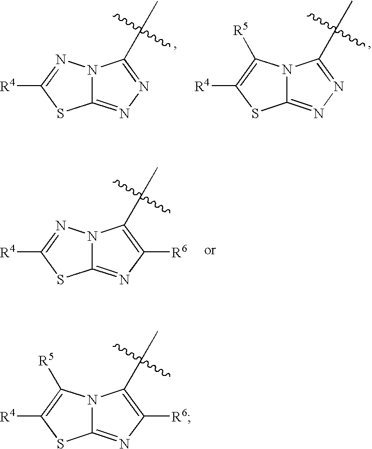

and

### B is:

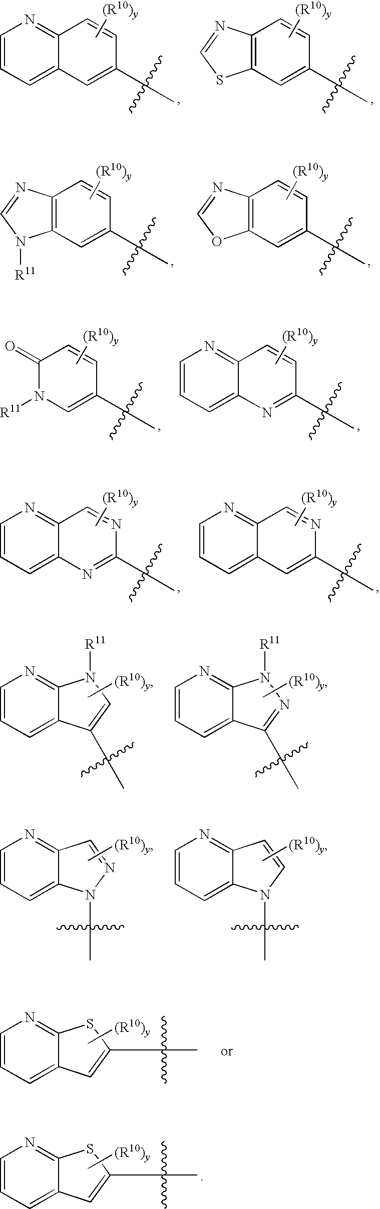

In another embodiment, the disclosure provides compounds of Formula (I), wherein:

### L is

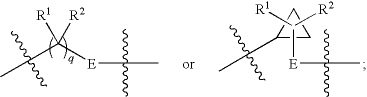

E is independently a direct bond, O, C═O, S, or NH;
q is 0 or 1;

### A is:

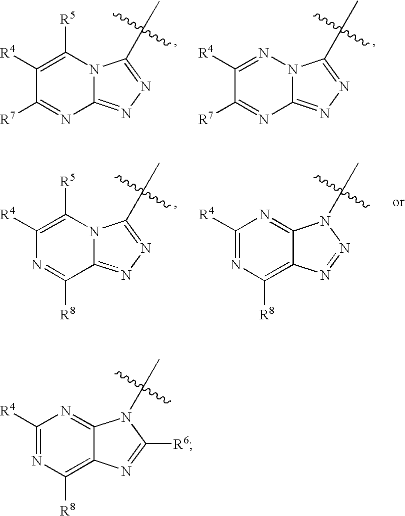

and

### B is:

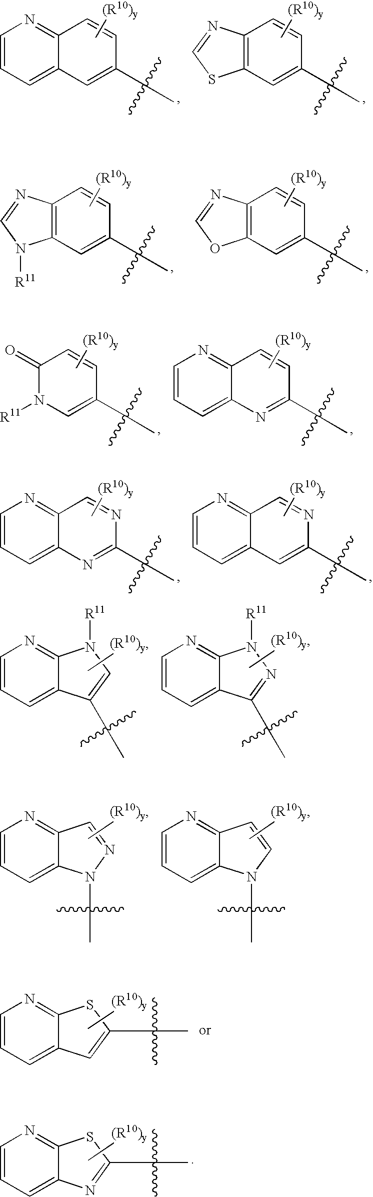

Provided herein are compounds having the structure of Formula (I), wherein:

### L is

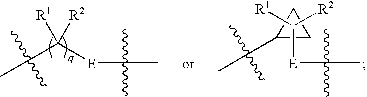

E is independently a direct bond, O, C═O, S, or NH;
q is 0 or 1;

### A is:

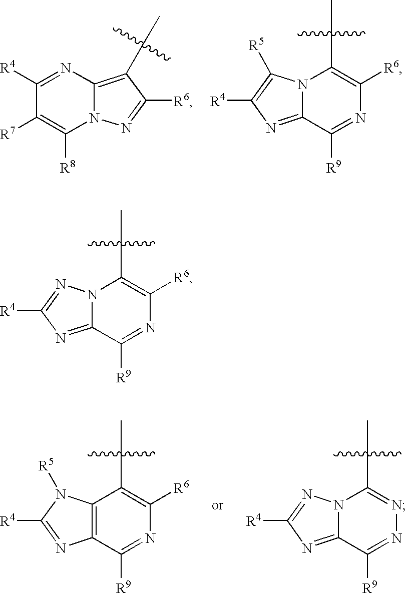

### B is:

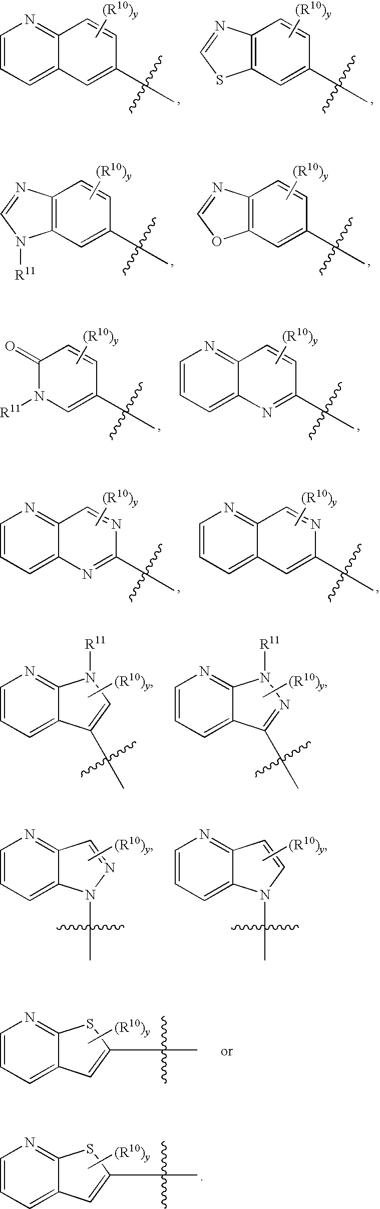

In one embodiment, the disclosure provides compounds of Formula (I), having formulae:

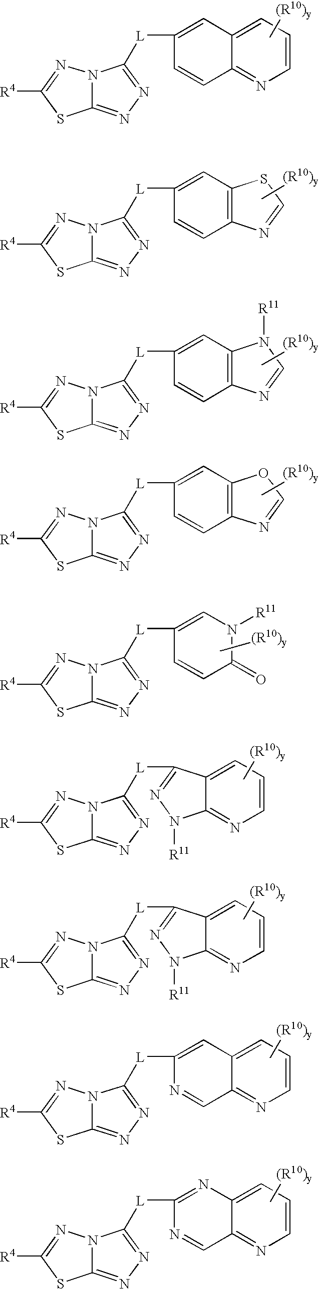

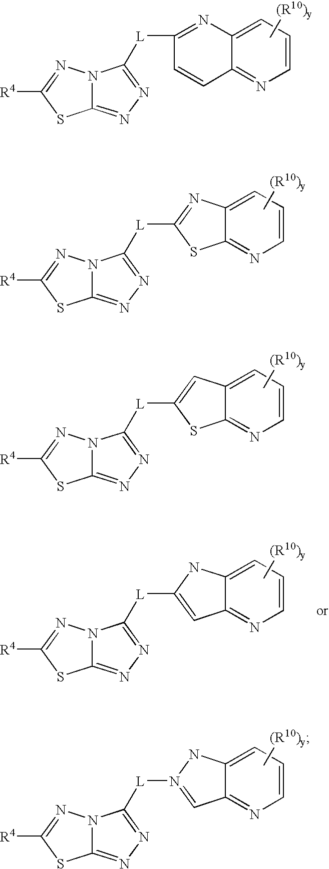

wherein:

### L is

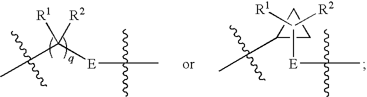

E is independently a direct bond, O, C═O, S, or NH; and
q is 0 or 1.

In another embodiment, the disclosure provides compounds of Formula (I), having formulae:

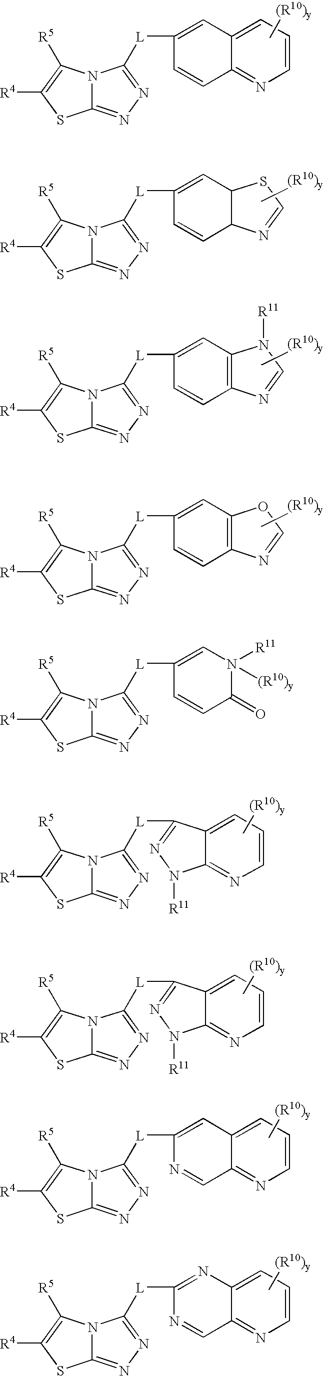

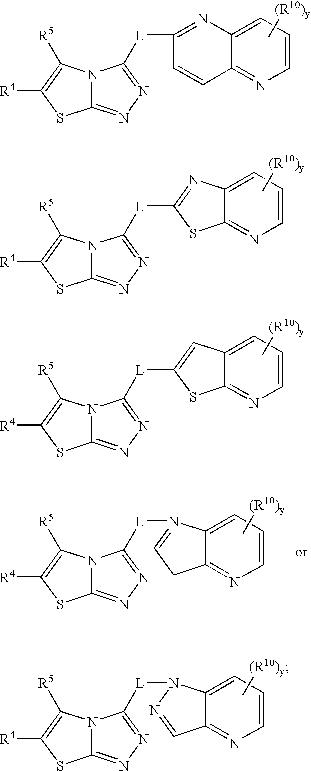

wherein:

### L is

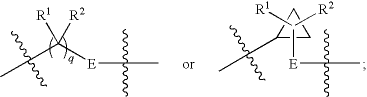

E is independently a direct bond, O, C═O, S, or NH; and
q is 0 or 1.

In yet another embodiment, the disclosure provides compounds of Formula (I), having formulae:

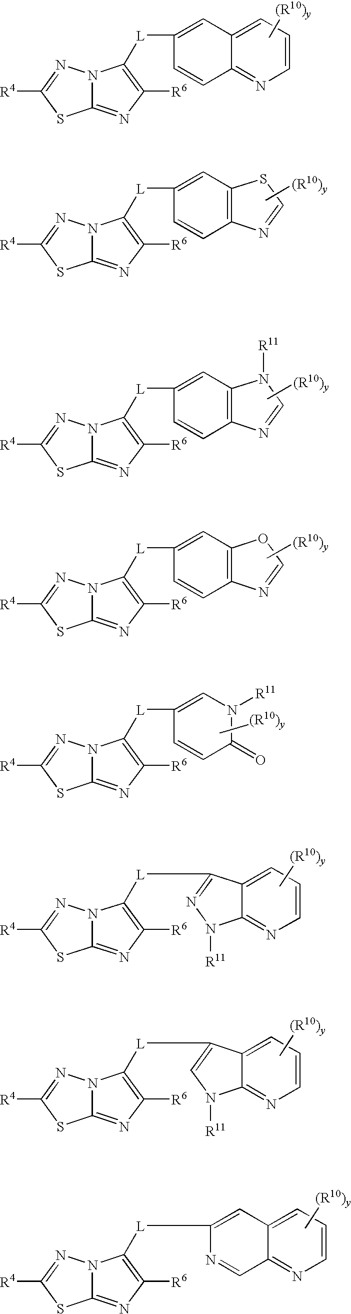

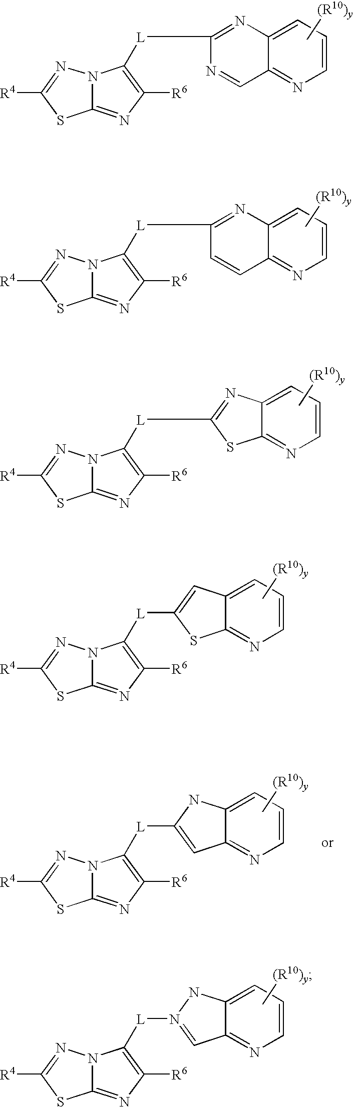

wherein:

### L is

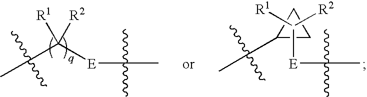

E is independently a direct bond, O, C═O, S, or NH; and
q is 0 or 1.

In some embodiments are compounds having the structure of Formula (I), having formulae:

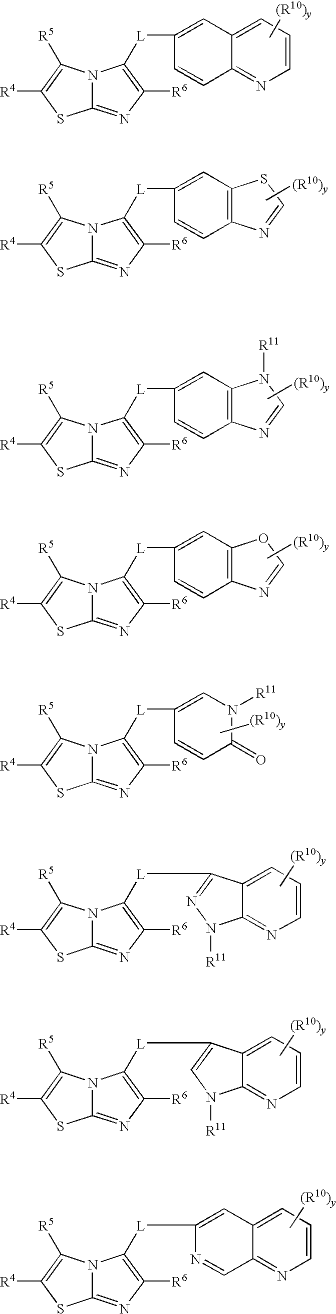

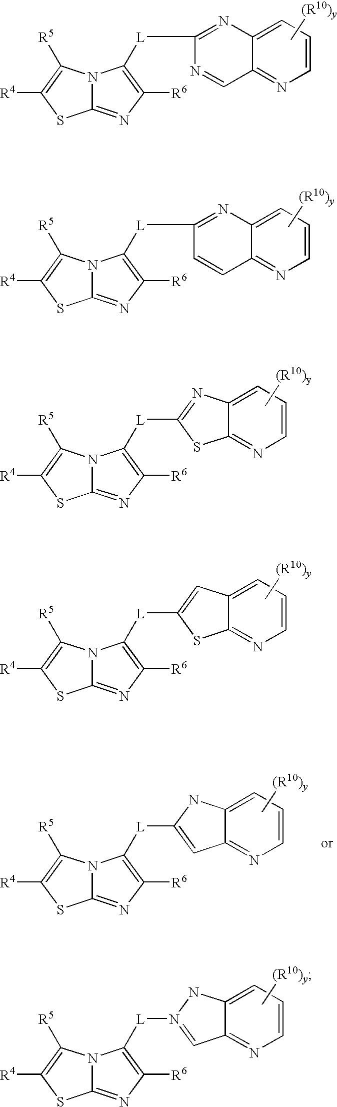

wherein:

### L is

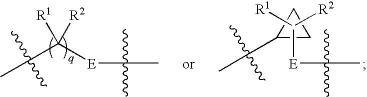

E is independently a direct bond, O, C═O, S, or NH; and
q is 0 or 1.

In one embodiment, the disclosure provides compounds of Formula (I), having formulae:

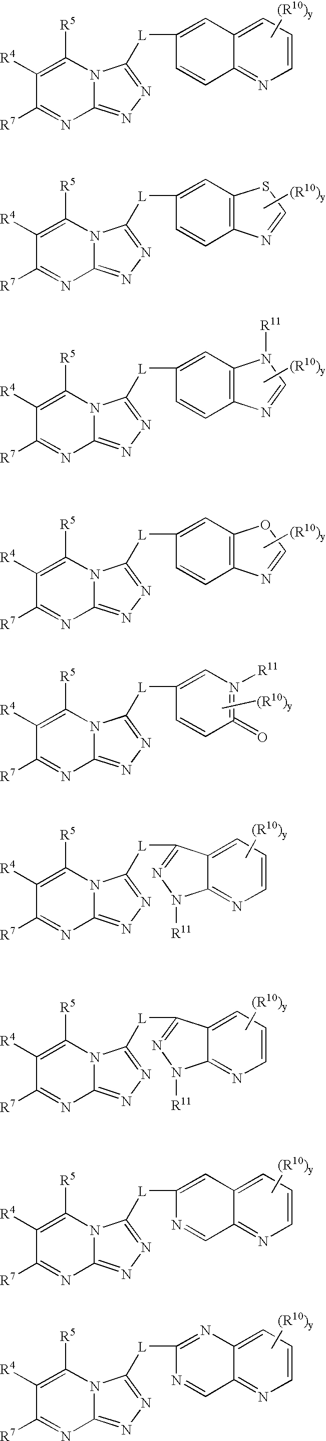

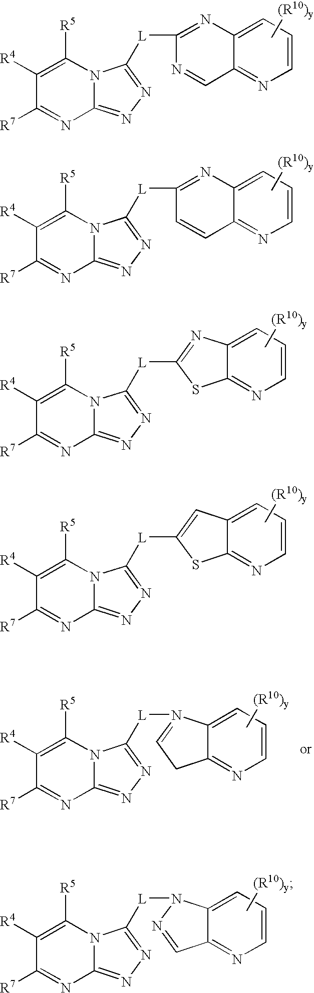

wherein:

### L is

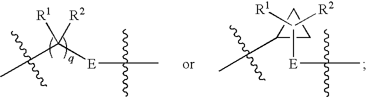

E is independently a direct bond, O, C═O, S, or NH; and
q is 0 or 1.

In some further embodiments are compounds of Formula (I), having formulae:

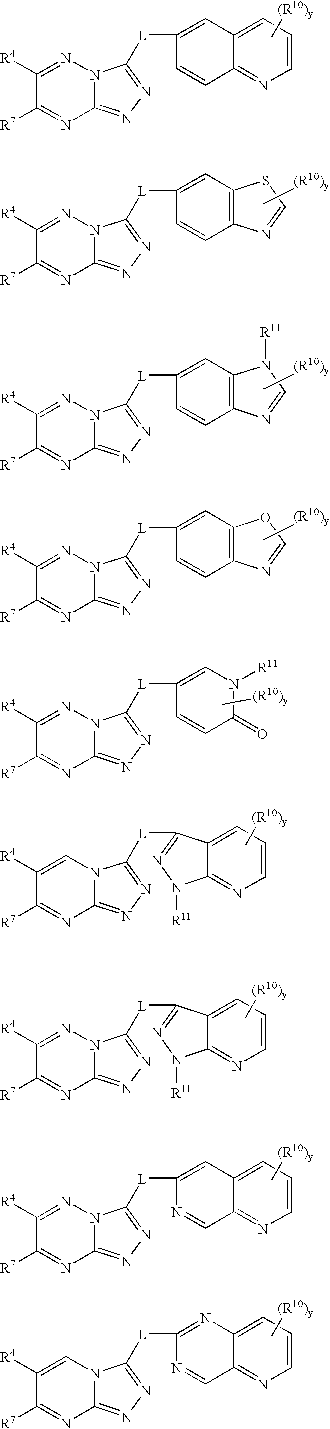

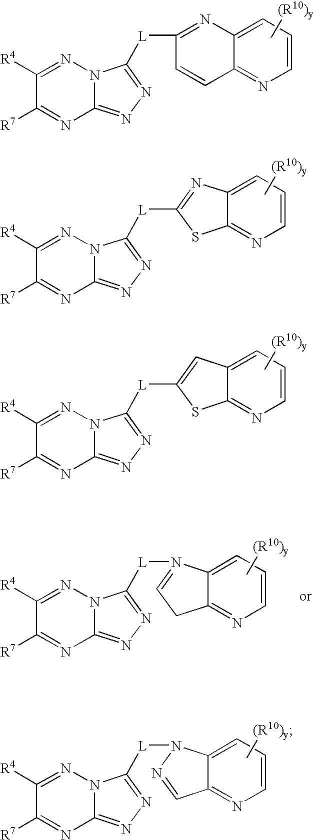

wherein:

### L is

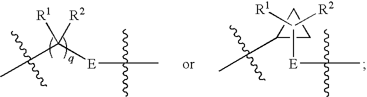

E is independently a direct bond, O, C═O, S, or NH; and
q is 0 or 1.

In one embodiment, the disclosure provides compounds of Formula (I), having formulae:

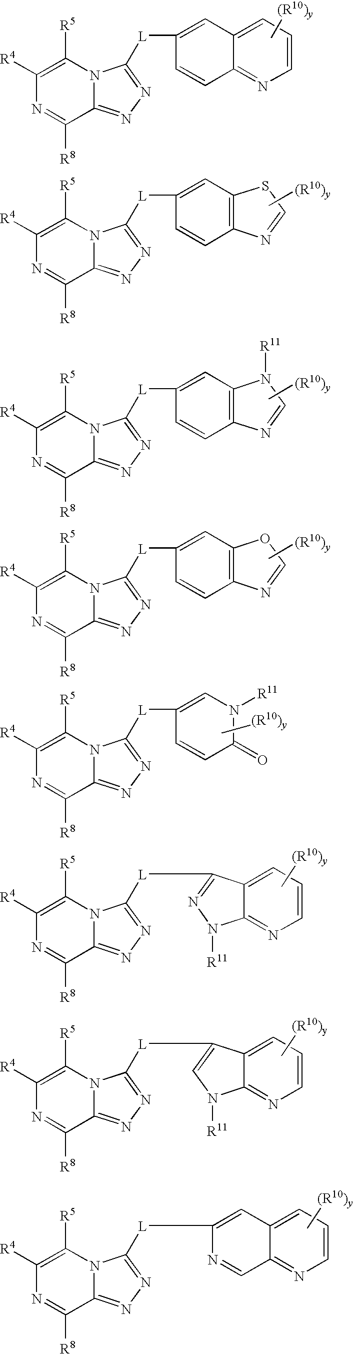

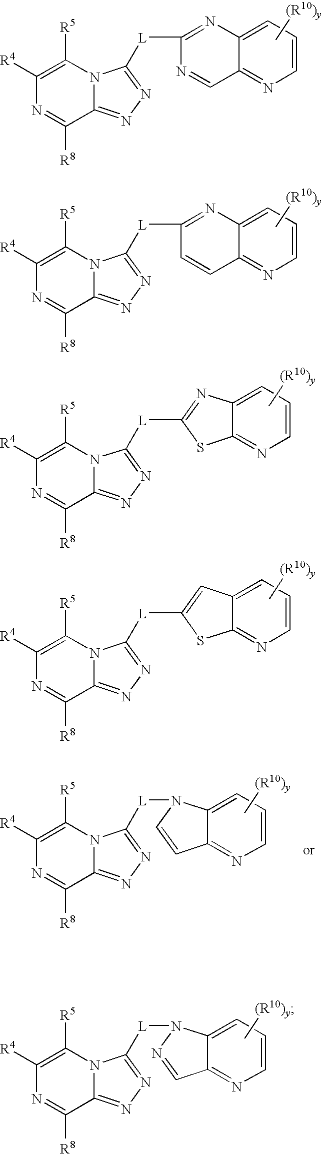

wherein:

### L is

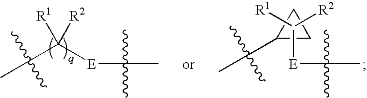

E is independently a direct bond, O, C═O, S, or NH; and
q is 0 or 1.

In another embodiment are compounds of Formula (I), having formulae:

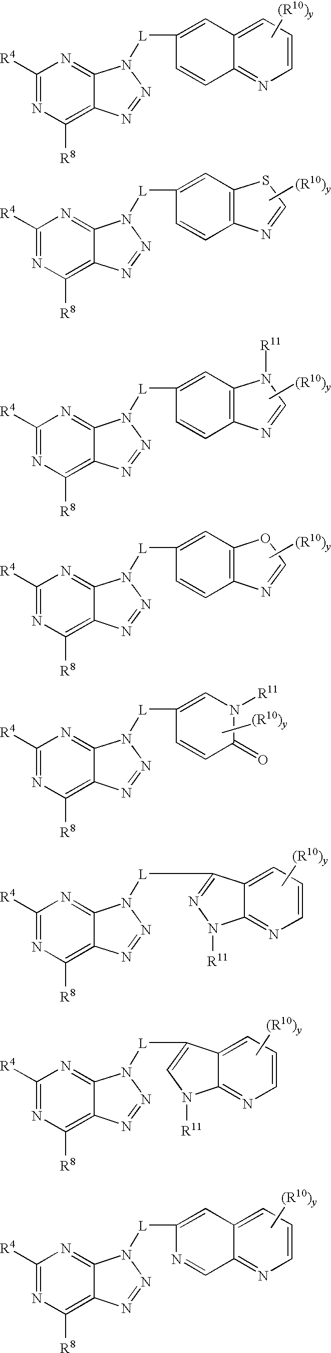

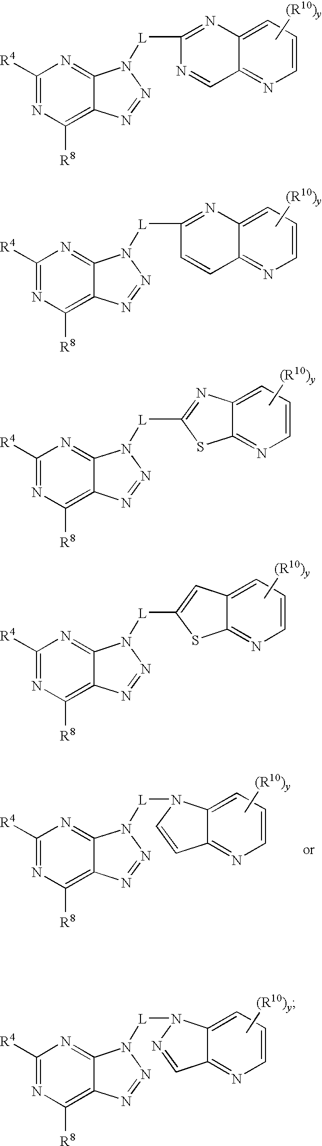

wherein:

### L is

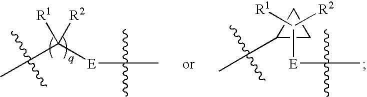

E is independently a direct bond, O, C═O, S; or NH; and
q is 0 or 1.

In a further embodiment are provided compounds of Formula (I), having formulae:

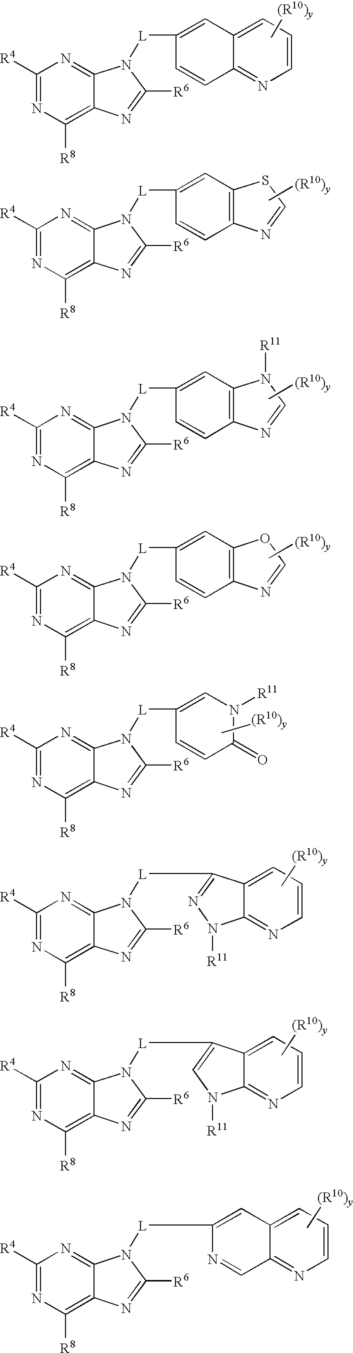

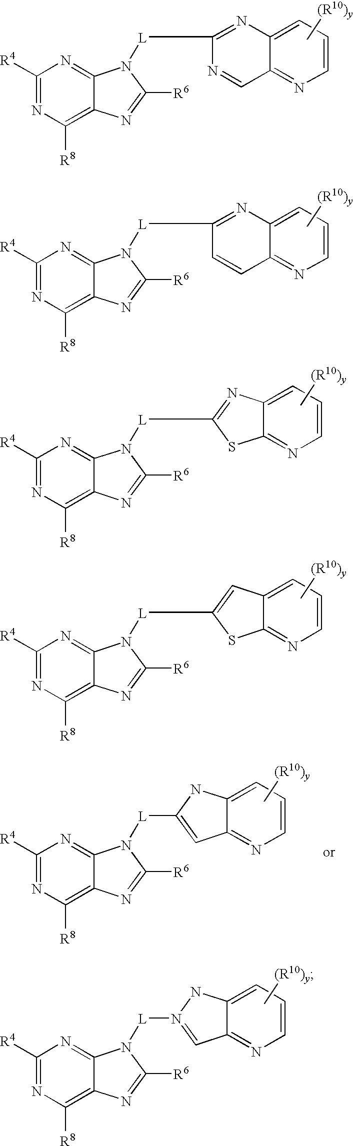

wherein:

### L is

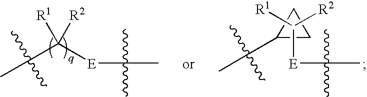

E is independently a direct bond, O, C═O, S, or NH; and
q is 0 or 1.

In some embodiments are provided compounds of Formula (I), having formulae:

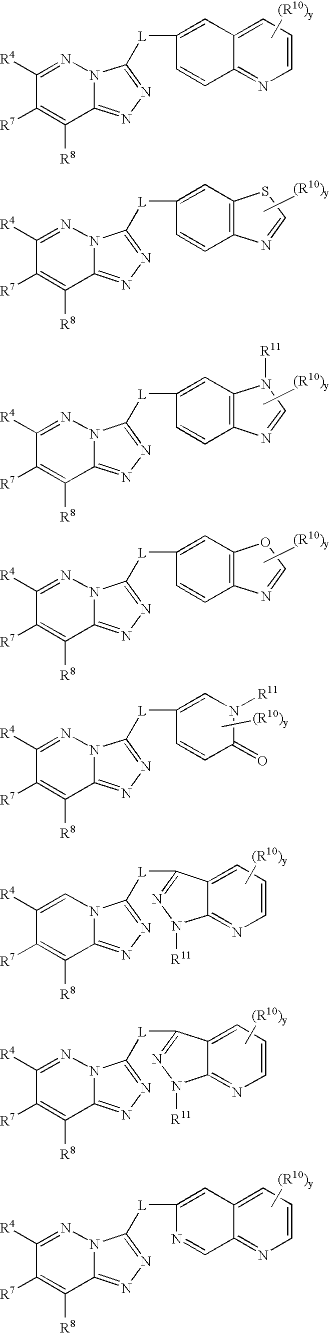

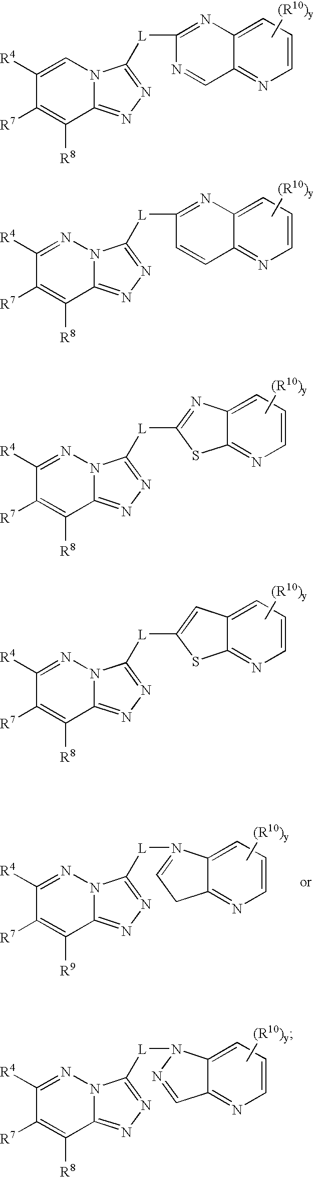

wherein:

### L is

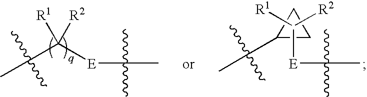

E is independently a direct bond, O, C═O, S, or NH; and
q is 0 or 1.

In yet a further embodiment the disclosure provides compounds of Formula (I), having formulae:

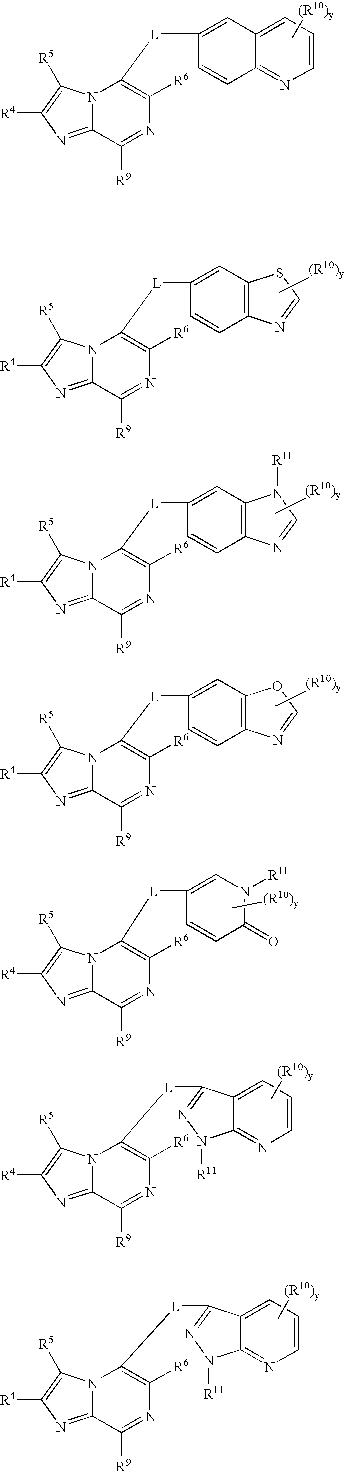

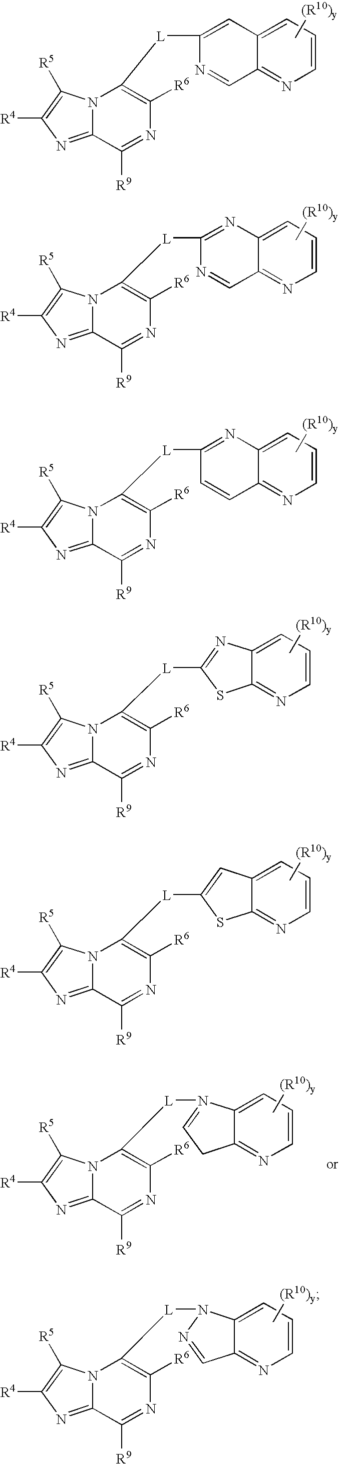

wherein:

### L is

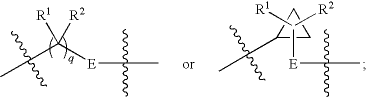

E is independently a direct bond, O, C═O, S, or NH; and
q is 0 or 1.

In other embodiments, the disclosure provides compounds of Formula (I), having formulae:

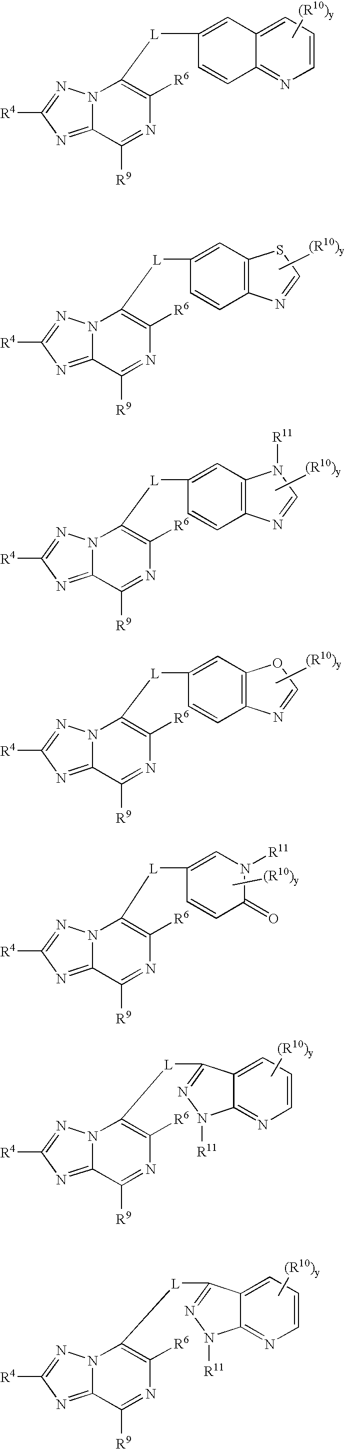

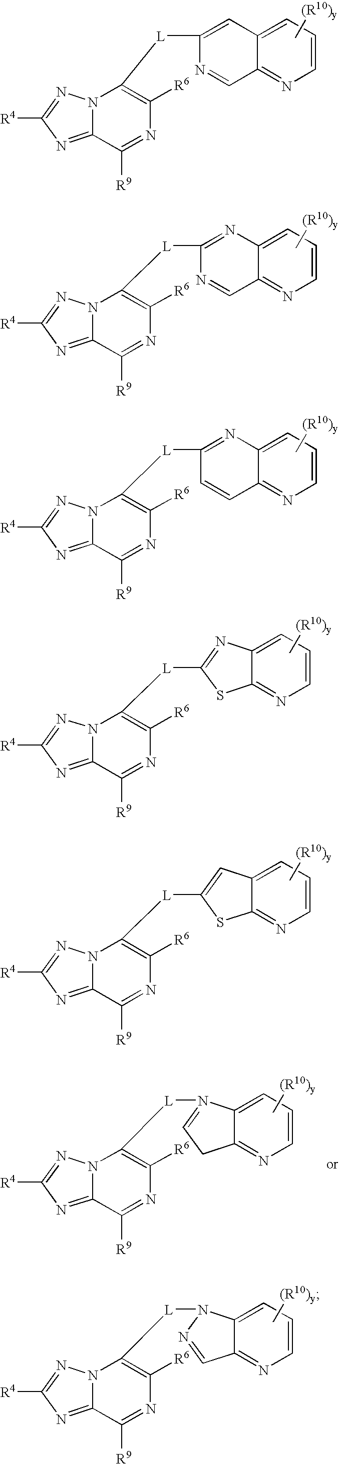

wherein:

### L is

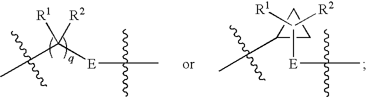

E is independently a direct bond, O, C═O, S, or NH; and
q is 0 or 1.

In yet other embodiments are provided compounds of Formula (I), having formulae:

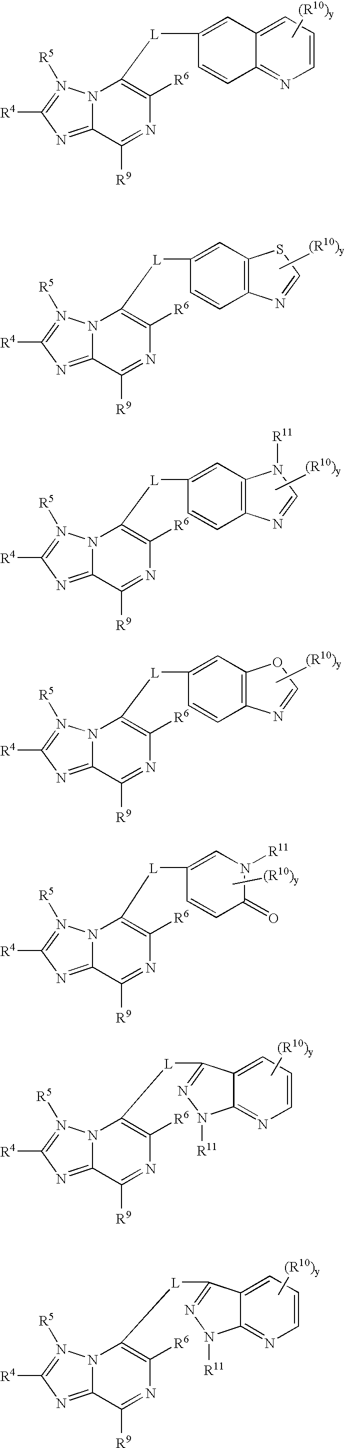

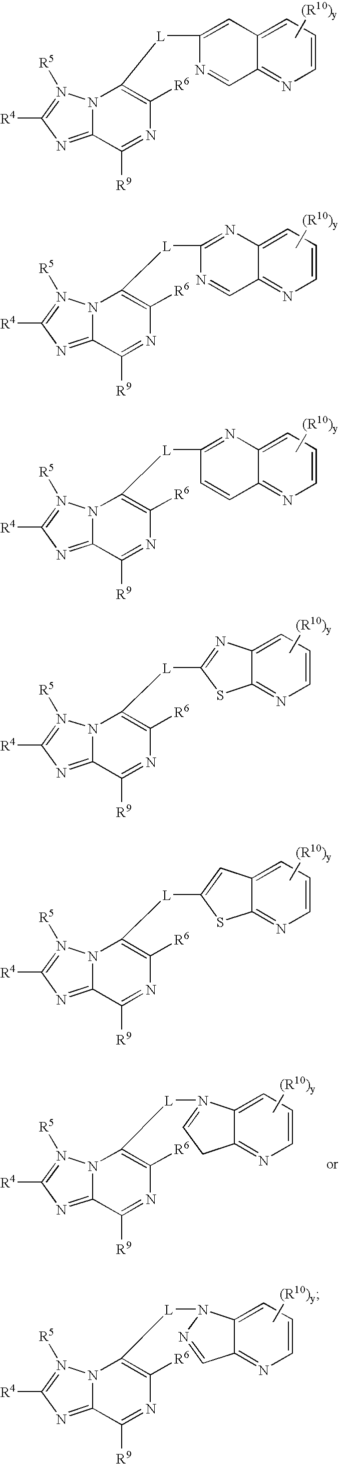

wherein:

### L is

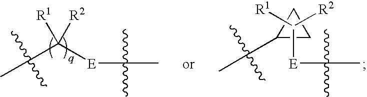

E is independently a direct bond, O, C═O, S, or NH; and
q is 0 or 1.

In some embodiments are provided compounds of Formula (I), having formulae:

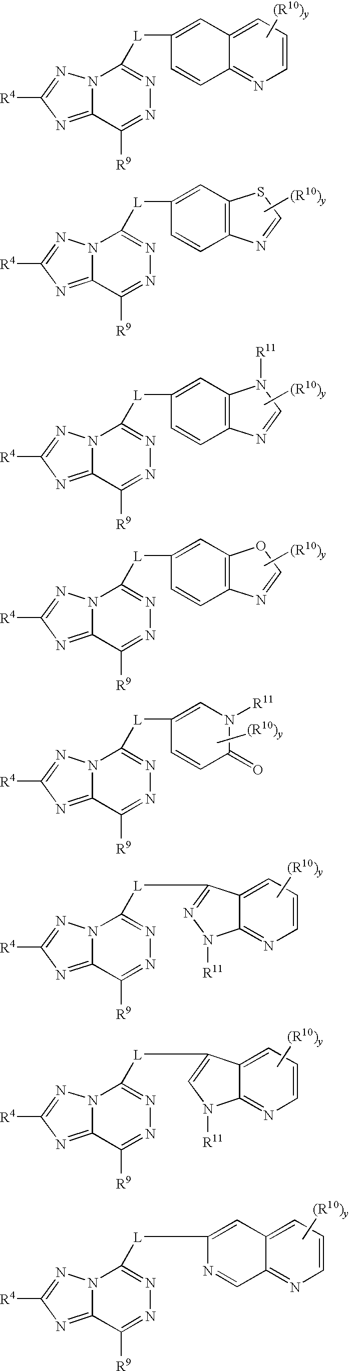

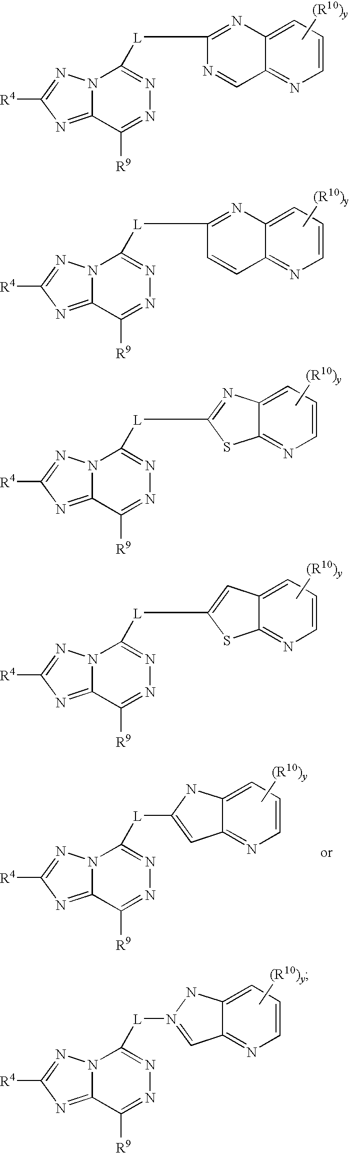

wherein:

### L is

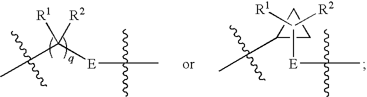

E is independently a direct bond, O, C═O, S, or NH; and
q is 0 or 1.

In one aspect is a compound having the formula:

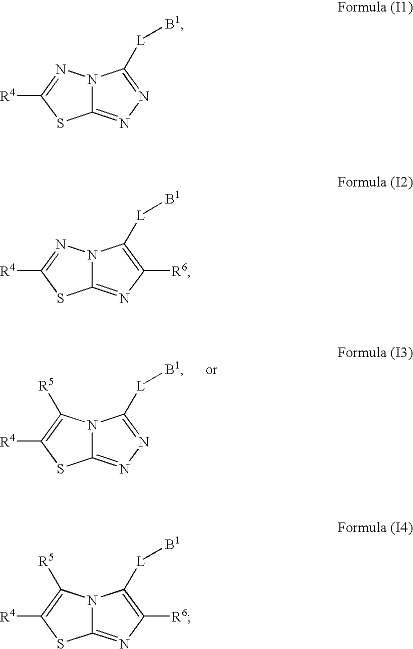

wherein:

L is

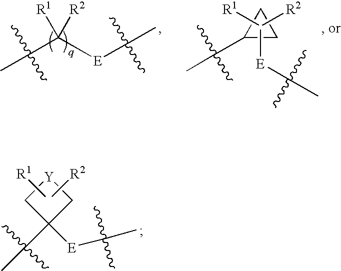

E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;

R⁴ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷—(CH₂)ⱼNR²⁰C(O)OR¹⁷, —(CH₂)ⱼNR²⁰C(O)NR¹⁸R¹⁹—(CH₂)ⱼS(O)ₘR²¹, —(CH₂)ⱼNR²⁰S(O)₂R²¹;

R⁵ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹;

R⁴ and R⁵ optionally form substituted or unsubstituted cycloalkyl, substituted or unsubstituted heterocycloalyl, substituted or unsubstituted aryl, or substituted or unsubstituted heteroaryl,

R⁶ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, (CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, (CH₂)ⱼNR²⁰C(O)NR¹⁸R¹⁹, (CH₂)ⱼS(O)ₘR²¹, (CH₂)ⱼNR²⁰S(O)₂R²¹, —(CH₂)ⱼS(O)₂NR¹⁸R¹⁹;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B¹ is

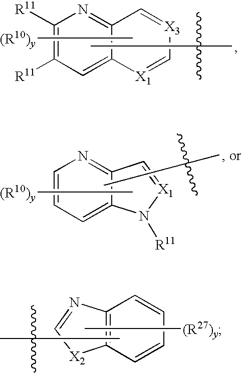

wherein:
X₁ is independently N or CR¹¹;
X₂ is NR¹¹, O, or S; and
X₃ is CR¹⁰ or N;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², (CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, (CH₂)ⱼS(O)ₘR²⁶, —(CH₂)ⱼNR²⁵S(O)₂R²⁶, —(CH₂)ⱼS(O)₂NR²³R²⁴; wherein each j is independently an integer from 0 to 6, and m is independently an integer from 0 to 2;

with the proviso that when R¹¹ is independently a direct bond, then R¹⁰ or R²⁷ cannot all be H; R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

In one embodiment is a compound having the formula:

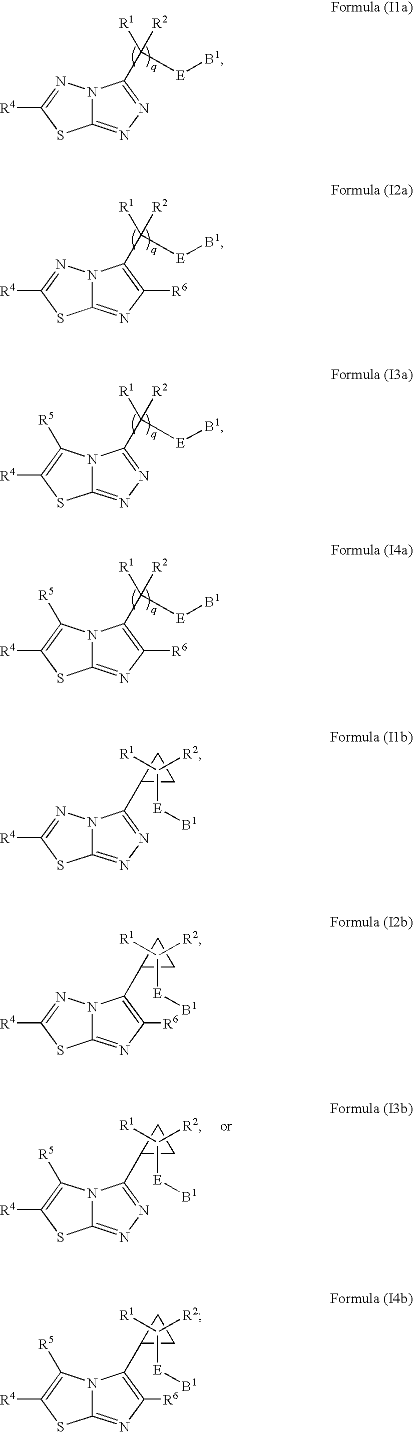

or an enantiomer, diastereomer, racemate, or pharmaceutically acceptable salt, or solvate thereof.

In one embodiment B is

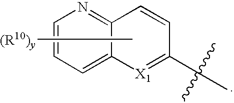

In another embodiment, X₁ is CH. In a further embodiment, R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)^{j}OC(O)NR²³R²⁴, —(CH₂)ⱼNR²⁵C(O)R²², —(CH₂)ⱼNR²⁵C(O)OR²², (CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, and y is independently an integer from 0 to 3. In yet a further embodiment, R¹⁰ is independently hydrogen or halogen.

In another embodiment, R⁴ is selected from a group consisting of a substituted or unsubstituted alkyl, perfluoroalkyl, substituted or unsubstituted alkylaminoalkyl, substituted or unsubstituted aminocycloalkyl, substituted or unsubstituted heterocycloalkyl, substituted or unsubstituted alkylheterocycloalkyl, substituted or unsubstituted heteroalkyl, substituted or unsubstituted aminoalkylenecycloalkyl, substituted or unsubstituted heteroaryl, substituted or unsubstituted aryl, and (CH₂)ⱼNR¹⁸R¹⁹.

In one embodiment R¹⁰ is independently a substituted or unsubstituted 2H-pyrrolyl, substituted or unsubstituted 2-pyrrolinyl, substituted or unsubstituted 3-pyrrolinyl, substituted or unsubstituted pyrrolidinyl, substituted or unsubstituted dioxolanyl, substituted or unsubstituted 2-imidazolinyl, substituted or unsubstituted imidazolidinyl, substituted or unsubstituted 2-pyrazolinyl, substituted or unsubstituted pyrazolidinyl, substituted or unsubstituted piperidinyl, substituted or unsubstituted morpholinyl, substituted or unsubstituted thiomorpholinyl, substituted or unsubstituted piperazinyl, substituted or unsubstituted phenyl, substituted or unsubstituted phenoxy, substituted or unsubstituted naphthyl, substituted or unsubstituted biphenyl, substituted or unsubstituted thiophenyl, substituted or unsubstituted pyrrolyl, substituted or unsubstituted pyrazolyl, substituted or unsubstituted imidazolyl, substituted or unsubstituted pyrazinyl, substituted or unsubstituted oxazolyl, substituted or unsubstituted isoxazolyl, substituted or unsubstituted thiazolyl, substituted or unsubstituted furyl, substituted or unsubstituted thienyl, substituted or unsubstituted pyridinyl, substituted or unsubstituted O-pyridinyl, substituted or unsubstituted pyrimidyl, substituted or unsubstituted benzothiazolyl, substituted or unsubstituted purinyl, substituted or unsubstituted benzimidazolyl, substituted or unsubstituted indolyl, substituted or unsubstituted isoquinolinyl, substituted or unsubstituted quinoxalinyl, substituted or unsubstituted quinolinyl, substituted or unsubstituted benzooxazolyl, substituted or unsubstituted[1,5]naphthyridinyl, substituted or unsubstituted pyrido[3,2-d]pyrimidinyl, substituted or unsubstituted[1,7]naphthyridinyl, substituted or unsubstituted 1H-pyrrolo[2,3-b]pyridinyl, substituted or unsubstituted pyrazolo[4,3-b]pyridinyl, substituted or unsubstituted pyrrolo[2,3-b]pyridinyl, substituted or unsubstituted thieno[2,3-b]pyridinyl, substituted or unsubstituted thiazolo[5,4-b]pyridinyl, substituted or unsubstituted pyridinyl-2-one, substituted or unsubstituted imidazo[1,2-b]pyridazinyl, substituted or unsubstituted pyrazolo[1,5-a]pyrimidinyl, substituted or unsubstituted pyridazinyl-3-one, substituted or unsubstituted imidazo[2,1-b][1,3,4]thiaciazolyl, substituted or unsubstituted imidazo[2,1-b]thiazolyl, substituted or unsubstituted isothiazolyl, substituted or unsubstituted triazolyl, or substituted or unsubstituted imidazo[4,5-b]pyridinyl.

In another embodiment R¹⁰ is substituted with 1 to 3 R²⁹ groups, wherein:

R²⁹ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR³⁰, —(CH₂)ⱼC(O)R³⁰, —(CH₂)ⱼC(O)OR³⁰, —(CH₂)ⱼNR³¹R³², —(CH₂)ⱼC(O)NR³¹R³², —(CH₂)ⱼOC(O)NR³¹R³², —(CH₂)ⱼNR³⁰(O)R³⁰, —(CH₂)ⱼNR³³C(O)OR³⁰, —(CH₂)ⱼNR³³C(O)NR³¹R³², —(CH₂)ⱼS(O)ₘR³⁴, —(CH₂)ⱼNR³³S(O)₂R³⁴, —(CH₂)ⱼS(O)₂NR³¹R³², wherein each j is independently an integer from 0 to 6; and m is independently an integer from 0 to 2;

R³⁰ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;

R³¹, R³², R³³, and R³⁴ are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or

R³¹ and R³² together with the N atom to which they are attached, independently form substituted or unsubstituted heterocycloalkyl, or substituted or unsubstituted heteroaryl, or

R³⁰ and R³³ together with the N atom to which they are attached, independently form substituted or unsubstituted heterocycloalkyl, or substituted or unsubstituted heteroaryl, or

R³³ and R³¹ or R³³ and R³² together with the N atom to which they are attached, each independently form substituted or unsubstituted heterocycloalkyl, or substituted or unsubstituted heteroaryl, or

R³³ and R³⁴ together with the N atom to which they are attached, independently form substituted or unsubstituted heterocycloalkyl, or substituted or unsubstituted heteroaryl;

wherein any of the R³⁰, R³¹, R³², R³³, and R³⁴ groups are each optionally independently substituted with 1 to 3 groups, each group independently selected from hydrogen, halogen, hydroxyl, amino, aminomonoalkyl, aminodialkyl, cyano, nitro, difluoromethyl, trifluoromethyl, oxo, alkyl, —O-alkyl, and —S-alkyl.

In yet another embodiment R¹⁰ is independently a substituted or unsubstituted pyrazolyl.

In yet another embodiment R¹⁰ is independently a substituted or unsubstituted pyrazolyl and wherein R⁴ is selected from a group consisting of a substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heterocycloalkyl, substituted or unsubstituted alkylheterocycloalkyl, substituted or unsubstituted heteroalkyl, substituted or unsubstituted heteroaryl, substituted or unsubstituted aryl, substituted or unsubstituted alkylaryl, and —(CH₂)ⱼNR¹⁸R¹⁹.

In another embodiment is a compound selected from:

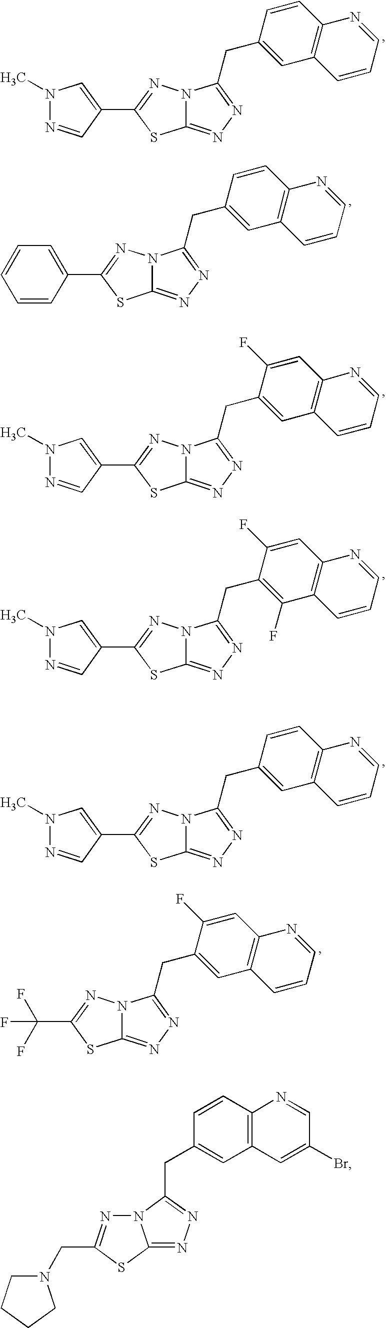

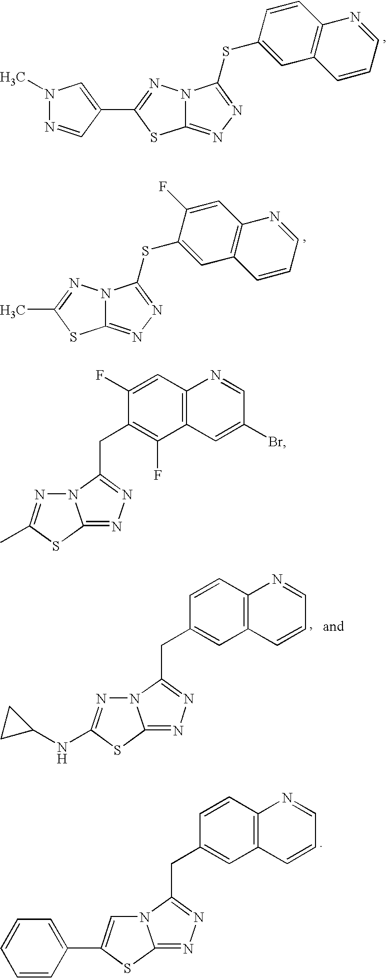

In a further embodiment is a compound selected from:

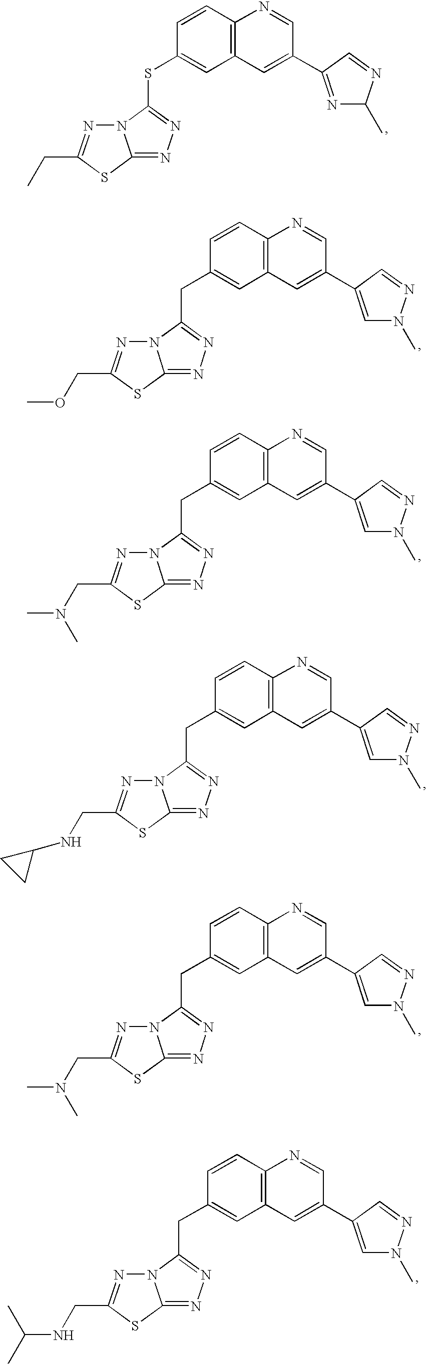

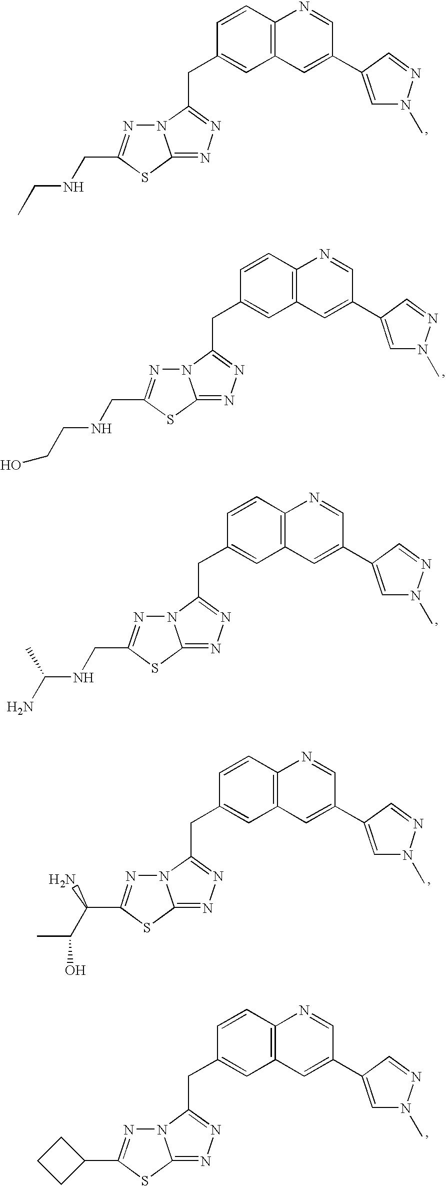

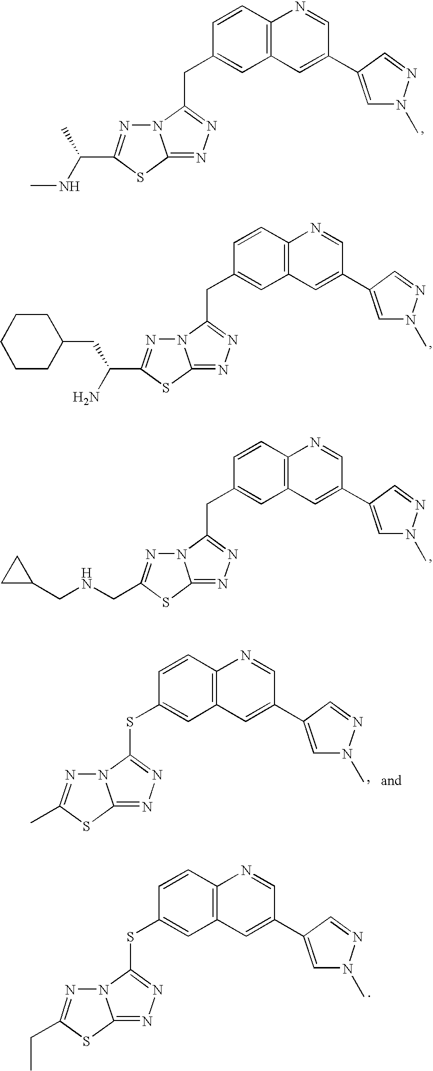

In another embodiment is a compound selected from:

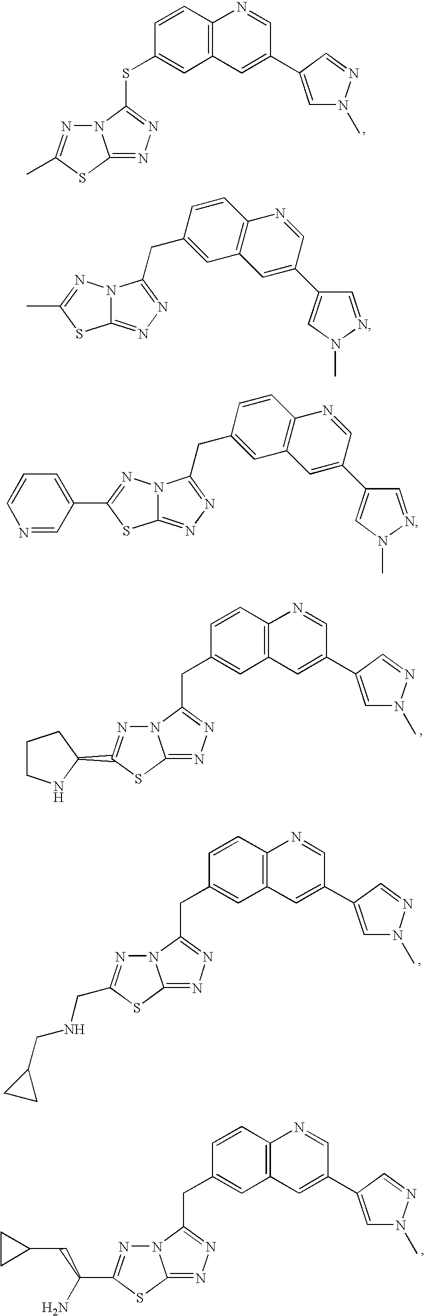

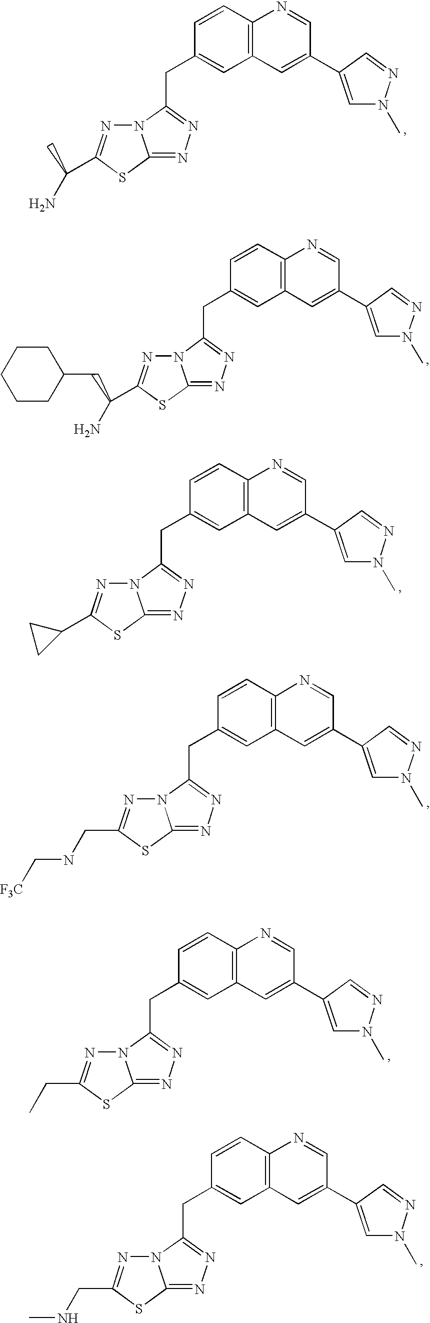

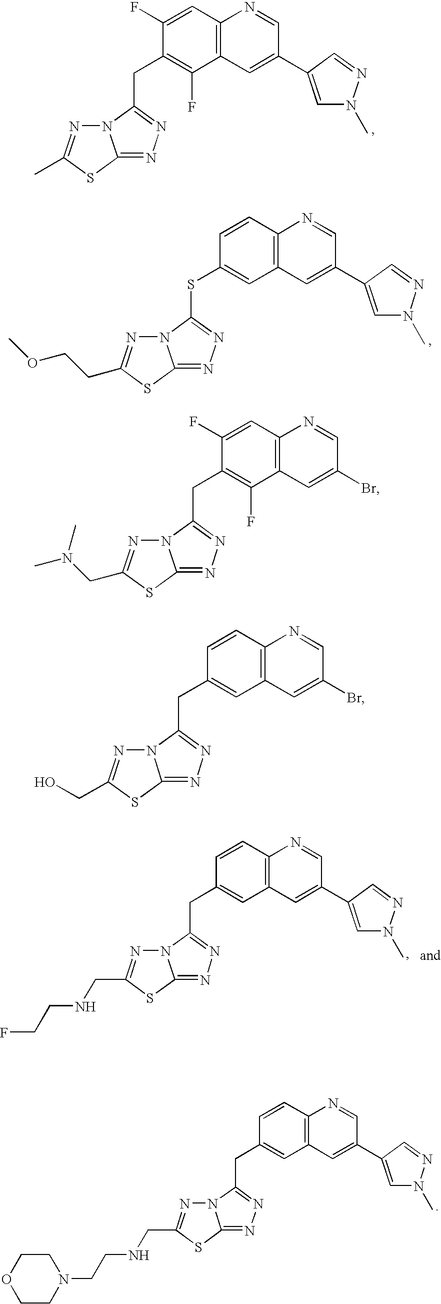

In one embodiment is a compound having the structure of Formulas (I1)-(14):

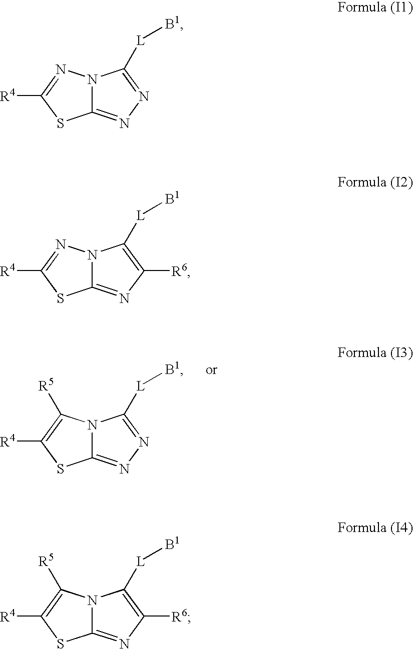

wherein:

L is

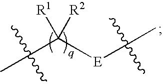

E is independently a direct bond or S;
q is an integer from 0 to 4;
u is an integer from 0 to 2;

R⁴ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷ (CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²¹C(O)NR¹⁸R¹⁹, —(CH₂)ⱼS(O)ₘR²¹, —(CH₂)ⱼNR²⁰S(O)₂R²¹;

R⁵ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹;

R⁴ and R⁵ optionally form substituted or unsubstituted cycloalkyl, substituted or unsubstituted heterocycloalkyl, substituted or unsubstituted aryl, or substituted or unsubstituted heteroaryl,

R⁶ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)NR²⁰C(O)OR¹⁷, —(CH₂)ⱼNR²⁰C(O)NR¹⁸R¹⁹, (CH₂)S(O)ₘR²¹ (CH₂)ⱼNR²⁰S(O)₂R²¹, —(CH₂)ⱼS(O)₂NR¹⁸R¹⁹;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼ NR¹⁵S(O)₂R¹⁶;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B¹ is

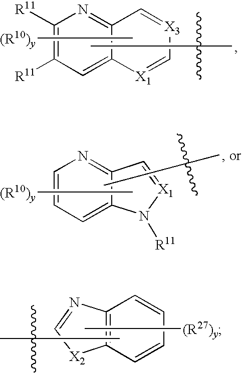

wherein:
X₁ is independently N or CR¹¹;
X₂ is NR¹¹, O, or S; and
X₃ is CR¹⁰ or N;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)OR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴; wherein each j is independently an integer from 0 to 6, and m is independently an integer from 0 to 2;

R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

In another embodiment, is a compound having the structure of Formulas (I1)-(I4):

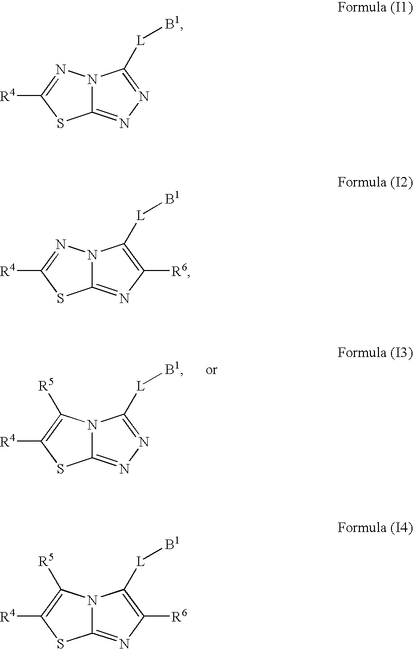

wherein: L is

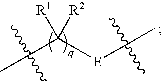

E is independently a direct bond or S; and

q is 0 or 1.

In a further embodiment, is a compound having the structure of Formulas (I1)-(I4):

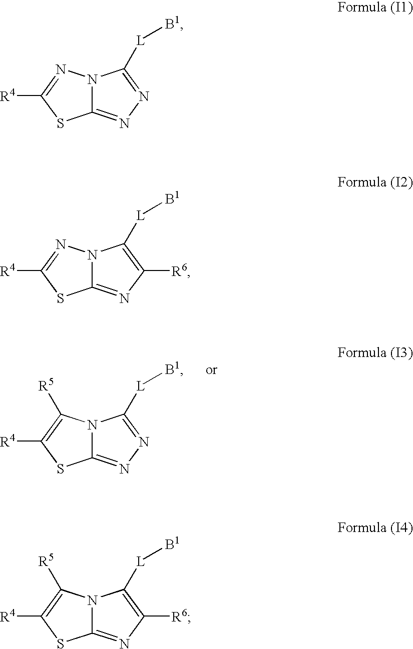

wherein L is

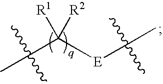

E is a direct bond;

q is 1; and

R¹ and R² are C₁-C₆ alkyl, halogen, or hydrogen. In one embodiment, R¹ and R² are both hydrogen.

In another embodiment, L is

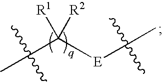

### E is S; and q is 0.

In one embodiment is a compound having the structure of Formulas (I1)-(I4) wherein R⁵ and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, each R⁵ and R⁶ are halogen. In another embodiment, each R⁵ and R⁶ are independently hydrogen, fluorine, bromine or chlorine. In a further embodiment, at least one of R⁵ and R⁶ are independently a C₁-C₃ alkyl group. In a further embodiment, at least one of R⁵ and R⁶ are independently methyl, ethyl, n-propyl, iso-propyl, n-butyl, iso-butyl, t-butyl, pentyl and hexyl. In yet a further embodiment, at least one of R⁵ and R⁶ are independently methyl. In yet a further embodiment, R⁵ and R⁶ are each independently hydrogen.

In one embodiment is a compound having the structure of Formulas (I1)-(I4) wherein R⁴ is substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl. In another embodiment, R⁴ is substituted or unsubstituted alkyl, substituted or unsubstituted cycloalkyl, substituted or unsubstituted heterocycloalkyl, substituted or unsubstituted aryl, or substituted or unsubstituted heteroaryl. In a further embodiment, R⁴ is substituted or unsubstituted aryl or substituted or unsubstituted heteroaryl. In a further embodiment, R⁴ is substituted or unsubstituted heteroaryl. In one embodiment, the substituted or unsubstituted heteroaryl group is thiophenyl, furanyl, pyranyl, isobenzofuranyl, chromenyl, xanthenyl, phenoxathiinyl, pyrrolyl, 2H-pyrrolyl, imidazolyl, isothiazolyl, isoxazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinzolinyl, cinnolinyl, pterdinyl, 4aH-carbazolyl, carbazolyl, carbolinyl, phenanthridinyl, acridinyl, perimidinyl, phenanthrolinyl, phenazinyl, phenarsazinyl, phenothiazinyl, furazanyl, or phenoxazinyl. In one embodiment, the substituted or unsubstituted heteroaryl group is pyridyl. In another embodiment, the pyridyl group is substituted with C₁-C₆ alkyl, halogen, cyano, hydroxyl, perfluoroalkyl, or SH. In another embodiment, the pyridyl group is substituted with methyl, ethyl, n-propyl, isopropyl, n-butyl, isobutyl, t-butyl, pentyl, and hexyl. In another embodiment, the substituted or unsubstituted heteroaryl group is pyrazolyl. In yet another embodiment, the pyrazole group is substituted with C₁-C₆ alkyl, halogen, cyano, hydroxyl, perfluoroalkyl, or SH. In another embodiment, the pyrazole group is substituted with C₁-C₆ alkyl. In another embodiment, the pyrazole group is substituted with methyl, ethyl, n-propyl, isopropyl, n-butyl, isobutyl, t-butyl, pentyl, and hexyl. In a further embodiment, the pyrazole group is substituted with methyl.

In a further embodiment, R⁴ is substituted or unsubstituted phenyl. In yet a further embodiment, the phenyl group is substituted with C₁-C₆ alkyl, halogen, cyano, hydroxyl, perfluoroalkyl, or SH. In another embodiment, the C₁-C₆ alkyl group is methyl, ethyl, n-propyl, isopropyl, n-butyl, isobutyl, t-butyl, pentyl, and hexyl.

In one embodiment is a compound having the structure of Formulas (I1)-(I4), wherein R⁴ is substituted or unsubstituted C₁-C₆ alkyl. In another embodiment, R⁴ is methyl, ethyl, n-propyl, isopropyl, n-butyl, isobutyl, t-butyl, pentyl, and hexyl. In a further embodiment, R⁴ is methyl. In yet a further embodiment, R⁴ is ethyl.

In one embodiment is a compound having the structure of Formulas (I1)-(I4) wherein R⁴ is hydrogen.

In another embodiment, R⁴ is substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, or substituted or unsubstituted heterocycloalkyl. In a further embodiment, R⁴ is substituted or unsubstituted alkylaminoalkyl or alkylaminocycloalkyl. In yet a further embodiment, the substituted or unsubstituted alkylaminoalkyl group is —(CH₂)ₐ(CH)NR^{a}(CH₂)ₘR^{b} wherein R^{a} is H or C₁-C₆ alkyl, and R^{b} is H, C₁-C₆ alkyl, halogen, hydroxy, NH₂, or SH, and n+m=0-4. In another embodiment, R⁴ is —(CH)NR^{a}R^{b} wherein R^{a} and R^{b} are H or C₁-C₆ alkyl. In a further embodiment, R⁴ is —(CH₂)ₙNH(CH₂)ₘC₁-C₈ cycloalkyl where n+m=0-4. In one embodiment, R⁴ is (CH₂)ₙNH(CH₂)ₘC₁-C₆ heterocycloalkyl where n+m=0-4. In another embodiment, R⁴ is (CH₂)ₙNR^{c}(CH₂)ₘR^{d} where R^{c} is hydrogen or C₁-C₃ alkyl, R^{d} is hydrogen, halogen, C₁-C₃ alkyl, and CF₃, and n+m=0-4. In a further embodiment, R⁴ is C₁-C₆ cycloalkyl. In yet a further embodiment, R⁴ is C₁-C₆ heterocycloalkyl. In one embodiment R⁴ is —(CH)NH₂(CH₂)ₘR^{e} wherein R^{e} is hydrogen, C₁-C₆ cycloalkyl, aryl, C₁-C₃ alkyl optionally substituted with halogen or hydroxy, and m is 0-3. In another embodiment, R⁴ is (CH₂), C₁-C₆ heteroalkyl wherein n is 0-6.

In one embodiment is a compound having the structure of Formulas (I1)-(I4) wherein B¹ is

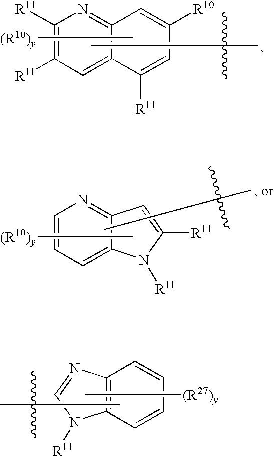

wherein R¹⁰ and R²⁷ are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl and R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, and y is independently an integer from 0 to 5.

In one embodiment, B′ is

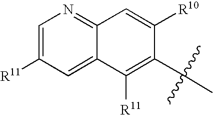

wherein R¹⁰ is hydrogen, fluorine, bromine, chlorine, C₁-C₂ alkyl, C₁-C₂ fluoroalkyl; and each R¹¹ is independently hydrogen, fluorine, bromine, chlorine, C₁-C₂ alkyl, C₁-C₂ fluoroalkyl and substituted or unsubstituted heteroaryl. In some embodiments, one R¹¹ is substituted or unsubstituted pyrazole. In further embodiments, the pyrazole is substituted with methyl, ethyl, n-propyl, isopropyl, n-butyl, isobutyl, and t-butyl. In even further embodiments, the pyrazole is substituted with methyl or ethyl.

In another embodiment, B¹ is

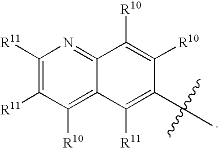

In a further embodiment, R¹⁰ and R¹¹ are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, and SH with the proviso that R¹⁰ and R¹¹ cannot all be H. In another embodiment, at least one of R¹⁰ and R¹¹ is C₁-C₆ alkyl. In yet another embodiment, at least one of R¹⁰ and R¹¹ is independently methyl, ethyl, n-propyl, iso-propyl, n-butyl, iso-butyl, t-butyl, pentyl, and hexyl. In one embodiment, at least one of R¹⁰ and R¹¹ is halogen. In another embodiment, at least one of R¹⁰ and R¹¹ is fluorine, chlorine, and bromine. In yet another embodiment, B¹ is

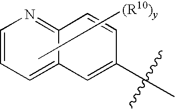

wherein at least one of R¹⁰ is fluorine and bromine and y is an integer from 1 to 6. In one embodiment, y is 2. In another embodiment, y is 3.

In one embodiment, B′ is

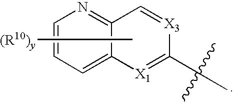

In another embodiment, X₁ is CR¹¹ and X₃ is CH, wherein R¹¹ is halogen, nitro, cyano, hydroxyl, substituted or unsubstituted alkyl, substituted or unsubstituted cycloalkyl, perfluoroalkyl, substituted or unsubstituted heteroalkyl, and SH. In a further embodiment, R¹¹ is halogen. In yet a further embodiment, X, is CH and X₃ is CF. In another embodiment, X₁ is CF and X₃ is CH. In yet another embodiment, X₁ and X₃ are CF.

In yet a further embodiment, B¹ is a substituted quinoline group. In yet another embodiment the quinoline group is substituted with R¹⁰ wherein each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², (CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)^{m}R²⁶, —(CH₂)ⱼNR²⁵S(O)₂R²⁶, —(CH₂)ⱼS(O)₂NR²³R²⁴, wherein y is independently an integer from 0 to 5.

In one embodiment R¹⁰ is a substituted or unsubstituted alkyl, substituted or unsubstituted aryl or a substituted or unsubstituted heteroaryl. In another embodiment, R¹⁰ is a substituted or unsubstituted heteroaryl having at least one N, S, or O atom. In yet another embodiment, R¹⁰ is a substituted heteroaryl having at least two nitrogen atoms. In yet a further embodiment, R¹⁰ is a substituted pyrazole group. In another embodiment, the pyrazole group is substituted with hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, the pyrazole group is substituted with a C₁-C₆ alkyl group. In a further embodiment, the pyrazole group is substituted with a C₁-C₃ alkyl group. In a further embodiment, the pyrazole group is substituted with methyl, ethyl, n-propyl, iso-propyl, n-butyl, iso-butyl, t-butyl, pentyl and hexyl. In yet a further embodiment, the pyrazole group is substituted with methyl.

In another embodiment is a compound having the structure of Formulas (I1)-(I4), wherein B¹ is

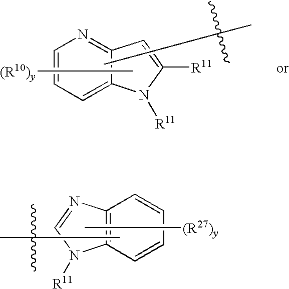

wherein R¹⁰ and R²⁷ are each independently hydrogen, halogen, nitro, cyano, hydroxyl, substituted or unsubstituted alkyl, substituted or unsubstituted cycloalkyl, perfluoroalkyl, and substituted or unsubstituted heteroalkyl, R¹¹ is independently a direct bond, hydrogen, halogen, nitro, cyano, hydroxyl, substituted or unsubstituted alkyl, substituted or unsubstituted cycloalkyl, perfluoroalkyl, and substituted or unsubstituted heteroalkyl and y is independently an integer from 0 to 5. In one embodiment, R¹⁰, R¹¹, and R²⁷ are each independently hydrogen and halogen. In another embodiment, at least one R¹⁰, R¹¹, and R²⁷ is independently fluorine, chlorine, and bromine. In yet another embodiment, at least one R¹⁰, R¹¹, and R²⁷ is independently C₁-C₆ alkyl. In some embodiments, at least one R¹⁰, R¹¹, and R²⁷ is independently methyl, ethyl, n-propyl, iso-propyl, n-butyl, isobutyl, t-butyl, pentyl and hexyl.

In yet a further embodiment is a compound having the structure of Formulas (I1c)-(I4c):

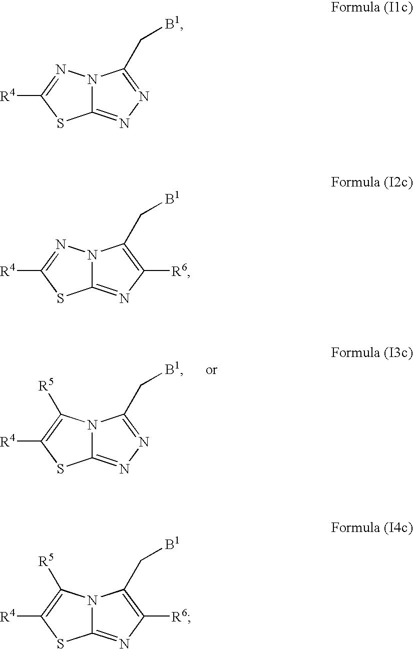

wherein R⁴ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)NR²⁰C(O)OR¹⁷, —(CH₂)ⱼNR²⁰C(O)NR¹⁸R¹⁹, —(CH₂)ⱼS(O)ₘR²¹, or —(CH₂)NR²⁰S(O)₂R²¹; and R⁵ and R⁶ are independently hydrogen or C₁-C₆ alkyl. In one embodiment, R⁴ is substituted or unsubstituted cycloalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl.

In one embodiment is a compound having the structure of Formulas (I1c)-(I4c) wherein R⁵ and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, each R⁵ and R⁶ are halogen. In another embodiment, each R⁵ and R⁶ are independently hydrogen, fluorine, bromine or chlorine. In a further embodiment, at least one of R⁵ and R⁶ are independently a C₁-C₃ alkyl group. In a further embodiment, at least one of R⁵ and R⁶ are independently methyl, ethyl, n-propyl, iso-propyl, n-butyl, iso-butyl, t-butyl, pentyl and hexyl. In yet a further embodiment, at least one of R⁵ and R⁶ are independently methyl. In yet a further embodiment, R⁵ and R⁶ are each independently hydrogen.

In one embodiment is a compound having the structure of Formulas (I1c)-(I4c) wherein R⁴ is substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl.

In another embodiment, R⁴ is substituted or unsubstituted alkyl, substituted or unsubstituted cycloalkyl, substituted or unsubstituted heterocycloalkyl, substituted or unsubstituted aryl, or substituted or unsubstituted heteroaryl. In a further embodiment, R⁴ is substituted or unsubstituted aryl or substituted or unsubstituted heteroaryl. In a further embodiment, R⁴ is substituted or unsubstituted heteroaryl. In one embodiment, the substituted or unsubstituted heteroaryl group is thiophenyl, furanyl, pyranyl, isobenzofuranyl, chromenyl, xanthenyl, phenoxathiinyl, pyrrolyl, 2H-pyrrolyl, imidazolyl, isothiazolyl, isoxazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinzolinyl, cinnolinyl, pterdinyl, 4aH-carbazolyl, carbazolyl, carbolinyl, phenanthridinyl, acridinyl, perimidinyl, phenanthrolinyl, phenazinyl, phenarsazinyl, phenothiazinyl, furazanyl, or phenoxazinyl. In one embodiment, the substituted or unsubstituted heteroaryl group is pyridyl. In another embodiment, the pyridyl group is substituted with C₁-C₆ alkyl, halogen, cyano, hydroxyl, perfluoroalkyl, or SH. In another embodiment, the pyridyl group is substituted with methyl, ethyl, n-propyl, isopropyl, n-butyl, isobutyl, t-butyl, pentyl, and hexyl. In another embodiment, the substituted or unsubstituted heteroaryl group is pyrazolyl. In yet another embodiment, the pyrazole group is substituted with C₁-C₆ alkyl, halogen, cyano, hydroxyl, perfluoroalkyl, or SH. In another embodiment, the pyrazole group is substituted with C₁-C₆ alkyl. In another embodiment, the pyrazole group is substituted with methyl, ethyl, n-propyl, isopropyl, n-butyl, isobutyl, t-butyl, pentyl, and hexyl. In a further embodiment, the pyrazole group is substituted with methyl.

In a further embodiment, R⁴ is substituted or unsubstituted phenyl. In yet a further embodiment, the phenyl group is substituted with C₁-C₆ alkyl, halogen, cyano, hydroxyl, perfluoroalkyl, or SH. In another embodiment, the C₁-C₆ alkyl group is methyl, ethyl, n-propyl, isopropyl, n-butyl, isobutyl, t-butyl, pentyl, and hexyl.

In one embodiment, R⁴ is substituted or unsubstituted C₁-C₆ alkyl. In another embodiment, R⁴ is methyl, ethyl, n-propyl, isopropyl, n-butyl, isobutyl, t-butyl, pentyl, and hexyl. In a further embodiment, R⁴ is methyl. In yet a further embodiment, R⁴ is ethyl.

In one embodiment is a compound having the structure of Formulas (I1c)-(I4c) wherein R⁴ is hydrogen.

In another embodiment, R⁴ is substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, or substituted or unsubstituted heterocycloalkyl. In a further embodiment, R⁴ is substituted or unsubstituted alkylaminoalkyl or alkylaminocycloalkyl. In yet a further embodiment, the substituted or unsubstituted alkylaminoalkyl group is —CH₂)ₙ(CH)NR^{a}(CH₂)ₘ wherein R^{a} is H or C₁-C₆ alkyl, and R^{b} is H, C₁-C₆ alkyl, halogen, hydroxy, NH₂, or SH, and n+m=0-4. In another embodiment, R⁴ is —(CH)NR^{a}R^{b} wherein R^{a} and R^{b} are H or C₁-C₆ alkyl. In a further embodiment, R⁴ is —(CH₂)ₙNH(CH₂)ₘC₁-C₈ cycloalkyl where n+m=0-4. In one embodiment, R⁴ is (CH₂)ₙNH(CH₂)ₘC₁-C₆ heterocycloalkyl where n+m=0-4. In another embodiment, R⁴ is (CH₂)ₙNR^{c}(CH₂)ₘR^{d} where R^{c} is hydrogen or C₁-C₃ alkyl, R^{d} is hydrogen, halogen, C₁-C₃ alkyl, and CF₃, and n+m=0-4. In a further embodiment, R⁴ is C₁-C₆ cycloalkyl. In yet a further embodiment, R⁴ is C₁-C₆ heterocycloalkyl. In one embodiment R⁴ is —(CH)NH₂(CH₂)ₘR^{e} wherein R^{e} is hydrogen, C₁-C₆ cycloalkyl, aryl, C₁-C₃ alkyl optionally substituted with halogen or hydroxy, and m is 0-3. In another embodiment, R⁴ is (CH₂)ₙC₁-C₆ heteroalkyl wherein n is 0-6.

In one embodiment is a compound having the structure of Formulas (I1c)-(I4c) wherein B¹ is

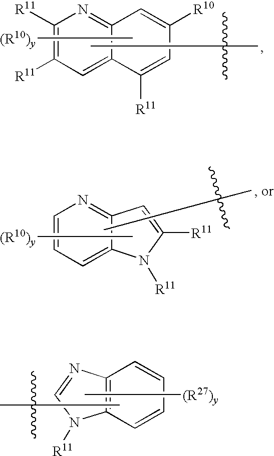

wherein R¹⁰ and R²⁷ are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl and R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, and y is independently an integer from 0 to 5.

In another embodiment is a compound having the structure of Formulas (I1c)-(I4c):

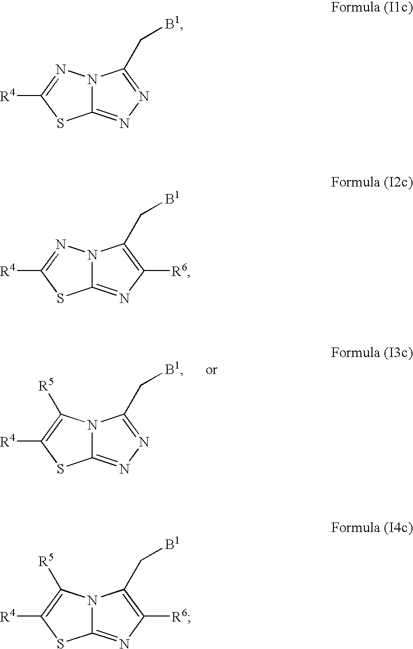

wherein R⁴ is a substituted or unsubstituted aryl or substituted or unsubstituted heteroaryl. In a further embodiment, R⁴ is a substituted or unsubstituted heteroaryl having at least one N, S, or O atom. In yet another embodiment, R⁴ is a substituted or unsubstituted heteroaryl having at least one N atom.

In another embodiment, is a compound having the structure of Formulas (I1d)-(I4d):

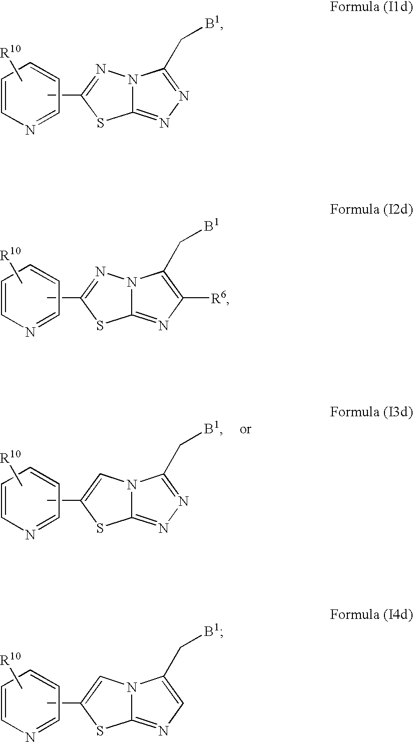

wherein R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 5. In a further embodiment, R¹⁰ is independently hydrogen, halogen, nitro, cyano, hydroxyl, substituted or unsubstituted alkyl, substituted or unsubstituted cycloalkyl, or perfluoroalkyl. In a further embodiment, at least one R¹⁰ is independently C₁-C₆ alkyl. In a further embodiment, at least one R¹⁰ is independently hydrogen. In yet a further embodiment, is a compound having the structures of Formulas (I1d)-(I4d) wherein B¹ is

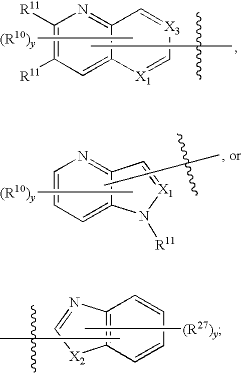

wherein:
X₁ is independently N or CR¹;
X₂ is NR¹¹, O, or S; and
X₃ is CR¹⁰ or N;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, (CH₂)ⱼS(O)R²⁶, —(CH₂)ⱼNR²⁵S(O)₂R²⁶, —(CH₂)ⱼS(O)₂NR²³R²⁴; wherein each j is independently an integer from 0 to 6, and m is independently an integer from 0 to 2;

with the proviso that when R¹¹ is independently a direct bond, then R¹⁰ or R²⁷ cannot all be H; R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl.

In one aspect is a compound having the structure of Formulas (I5), (I6), (I7), or (I8):

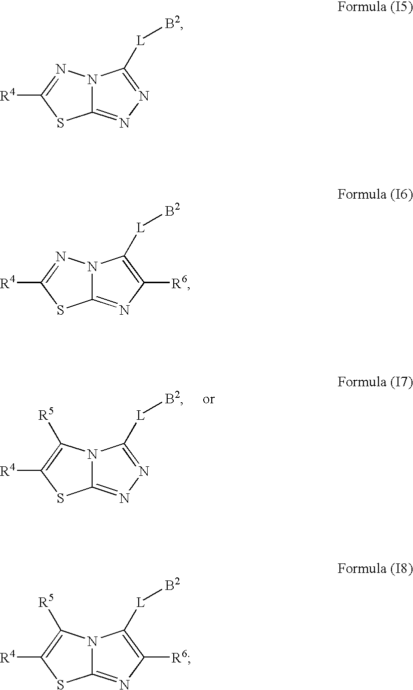

wherein:

L is

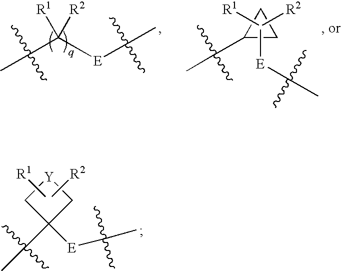

E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;

R⁴, R⁵, and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, (CH₂)ⱼNR²⁰C(O)NR¹⁸R¹⁹ (CH₂)ⱼS(O)ₘR²¹, —(CH₂)ⱼNR²⁰S(O)₂R²¹, —(CH₂)ⱼS(O)₂NR¹⁸R¹⁹;

wherein each j is independently an integer from 0 to 6; and m is independently an integer from 0 to 2;

R⁴ and R⁵ optionally form substituted or unsubstituted cycloalkyl, substituted or unsubstituted heterocycloalkyl, substituted or unsubstituted aryl, or substituted or unsubstituted heteroaryl;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, (CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶, wherein each j is independently an integer from 0 to 6, and m is independently an integer from 0 to 2;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B² is selected from:

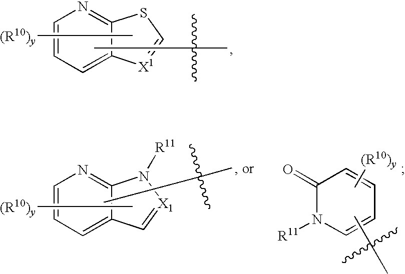

wherein:

X₁ is independently N or CR¹¹;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH)ⱼOR¹², —(CH)ⱼC(O)R²², —(CH₂)ⱼC(O)OR²², —(CH₂)ⱼNR²³R²⁴, —(CH₂)ⱼc(O)NR²³R²⁴, —(CH₂)ⱼOC(O)N²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

y is independently an integer from 0 to 4;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²S(O)₂R²⁶—(CH₂)ⱼS(O)₂NR²³R²⁴ wherein each j is independently an integer from 0 to 6; and m is independently an integer from 0 to 2;

R¹², R¹³, R¹⁴, R¹⁵, R¹⁶, R¹⁷, R¹⁸, R¹⁹, R²⁰, R²¹, R²², R²³, R²⁴, R²⁵ and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

In one embodiment is a compound having the structure of Formulas (I5), (I6), (I7), or (I8), wherein L is

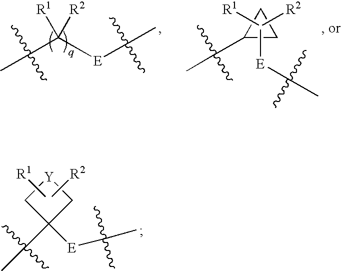

E is independently a direct bond or S; and q is an integer from 0 to 4. In another embodiment, E is independently S and q is 0. In a further embodiment, E is a direct bond and q is 1 or 2. In yet a further embodiment, q is 1.

In one embodiment is a compound having the structure of Formulas (I5a)-(I8b):

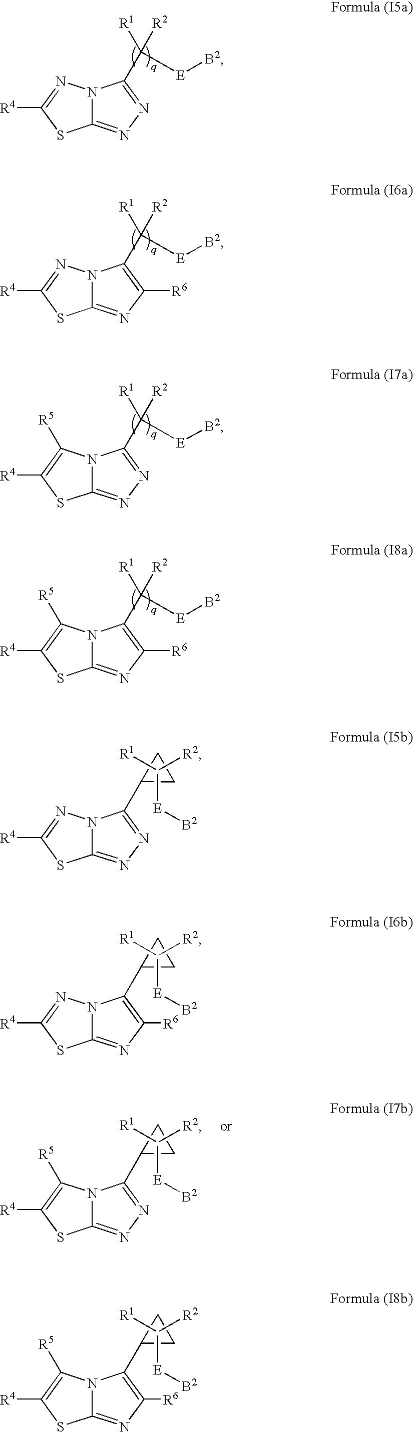

or an enantiomer, diastereomer, racemate, or pharmaceutically acceptable salt, or solvate thereof. In one embodiment is a compound having the structure of Formulas (I5a), (I5b), (I6a), (I6b), (I7a), (I7b), (I8a), and (I8b) wherein R⁵ and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In yet a further embodiment, at least one of R⁵ and R⁶ are independently a halogen. In another embodiment, at least one of R⁵ and R⁶ are independently fluorine, bromine, and chlorine. In a further embodiment, at least one of R⁵ and R⁶ are independently a C₁-C₃ alkyl group. In a further embodiment, at least one of R⁵ and R⁶ are independently methyl, ethyl, n-propyl, iso-propyl, n-butyl, iso-butyl, t-butyl, pentyl and hexyl. In yet a further embodiment, at least one of R⁵ and R⁶ are independently methyl. In yet a further embodiment, R⁵ and R⁶ are each independently hydrogen.

In one embodiment is a compound having the structure of Formulas (I5a), (I5b), (I6a), (I6b), (I7a), (I7b), (I8a), and (I8b) wherein R⁴ is substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl.

In another embodiment, R⁴ is substituted or unsubstituted alkyl, substituted or unsubstituted cycloalkyl, substituted or unsubstituted heterocycloalkyl, substituted or unsubstituted aryl, or substituted or unsubstituted heteroaryl.

In a further embodiment, R⁴ is substituted or unsubstituted aryl or substituted or unsubstituted heteroaryl. In one embodiment, the substituted or unsubstituted heteroaryl group is thiophenyl, furanyl, pyranyl, isobenzofuranyl, chromenyl, xanthenyl, phenoxathiinyl, pyrrolyl, 2H-pyrrolyl, imidazolyl, isothiazolyl, isoxazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinzolinyl, cinnolinyl, pterdinyl, 4aH-carbazolyl, carbazolyl, carbolinyl, phenanthridinyl, acridinyl, perimidinyl, phenanthrolinyl, phenazinyl, phenarsazinyl, phenothiazinyl, furazanyl, or phenoxazinyl.

In one embodiment, the substituted or unsubstituted heteroaryl group is pyridyl. In another embodiment, the pyridyl group is substituted with C₁-C₆ alkyl, halogen, cyano, hydroxyl, perfluoroalkyl, or SH. In another embodiment, the pyridyl group is substituted with methyl, ethyl, n-propyl, isopropyl, n-butyl, isobutyl, t-butyl, pentyl, and hexyl. In another embodiment, the substituted or unsubstituted heteroaryl group is pyrazolyl. In yet another embodiment, the pyrazole group is substituted with C₁-C₆ alkyl, halogen, cyano, hydroxyl, perfluoroalkyl, or SH. In another embodiment, the pyrazole group is substituted with C₁-C₆ alkyl. In another embodiment, the pyrazole group is substituted with methyl, ethyl, n-propyl, isopropyl, n-butyl, isobutyl, t-butyl, pentyl, and hexyl. In a further embodiment, the pyrazole group is substituted with methyl. In a further embodiment, R⁴ is substituted or unsubstituted phenyl. In yet a further embodiment, the phenyl group is substituted with C₁-C₆ alkyl, halogen, cyano, hydroxyl, perfluoroalkyl, or SH. In another embodiment, the C₁-C₆ alkyl group is methyl, ethyl, n-propyl, isopropyl, n-butyl, isobutyl, t-butyl, pentyl, and hexyl.

In one embodiment is a compound having the structure of Formulas (I5a), (I5b), (I6a), (I6b), (I7a), (I7b), (I8a), and (I8b) wherein R⁴ is substituted or unsubstituted C₁-C₆ alkyl. In another embodiment, R⁴ is methyl, ethyl, n-propyl, isopropyl, n-butyl, isobutyl, t-butyl, pentyl, and hexyl. In a further embodiment, R⁴ is methyl. In yet a further embodiment, R⁴ is ethyl.

In another embodiment, R⁴ is substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, or substituted or unsubstituted heterocycloalkyl. In a further embodiment, R⁴ is substituted or unsubstituted alkylaminoalkyl or alkylaminocycloalkyl. In yet a further embodiment, the substituted or unsubstituted alkylaminoalkyl group is —(CH₂)ₙ(CH)NR^{a}(CH₂)ₘR^{b} wherein R¹ is H or C₁-C₆ alkyl, and R^{b} is H, C₁-C₆ alkyl, halogen, hydroxy, NH₂, or SH, and n+m=0-4. In another embodiment, R⁴ is —(CH)NR^{a}R^{b}.

In one embodiment, is a compound having the structure of Formulas (I5a), (I5b), (I6a), (I6b), (I7a), (I7b), (I8a), and (I8b) wherein B² is

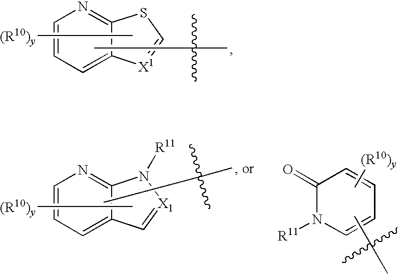

each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl and each R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, and y is independently an integer from 0 to 4.

In a further embodiment, R¹⁰ and R¹¹ are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, and SH. In another embodiment, at least one of R¹⁰ and R¹¹ is C₁-C₆ alkyl. In yet another embodiment, at least one of R¹⁰ and R¹¹ is methyl, ethyl, n-propyl, iso-propyl, n-butyl, iso-butyl, t-butyl, pentyl, and hexyl. In one embodiment, at least one of R¹⁰ and R¹¹ is halogen. In another embodiment, at least one of R¹⁰ and R¹¹ is fluorine, chlorine, and bromine. In one embodiment, at least one of R¹⁰ and R¹¹ is a substituted or unsubstituted heteroaryl having at least one N, S, or O atom. In yet another embodiment, at least one R¹⁰ is a substituted heteroaryl having at least two nitrogen atoms. In yet a further embodiment, at least one R¹⁰ is a substituted pyrazole group. In another embodiment, the pyrazole group is substituted with hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, the pyrazole group is substituted with a C₁-C₆ alkyl group. In a further embodiment, the pyrazole group is substituted with a C₁-C₃ alkyl group. In a further embodiment, the pyrazole group is substituted with methyl, ethyl, n-propyl, iso-propyl, n-butyl, iso-butyl, t-butyl, pentyl and hexyl. In yet a further embodiment, the pyrazole group is substituted with methyl.

In another aspect is a compound having the formula:

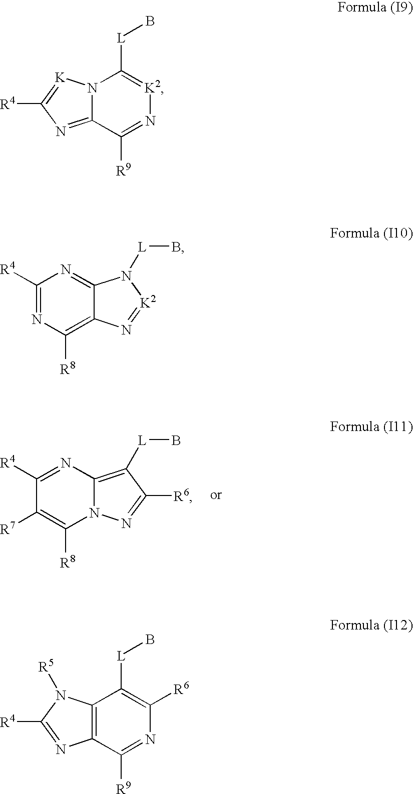

wherein:

K is N or CR⁵,

K₂ is N or CR⁶;

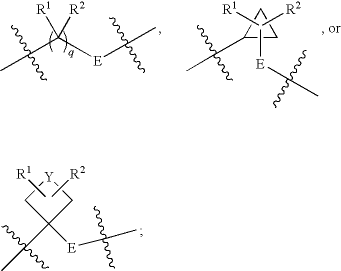

L is

wherein:

E is independently a direct bond, O, C═O, S(O)ᵤ, or NR³;

Y is CH₂, CF₂, O, C(O)—, OC(O)—, NR³, or S(O)ᵤ;

q is an integer from 0 to 4;

u is an integer from 0 to 2;

R⁴, R⁵, R⁶, R⁷, R⁸, and R⁹ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aminoalkylenecycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2;

R⁴ and R⁵ optionally form substituted or unsubstituted cycloalkyl, substituted or unsubstituted heterocycloalkyl, substituted or unsubstituted aryl, or substituted or unsubstituted heteroaryl, or

R⁴ and R⁷ optionally form substituted or unsubstituted cycloalkyl, substituted or unsubstituted heterocycloalkyl, substituted or unsubstituted aryl, or substituted or unsubstituted heteroaryl, or

R⁷ and R⁸ optionally form substituted or unsubstituted cycloalkyl, substituted or unsubstituted heterocycloalkyl, substituted or unsubstituted aryl, or substituted or unsubstituted heteroaryl;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶, wherein each j is independently an integer from 0 to 6, and m is independently an integer from 0 to 2;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;

B is a substituted or unsubstituted heteroaryl selected from:

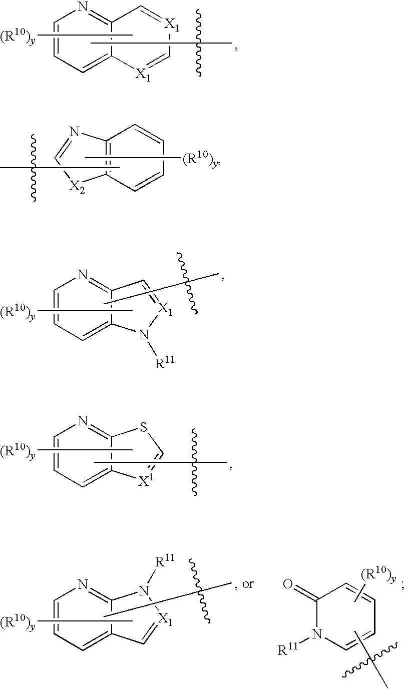

wherein:

X₁ is independently N or C; and

X₂ is N(R¹¹), O, or S;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²¹R²⁴, wherein each j is independently an integer from 0 to 6; and m is independently an integer from 0 to 2;

R¹², R¹⁷ and R²² are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;

R¹³, R¹⁴, R¹⁵, R¹⁶, R¹⁸, R¹⁹, R²⁰, R²¹ R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or

R¹³ and R¹⁴, R¹⁸ and R¹⁹, and R²³ and R²⁴ together with the N atom to which they are attached, each independently form substituted or unsubstituted heterocycloalkyl, or substituted or unsubstituted heteroaryl, or

R¹² and R¹⁵, R¹⁷ and R²⁰, and R²² and R²⁵ together with the N atom to which they are attached, each independently form substituted or unsubstituted heterocycloalkyl, or substituted or unsubstituted heteroaryl, or

R¹³ and R¹⁵ or R¹⁴ and R¹⁵, R¹⁸ and R²⁰ or R¹⁹ and R²⁰, and R²³ and R²⁵ or R²⁴ and R²⁵ together with the N atom to which they are attached, each independently form substituted or unsubstituted heterocycloalkyl, or substituted or unsubstituted heteroaryl, or

R¹⁵ and R¹⁶, R²⁰ and R²¹, and R²⁵ and R²⁶ together with the N atom to which they are attached, each independently form substituted or unsubstituted heterocycloalkyl, or substituted or unsubstituted heteroaryl; wherein any of the R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, and R²⁴ groups are each optionally independently substituted with 1 to 3 groups, each group independently selected from hydrogen, halogen, hydroxyl, amino, aminomonoalkyl, aminodialkyl, cyano, morpholine, nitro, difluoromethyl, trifluoromethyl, oxo, alkyl, —O-alkyl, and —S-alkyl; or an enantiomer, diastereomer, racemate, or pharmaceutically acceptable salt, or solvate thereof.

In one embodiment is a compound having the structure of Formulas (I9), (I10), (I11), and (I12) wherein L is

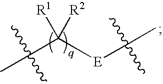

and wherein E is independently a direct bond or S, and q is 0 or 1. In another embodiment, E is a direct bond and q is 1. In a further embodiment, E is S and q is 0. In yet a further embodiment, R¹ and R² are each H. In another embodiment, R¹ and R² are independently H or C₁-C₃ alkyl.

In one embodiment is a compound having the structure of Formulas (I9), (I10), (I11), and (I12) wherein R⁴, R⁵, R⁶, R⁷, R⁸, and R⁹ are each independently hydrogen, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl.

In another embodiment, R⁴, R⁵, R⁶, R⁷, R⁸, and R⁹ are each independently hydrogen, substituted or unsubstituted alkyl, substituted or unsubstituted cycloalkyl, substituted or unsubstituted heterocycloalkyl, substituted or unsubstituted aryl, or substituted or unsubstituted heteroaryl.

In a further embodiment, R⁵, R⁶, R⁷, R⁸, and R⁹ are each independently hydrogen and each R⁴ is independently substituted or unsubstituted aryl or substituted or unsubstituted heteroaryl. In one embodiment, the substituted or unsubstituted heteroaryl group is thiophenyl, furanyl, pyranyl, isobenzofuranyl, chromenyl, xanthenyl, phenoxathiinyl, pyrrolyl, 2H-pyrrolyl, imidazolyl, isothiazolyl, isoxazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinzolinyl, cinnolinyl, pterdinyl, 4aH-carbazolyl, carbazolyl, carbolinyl, phenanthridinyl, acridinyl, perimidinyl, phenanthrolinyl, phenazinyl, phenarsazinyl, phenothiazinyl, furazanyl, or phenoxazinyl. In one embodiment, the substituted or unsubstituted heteroaryl group is pyridyl. In another embodiment, the pyridyl group is substituted with C₁-C₆ alkyl, halogen, cyano, hydroxyl, perfluoroalkyl, or SH. In another embodiment, the pyridyl group is substituted with methyl, ethyl, n-propyl, isopropyl, n-butyl, isobutyl, t-butyl, pentyl, and hexyl. In another embodiment, the substituted or unsubstituted heteroaryl group is pyrazolyl. In yet another embodiment, the pyrazole group is substituted with C₁-C₆ alkyl, halogen, cyano, hydroxyl, perfluoroalkyl, or SH. In another embodiment, the pyrazole group is substituted with C₁-C₆ alkyl. In another embodiment, the pyrazole group is substituted with methyl, ethyl, n-propyl, isopropyl, n-butyl, isobutyl, t-butyl, pentyl, and hexyl. In a further embodiment, the pyrazole group is substituted with methyl. In a further embodiment R⁵, R⁶, R⁷, R⁸, and R⁹ are each independently hydrogen and each R⁴ is independently substituted or unsubstituted phenyl. In yet a further embodiment, the phenyl group is substituted with C₁-C₆ alkyl, halogen, cyano, hydroxyl, perfluoroalkyl, or SH. In another embodiment, the C₁-C₆ alkyl group is methyl, ethyl, n-propyl, isopropyl, n-butyl, isobutyl, t-butyl, pentyl, and hexyl.

In one embodiment is a compound having the structure of Formulas (I9), (I10), (I11), and (I12) wherein R⁴, R⁵, R⁶, R⁷, R⁸, and R⁹ are each independently hydrogen or substituted or unsubstituted C₁-C₆ alkyl. In another embodiment, R⁴, R⁵, R⁶, R⁷, R⁸, and R⁹ are each independently hydrogen, methyl, ethyl, n-propyl, isopropyl, n-butyl, isobutyl, t-butyl, pentyl, and hexyl. In a further embodiment, R⁴, R⁵, R⁶, R⁷, R⁸, and R⁹ are each independently hydrogen or methyl. In yet a further embodiment, R⁴, R⁵, R⁶, R⁷, R⁸, and R⁹ are each independently hydrogen or ethyl.

In one embodiment is a compound having the structure of Formulas (I9), (I10), (I11), and (I12) wherein R⁴ is hydrogen.

In another embodiment, R⁴, R⁵, R⁶, R⁷, R⁸, and R⁹ are each independently hydrogen, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, or substituted or unsubstituted heterocycloalkyl.

In a further embodiment R⁵, R⁶, R⁷, R⁸, and R⁹ are each independently hydrogen, and each R⁴ is independently substituted or unsubstituted alkylaminoalkyl or alkylaminocycloalkyl. In yet a further embodiment, the substituted or unsubstituted alkylaminoalkyl group is —CH₂)ₙ(CH)NR^{a}(CH₂)ₘR^{b} wherein R^{a} is H or C₁-C₆ alkyl, and R^{b} is H, C₁-C₆ alkyl, halogen, hydroxy, NH₂, or SH, and n+m=0-4. In another embodiment R⁵, R⁶, R⁷, R⁸, and R⁹ are each independently hydrogen and each R⁴ is independently —(CH)NR^{a}R^{b} wherein R^{a} and R^{b} is H or C₁-C₆ alkyl. In a further embodiment R⁵, R⁶, R⁷, R⁸, and R⁹ are each independently hydrogen and each R⁴ is independently —(CH₂)ₙNH(CH₂)ₘC₁-C₈ cycloalkyl where n+m=0-4. In one embodiment R⁵, R⁶, R⁷, R⁸, and R⁹ are each independently hydrogen and each R⁴ is independently (CH₂)ₙNH(CH₂)ₘC₁-C₆ heterocycloalkyl where n+m=0-4. In another embodiment R⁵, R⁶, R⁷, R⁸, and R⁹ are each independently hydrogen and each R⁴ is independently (CH₂)ᵤNR^{c}(CH₂)ₘR^{d} wherein R^{c} is hydrogen or C₁-C₃ alkyl, R^{d} is hydrogen, halogen, C₁-C₃ alkyl, and CF₃, and n+m=0-4. In a further embodiment R⁵, R⁶, R⁷, R⁸, and R⁹ are each independently hydrogen and each R⁴ is independently C₁-C₆ cycloalkyl. In yet a further embodiment R⁵, R⁶, R⁷, R⁸, and R⁹ are each independently hydrogen and each R⁴ is independently C₁-C₆ heterocycloalkyl. In one embodiment R⁵, R⁶, R⁷, R⁸, and R⁹ are each independently hydrogen and each R⁴ is independently —(CH)NH₂(CH₂)ₘR^{e} wherein R^{e} is hydrogen, C₁-C₆ cycloalkyl, aryl, C₁-C₃ alkyl optionally substituted with halogen or hydroxy, and m is 0-3. In another embodiment R⁵, R⁶, R⁷, R⁸, and R⁹ are each independently hydrogen and each R⁴ is independently (CH₂)ₙC₁-C₆ heteroalkyl wherein n is 0-6.

In one embodiment is a compound having the structure of Formulas (I9), (I10), (I11), and (I12) wherein B is selected from:

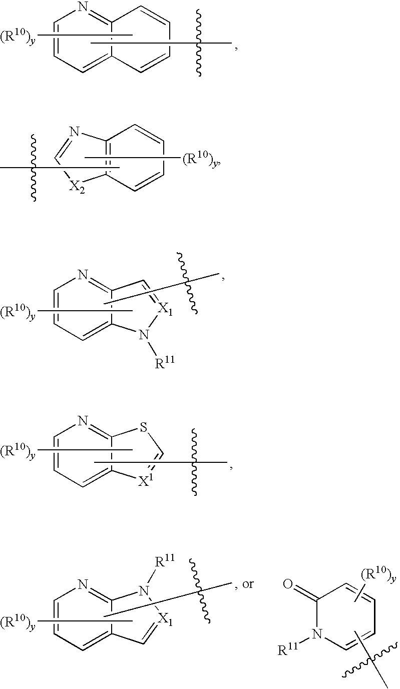

wherein X₁ is N or C; and X₂ is N(R¹¹), S, or O.

In another embodiment is a compound having the structure of Formulas (I9), (I10), (I11), and (I12) wherein each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl and each R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, and y is independently an integer from 0 to 6.

In another embodiment is a compound having the structure of Formulas (I9), (I10), (I11), and (I12) wherein B is

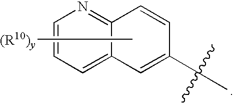

In a further embodiment, each R¹⁰ is independently hydrogen, substituted or unsubstituted aryl or a substituted or unsubstituted heteroaryl. In another embodiment, at least one of R¹⁰ is a substituted or unsubstituted heteroaryl having at least one N, S, or O atom. In yet another embodiment, at least one of R¹⁰ is a substituted heteroaryl having at least two nitrogen atoms. In yet a further embodiment, at least one of R¹⁰ is a substituted pyrazole group. In another embodiment, the pyrazole group is substituted with hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, the pyrazole group is substituted with a C₁-C₆ alkyl group. In a further embodiment, the pyrazole group is substituted with a C₁-C₃ alkyl group. In a further embodiment, the pyrazole group is substituted with methyl, ethyl, n-propyl, iso-propyl, n-butyl, iso-butyl, t-butyl, pentyl and hexyl. In yet a further embodiment, the pyrazole group is substituted with methyl. In one embodiment, each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, and SH. In another embodiment, each R¹⁰ is independently hydrogen and C₁-C₆ alkyl. In yet another embodiment, each R¹⁰ is independently hydrogen, methyl, ethyl, n-propyl, iso-propyl, n-butyl, iso-butyl, t-butyl, pentyl, and hexyl. In one embodiment each R¹⁰ is independently hydrogen and halogen. In another embodiment each R¹⁰ is independently hydrogen, fluorine, chlorine, and bromine.

In another embodiment is a compound having the formula:

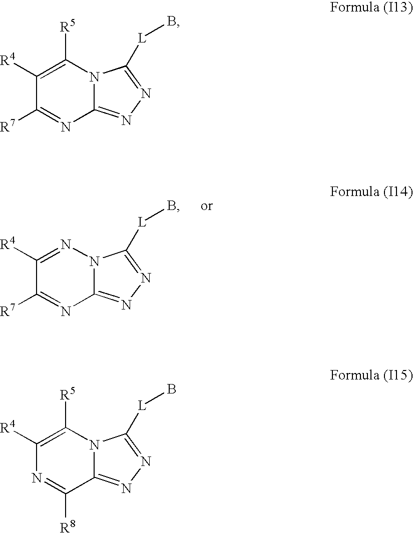

wherein:

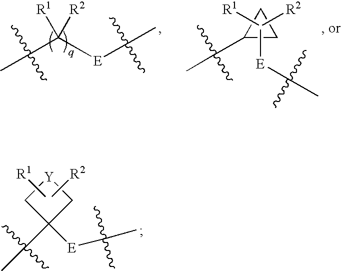

L is

wherein:

E is independently a direct bond, O, C═O, S(O)ᵤ, or NR³;

Y is CH₂, CF₂, O, C(O)—, OC(O)—, NR³, or S(O)ᵤ;

q is an integer from 0 to 4;

u is an integer from 0 to 2;

R⁴, R⁵, R⁷, and R⁸ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹,

wherein each j is independently an integer from 0 to 6; and m is independently an integer from 0 to 2;

R⁴ and R⁵ optionally form substituted or unsubstituted cycloalkyl, substituted or unsubstituted heterocycloalkyl, substituted or unsubstituted aryl, or substituted or unsubstituted heteroaryl, or

R⁴ and R⁷ optionally form substituted or unsubstituted cycloalkyl, substituted or unsubstituted heterocycloalkyl, substituted or unsubstituted aryl, or substituted or unsubstituted heteroaryl, or

R⁷ and R⁸ optionally form substituted or unsubstituted cycloalkyl, substituted or unsubstituted heterocycloalkyl, substituted or unsubstituted aryl, or substituted or unsubstituted heteroaryl;

R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼ NR¹⁵S(O)₂R¹⁶, wherein each j is independently an integer from 0 to 6, and m is independently an integer from 0 to 2;

R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;
B is selected from:

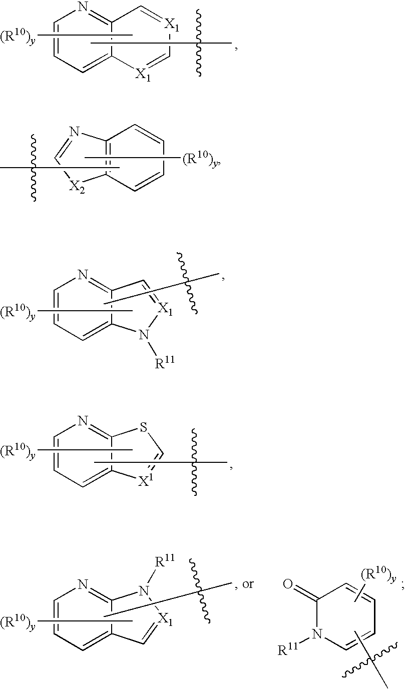

wherein:

X₁ is independently N or C; and

X₂ is N(R¹¹), S, or O;

R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²C(O)OR²², (CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

y is independently an integer from 0 to 5;

R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, (CH)ⱼS(O)R²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;

R¹², R¹⁷ and R²² are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;

R¹³, R¹⁴, R¹⁵, R¹⁶, R¹⁸, R¹⁹, R²⁰, R²¹, R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or

R¹³ and R¹⁴, R¹⁸ and R¹⁹, and R²³ and R²⁴ together with the N atom to which they are attached, each independently form substituted or unsubstituted heterocycloalkyl, or substituted or unsubstituted heteroaryl, or

R¹² and R¹⁵, R¹⁷ and R²⁰, and R²² and R²⁵ together with the N atom to which they are attached, each independently form substituted or unsubstituted heterocycloalkyl, or substituted or unsubstituted heteroaryl, or

R¹³ and R¹⁵ or R¹⁴ and R¹⁵, R¹⁸ and R²⁰ or R¹⁹ and R²⁰, and R²³ and R²⁵ or R²⁴ and R²⁵ together with the N atom to which they are attached, each independently form substituted or unsubstituted heterocycloalkyl, or substituted or unsubstituted heteroaryl, or

R¹⁵ and R¹⁶, R²⁰ and R²¹, and R²⁵ and R²⁶ together with the N atom to which they are attached, each independently form substituted or unsubstituted heterocycloalkyl, or substituted or unsubstituted heteroaryl;

wherein any of the R¹, R², R³, R⁴, R⁵, R⁷, R⁸, R¹⁰, R¹¹, R¹², R¹³, R¹⁴, R¹⁵, R¹⁶, R¹⁷, R¹⁸, R¹⁹, R²⁰, R²¹, R²², R²³, and R²⁴ groups are each optionally independently substituted with 1 to 3 groups, each group independently selected from hydrogen, halogen, hydroxyl, amino, aminomonoalkyl, aminodialkyl, cyano, morpholine, nitro, difluoromethyl, trifluoromethyl, oxo, alkyl, —O-alkyl, and —S-alkyl;

with the proviso that when the core structure of the compound having a structure of Formula (I14) is [1,2,4]triazolo-[4,3-b][1,2,4]triazine, then R¹⁰ is not hydrogen, halogen, nitro, cyano, hydroxyl, substituted or unsubstituted alkyl, perfluoroalkyl, —(CH₂)ⱼOR²², —(CH₂)ⱼC(O)R²², —(CH₂)ⱼC(O)OR²², —(CH₂)ⱼNR²³R²⁴, —(CH₂)ⱼS(O)ₘR²⁶(CH₂)ⱼC(O)NR²³R²⁴, —(CH₂)ⱼS(O)₂NR²³R²⁴; or when the core structure of the compound having a structure of Formula (I13) is [1,2,4]triazolo[4,3-a]pyrimidine then all R¹⁰ are not H; or an enantiomer, diastereomer, racemate, or pharmaceutically acceptable salt, or solvate thereof.

In one embodiment is a compound having the structure of Formulas (I13), (I14), and (I15) wherein L is

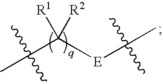

wherein E is independently a direct bond or S, and q is 0 or 1. In another embodiment, E is a direct bond and q is 1. In a further embodiment, E is S and q is 0. In yet a further embodiment, R¹ and R² are each H. In another embodiment, R¹ and R² are independently H or C₁-C₃ alkyl.

In one embodiment is a compound having the structure of Formulas (I13), (I14) and (I15) wherein R⁴, R⁵, R⁷, and R⁸ are each independently hydrogen, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, or substituted or unsubstituted heteroarylalkyl.

In another embodiment, R⁴, R⁵, R⁷, and R⁸ are each independently hydrogen, substituted or unsubstituted alkyl, substituted or unsubstituted cycloalkyl, substituted or unsubstituted heterocycloalkyl, substituted or unsubstituted aryl, or substituted or unsubstituted heteroaryl.

In a further embodiment, R⁵, R⁷, and R⁸ are each independently hydrogen and each R⁴ is independently substituted or unsubstituted aryl or substituted or unsubstituted heteroaryl. In one embodiment, the substituted or unsubstituted heteroaryl group is thiophenyl, furanyl, pyranyl, isobenzofuranyl, chromenyl, xanthenyl, phenoxathiinyl, pyrrolyl, 2H-pyrrolyl, imidazolyl, isothiazolyl, isoxazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinoxalinyl, quinzolinyl, cinnolinyl, pterdinyl, 4aH-carbazolyl, carbazolyl, carbolinyl, phenanthridinyl, acridinyl, perimidinyl, phenanthrolinyl, phenazinyl, phenarsazinyl, phenothiazinyl, furazanyl, or phenoxazinyl.

In one embodiment, the substituted or unsubstituted heteroaryl group is pyridyl. In another embodiment, the pyridyl group is substituted with C₁-C₆ alkyl, halogen, cyano, hydroxyl, perfluoroalkyl, or SH. In another embodiment, the pyridyl group is substituted with methyl, ethyl, n-propyl, isopropyl, n-butyl, isobutyl, t-butyl, pentyl, and hexyl.

In another embodiment, the substituted or unsubstituted heteroaryl group is pyrazolyl. In yet another embodiment, the pyrazole group is substituted with C₁-C₆ alkyl, halogen, cyano, hydroxyl, perfluoroalkyl, or SH. In another embodiment, the pyrazole group is substituted with C₁-C₆ alkyl. In another embodiment, the pyrazole group is substituted with methyl, ethyl, n-propyl, isopropyl, n-butyl, isobutyl, t-butyl, pentyl, and hexyl. In a further embodiment, the pyrazole group is substituted with methyl.

In a further embodiment R⁵, R⁷, and R⁸ are each independently hydrogen and each R⁴ is independently substituted or unsubstituted phenyl. In yet a further embodiment, the phenyl group is substituted with C₁-C₆ alkyl, halogen, cyano, hydroxyl, perfluoroalkyl, or SH. In another embodiment, the C₁-C₆ alkyl group is methyl, ethyl, n-propyl, isopropyl, n-butyl, isobutyl, t-butyl, pentyl, and hexyl.

In one embodiment is a compound having the structure of Formulas (I13), (I14), and (I15) wherein R⁴, R⁵, R⁷, and R⁸ are each independently hydrogen or substituted or unsubstituted C₁-C₆ alkyl. In another embodiment, R⁴, R⁵, R⁷, and R⁸ are each independently hydrogen, methyl, ethyl, n-propyl, isopropyl, n-butyl, isobutyl, t-butyl, pentyl, and hexyl. In a further embodiment, R⁴, R⁵, R⁷, and R⁸ are each independently hydrogen or methyl. In yet a further embodiment, R⁴, R⁵, R⁷, and R⁸ are each independently hydrogen or ethyl.

In one embodiment is a compound having the structure of Formulas (I13), (I14), and (I15) wherein R⁴ is hydrogen.

In another embodiment, R⁴, R⁵, R⁷, and R⁸ are each independently hydrogen, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, or substituted or unsubstituted heterocycloalkyl. In a further embodiment R⁵, R⁷, and R⁸ are each independently hydrogen, and each R⁴ is independently substituted or unsubstituted alkylaminoalkyl or alkylaminocycloalkyl. In yet a further embodiment, the substituted or unsubstituted alkylaminoalkyl group is (CH₂)ₙ(CH)NR^{a}(CH₂)ₘR^{b} wherein R^{a} is H or C₁-C₆ alkyl, and R^{b} is H, C₁-C₆ alkyl, halogen, hydroxy, NH₂, or SH, and n+m=0-4. In another embodiment R⁵, R⁷, and R⁸ are each independently hydrogen and each R⁴ is independently —(CH)NR^{a}R^{b} wherein R^{a} and R^{b} are independently H or C₁-C₆ alkyl. In a further embodiment R⁵, R⁷, and R⁸ are each independently hydrogen and each R⁴ is independently —(CH₂)ₙNH(CH₂)ₘC₁-C₈ cycloalkyl where n+m=0-4. In one embodiment R⁵, R⁷, and R⁸ are each independently hydrogen and each R⁴ is independently (CH₂)ₙNH(CH₂)ₘC₁-C₆ heterocycloalkyl where n+m=0-4. In another embodiment R⁵, R⁷, and R⁸ are each independently hydrogen and each R⁴ is independently (CH₂)ₙNR^{c}(CH₂)ₘR^{d} wherein R^{c} is hydrogen or C₁-C₃ alkyl, R^{d} is hydrogen, halogen, C₁-C₃ alkyl, and CF₃, and n+m=0-4. In a further embodiment R⁵, R⁷, and R⁸ are each independently hydrogen and each R⁴ is independently C₁-C₆ cycloalkyl. In yet a further embodiment R⁵, R⁷, and R⁸ are each independently hydrogen and each R⁴ is independently C₁-C₆ heterocycloalkyl. In one embodiment R⁵, R⁷, and R⁸ are each independently hydrogen and each R⁴ is independently —(CH)NH₂(CH₂)ₘR^{e} wherein R^{e} is hydrogen, C₁-C₆ cycloalkyl, aryl, C₁-C₃ alkyl optionally substituted with halogen or hydroxy, and m is 0-3. In another embodiment R⁵, R⁷, and R⁸ are each independently hydrogen and each R⁴ is independently (CH₂)ₙC₁-C₆ heteroalkyl wherein n is 0-6.

In one embodiment is a compound having the structure of Formulas (I13), (I14), and (I15) wherein B is selected from:

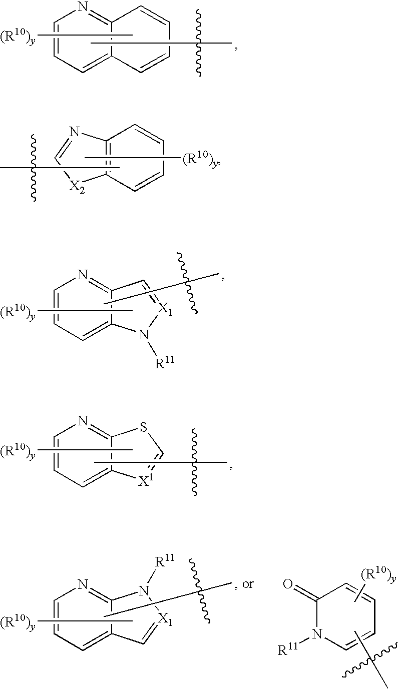

wherein X₁ is N or C; and X₂ is N(R¹¹), S, or O.

In another embodiment is a compound having the structure of Formulas (I13), (I14), and (I15) wherein each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl and each R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, and y is independently an integer from 0 to 6.

In another embodiment is a compound having the structure of Formulas (I13), (I14), and (I15) wherein B is

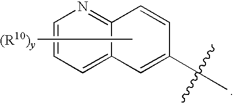

In a further embodiment, each R¹⁰ is independently hydrogen, substituted or unsubstituted aryl or a substituted or unsubstituted heteroaryl. In another embodiment, at least one of R¹⁰ is a substituted or unsubstituted heteroaryl having at least one N, S, or O atom. In yet another embodiment, at least one of R¹⁰ is a substituted heteroaryl having at least two nitrogen atoms. In yet a further embodiment, at least one of R¹⁰ is a substituted pyrazole group. In another embodiment, the pyrazole group is substituted with hydrogen, halogen, nitro, cyano, hydroxyl, substituted or unsubstituted alkyl, substituted or unsubstituted cycloalkyl, perfluoroalkyl, substituted or unsubstituted alkylaminoalkyl or amine. In another embodiment, the pyrazole group is substituted with a C₁-C₆ alkyl group. In a further embodiment, the pyrazole group is substituted with a C₁-C₃ alkyl group. In a further embodiment, the pyrazole group is substituted with methyl, ethyl, n-propyl, iso-propyl, n-butyl, iso-butyl, t-butyl, pentyl and hexyl. In yet a further embodiment, the pyrazole group is substituted with methyl.

In one embodiment, each R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, and SH. In another embodiment, each R¹⁰ is independently hydrogen and C₁-C₆ alkyl. In yet another embodiment, each R¹⁰ is independently hydrogen, methyl, ethyl, n-propyl, iso-propyl, n-butyl, iso-butyl, t-butyl, pentyl, and hexyl. In one embodiment each R¹⁰ is independently hydrogen and halogen. In another embodiment each R¹⁰ is independently hydrogen, fluorine, chlorine, and bromine.

In another embodiment is a compound having the structure:

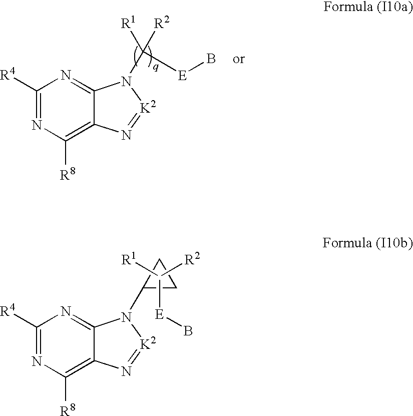

or an enantiomer, diastereomer, racemate, or pharmaceutically acceptable salt, or solvate thereof, wherein the substituents are as defined herein.

In another embodiment is a compound having the structure:

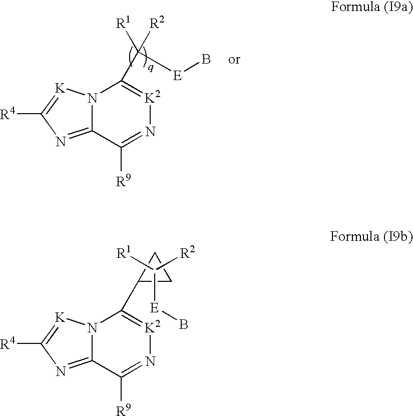

or an enantiomer, diastereomer, racemate, or pharmaceutically acceptable salt, or solvate thereof, wherein the substituents are as defined herein.

In another embodiment is a compound having the structure:

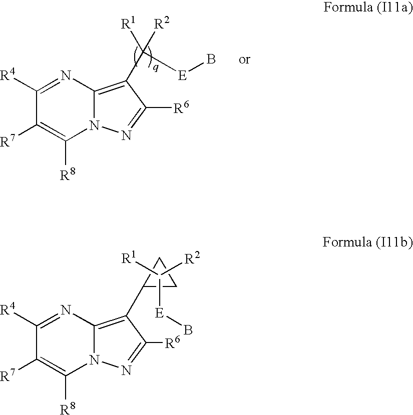

or an enantiomer, diastereomer, racemate, or pharmaceutically acceptable salt, or solvate thereof, wherein the substituents are as defined herein.

In yet another embodiment is a compound having the structure:

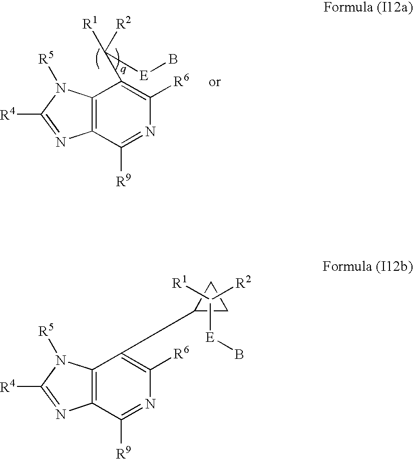

or an enantiomer, diastereomer, racemate, or pharmaceutically acceptable salt, or solvate thereof, wherein the substituents are as defined herein.

In a further embodiment is a compound having the structure:

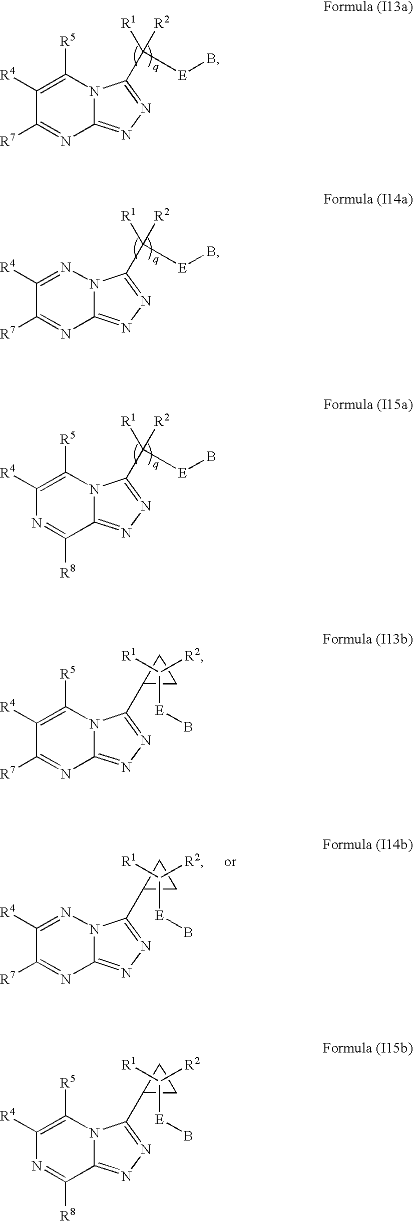

or an enantiomer, diastereomer, racemate, or pharmaceutically acceptable salt, or solvate thereof, wherein the substituents are as defined herein.

In one embodiment B is

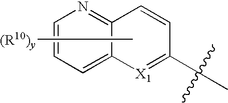

and R¹⁰ is independently a substituted or unsubstituted pyrazolyl.

In a further embodiment is a compound described herein, such as by way of example only, a compound of Formula (I) wherein R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, (CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2.

In yet a further embodiment is a compound described herein, such as by way of example only, a compound of Formula (I) wherein R¹⁰ is independently hydrogen or halogen.

In one embodiment is a compound described herein, such as by way of example only, a compound of Formula (I) wherein R⁴ is selected from the group consisting of hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2.

In a further embodiment is a compound described herein, such as by way of example only, a compound of Formula (I) wherein R⁴ is selected from a group consisting of a substituted or unsubstituted alkyl, perfluoroalkyl, substituted or unsubstituted alkylaminoalkyl, substituted or unsubstituted aminocycloalkyl, substituted or unsubstituted aminoalkylenecycloalkyl, substituted or unsubstituted heterocycloalkyl, substituted or unsubstituted heteroalkyl, substituted or unsubstituted alkylheterocycloalkyl, substituted or unsubstituted heteroaryl, substituted or unsubstituted aryl, and —(CH₂)ⱼNR¹⁸R¹⁹.

In another embodiment is a compound described herein, such as by way of example only, a compound of Formula (I) wherein R⁴ is a substituted or unsubstituted heteroaryl.

In yet a further embodiment is a compound described herein, such as by way of example only, a compound of Formula (I) wherein R⁴ is a substituted or unsubstituted pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, phenyl, isoxazolyl, and oxazolyl group.

In another embodiment is a compound described herein, such as by way of example only, a compound of Formula (I) wherein R⁴ is a substituted or unsubstituted pyridinyl group.

In one embodiment is a compound described herein, such as by way of example only, a compound of Formula (I) wherein R⁴ is a substituted or unsubstituted alkyl. In another embodiment is a compound of Formula (I) wherein the alkylaminoalkyl is substituted with an optionally substituted amino group. In a further embodiment is a compound of Formula (I) wherein R⁴ is a substituted or unsubstituted heterocycloalkyl. In one embodiment is a compound of Formula (I) wherein R⁴ is a substituted or unsubstituted alkylaminoalkyl. In a further embodiment is a compound of Formula (I) wherein alkylaminoalkyl is substituted with a halogen or a hydroxy group.

In another embodiment is a compound of Formula (I) wherein at least one of R¹⁰ is independently substituted or unsubstituted 2H-pyrrolyl, substituted or unsubstituted 2-pyrrolinyl, substituted or unsubstituted 3-pyrrolinyl, substituted or unsubstituted pyrrolidinyl, substituted or unsubstituted dioxolanyl, substituted or unsubstituted 2-imidazolinyl, substituted or unsubstituted imidazolidinyl, substituted or unsubstituted 2-pyrazolinyl, substituted or unsubstituted pyrazolidinyl, substituted or unsubstituted piperidinyl, substituted or unsubstituted morpholinyl, substituted or unsubstituted thiomorpholinyl, substituted or unsubstituted piperazinyl, substituted or unsubstituted phenyl, substituted or unsubstituted phenoxy, substituted or unsubstituted naphthyl, substituted or unsubstituted biphenyl, substituted or unsubstituted thiophenyl, substituted or unsubstituted pyrrolyl, substituted or unsubstituted pyrazolyl, substituted or unsubstituted imidazolyl, substituted or unsubstituted pyrazinyl, substituted or unsubstituted oxazolyl, substituted or unsubstituted isoxazolyl, substituted or unsubstituted thiazolyl, substituted or unsubstituted furyl, substituted or unsubstituted thienyl, substituted or unsubstituted pyridinyl, substituted or unsubstituted O-pyridinyl, substituted or unsubstituted pyrimidyl, substituted or unsubstituted benzothiazolyl, substituted or unsubstituted purinyl, substituted or unsubstituted benzimidazolyl, substituted or unsubstituted indolyl, substituted or unsubstituted isoquinolinyl, substituted or unsubstituted quinoxalinyl, substituted or unsubstituted quinolinyl, substituted or unsubstituted benzooxazolyl, substituted or unsubstituted [1,5]naphthyridinyl, substituted or unsubstituted pyrido[3,2-d]pyrmidinyl, substituted or unsubstituted [1,7]naphthyridinyl, substituted or unsubstituted 1H-pyrrolo[2,3-b]pyridinyl, substituted or unsubstituted pyrazolo[4,3-b]pyridinyl, substituted or unsubstituted pyrrolo[2,3-b]pyridinyl, substituted or unsubstituted thieno[2,3-b]pyridinyl, substituted or unsubstituted thiazolo[5,4-b]pyridinyl, substituted or unsubstituted pyridinyl-2-one, substituted or unsubstituted imidazo[1,2-b]pyridazinyl, substituted or unsubstituted pyrazolo[1,5-a]pyrimidinyl, substituted or unsubstituted pyridazinyl-3-one, substituted or unsubstituted imidazo[2,1-b][1,3,4]thiaciazolyl, substituted or unsubstituted iridazo[2,1-b]thiazolyl, substituted or unsubstituted isothiazolyl, substituted or unsubstituted triazolyl, or substituted or unsubstituted imidazo[4,5-b]pyridinyl.

In some embodiments are compounds of Formula (I) wherein at least one of R¹⁰ is independently substituted or unsubstituted pyrazolyl.

In one embodiment is a compound of Formula (I) wherein at least one of R¹⁰ is independently a substituted or unsubstituted pyridinyl.

The compounds presented herein contain substituents of various moieties. It is recognized that one of ordinary skill in the art could interchange substituents of compounds belonging to one formula with substituents of compounds of another formula.

### Methods of Inhibiting Kinases

In another aspect, the present disclosure provides methods of modulating protein kinase activity using the heterocyclic kinase modulators described herein. The term “modulating kinase activity,” as used herein, means that the activity of the protein kinase is increased or decreased when contacted with a heterocyclic kinase modulator described herein relative to the activity in the absence of the heterocyclic kinase modulator. Therefore, the present disclosure provides a method of modulating protein kinase activity by contacting the protein kinase with a heterocyclic kinase modulator described herein.

In one embodiment, the heterocyclic kinase modulator inhibits kinase activity. The term “inhibit,” as used herein in reference to kinase activity, means that the kinase activity is decreased when contacted with a heterocyclic kinase modulator relative to the activity in the absence of the heterocyclic kinase modulator. Therefore, the present disclosure further provides a method of inhibiting protein kinase activity by contacting the protein kinase with a heterocyclic kinase modulator described herein.

In certain embodiments, the protein kinase is a protein tyrosine kinase. A protein tyrosine kinase, as used herein, refers to an enzyme that catalyzes the phosphorylation of tyrosine residues in proteins with phosphate donors (e.g. a nucleotide phosphate donor such as ATP). Protein tyrosine kinases include, for example, Abelson tyrosine kinases (“Abl”) (e.g. c-Abl and v-Abl), Ron receptor tyrosine kinases (“RON”), Met receptor tyrosine kinases (“MET”), Fms-like tyrosine kinases (“FLT”) (e.g. FLT3), src-family tyrosine kinases (e.g. lyn, CSK), and p21-activated kinase-4 (“PAK”), FLT3, aurora-A kinases, B-lymphoid tyrosine kinases (“Blk”), cyclin-dependent kinases (“CDK”) (e.g. CDK1 and CDK5), src-family related protein tyrosine kinases (e.g. Fyn kinase), glycogen synthase kinases (“GSK”) (e.g. GSK3a and GSK30), lymphocyte protein tyrosine kinases (“Lck”), ribosomal S6 kinases (e.g. Rsk1, Rsk2, and Rsk3), sperm tyrosine kinases (e.g. Yes), and subtypes and homologs thereof exhibiting tyrosine kinase activity. In certain embodiments, the protein tyrosine kinase is Abl, RON, MET, PAK, or FLT3. In other embodiments, the protein tyrosine kinase is a FLT3 or Abl family member.

In another embodiment, the kinase is a mutant kinase, such as a mutant Abl kinase or FLT3 kinase. Useful mutant Abl kinases include, for example, Bcr-Abl and Abl kinases having one of more of the following mutations: Glu255Lys, Thr315Ile, Tyr293Phe, or Met351Thr. In some embodiments, the mutant Abl kinase has a Y393F mutation or a T315I mutation. In another embodiment, the mutant Abl kinase has a Thr315Ile mutation.

In one aspect are methods for modulating the activity of a protein tyrosine kinase comprising contacting the protein tyrosine kinase with a compound of Formula (I).

In one embodiment is a method for modulating the activity of Met receptor tyrosine kinase comprising contacting Met receptor tyrosine kinase with a compound of Formula (I).

In one aspect is a method for treating a disease, disorder, or condition ameliorated by the inhibition of a tyrosine kinase comprising administering to a subject in need of treatment a therapeutically effective amount of a compound of Formula (I).

In one embodiment, the disease, disorder, or condition is Listeria invasion, Osteolysis associated with multiple myeloma, Malaria infection, diabetic retinopathy, psoriasis, and arthritis.

In one embodiment, the disclosure provides methods for modulating the activity of a protein kinase comprising contacting the protein kinase with a compound of Formula (I), wherein protein kinase is Abelson tyrosine kinase, Ron receptor tyrosine kinase, Met receptor tyrosine kinase, Fms-like tyrosine kinase-3, or p21-activated kinase-4.

In some embodiments, the kinase is homologous to a known kinase (also referred to herein as a “homologous kinase”). In some embodiments, compounds and compositions useful for inhibiting the biological activity of homologous kinases are initially screened, for example, in binding assays. Homologous enzymes comprise an amino acid sequence of the same length that is at least about 50%, at least about 60%, at least about 70%, at least about 80%, or at least about 90% identical to the amino acid sequence of full length known kinase, or about 70%, about 80%, or about 90% homology to the known kinase active domains. In further embodiments, homology is determined using, for example, a PSI BLAST search, such as, but not limited to that described in Altschul, et al., Nuc. Acids Rec. 25:3389-3402 (1997). In other embodiments, at least about 50%, or at least about 70% of the sequence is aligned in this analysis. Other tools for performing the alignment include, for example, DbClustal and ESPript, which in some embodiments is used to generate the PostScript version of the alignment. In further embodiments, homologs, for example, have a BLAST E-value of 1×10⁻⁶ over at least 100 amino acids with FLT3, Abl, or another known kinase, or any functional domain of FLT3, Abl, or another known kinase.

In other embodiments, homology is also determined by comparing the active site binding pocket of the enzyme with the active site binding pockets of a known kinase. For example, in homologous enzymes, at least about 50%, about 60%, about 70%, about 80%, or about 90% of the amino acids of the molecule or homolog have amino acid structural coordinates of a domain comparable in size to the kinase domain that have a root mean square deviation of the alpha carbon atoms of up to about 1.5 Å, about 1.25 Å, about 1 Å, about 0.75 Å, about 0.5 Å, and or about 0.25 Å.

The compounds and compositions of the present disclosure are useful for inhibiting kinase activity and also for inhibiting other enzymes that bind ATP. They are thus useful in some embodiments for the treatment of diseases and disorders that are alleviated by inhibiting such ATP-binding enzyme activity. Methods of determining such ATP binding enzymes include those discussed herein relating to selecting homologous enzymes, and by the use of the database PROSITE, where enzymes containing signatures, sequence patterns, motifs, or profiles of protein families or domains are identified.

In some embodiments, the compounds of the present disclosure, and their derivatives, are also used as kinase-binding agents. In further embodiments, are binding agents, such as compounds and derivatives described herein which are bound to a stable resin as a tethered substrate for affinity chromatography applications. In other embodiments, the compounds described herein, and their derivatives, are also modified (e.g., radiolabeled or affinity labeled, etc.) in order to utilize them in the investigation of enzyme or polypeptide characterization, structure, and/or function.

In one embodiment, the heterocyclic kinase modulator of the present disclosure is a kinase inhibitor. In some embodiments, the kinase inhibitor has an IC₅₀ of inhibition constant (Kᵢ) of less than about 1 micromolar. In another embodiment, the kinase inhibitor has an IC₅₀ or inhibition constant (Kᵢ) of less than about 500 micromolar.

In another embodiment, the kinase inhibitor has an IC₅₀ or Kᵢ of less than about 10 micromolar. In another embodiment, the kinase inhibitor has an IC₅₀ or Kᵢ of less than about 1 micromolar. In another embodiment, the kinase inhibitor has an IC₅₀ or Kᵢ of less than about 500 nanomolar. In another embodiment, the kinase inhibitor has an IC₅₀ or Kᵢ of less than about 10 nanomolar.

In another embodiment, the kinase inhibitor has an IC₅₀ or Kᵢ of less than about 1 nanomolar. In another embodiment, the kinase inhibitor has an IC₅₀ or inhibition constant (Kᵢ) of between about 1 micromolar and about 500 micromolar. In another embodiment, the kinase inhibitor has an IC₅₀ or Kᵢ of between about 500 micromolar and about 10 micromolar.

In another embodiment, the kinase inhibitor has an IC₅₀ or Kᵢ of between about 400 micromolar and about 100 micromolar. In another embodiment, the kinase inhibitor has an IC₅₀ or Kᵢ of between about 300 micromolar and about 200 micromolar.

In another embodiment, the kinase inhibitor has an IC₅₀ or Kᵢ of between about 10 micromolar and about 1 micromolar. In another embodiment, the kinase inhibitor has an IC₅₀ or Kᵢ of between about 1 micromolar and about 500 nanomolar. In another embodiment, the kinase inhibitor has an IC₅₀ or K; of between about 900 nanomolar and about 500 nanomolar.

In another embodiment, the kinase inhibitor has an IC₅₀ or Kᵢ of between about 750 nanomolar and about 500 nanomolar. In another embodiment, the kinase inhibitor has an IC₅₀ or Kᵢ of between about 500 nanomolar and about 10 nanomolar. In another embodiment, the kinase inhibitor has an IC₅₀ or Kᵢ of between about 500 nanomolar and about 100 nanomolar.

In another embodiment, the kinase inhibitor has an IC₅₀ or K; of between about 300 nanomolar and about 200 nanomolar. In another embodiment, the kinase inhibitor has an IC₅₀ or K; of between about 10 nanomolar and about 1 nanomolar.

### Methods of Treatment

In another aspect, the present disclosure provides methods of treating a disease mediated by kinase activity (kinase-mediated disease or disorder) in an organism (e.g. mammals, such as humans). In some embodiments, “kinase-mediated” or “kinase-associated” diseases includes diseases in which the disease or symptom is alleviated by inhibiting kinase activity (e.g. where the kinase is involved in signaling, mediation, modulation, or regulation of the disease process). By “diseases” is meant diseases, or disease symptoms.

The disclosure provides methods for treating cancer in a human patient in need of such treatment, the method comprising administering to the patient a therapeutically effective amount of a compound of Formula (I).

Examples of kinase associated diseases include cancer (e.g. leukemia, tumors, and metastases), allergy, asthma, inflammation (e.g. inflammatory airways disease), obstructive airways disease, autoimmune diseases, metabolic diseases, infection (e.g. bacterial, viral, yeast, fungal), CNS diseases, brain tumors, degenerative neural diseases, cardiovascular diseases, and diseases associated with angiogenesis, neovascularization, and vasculogenesis. In one embodiment, the compounds are useful for treating cancer, including leukemia, and other diseases or disorders involving abnormal cell proliferation, myeloproliferative disorders, hematological disorders, asthma, inflammatory diseases or obesity.

In further embodiments, examples of cancers treated with the compounds of the present disclosure include bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia (e.g. myeloid, chronic myeloid, acute lymphoblastic, chronic lymphoblastic, Hodgkins, and other leukemias and hematological cancers), liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

Other specific examples of diseases or disorders for which treatment by the compounds or compositions described herein are useful for treatment or prevention include, but are not limited to transplant rejection (for example, kidney, liver, heart, lung, islet cells, pancreas, bone marrow, cornea, small bowel, skin allografts or xenografts and other transplants), graft vs. host disease, osteoarthritis, rheumatoid arthritis, multiple sclerosis, diabetes, diabetic retinopathy, inflammatory bowel disease (for example, Crohn's disease, ulcerative colitis, and other bowel diseases), renal disease, cachexia, septic shock, lupus, myasthenia gravis, psoriasis, dermatitis, eczema, seborrhea, Alzheimer's disease, Parkinson's disease, stem cell protection during chemotherapy, ex vivo selection or ex vivo purging for autologous or allergenic bone marrow transplantation, ocular disease, retinopathies (for example, macular degeneration, diabetic retinopathy, and other retinopathies), corneal disease, glaucoma, infections (for example bacterial, viral, or fungal), heart disease, including, but not limited to, restenosis.

### Assays

In other embodiments, the compounds of the present disclosure are easily assayed to determine their ability to modulate protein kinases, bind protein kinases, and/or prevent cell growth or proliferation. Some examples of useful assays are presented below.

### Kinase Inhibition and Binding Assays

In some embodiments, inhibition of various kinases is measured by methods such as the various methods presented herein, and those discussed in the Upstate KinaseProfiler Assay Protocols June 2003 publication.

For example, where in vitro assays are performed, the kinase is typically diluted to the appropriate concentration to form kinase solution. A kinase substrate and phosphate donor, such as ATP, is added to the kinase solution. The kinase is allowed to transfer a phosphate to the kinase substrate to form phosphorylated substrate. In some embodiments, the formation of a phosphorylated substrate is detected directly by any appropriate means, such as radioactivity (e.g. [γ-³²P-ATP]), or the use of detectable secondary antibodies (e.g. ELISA). Alternatively, the formation of a phosphorylated substrate is detected using any appropriate technique, such as the detection of ATP concentration (e.g. Kinase-Glo® assay system (Promega)). Kinase inhibitors are identified by detecting the formation of a phosphorylated substrate in the presence and absence of a test compound (see Examples section below).

In some embodiments, the ability of the compound to inhibit a kinase in a cell is assayed using methods described herein. For example, in some other embodiments, cells containing a kinase are contacted with an activating agent (such as a growth factor) that activates the kinase. In further embodiments, the amount of intracellular phosphorylated substrate formed in the absence and the presence of the test compound is determined by lysing the cells and detecting the presence phosphorylated substrate by any appropriate method (e.g. ELISA). Where the amount of phosphorylated substrate produced in the presence of the test compound is decreased relative to the amount produced in the absence of the test compound, kinase inhibition is indicated. More detailed cellular kinase assays are discussed in the Examples section below.

In some embodiments are methods to measure the binding of a compound to a kinase. For example, in some other embodiments, a test kit manufactured by Discoverx (Fremont, Calif.), ED-Staurosporine NSIP™ Enzyme Binding Assay Kit is used. In other embodiments, kinase activity is assayed as disclosed in U.S. Pat. No. 6,589,950, of which the assay method described therein is incorporated by reference.

In further embodiments, kinase inhibitors are selected from the compounds of the present disclosure through protein crystallographic screening, as described in, for example Antonysamy, et al., PCT Publication No. WO03087816A1, which is incorporated herein by reference for this purpose.

In other embodiments, the compounds of the present disclosure are computationally screened to assay and visualize their ability to bind to and/or inhibit various kinases. In other embodiments, the structure is computationally screened with a plurality of compounds described herein to determine their ability to bind to a kinase at various sites. In yet other embodiments, such compounds are used as targets or leads in medicinal chemistry efforts to identify, for example, inhibitors of potential therapeutic importance. The three dimensional structures of such compounds are superimposed on a three dimensional representation of kinases or an active site or binding pocket thereof to assess whether the compound fits spatially into the representation and hence the protein. In this screening, the quality of fit of such entities or compounds to the binding pocket is judged either by shape complementarity or by estimated interaction energy.

The screening of compounds of the present disclosure that bind to and/or modulate kinases (e.g. inhibit or activate kinases) generally involves consideration of two factors. First, the compound must be capable of physically and structurally associating, either covalently or non-covalently with kinases. For example, in some embodiments, covalent interactions are important for designing irreversible or suicide inhibitors of a protein. Non-covalent molecular interactions important in the association of kinases with the compound include hydrogen bonding, ionic interactions, van der Waals, and hydrophobic interactions. Second, the compound must be able to assume a conformation and orientation in relation to the binding pocket, which allows it to associate with kinases. Although certain portions of the compound will not directly participate in this association with kinases, in some embodiments, those portions will still influence the overall conformation of the molecule and will have a significant impact on potency. Conformational requirements include the overall three-dimensional structure and orientation of the chemical group or compound in relation to all or a portion of the binding pocket, or the spacing between functional groups of a compound comprising several chemical groups that directly interact with kinases.

Docking programs described herein, such as, for example, DOCK, or GOLD, are used to identify compounds that bind to the active site and/or binding pocket. In further embodiments, compounds are screened against more than one binding pocket of the protein structure, or more than one set of coordinates for the same protein, taking into account different molecular dynamic conformations of the protein. In other embodiments, consensus scoring is used to identify the compounds that are the best fit for the protein. In yet other embodiments, data obtained from more than one protein molecule structure are also scored according to the methods described in Klingler et al., U.S. Utility Application, filed May 3, 2002, entitled “Computer Systems and Methods for Virtual Screening of Compounds.” Compounds having the best fit are then obtained from the producer of the chemical library, or synthesized, and used in binding assays and bioassays.

In further embodiments, computer modeling techniques are used to assess the potential modulating or binding effect of a chemical compound on kinases. In yet further embodiments, if computer modeling indicates a strong interaction, the molecule is synthesized and tested for its ability to bind to kinases and affect (by inhibiting or activating) its activity.

In other embodiments, modulating or other binding compounds of kinases is computationally evaluated by means of a series of steps in which chemical groups or fragments are screened and selected for their ability to associate with the individual binding pockets or other areas of kinases. In further embodiments, the process begins by visual inspection of, for example, the active site on the computer screen based on the kinases coordinates. Selected fragments or chemical groups are then positioned in a variety of orientations, or docked, within an individual binding pocket of kinases. In yet further embodiments, manual docking is accomplished using software such as Insight II (Accelrys, San Diego, Calif.) MOE (Chemical Computing Group, Inc., Montreal, Quebec, Canada); and SYBYL (Tripos, Inc., St. Louis, Mo., 1992), followed by energy minimization and/or molecular dynamics with standard molecular mechanics force fields, such as CHARMM, AMBER and C² MMFF (Merck Molecular Force Field; Accelrys, San Diego, Calif.). In other embodiments, further automated docking is accomplished by using programs such as DOCK; DOCK is available from University of California, San Francisco, Calif.); AUTODOCK; AUTODOCK is available from Scripps Research Institute, La Jolla, Calif.); GOLD; and FLEXX. Other appropriate programs are described in, for example, Halperin, et al.

In some embodiments, during selection of compounds by the above methods, the efficiency with which that compound binds to kinases is tested and optimized by computational evaluation. For example, in other embodiments, a compound that has been designed or selected to function as a kinases inhibitor occupies a volume not overlapping the volume occupied by the active site residues when the native substrate is bound. In some other embodiments, rearrangement of the main chains and the side chains occurs. In addition, the present disclosure provides for protein rearrangement upon binding, such as, for example, resulting in an induced fit. In other embodiments, an effective kinase inhibitor demonstrates a relatively small difference in energy between its bound and free states (i.e., it must have a small deformation energy of binding and/or low conformational strain upon binding). Thus, in some other embodiments, the most efficient kinase inhibitors are, for example, designed with a deformation energy of binding of not greater than about 10 kcal/mol, not greater than about 7 kcal/mol, not greater than about 5 kcal/mol, or not greater than about 2 kcal/mol. In further embodiments, kinase inhibitors interact with the protein in more than one conformation that is similar in overall binding energy. In those cases, the deformation energy of binding is taken to be the difference between the energy of the free compound and the average energy of the conformations observed when the inhibitor binds to the enzyme.

Specific computer software is available to evaluate compound deformation energy and electrostatic interaction. Examples of programs designed for such uses include: Gaussian 94, revision C (Frisch, Gaussian, Inc., Pittsburgh, Pa. ©1995); AMBER, version 7. (Kollman, University of California at San Francisco, ©2002); QUANTA/CHARMM (Accelrys, Inc., San Diego, Calif., ©1995); Insight II/Discover (Accelrys, Inc., San Diego, Calif., ©1995); Delphi (Accelrys, Inc., San Diego, Calif., ©1995); and AMSOL (University of Minnesota) (Quantum Chemistry Program Exchange, Indiana University). These programs are implemented, for instance, using a computer workstation, for example, a LINUX, SGI or Sun workstation. The present disclosure is not limited to these hardware systems and software packages and includes other systems and packages employed for such uses.

In some embodiments, are kinase protein expression using methods disclosed herein. In further embodiments, the native and mutated kinase polypeptides described herein are chemically synthesized in whole or part using techniques that are described herein (see, e.g., Creighton, Proteins: Structures and Molecular Principles, W.H. Freeman & Co., NY, 1983).

In other embodiments, gene expression systems are used for the synthesis of native and mutated polypeptides. Expression vectors containing the native or mutated polypeptide coding sequence and appropriate transcriptional/translational control signals, are constructed. These methods include in vitro recombinant DNA techniques, synthetic techniques and in vivo recombination/genetic recombination. See, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

In other embodiments, host-expression vector systems are used to express kinase. These include, but are not limited to, microorganisms such as bacteria transformed with recombinant bacteriophage DNA, plasmid DNA or cosmid DNA expression vectors containing the coding sequence; yeast transformed with recombinant yeast expression vectors containing the coding sequence; insect cell systems infected with recombinant virus expression vectors (e.g., baculovirus) containing the coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence; or animal cell systems. In further embodiments, the protein is expressed in human gene therapy systems, including, for example, expressing the protein to augment the amount of the protein in an individual, or to express an engineered therapeutic protein. The expression elements of these systems vary in their strength and specificities.

Specifically designed vectors allow the shuttling of DNA between hosts such as bacteria-yeast or bacteria-animal cells. In some other embodiments, an appropriately constructed expression vector contains: an origin of replication for autonomous replication in host cells, one or more selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

In further embodiments, the expression vector also comprises various elements that affect transcription and translation, including, for example, constitutive and inducible promoters. These elements are often host and/or vector dependent. For example, in other embodiments, when cloning in bacterial systems, inducible promoters such as the T7 promoter, pL of bacteriophage λ, plac, ptrp, ptac (ptrp-lac hybrid promoter) and the like are used; when cloning in insect cell systems, promoters such as the baculovirus polyhedrin promoter are used; when cloning in plant cell systems, promoters derived from the genome of plant cells (e.g., heat shock promoters; the promoter for the small subunit of RUTBISCO; the promoter for the chlorophyll a/b binding protein) or from plant viruses (e.g., the 35S RNA promoter of CaMV; the coat protein promoter of TMV) are used; when cloning in mammalian cell systems, mammalian promoters (e.g., metallothionein promoter) or mammalian viral promoters, (e.g., adenovirus late promoter; vaccinia virus 7.5K promoter; SV40 promoter; bovine papilloma virus promoter; and Epstein-Barr virus promoter) are used.

In yet some other embodiments, various methods are used to introduce the vector into host cells, for example, transformation, transfection, infection, protoplast fusion, and electroporation. The expression vector-containing cells are clonally propagated and individually analyzed to determine whether they produce the appropriate polypeptides. Various selection methods, including, for example, antibiotic resistance, are used to identify host cells that have been transformed. Identification of polypeptide expressing host cell clones are done by several means, including but not limited to immunological reactivity with anti-kinase antibodies, and the presence of host cell-associated activity.

In further embodiments, expression of cDNA are performed using in vitro produced synthetic mRNA. In yet further embodiments, synthetic mRNA is efficiently translated in various cell-free systems, including but not limited to wheat germ extracts and reticulocyte extracts, as well as efficiently translated in cell-based systems, including, but not limited, to microinjection into frog oocytes.

To determine the cDNA sequence(s) that yields optimal levels of activity and/or protein, modified cDNA molecules are constructed. A non-limiting example of a modified cDNA is where the codon usage in the cDNA has been optimized for the host cell in which the cDNA will be expressed. Host cells are transformed with the cDNA molecules and the levels of kinase RNA and/or protein are measured.

Levels of kinase protein in host cells are quantitated by a variety of methods such as immunoaffinity and/or ligand affinity techniques, kinase-specific affinity beads or specific antibodies are used to isolate ³⁵S-methionine labeled or unlabeled protein. Labeled or unlabeled protein is analyzed by SDS-PAGE. Unlabeled protein is detected by Western blotting, ELISA or RIA employing specific antibodies.

Following expression of kinase in a recombinant host cell, in other embodiments, polypeptides are recovered to provide the protein in active form. Several purification procedures are available and suitable for use. In further embodiments, recombinant kinase is purified from cell lysates or from conditioned culture media, by various combinations of, or individual application of, fractionation, or chromatography steps described herein.

In addition, in other embodiments, recombinant kinase is separated from other cellular proteins by use of an immuno-affinity column made with monoclonal or polyclonal antibodies specific for full length nascent protein or polypeptide fragments thereof. In yet further embodiments, other affinity based purification techniques is also used.

In some embodiments, the polypeptides are recovered from a host cell in an unfolded, inactive form, e.g., from inclusion bodies of bacteria. In yet other embodiments, proteins recovered in this form are solubilized using a denaturant, e.g., guanidinium hydrochloride, and then refolded into an active form using methods, such as, but not limited to, dialysis.

### Cell Growth Assays

A variety of cell growth assays are known and are useful in identifying heterocyclic compounds (i.e. “test compounds”) capable of inhibiting (e.g. reducing) cell growth and/or proliferation.

For example, a variety of cells are known to require specific kinases for growth and/or proliferation. In some embodiments, the ability of such a cell to grow in the presence of a test compound is assessed and compared to the growth in the absence of the test compound thereby identifying the anti-proliferative properties of the test compound. One common method of this type is to measure the degree of incorporation of label, such as tritiated thymidine, into the DNA of dividing cells. In further embodiments, inhibition of cell proliferation is assayed by determining the total metabolic activity of cells with a surrogate marker that correlates with cell number. In further embodiments, cells are treated with a metabolic indicator in the presence and absence of the test compound. Viable cells metabolize the metabolic indicator thereby forming a detectable metabolic product. Where detectable metabolic product levels are decreased in the presence of the test compound relative to the absence of the test compound, inhibition of cell growth and/or proliferation is indicated. Metabolic indicators include, for example tetrazolium salts and AlamorBlue® (see Examples section below).

An assay for kinases that stimulate cell migration is the scratch assay. This assay is used to evaluate inhibitors of kinases by mimicking events such as wound healing. In one variant of this assay used to test MET inhibitors, a confluent monolayer of cells is allowed to form on a cell plate. After formation of the monolayer, a linear wound on the monolayer is generated by mechanically scraping the monolayer thereby forming a cell-free channel. A growth factor required by the kinase for cell growth is added in the presence or absence of the test compound. The closure of the channel in the presence of the test compound indicates a failure of the test compound to inhibit the kinase thereby allowing cell migration and growth to close the channel. Conversely, the presence of the channel after adding the test compound indicates that test compound inhibited the kinase thereby preventing cell growth. The selection of the appropriate cells, growth conditions, and growth factors are well within the abilities of one skilled in the art (see Examples section below).

### Pharmaceutical Compositions and Administration

In another aspect, the present disclosure provides a pharmaceutical composition including a heterocyclic kinase modulator in admixture with a pharmaceutically acceptable carrier, excipient, binder or diluent. In one embodiment, the pharmaceutical compositions include the pharmaceutically acceptable salts of the heterocyclic kinase modulators described above.

In one aspect, pharmaceutical compositions are formulated in a conventional manner using one or more physiologically acceptable carriers comprising excipients and auxiliaries which facilitate processing of the active compounds into preparations which are used pharmaceutically. Proper formulation is dependent upon the route of administration chosen.

Provided herein are pharmaceutical compositions that include a compound of Formula (I) described herein and a pharmaceutically acceptable diluent(s), excipient(s), or carrier(s). In one embodiment, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease or condition to be treated. In one embodiment, the mammal is a human. In another embodiment, the therapeutically effective amount varies widely depending on the severity of the disease, the age and relative health of the subject, the potency of the compound used and other factors. In another embodiment, the compounds are used singly or in combination with one or more therapeutic agents as components of mixtures.

In some embodiments, administration of the compounds and compositions described herein are effected by any method that enables delivery of the compounds to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compound, and other suitable methods commonly known in the art. For example, in other embodiments, compounds described herein are administered locally to the area in need of treatment. In some other embodiments, this is achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. In some embodiments, the administration is by direct injection at the site (or former site) of a tumor or neoplastic or pre-neoplastic tissue. Those of ordinary skill in the art are familiar with formulation and administration techniques that can be employed with the compounds and methods of the present disclosure, e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

In some embodiments, the formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration although in other embodiments the most suitable route depends upon for example the condition and disorder of the recipient. In yet other embodiments, the formulations are conveniently presented in unit dosage form and may be prepared by any of the methods well known in the art of pharmacy. All methods include the step of bringing into association the compound of the subject disclosure or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Salts

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, the compounds described herein also exist as their pharmaceutically acceptable salts, which in other embodiments are useful for treating disorders. For example, the disclosure provides for methods of treating diseases, by administering pharmaceutically acceptable salts of the compounds described herein. In some embodiments, the pharmaceutically acceptable salts are administered as pharmaceutical compositions.

Thus, in some embodiments, the compounds described herein are prepared as pharmaceutically acceptable salts formed when an acidic proton present in the parent compound either is replaced by a metal ion, for example an alkali metal ion, an alkaline earth ion, or an aluminum ion; or coordinates with an organic base. In other embodiments, base addition salts are also prepared by reacting the free acid form of the compounds described herein with a pharmaceutically acceptable inorganic or organic base, including, but not limited to organic bases such as ethanolamine, diethanolamine, triethanolamine, N-methylglucamine, and the like and inorganic bases such as aluminum hydroxide, calcium hydroxide, potassium hydroxide, sodium carbonate, sodium hydroxide, and the like. In addition, in further embodiments, the salt forms of the disclosed compounds are prepared using salts of the starting materials or intermediates.

Further, in some embodiments, the compounds described herein are prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

### Solvates

In other embodiments, the compounds described herein also exist in various solvated forms, which in further embodiments are useful for treating disorders. For example, the disclosure provides for methods of treating diseases, by administering solvates of the compounds described herein. In some embodiments, the solvates are administered as pharmaceutical compositions. In other embodiments, the solvates are pharmaceutically acceptable solvates.

Solvates contain either stoichiometric or non-stoichiometric amounts of a solvent, and in further embodiments are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, in some embodiments, hydrates of the compounds described herein are conveniently prepared by recrystallization from an aqueous/organic solvent mixture, using organic solvents including, but not limited to, dioxane, tetrahydrofuran or methanol. In addition, in other embodiments, the compounds provided herein exist in unsolvated as well as solvated forms. In general, the solvated forms are considered equivalent to the unsolvated forms for the purposes of the compounds and methods provided herein.

### Polymorphs

In some embodiments, the compounds described herein also exist in various polymorphic states, all of which are herein contemplated, and in other embodiments, are useful for treating disorders. For example, the disclosure provides for methods of treating diseases, by administering polymorphs of the compounds described herein. In some embodiments, the various polymorphs are administered as pharmaceutical compositions.

Thus, the compounds described herein include all their crystalline forms, known as polymorphs. Polymorphs include the different crystal packing arrangements of the same elemental composition of the compound. In some embodiments, polymorphs have different x-ray diffraction patterns, infrared spectra, melting points, density, hardness, crystal shape, optical and electrical properties, stability, solvates and solubility. In other embodiments, various factors such as the recrystallization solvent, rate of crystallization, and storage temperature cause a single crystal form to dominate.

### Prodrugs

In some embodiments, the compounds described herein also exist in prodrug form, which in other embodiments, are useful for treating disorders. For example, the disclosure provides for methods of treating diseases, by administering prodrugs of the compounds described herein. In some embodiments, the prodrugs are administered as pharmaceutical compositions.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some embodiments, they are easier to administer than the parent drug. In further embodiments, they are bioavailable by oral administration whereas the parent is not. In some embodiments, the prodrug has improved solubility in pharmaceutical compositions over the parent drug. An example, without limitation, of a prodrug would be the compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. In some embodiments, the prodrug is a short peptide (polyamino acid) bonded to an acid group where the peptide is metabolized to reveal the active moiety.

In other embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:g210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-mannich bases, schiff bases, amino acid conjugates, phosphate esters, metal salts and sulfonate esters. Various forms of prodrugs are known. See for example design of prodrugs, Bundgaard, A. Ed., Elseview, 1985 and Method in Enzymology, Widder, K. Et al., ed.; Academic, 1985, Vol. 42, p. 309-396; Bundgaard, H. “Design and Application of Prodrugs” in A Textbook of Drug Design and Development, Krosgaard-Larsen and H. Bundgaard, ed., 1991, chapter 5, p. 113-191; and Bundgaard, H., Advanced Drug Delivery Review, 1992, 8, 1-38, each of which is incorporated herein by reference. The prodrugs described herein include, but are not limited to, the following groups and combinations of these groups; amine derived prodrugs:

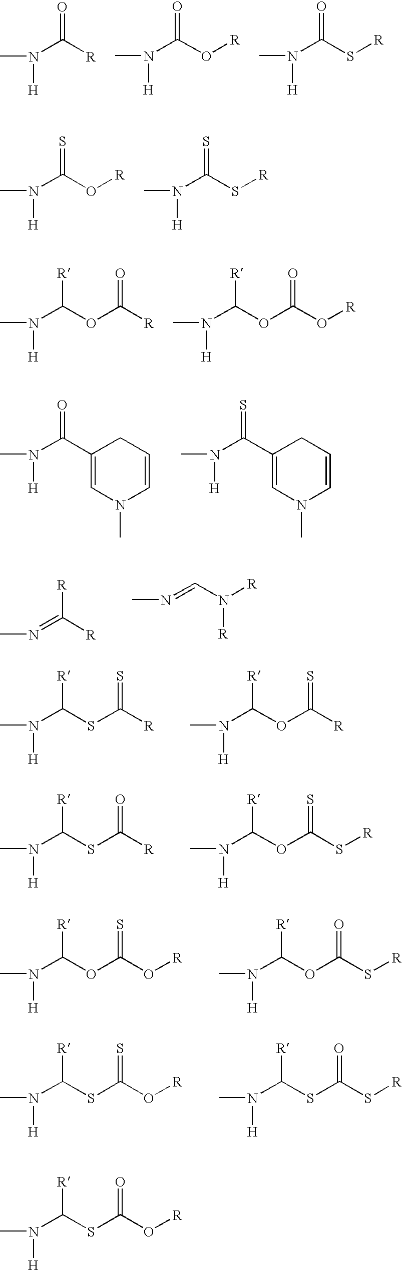

Hydroxy prodrugs include, but are not limited to acyloxyalkyl esters, alkoxycarbonyloxyalkyl esters, alkyl esters, aryl esters and disulfide containing esters.

In some embodiments, prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present disclosure. The amino acid residues include but are not limited to the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. Additional types of prodrugs are also encompassed.

Prodrug derivatives of compounds described herein can be prepared by methods described herein (e.g., for further details see Saulnier et al., (1994), Bioorganic and Medicinal Chemistry Letters, Vol. 4, p. 1985). By way of example only, in some embodiments, appropriate prodrugs are prepared by reacting a non-derivatized compound as described herein with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, in some embodiments, some of the herein-described compounds are a prodrug for another derivative or active compound.

In some embodiments, compounds as described herein having free amino, amido, hydroxy or carboxylic groups are converted into prodrugs. For instance, in some embodiments, free carboxyl groups are derivatized as amides or alkyl esters. In other embodiments, free hydroxy groups are derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups.

Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. In some embodiments, free amines are derivatized as amides, sulfonamides or phosphonamides. In some embodiments, all of these prodrug moieties incorporate groups including but not limited to ether, amine and carboxylic acid functionalities. In other embodiments, phosphate ester functionalities are used as prodrug moieties.

In some other embodiments, sites on the aromatic ring portions of the compounds described herein are susceptible to various metabolic reactions, therefore incorporation of appropriate substituents on the aromatic ring structures, reduces, minimizes or eliminates this metabolic pathway.

In some embodiments, administration of the compounds and compositions described herein are effected by any method that enables delivery of the compounds to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compound, and other suitable methods commonly known in the art. For example, in other embodiments, compounds described herein are administered locally to the area in need of treatment. In some other embodiments, this is achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. In some embodiments, the administration is by direct injection at the site (or former site) of a tumor or neoplastic or pre-neoplastic tissue. Those of ordinary skill in the art are familiar with formulation and administration techniques that can be employed with the compounds and methods of the present disclosure, e.g., as discussed in Goodman and Gilman, The Pharmacological Basis of Therapeutics, current ed.; Pergamon; and Remington's, Pharmaceutical Sciences (current edition), Mack publishing co., Easton, Pa.

In some embodiments, the formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration although in other embodiments the most suitable route depends upon for example the condition and disorder of the recipient. In yet other embodiments, the formulations are conveniently presented in unit dosage form and may be prepared by any of the methods well known in the art of pharmacy. All methods include the step of bringing into association the compound of the subject disclosure or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

In some embodiments, in therapeutic and/or diagnostic applications, the compounds of the disclosure are formulated for a variety of modes of administration, including systemic and topical or localized administration. In further embodiments, techniques and formulations generally are found in Remington: The Science and Practice of Pharmacy (20th ed.) Lippincott, Williams & Wilkins (2000).

According to another aspect, the disclosure provides pharmaceutical compositions including compounds of the formulas described herein, and a pharmaceutically acceptable carrier, adjuvant, or vehicle. The amount of compound in the compositions of the disclosure is such that is effective to detectably inhibit a protein kinase in a biological sample or in a patient.

Pharmaceutically acceptable salts are generally known, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20th ed.) Lippincott, Williams & Wilkins (2000). In some embodiments, pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the invention may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a subject (e.g. patient) to be treated.

For nasal or inhalation delivery, the agents of the invention may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

In some embodiments, depending on the specific conditions being treated, such agents are formulated into liquid or solid dosage forms and administered systemically or locally. In further embodiments, the agents are delivered, for example, in a timed- or sustained-low release forms is known to those skilled in the art. In further embodiments, techniques for formulation and administration are found in Remington: The Science and Practice of Pharmacy (20th ed.) Lippincott, Williams & Wilkins (2000). In other embodiments, suitable routes include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

In other embodiments, for injection, the agents of the disclosure are formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, in other embodiments, the compositions of the present disclosure, in particular, those formulated as solutions, are administered parenterally, such as by intravenous injection. In yet other embodiments, the compounds are formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a patient to be treated.

In other embodiments, for nasal or inhalation delivery, the agents of the disclosure are also formulated by methods known to those of skill in the art, and include, for example, but not limited to, examples of solubilizing, diluting, or dispersing substances such as, saline, preservatives, such as benzyl alcohol, absorption promoters, and fluorocarbons.

Pharmaceutical compositions suitable for use in the present disclosure include compositions wherein active ingredients are contained in an effective amount to achieve its intended purpose. Determination of the effective amounts is well within the capability of those skilled in the art, especially in light of the detailed disclosure provided herein.

In addition to the active ingredients, in other embodiments, these pharmaceutical compositions contain suitable pharmaceutically acceptable carriers comprising excipients and auxiliaries which facilitate processing of the active compounds into preparations which are used pharmaceutically. In some embodiments, the preparations formulated for oral administration are in the form of tablets, dragees, capsules, or solutions.

In other embodiments, pharmaceutical preparations for oral use are obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, in some other embodiments, disintegrating agents are added, such as the cross-linked polyvinylpyrrolidone, agar, or alginic acid or a salt thereof such as sodium alginate.

Dragee cores are provided with suitable coatings. For this purpose, concentrated sugar solutions may be used, which in some embodiments optionally contain gum arabic, talc, polyvinylpyrrolidone, carbopol gel, polyethylene glycol (peg), and/or titanium dioxide, lacquer solutions, and suitable organic solvents or solvent mixtures. In further embodiments, dye-stuffs or pigments are added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

In yet other embodiments, pharmaceutical preparations that are used orally include push-fit capsules made of gelatin, as well as soft, sealed capsules made of gelatin, and a plasticizer, such as glycerol or sorbitol. In some other embodiments, push-fit capsules contain the active ingredients in admixture with filler such as lactose, binders such as starches, and/or lubricants such as talc or magnesium stearate and, optionally, stabilizers. In other embodiments, with soft capsules, the active compounds are dissolved or suspended in suitable liquids, such as fatty oils, liquid paraffin, or liquid polyethylene glycols (PEGs). In addition, stabilizers may be added.

In some embodiments, pharmaceutical preparations are formulated as a depot preparation. In other embodiments, such long acting formulations are administered by implantation (for example, subcutaneously or intramuscularly) or by intramuscular injection. Thus, for example in further embodiments, the compounds are formulated with suitable polymeric or hydrophobic materials (for example, as an emulsion in an acceptable oil) or ion exchange resins, or as sparingly soluble derivatives, for example, as a sparingly soluble salt.

In some other embodiments, for buccal or sublingual administration, the compositions take the form of tablets, lozenges, pastilles, or gels formulated in conventional manner. In further embodiments, such compositions comprise the active ingredient in a flavored basis such as sucrose and acacia or tragacanth.

In yet other embodiments, pharmaceutical preparations are formulated in rectal compositions such as suppositories or retention enemas, e.g., containing conventional suppository bases such as cocoa butter, polyethylene glycol, or other glycerides.

In some other embodiments, pharmaceutical preparations are administered topically, that is by non-systemic administration. This includes the application of the compound of the present disclosure externally to the epidermis or the buccal cavity and the instillation of such the compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. Alternatively, a formulation may comprise a patch or a dressing such as a bandage or adhesive plaster impregnated with active ingredients and optionally one or more excipients or diluents. The amount of active ingredient present in the topical formulation may vary widely. The active ingredient may comprise, for topical administration, from about 0.001% to about 10% w/w, for instance from about 1% to about 2% by weight of the formulation. It may however comprise as much as about 10% w/w but in other embodiments will comprise less than about 5% w/w, in yet other embodiments from about 0.1% to about 1% w/w of the formulation.

Formulations suitable for topical administration in the mouth include losenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Depending upon the particular condition, or disease state, to be treated or prevented, additional therapeutic agents, which are normally administered to treat or prevent that condition, in other embodiments are administered together with the inhibitors of this disclosure.

The present disclosure is not to be limited in scope by the exemplified embodiments, which are intended as illustrations of single aspects of the disclosure. Indeed, various modifications of the disclosure in addition to those described herein will become apparent to those having skill in the art from the foregoing description. Such modifications are intended to fall within the scope of the disclosure. Moreover, any one or more features of any embodiment of the disclosure may be combined with any one or more other features of any other embodiment of the disclosure, without departing from the scope of the disclosure. References cited throughout this application are examples of the level of skill in the art and are hereby incorporated by reference herein in their entirety for all purposes, whether previously specifically incorporated or not.

### Methods of Dosing and Treatment Regimens

In one aspect, the compounds described herein are used in the preparation of medicaments for the treatment of diseases or conditions that are mediated by kinase activity or in which protein kinase modulation ameliorates the disease or condition. In addition, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In some embodiments, the compositions containing the compound(s) described herein are administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered appropriate for the caregiver to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered appropriate for the caregiver to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In some embodiments, wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In other embodiments, wherein the patient's status does improve, upon the doctor's discretion the administration of the compounds are given continuously; alternatively, the dose of drug being administered may be temporarily reduced or temporarily suspended for a certain length of time (i.e., a “drug holiday”). The length of the drug holiday can vary between about 2 days and about 1 year, including by way of example only, about 2 days, about 3 days, about 4 days, about 5 days, about 6 days, about 7 days, about 10 days, about 12 days, about 15 days, about 20 days, about 28 days, about 35 days, about 50 days, about 70 days, about 100 days, about 120 days, about 150 days, about 180 days, about 200 days, about 250 days, about 280 days, about 300 days, about 320 days, about 350 days, or about 365 days. In further embodiments, the dose reduction during a drug holiday is from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, in other embodiments, the dosage or the frequency of administration, or both, is reduced, as a function of the symptoms, to a level at which the improved disease, disorder or condition is retained. In further embodiments, patients will require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, and in some embodiments is nevertheless determined according to the particular circumstances surrounding the case, including, e.g., the specific agent being administered, the route of administration, the condition being treated, and the subject or host being treated. In general, however, doses employed for adult human treatment will typically be in the range of about 0.02 to about 5000 mg per day, in one embodiment about 1 to about 1500 mg per day. In further embodiments, the desired dose is conveniently presented in a single dose or as divided doses administered simultaneously (or over a short period of time) or at appropriate intervals, for example as two, three, four or more sub-doses per day.

In one embodiment, the pharmaceutical composition described herein is in unit dosage forms suitable for single administration of precise dosages. In unit dosage form, the formulation is divided into unit doses containing appropriate quantities of one or more compound. In another embodiment, the unit dosage is in the form of a package containing discrete quantities of the formulation. Non-limiting examples are packaged tablets or capsules, and powders in vials or ampoules. In a further embodiment, aqueous suspension compositions are packaged in single-dose non-reclosable containers. In another embodiment, multiple-dose reclosable containers are used, in which case includes a preservative in the composition. By way of example only, in some embodiments, formulations for parenteral injection are presented in unit dosage form, which include, but are not limited to ampoules, or in multi-dose containers, with an added preservative.

The daily dosages appropriate for the compounds described herein described herein are from about 0.01 to about 2.5 mg/kg per body weight. An indicated daily dosage in the larger subject, including, but not limited to, humans, is in the range from about 0.5 mg to about 100 mg, conveniently administered in divided doses, including, but not limited to, up to four times a day or in extended release form. Suitable unit dosage forms for oral administration comprise from about 1 to about 50 mg active ingredient. The foregoing ranges are merely suggestive, as the number of variables in regard to an individual treatment regime is large, and considerable excursions from these recommended values are not uncommon. In another embodiment, dosages are altered depending on a number of variables, not limited to the activity of the compound used, the disease or condition to be treated, the mode of administration, the requirements of the individual subject, the severity of the disease or condition being treated, and the judgment of the practitioner.

In a further embodiment, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are contemplated herein. In another embodiment, the data obtained from cell culture assays and animal studies are used in formulating a range of dosage for use in human. In yet a further embodiment, the dosage of such compounds lies within a range of circulating concentrations that include the ED₅₀ with minimal toxicity. In another embodiment, the dosage varies within this range depending upon the dosage form employed and the route of administration utilized.

One aspect disclosed herein provides for the administration of at least one compound described herein in combination with another therapeutic agent. By way of example only, if one of the side effects experienced by a patient upon receiving one of the compounds herein is inflammation, then in some embodiments, it is appropriate to administer an anti-inflammatory agent in combination with the initial therapeutic agent. Or, by way of example only, in some embodiments, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, by way of example only, in some embodiments, the benefit experienced by a patient is increased by administering one of the compounds described herein with another therapeutic agent (which also includes a therapeutic regimen) that also has therapeutic benefit. In some embodiments, regardless of the disease, disorder or condition being treated, the overall benefit experienced by the patient is additive of the two therapeutic agents or, in other embodiments, the patient experiences a synergistic benefit.

In other embodiments, therapeutically-effective dosages vary when the drugs are used in treatment combinations. Methods for experimentally determining therapeutically-effective dosages of drugs and other agents for use in combination treatment regimens are described in the literature, e.g., the use of metronomic dosing, i.e., providing more frequent, lower doses in order to minimize toxic side effects. In a further embodiment, a combination treatment regimen encompasses treatment regimens in which administration of a compound of Formula (I) described herein is initiated prior to, during, or after treatment with a second agent described above, and continues until any time during treatment with the second agent or after termination of treatment with the second agent. It also includes treatments in which a kinase activity modulator such as the compounds of Formula (I) described herein and the second agent being used in combination are administered simultaneously or at different times and/or at decreasing or increasing intervals during the treatment period. Combination treatment further includes periodic treatments that start and stop at various times to assist with the clinical management of the patient. For example, a compound of Formula (I) described herein in the combination treatment is administered weekly at the onset of treatment, decreasing to biweekly, and decreasing further as appropriate.

### Combination Therapy

Compositions and methods for combination therapy are provided herein. In accordance with one aspect, the pharmaceutical compositions disclosed herein are used to a kinase activity mediated disease or condition or a disease or condition that is ameliorated by kinase modulation.

Depending upon the particular condition, or disease state, to be treated or prevented, additional therapeutic agents, which are normally administered to treat or prevent that condition, may be administered together with the inhibitors of this invention. For example, chemotherapeutic agents or other anti-proliferative agents may be combined with the inhibitors of this invention to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

Other examples of agents the inhibitors of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

These additional agents may be administered separately, as part of a multiple dosage regimen, from the inhibitor-containing composition. Alternatively, these agents may be part of a single dosage form, mixed together with the inhibitor in a single composition.

The present invention is not to be limited in scope by the exemplified embodiments, which are intended as illustrations of single aspects of the invention. Indeed, various modifications of the invention in addition to those described herein will become apparent to those having skill in the art from the foregoing description. Such modifications are intended to fall within the scope of the invention. Moreover, any one or more features of any embodiment of the invention may be combined with any one or more other features of any other embodiment of the invention, without departing from the scope of the invention. For example, the kinase modulators described in the Fused Ring Heterocycles as Kinase Modulators section are equally applicable to the methods of treatment and methods of inhibiting kinases described herein. References cited throughout this application are examples of the level of skill in the art and are hereby incorporated by reference herein in their entirety for all purposes, whether previously specifically incorporated or not.

In another aspect, the disclosure provides combination therapies for treating or inhibiting the onset of a cell proliferative disorder or a disorder related to kinase signaling in a subject. The combination therapy comprises continuously or discontinuously dosing or administering to the subject a therapeutically or prophylactically effective amount of a compound of the formulas described herein, and one or more other anti-cell proliferation therapy including chemotherapy, radiation therapy, gene therapy and immunotherapy.

In another aspect, the compounds of the disclosure are continuously or discontinuously administered in combination with chemotherapy. As used herein, chemotherapy refers to a therapy involving a chemotherapeutic agent. In some embodiments, a variety of chemotherapeutic agents are used in the combined treatment methods disclosed herein. Chemotherapeutic agents contemplated as exemplary, include, but are not limited to: platinum compounds (e.g., cisplatin, carboplatin, oxaliplatin); taxane compounds (e.g., paclitaxcel, docetaxol); campotothecin compounds (irinotecan, topotecan); vinca alkaloids (e.g., vincristine, vinblastine, vinorelbine); anti-tumor nucleoside derivatives (e.g., 5-fluorouracil, leucovorin, gemcitabine, capecitabine) alkylating agents (e.g., cyclophosphamide, carmustine, lomustine, thiotepa); epipodophyllotoxins/podophyllotoxins (e.g. Etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologics (e.g., gemtuzamab, cetuximab, rituximab, pertuzumab, trastuzumab, bevacizumab); antibiotics/anthracylines (e.g. Idarubicin, actinomycin D, bleomycin, daunorubicin, doxorubicin, mitomycin C, dactinomycin, caminomycin, daunomycin); antimetabolites (e.g., clofarabine, aminopterin, cytosine arabinoside, methotrexate); tubulin-binding agents (e.g. Combretastatin, colchicine, nocodazole); topoisomerase inhibitors (e.g., camptothecin); differentiating agents (e.g., retinoids, vitamin D and retinoic acid); retinoic acid metabolism blocking agents (RAMBA) (e.g., accutane); kinase inhibitors (e.g., flavoperidol, imatinib mesylate, gefitinib, erlotinib, sunitinib, lapatinib, sorafinib, temsirolimus, dasatinib); farnesyltransferase inhibitors (e.g., tipifarnib); histone deacetylase inhibitors; inhibitors of the ubiquitin-proteasome pathway (e.g., bortezomib, yondelis).

Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. See Simpson W. G., The Calcium Channel Blocker Verapamil and Cancer Chemotherapy. Cell Calcium. December 1985; 6(6):449-67. Additionally, yet to emerge chemotherapeutic agents are contemplated as being useful in combination with the compound of the present disclosure.

In further embodiments, specific, non-limiting examples of combination therapies include use of the compounds of the present disclosure with agents found in the following pharmacotherapeutic classifications as indicated below. These lists should not be construed to be closed, but should instead serve as illustrative examples common to the relevant therapeutic area at present. Moreover, in other embodiments, combination regimens include a variety of routes of administration and should include oral, intravenous, intraocular, subcutaneous, dermal, and inhaled topical.

In some embodiments, therapeutic agents include chemotherapeutic agents, but are not limited to, anticancer agents, alkylating agents, cytotoxic agents, antimetabolic agents, hormonal agents, plant-derived agents, and biologic agents.

Examples of anti-tumor substances, for example those selected from, mitotic inhibitors, for example vinblastine; alkylating agents, for example cis-platin, carboplatin and cyclophosphamide; anti-metabolites, for example 5-fluorouracil, cytosine arabinside and hydroxyurea, or, for example, one of the preferred anti-metabolites disclosed in European Patent application No. 239362 such as N-(5-[N-(3,4-dihydro-2-methyl-4-oxoquinazolin-6-ylmethyl)-n-methylamino]-2-thenoyl)-L-glutamic acid; growth factor inhibitors; cell cycle inhibitors; intercalating antibiotics, for example adriamycin and bleomycin; enzymes, for example, interferon; and anti-hormones, for example anti-estrogens such as nolvadextm (tamoxifen) or, for example anti-androgens such as casodextm (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of treatment.

Alkylating agents are polyfunctional compounds that have the ability to substitute alkyl groups for hydrogen ions. Examples of alkylating agents include, but are not limited to, bischloroethylamines (nitrogen mustards, e.g. Chlorambucil, cyclophosphamide, ifosfamide, mechlorethamtine, melphalan, uracil mustard), aziridines (e.g. Thiotepa), alkyl alkone sulfonates (e.g. Busulfan), nitrosoureas (e.g. Carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfhydryl, carboxyl, and imidazole groups. Under physiological conditions, these drugs ionize and produce positively charged ion that attach to susceptible nucleic acids and proteins, leading to cell cycle arrest and/or cell death. In some embodiments, combination therapy including a kinase modulator as described herein and an alkylating agent has therapeutic synergistic effects on cancer and reduces side effects associated with these chemotherapeutic agents.

Cytotoxic agents are a group of drugs that produced in a manner similar to antibiotics as a modification of natural products. Examples of cytotoxic agents include, but are not limited to, anthracyclines (e.g. Doxorubicin, daunorubicin, epirubicin, idarubicin and anthracenedione), mitomycin C, bleomycin, dactinomycin, plicatomycin. These cytotoxic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. In some embodiments, combination therapy including a kinase modulator as described herein and a cytotoxic agent has therapeutic synergistic effects on cancer and reduces side effects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUDR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. In other embodiments, combination therapy including a kinase modulator as described herein and an antimetabolic agent has therapeutic synergistic effects on cancer and reduces side effects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. Diethylstibestrol), antiestrogens (e.g. Tamoxifen, toremifene, fluoxymesterol and raloxifene), antiandrogens (bicalutamide, nilutamide, flutamide), aromatase inhibitors (e.g., aminoglutethimide, anastrozole and letrazole), ketoconazole, goserelin acetate, leuprolide, megestrol acetate and mifepristone. In other embodiments, combination therapy including a kinase modulator as described herein and a hormonal agent has therapeutic synergistic effects on cancer and reduces side effects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (vp-16) and teniposide (vm-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. In other embodiments, combination therapy including a kinase modulator as described herein and a plant-derived agent having therapeutic synergistic effects on cancer and reducing side effects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. In another embodiment is a combination therapy including a kinase modulator as described herein and a biologic agent having therapeutic synergistic effects on cancer, enhance the patient's immune responses to tumorigenic signals, and reduce potential side effects associated with this chemotherapeutic agent.

For the treatment of oncologic diseases, proliferative disorders, and cancers, compounds according to the present disclosure may be administered with an agent selected from the group comprising: aromatase inhibitors, antiestrogen, anti-androgen, corticosteroids, gonadorelin agonists, topoisomerase I and II inhibitors, microtubule active agents, alkylating agents, nitrosoureas, antineoplastic antimetabolites, platinum containing compounds, lipid or protein kinase targeting agents, imids, protein or lipid phosphatase targeting agents, anti-angiogenic agents, AKT inhibitors, IGF-I inhibitors, FGF3 modulators, mTOR inhibitors, smac mimetics, hdac inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, p38 MAP kinase inhibitors, hsp90 inhibitors, multi-kinase inhibitors, bisphosphanates, rapamycin derivatives, anti-apoptotic pathway inhibitors, apoptotic pathway agonists, PPAR agonists, inhibitors of ras isoforms, telomerase inhibitors, protease inhibitors, metalloproteinase inhibitors, aminopeptidase inhibitors, dacarbazine (dtic), actinomycins C2, C3, D, and F1, cyclophosphamide, melphalan, estramustine, maytansinol, rifamycin, streptovaricin, doxorubicin, daunorubicin, epirubicin, idarubicin, detorubicin, caminomycin, idarubicin, epirubicin, esormbicin, mitoxantrone, bleomycins A, A2, and B, camptothecin, Irinotecan®, Topotecan®, 9-aminocamnptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A, B, or C, and semi-synthetic variants, Herceptin®, Rituxan®, cd40 antibodies, asparaginase, interleukins, interferons, leuprolide, and pegaspargase, 5-fluorouracil, fluorodeoxyuridine, ptorafur, 5′-deoxyfluorouridine, uft, mitc, s-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-dach, orrnaplatin, thalidomide, lenalidomide, CI-973, telomestatin, CHIR258, rad 001, saha, tubacin, 17-aag, sorafenib, JM-216, podophyllotoxin, epipodophyllotoxin, etoposide, teniposide, tarcevag, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. In some embodiments, examples of interleukins that are used in conjunction with a RON receptor tyrosine kinase or an abl tyrosine kinase modulator include, but are not limited to, interleukin 2 (IL-2), and interleukin 4 (IL-4), interleukin 12 (IL-12).

Interferons include more than 23 related subtypes with overlapping activities, all of the IFN subtypes within the scope of the present disclosure. IFN has demonstrated activity against many solid and hematologic malignancies, the later appearing to be particularly sensitive.

In further embodiments, other cytokines that are used in conjunction with a kinase modulator as described herein include those cytokines that exert profound effects on hematopoiesis and immune functions. Examples of such cytokines include, but are not limited to erythropoietin, granulocyte-csf (filgrastin), and granulocyte, macrophage-csf (sargramostim). In further embodiments, these cytokines are used in conjunction with a kinase modulator as described herein to reduce chemotherapy-induced myelopoietic toxicity.

In yet other embodiments, other immuno-modulating agents other than cytokines are used in conjunction with a kinase modulator as described herein to inhibit abnormal cell growth. Examples of such immuno-modulating agents include, but are not limited to bacillus calmette-guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody Herceptin® (trastruzumab) is raised against human epidermal growth factor receptor-2 (her2) that is overexpressed in some breast tumors including metastatic breast cancer. Overexpression of her2 protein is associated with more aggressive disease and poorer prognosis in the clinic. Herceptin® is used as a single agent for the treatment of patients with metastatic breast cancer whose tumors over express the her2 protein. In some embodiments are combination therapy including a kinase modulator as described herein and Herceptin® having therapeutic synergistic effects on tumors, especially on metastatic cancers.

Another example of monoclonal antibodies against tumor antigens is Rituxan® (rituximab) that is raised against cd20 on lymphoma cells and selectively deplete normal and malignant cd20+pre-b and mature b cells. Rituxan®P is used as single agent for the treatment of patients with relapsed or refractory low-grade or follicular, cd20+, b cell non-hodgkin's lymphoma. In another embodiment is a combination therapy including a kinase modulator as described herein and Rituxan® having therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, dpc-4, nf-1, nf-2, rb, p53, wtl, brca1 and brca2.

Dpc-4 is involved in pancreatic cancer and participates in a cytoplasmic pathway that inhibits cell division. Nf-1 codes for a protein that inhibits ras, a cytoplasmic inhibitory protein. Nf-1 is involved in neurofibroma and pheochromocytomas of the nervous system and myeloid leukemia. Nf-2 encodes a nuclear protein that is involved in meningioma, schwanoma, and ependymoma of the nervous system. Rb codes for the prb protein, a nuclear protein that is a major inhibitor of cell cycle. Rb is involved in retinoblastoma as well as bone, bladder, small cell lung and breast cancer. P53 codes for p53 protein that regulates cell division and can induce apoptosis. Mutation and/or inaction of p53 is found in a wide ranges of cancers. Wtl is involved in Wilms tumor of the kidneys. Brca1 is involved in breast and ovarian cancer, and brca2 is involved in breast cancer. The tumor suppressor gene can be transferred into the tumor cells where it exerts its tumor suppressing functions. In another embodiment is a combination therapy including a kinase modulator as described herein and a tumor suppressor having therapeutic synergistic effects on patients suffering from various forms of cancer.

Cancer vaccines are a group of agents that induce the body's specific immune response to tumors. Most of cancer vaccines under research and development and clinical trials are tumor-associated antigens (TAAs). TAAs are structures (i.e. proteins, enzymes or carbohydrates) which are present on tumor cells and relatively absent or diminished on normal cells. By virtue of being fairly unique to the tumor cell, taas provide targets for the immune system to recognize and cause their destruction. Example of TAAs include, but are not limited to gangliosides (gm2), prostate specific antigen (psa), alpha-fetoprotein (afp), carcinoembryonic antigen (cea) (produced by colon cancers and other adenocarcinomas, e.g. Breast, lung, gastric, and pancreas cancer s), melanoma associated antigens (mart-1, gp 100, mage 1,3 tyrosinase), papillomavirus e6 and e7 fragments, whole cells or portions/lysates of antologous tumor cells and allogeneic tumor cells.

In some embodiments, an additional component is used in the combination to augment the immune response to TAAs. Examples of adjuvants include, but are not limited to, bacillus calmette-guerin (bcg), endotoxin lipopolysaccharides, keyhole limpet hemocyanin (gklh), interleukin-2 (IL-2), granulocyte-macrophage colony-stimulating factor (gm-csf) and cytoxan, a chemotherapeutic agent which is believe to reduce tumor-induced suppression when given in low doses.

In another aspect, the disclosure provides compounds which are continuously or discontinuously administered in combination with radiation therapy. As used herein, “radiation therapy” refers to a therapy comprising exposing the subject in need thereof to radiation. Such therapy is known to those skilled in the art. In other embodiments, the appropriate scheme of radiation therapy is similar to those already employed in clinical therapies wherein the radiation therapy is used alone or in combination with other chemotherapeutics.

In another aspect, the disclosure provides compounds which are continuously or discontinuously administered in combination with a gene therapy. As used herein, “gene therapy” refers to a therapy targeting on particular genes involved in tumor development. Possible gene therapy strategies include the restoration of defective cancer-inhibitory genes, cell transduction or transfection with antisense dna corresponding to genes coding for growth factors and their receptors, RNA-based strategies such as ribozymes, RNA decoys, antisense messenger RNAs and small interfering RNA (sirna) molecules and the so-called ‘suicide genes’.

In other aspect, the disclosure provides compounds which are continuously or discontinuously administered in combination with an immunotherapy. As used herein, “immunotherapy” refers to a therapy targeting particular protein involved in tumor development via antibodies specific to such protein. For example, monoclonal antibodies against vascular endothelial growth factor have been used in treating cancers.

In other embodiments, where a second pharmaceutical is used in addition to a compound of the disclosure, the two pharmaceuticals are continuously or discontinuously administered simultaneously (e.g. In separate or unitary compositions) sequentially in either order, at approximately the same time, or on separate dosing schedules. In further embodiments, the two compounds are continuously or discontinuously administered within a period and in an amount and manner that is sufficient to ensure that an advantageous or synergistic effect is achieved. It will be appreciated that in some embodiments, the method and order of administration and the respective dosage amounts and regimes for each component of the combination will depend on the particular chemotherapeutic agent being administered in conjunction with the compound of the present disclosure, their route of administration, the particular tumor being treated and the particular host being treated.

In certain embodiments, the kinase modulators as described herein are taken alone or in combination with other compounds. In one embodiment, a mixture of two or more kinase modulating compounds are administered to a subject in need thereof.

In yet another embodiment, one or more kinase modulators as described herein are administered with one or more therapeutic agents for the treatment or prevention of various diseases, including, for example, cancer, diabetes, neurodegenerative diseases, cardiovascular disease, blood clotting, inflammation, flushing, obesity, ageing, stress, etc. In various embodiments, combination therapies comprising a kinase modulating compound refer to (1) pharmaceutical compositions that comprise one or more kinase modulating compounds in combination with one or more therapeutic agents (e.g., one or more therapeutic agents described herein); and (2) co-administration of one or more kinase modulating compounds with one or more therapeutic agents wherein the kinase modulating compound and therapeutic agent have not been formulated in the same compositions (but in some embodiments, are present within the same kit or package, such as a blister pack or other multi-chamber package; connected, separately sealed containers (e.g., foil pouches) that in further embodiments are separated by the user; or a kit where the kinase modulating compound(s) and other therapeutic agent(s) are in separate vessels). In further embodiments, when using separate formulations, the kinase modulator as described herein is administered at the same, intermittent, staggered, prior to, subsequent to, or combinations thereof, with the administration of another therapeutic agent.

In certain embodiments, the compounds described herein, their pharmaceutically acceptable salts, prodrug, solvates, polymorphs, tautomers or isomers are administered in combination with another cancer therapy or therapies. In other embodiments, these additional cancer therapies are for example, surgery, and the methods described herein and combinations of any or all of these methods. In further embodiments, combination treatments occur sequentially or concurrently and the combination therapies are neoadjuvant therapies or adjuvant therapies.

In some embodiments, the compounds described herein are administered with an additional therapeutic agent. In these embodiments, the compounds described herein are in a fixed combination with the additional therapeutic agent or a non-fixed combination with the additional therapeutic agent.

By way of example only, if one of the side effects experienced by a patient upon receiving one of the compounds described herein is hypertension, then in some embodiments, it is appropriate to administer an anti-hypertensive agent in combination with the compound. Or, by way of example only, the therapeutic effectiveness of one of the compounds described herein is enhanced by administration of another therapeutic agent, the overall therapeutic benefit to the patient is enhanced. Or, by way of example only, in other embodiments, the benefit experienced by a patient is increased by administering one of the compounds described herein with another therapeutic agent (which also includes a therapeutic regimen) that also has therapeutic benefit. In any case, in some embodiments, regardless of the disease, disorder or condition being treated, the overall benefit experienced by the patient is simply additive of the two therapeutic agents or in further embodiments, the patient experiences a synergistic benefit.

In some embodiments, the appropriate doses of chemotherapeutic agents is generally similar to or less than those already employed in clinical therapies wherein the chemotherapeutics are administered alone or in combination with other chemotherapeutics.

By way of example only, platinum compounds are advantageously administered in a dosage of about 1 to about 500 mg per square meter (mg/m²) of body surface area, for example about 50 to about 400 mg/m², particularly for cisplatin in a dosage of about 75 mg/m² and for carboplatin in about 300 mg/m² per course of treatment. Cisplatin is not absorbed orally and must therefore be delivered via injection intravenously, subcutaneously, intratumorally or intraperitoneally.

By way of example only, taxane compounds are advantageously continuously or discontinuously administered in a dosage of about 50 to about 400 mg per square meter (mg/m²) of body surface area, for example about 75 to about 250 mg/m², particularly for paclitaxel in a dosage of about 175 to about 250 mg/m² and for docetaxel in about 75 to about 150 mg/m² per course of treatment.

By way of example only, camptothecin compounds are advantageously continuously or discontinuously administered in a dosage of about 0.1 to about 400 mg per square meter (mg/m²) of body surface area, for example about 1 to about 300 mg/m², particularly for irinotecan in a dosage of about 100 to about 350 mg/m² and for topotecan in about 1 to about 2 mg/m² per course of treatment.

By way of example only, in some embodiments, vinca alkaloids are advantageously continuously or discontinuously administered in a dosage of about 2 to about 30 mg per square meter (mg/m²) of body surface area, particularly for vinblastine in a dosage of about 3 to about 12 mg/m², for vincristine in a dosage of about 1 to about 2 mg/m², and for vinorelbine in dosage of about 10 to about 30 mg/m² per course of treatment.

By way of example only, in further embodiments, anti-tumor nucleoside derivatives are advantageously continuously or discontinuously administered in a dosage of about 200 to about 2500 mg per square meter (mg/m²) of body surface area, for example about 700 to about 1500 mg/m². 5-fluorouracil (5-FU) is commonly used via intravenous administration with doses ranging from about 200 to about 500 mg/m² (in some embodiments from about 3 to about 15 mg/kg/day). Gemcitabine is advantageously continuously or discontinuously administered in a dosage of about 800 to about 1200 mg/m² and capecitabine is advantageously continuously or discontinuously administered in about 1000 to about 2500 mg/m² per course of treatment.

By way of example only, in other embodiments, alkylating agents are advantageously continuously or discontinuously administered in a dosage of about 100 to about 500 mg per square meter (mg/m²) of body surface area, for example about 120 to about 200 mg/m², in other embodiments for cyclophosphamide in a dosage of about 100 to about 500 mg/m², for chlorambucil in a dosage of about 0.1 to about 0.2 mg/kg of body weight, for carmustine in a dosage of about 150 to about 200 mg/m², and for lomustine in a dosage of about 100 to about 150 mg/m² per course of treatment.

By way of example only, in yet other embodiments podophyllotoxin derivatives are advantageously continuously or discontinuously administered in a dosage of about 30 to about 300 mg per square meter (mg/m²) of body surface area, for example about 50 to about 250 mg/m², particularly for etoposide in a dosage of about 35 to about 100 mg/m² and for teniposide in about 50 to about 250 mg/m² per course of treatment.

By way of example only, in other embodiments, anthracycline derivatives are advantageously continuously or discontinuously administered in a dosage of about 10 to about 75 mg per square meter (mg/m²) of body surface area, for example about 15 to about 60 mg/m², particularly for doxorubicin in a dosage of about 40 to about 75 mg/m², for daunorubicin in a dosage of about 25 to about 45 mg/m², and for idarubicin in a dosage of about 10 to about 15 mg/r² per course of treatment.

By way of example only, in further embodiments, anti-estrogen compounds are advantageously continuously or discontinuously administered in a dosage of about 1 to about 100 mg daily depending on the particular agent and the condition being treated. Tamoxifen is advantageously administered orally in a dosage of about 5 to about 50 mg, about 10 to about 20 mg twice a day, continuing the therapy for sufficient time to achieve and maintain a therapeutic effect. Toremifene is advantageously continuously or discontinuously administered orally in a dosage of about 60 mg once a day, continuing the therapy for sufficient time to achieve and maintain a therapeutic effect. Anastrozole is advantageously continuously or discontinuously administered orally in a dosage of about 1 mg once a day. Droloxifene is advantageously continuously or discontinuously administered orally in a dosage of about 20-100 mg once a day. Raloxifene is advantageously continuously or discontinuously administered orally in a dosage of about 60 mg once a day. Exemestane is advantageously continuously or discontinuously administered orally in a dosage of about 25 mg once a day.

By way of example only, in further embodiments, biologics are advantageously continuously or discontinuously administered in a dosage of about 1 to about 5 mg per square meter (mg/m²) of body surface area, or as known in the art, if different. For example, trastuzumab is advantageously administered in a dosage of 1 to about 5 mg/m², in other embodiments, from about 2 to about 4 mg/m² per course of treatment.

In other embodiments, when a compound is administered with an additional treatment such as radiotherapy, the radiotherapy is administered at 1 day, 2 days, 3 days, 4 days, 5 days, 6 days, 7 days, 14 days, 21 days, or 28 days after administration of at least one cycle of a compound. In some embodiments, the radiotherapy is administered at 1 day, 2 days, 3 days, 4 days, 5 days, 6 days, 7 days, 14 days, 21 days, or 28 days before administration of at least one cycle of a compound. In additional embodiments, the radiotherapy is administered in any variation of timing with any variation of the aforementioned cycles for a compound. In other embodiments, additional schedules for co-administration of radiotherapy with cycles of a compound are further determined by appropriate testing, clinical trials, or in some embodiments are determined by qualified medical professionals.

When a compound is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. In yet further embodiments, additional variations of administering compound cycles in anticipation of surgery, or after the occurrence of surgery, are further determined by appropriate testing and/or clinical trials, or in some embodiments are determined by assessment of qualified medical professionals.

Other therapies include, but are not limited to administration of other therapeutic agents, radiation therapy or both. In the instances where the compounds described herein are administered with other therapeutic agents, the compounds described herein need not be administered in the same pharmaceutical composition as other therapeutic agents, and may, because of different physical and chemical characteristics, be administered by a different route. For example, in some embodiments, the compounds/compositions are administered orally to generate and maintain good blood levels thereof, while the other therapeutic agent is administered intravenously. The determination of the mode of administration and the advisability of administration, where possible, in the same pharmaceutical composition, is within the knowledge of the skilled clinician with the teachings described herein. In some embodiments, the initial administration is made according to established protocols, and then, based upon the observed effects, the dosage, modes of administration and times of administration in other embodiments, is modified by the skilled clinician. The particular choice of compound (and where appropriate, other therapeutic agent and/or radiation) will depend upon the diagnosis of the attending physicians and their judgment of the condition of the patient and the appropriate treatment protocol.

In other embodiments, the compounds and compositions described herein (and where appropriate chemotherapeutic agent and/or radiation) is administered concurrently (e.g., simultaneously, essentially simultaneously or within the same treatment protocol) or sequentially, depending upon the nature of the disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation need not be administered simultaneously or essentially simultaneously, and the initial order of administration of the compound/composition, and in other embodiments, the chemotherapeutic agent and/or radiation, is not important. Thus, in some embodiments, the compounds/compositions of the present disclosure are administered first followed by the administration of the chemotherapeutic agent and/or radiation; or the chemotherapeutic agent and/or radiation is administered first followed by the administration of the compounds/compositions described herein. In further embodiments, this alternate administration is repeated during a single treatment protocol. With the teachings described herein, the determination of the order of administration, and the number of repetitions of administration of each therapeutic agent during a treatment protocol, would be within the knowledge of the skilled physician after evaluation of the disease being treated and the condition of the patient. For example, in some embodiments, the chemotherapeutic agent and/or radiation is administered first, especially if it is a cytotoxic agent, and then the treatment continued with the administration of the compounds/compositions of the present disclosure followed, where determined advantageous, by the administration of the chemotherapeutic agent and/or radiation, and so on until the treatment protocol is complete. Thus, in other embodiments and in accordance with experience and knowledge, the practicing physician modifies each protocol for the administration of the compound/composition for treatment according to the individual patient's needs, as the treatment proceeds. The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. In further embodiments, relief of disease-related symptoms such as pain, and improvement in overall condition is used to help judge effectiveness of treatment.

In some embodiments, a composition described herein is administered before the administration of one or more chemotherapeutic agents. As non-limiting examples of this embodiment, the chemotherapeutic agent is administered hours (e.g. one, five, ten, etc.) or days (e.g., one, two, three, etc.) After administration of the composition described herein. In some embodiments, the subsequent administration is shortly after (e.g., within an hour) administration of the compound described herein.

Anti-emetic agents are a group of drugs effective for treatment of nausea and emesis (vomiting). Cancer therapies frequently cause urges to vomit and/or nausea. Many anti-emetic drugs target the 5-HT3 seratonin receptor which is involved in transmitting signals for emesis sensations. These 5-HT3 antagonists include, but are not limited to, dolasetron (Anzemet®), granisetron Kytril®), ondansetron (Zofran®), palonosetron and tropisetron. Other anti-emetic agents include, but are not limited to, the dopamine receptor antagonists such as chlorpromazine, domperidone, droperidol, haloperidol, metaclopramide, promethazine, and prochlorperazine; antihistamines such as cyclizine, diphenhydramine, dimenhydrinate, meclizine, promethazine, and hydroxyzine; lorazepram, scopolamine, dexamethasone, Emetrol®, propofol, and trimethobenzamide. Administration of these anti-emetic agents in addition to the above described combination treatment will manage the potential nausea and emesis side effects caused by the combination treatment.

Immuno-restorative agents are a group of drugs that counter the immuno-suppressive effects of many cancer therapies. The therapies often cause myelosuppression, a substantial decrease in the production of leukocytes (white blood cells). The decreases subject the patient to a higher risk of infections. Neutropenia is a condition where the concentration of neutrophils, the major leukocyte, is severely depressed. Immuno-restorative agents are synthetic analogs of the hormone, granulocyte colony stimulating factor (g-csf), and act by stimulating neutrophil production in the bone marrow. These include, but are not limited to, filgrastim (Neupogen®), peg-filgrastim (Neulasta®) and lenograstim. Administration of these immuno-restorative agents in addition to the above described combination treatment will manage the potential myelosupression effects caused by the combination treatment.

Antibiotic agents are a group of drugs that have anti-bacterial, anti-fungal, and anti-parasite properties. Antibiotics inhibit growth or causes death of the infectious microorganisms by various mechanisms such as inhibiting cell wall production, preventing DNA replication, or deterring cell proliferation. Potentially lethal infections occur from the myelosupression side effects due to cancer therapies. The infections can lead to sepsis where fever, widespread inflammation, and organ dysfunction arise. Antibiotics manage and abolish infection and sepsis and include, but are not limited to, amikacin, gentamicin, kanamycin, neomycin, netilmicin, streptomycin, tobramycin, loracarbef, ertapener, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, fulrazolidone, isoniazid, linezolid, metronidazole, mupirocin, nitrofurantoin, platesimycin, pyrazinamide, dalfopristin, rifampin, spectinomycin, and telithromycin. Administration of these antibiotic agents in addition to the above described combination treatment will manage the potential infection and sepsis side effects caused by the combination treatment.

Anemia treatment agents are compounds directed toward treatment of low red blood cell and platelet production. In addition to myelosuppression, many cancer therapies also cause anemias, deficiencies in concentrations and production of red blood cells and related factors. Anemia treatment agents are recombinant analogs of the glycoprotein, erythropoietin, and function to stimulate erythropoesis, the formation of red blood cells. Anemia treatment agents include, but are not limited to, recombinant erythropoietin (epogeno, Dynopro®) and darbepoetin alfa (Aranesp®). Administration of these anemia treatment agents in addition to the above described combination treatment will manage the potential anemia side effects caused by the combination treatment.

In some embodiments, pain and inflammation side effects arising from the described herein combination treatment are treated with compounds selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

In some embodiments, for the treatment of pain and inflammation side effects, compounds according to the present disclosure are administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-d-glucose, prilocaine, emla cream (eutectic mixture of local anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxarine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. Administration of these pain and inflammation analgesic agents in addition to the above described combination treatment will manage the potential pain and inflammation side effects caused by the combination treatment.

### Preparation of Protein Kinase Modulator Compounds

### SYNTHETIC EXAMPLE

The compounds described herein are synthesized by an appropriate combination of synthetic methods. Techniques useful in synthesizing the compounds disclosed herein are readily contemplated. The discussion below is offered to illustrate certain of the diverse methods available for use in assembling the compounds presented herein. However, the discussion is not intended to define the scope of reactions or reaction sequences that are useful in preparing the compounds of the present disclosure. The compounds described herein are made by the procedures and techniques disclosed in the Examples section below, as well as by organic synthetic techniques.

### Protecting Groups

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups are known in the art and include, but are not limited to the following moieties:

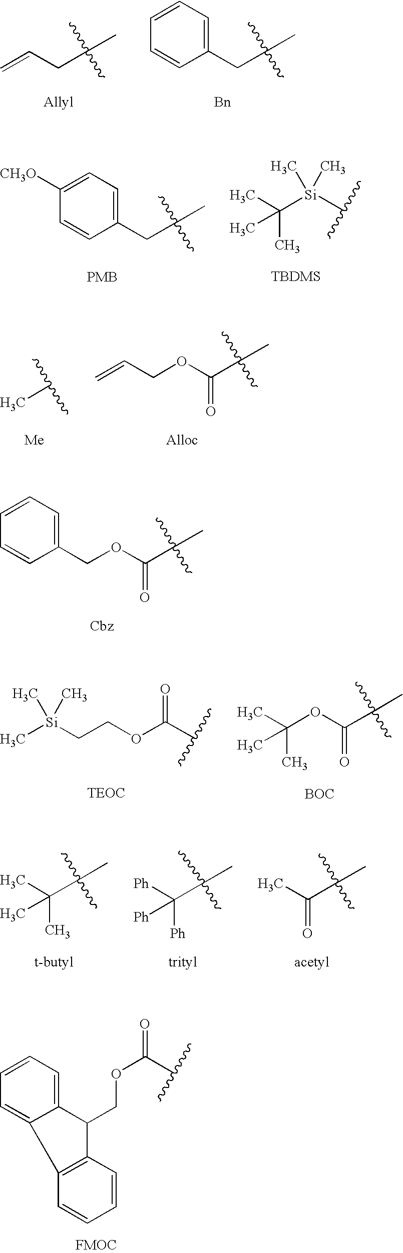

### EXAMPLES

The following examples are offered to illustrate, but not to limit what is claimed herein. The preparation of embodiments of the present disclosure is described in the following examples. In some embodiments, the chemical reactions and synthetic methods provided herein are modified to prepare many of the other compounds described herein. In further embodiments, where compounds of the present disclosure have not been exemplified, these compounds are prepared by modifying synthetic methods presented herein.

### Intermediate 1: (7-Fluoro-quinolin-6-yl)-acetic acid

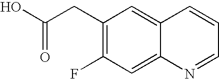

### Step 1: 6-bromo-7-fluoro-quinoline

A mixture of 4-bromo-3-fluoro-phenylamine (2.85 g, 15 m mole), ferrous sulfate (0.95 g), glycerol (5.658 g, 4.5 ml), nitrobenzene (1.125 g, 0.93 ml) and concentrated sulfuric acid (2.61 mL) were heated gently. After the first vigorous reaction, the mixture was heated to reflux for 7 hours. Nitrobenzene was evaporated in vacuo. The aqueous solution was acidified with glacial acetic acid and dark brown precipitate separated, which was purified by flash chromatography (silica gel, petroleum:ethyl acetate=8:1) to return compound title as white crystals (1.44 g, 42.5%).

### Step 2: (7-fluoro-quinolin-6-yl)-acetic acid tert-butyl ester

To a solution of 6-bromo-7-fluoro-quinoline (1.04 g, 4.6 mmol) in THF (1 mL) was added a solution of tert-butylzincbromide acetate (20 mL, 10.4 M in THF) followed by Pd(PPh₃)₄ (0.58 g, 0.5 mmol). The mixture was heated in a microwave reactor for 35 min at 120° C. The reaction mixture was quenched with a saturated ammonium chloride (60 mL), and extracted with ethyl acetate. The organic layer was dried over anhydrous Na₂SO₄, filtered and concentrated. The residue was purified by column chromatography to afford the title compound (0.75 g, 62.5%).

### Step 3: (7-fluoro-quinolin-6-yl)-acetic acid

A mixture of (7-fluoro-quinolin-6-yl)-acetic acid tert-butyl ester (3.67 g) and 4N aqueous sodium hydroxide (14.8 mL) were heated at 90° C. for 3 h. The solution was extracted with ethyl acetate. The aqueous layer was adjusted to acidic pH with acetic acid and filtered to afford the title compound (2.3 g, 79.8%). ¹H NMR (300 MHz, DMSO-d6): 12.52 (1H, s), 8.88˜8.90 (d, 1H), 8.34˜8.38 (d, 1H), 7.97˜7.99 (d, 1H), 7.73˜7.76 (d, 1H), 7.50˜7.54 (m, 1H), 3.85 (s, 2H). ES-MS m/z: 206.2 (M+1).

### Intermediate 2: (5,7-Difluoro-quinolin-6-yl)-acetic acid

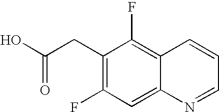

### Step 1: 6-Bromo-5,7-difluoro-quinoline

A mixture of 4-bromo-3,5-difluoro-phenylamine (6.0 g, 28.8 mmol), ferrous sulfate (1.82 g), glycerol (8.6 mL), nitrobenzene (1.79 mL) and 5.0 ml of concentrated sulfuric acid (5 mL) was heated gently. After the first vigorous reaction, the mixture was heated to reflux for five hours. Nitrobenzene was removed by distillation in vacuo. The aqueous solution was acidified with glacial acetic acid, and dark brown precipitate separated, which was purified by flash chromatography (silica gel, petroleum:ethyl acetate=12:1) to return title compound as a white solid (3.5 g, 49.8%).

### Step 2: 2-(5,7-Difluoro-quinolin-6-yl)-malonic acid diethyl ester

Ethyl malonate (9.28 g, 8.8 mL, 58.0 mmol) was added dropwise to a mixture of sodium hydride (60 percent in mineral oil, 2.32 g, 58.0 mmol) in 1,4-dioxane (29 mL) at 60° C. CuBr (4.176 g, 29.0 mmol) and 6-bromo-5,7-difluoro-quinoline (7.07 g, 29.0 mmol) were then added and the mixture heated to reflux for 16 h. After such time concentrated hydrochloric acid was added under ice-cooling and then tert-butyl methyl ether and water were added. The separated organic layer was washed with (10%) hydrochloric acid and water sequentially. Dried over sodium sulfate and concentrated. The residue was purified by column chromatography to afford the title compound (3.13 g, 35.4%).

### Step 3: (5,7-Difluoro-quinolin-6-yl)-acetic acid

To a round-bottom flask containing 2-(5,7-difluoro-quinolin-6-yl)-malonic acid diethyl ester (2.48 g, 7.68 mmol) were added ethanol (77 mL) and 10% aqueous NaOH (103.2 mL). The solution was refluxed for 3 h. After such time the ethanol was removed under reduced pressure to form a yellow suspension and THF (49.6 mL) was added to give a clear yellow solution which was placed in an ice bath and stirred. 6N HCl (49.6 ml) was slowly added to the solution to reach pH 1. The light orange solution was refluxed for another hour, at which time two layers formed. The top THF layer was collected and the aqueous solution was extracted with CH₂Cl₂. The organic layers were brined and dried with anhydrous sodium sulfate. The solution was then filtered and the filtrate was concentrated to obtain title compound (1.20 g, 70.1%). ¹H NMR (300 MHz, DMSO-d6): 12.82 (s, 1H), 8.98˜9.00 (m, 1H), 8.47˜8.50 (d, 1H), 7.61˜7.74 (m, 2H), 3.86 (s, 2H). ES-MS m/z: 224.2 (M⁺+1).

### Intermediate 3: 2-chloro-3-quinolin-6-yl-propionaldehyde

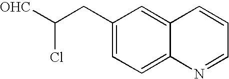

### Step 1: 3-Quinolin-6-yl-acrylic acid ethyl ester

To stirring solution of 6-bromoquinoline (10 g, 48.06 mmol) in DMF (100 mL) under nitrogen was added successively ethyl acrylate (15.7 mL, 144.2 mmol), triethylamine (48.6 mL, 480.6 mmol) and palladium(II) acetate (324 mg, 0.480 mmol). The reaction mixture was stirred at 100° C. for 24 h, then it was cooled to room temperature and concentrated in vacuo. The residue was diluted in ethyl acetate The organic layer was washed with saturated aqueous ammonium chloride (2×) and brine, dried over sodium sulfate, filtered, and adsorbed on silica gel. Purification by flash chromatography on silica gel using a gradient of 0-80% EtOAc:Hexane afforded 6.0 g of 3-quinolin-6-yl-acrylic acid ethyl ester as an orange oil (55% yield): ¹H NMR (DMSO-d6) δ 1.28 (t, 3H), 4.22 (q, 2H), 6.82 (d, 1H), 7.58 (dd, 1H), 7.83 (d, 1H), 8.01 (d, 1H), 8.17 (dd, 1H), 8.30 (d, 1H), 8.37 (dd, 1H), 8.93 (dd, 1H); MS (m/z) 228 [M+H⁺]⁺.

### Step 2: 3-Quinolin-6-yl-prop-2-en-1-ol

To a stirring solution of 3-quinolin-6-yl-acrylic acid ethyl ester (3.0 g, 13.2 mmol) in THF (48 mL) under nitrogen was added a 1 M solution of DIBAL-H in THF (58 mL) dropwise at −78° C. The reaction mixture was stirred at −78° C., adding 30 mL of DIBAL-H after 4 h and another 20 mL after 6 h to drive the reaction to completion. After 7 h, the reaction was quenched at −78° C. with a saturated solution of ammonium chloride (10 mL), and the mixture was left to warm up to room temperature overnight. More solution of ammonium chloride was added until appearance of a white paste. The organic layer was separated and adsorbed on silica gel. Purification by flash chromatography on silica gel using a gradient of 20-90% EtOAc:Hexane afforded 1.84 g of 3-quinolin-6-yl-prop-2-en-1-ol as off white crystals (75% yield): ¹H NMR (DMSO-d6) δ 4.19 (dt, 2H), 4.97 (t, 1H), 6.59 (dt, 1H), 6.78 (dt, 1H), 7.52 (dd, 1H), 7.92 (d, 1H), 7.95 (s, 1H), 8.31 (dd, 1H), 8.83 (dd, 1H); MS (m/z) 186 [M+H⁺]⁺.

### Step 3: 3-Quinolin-6-yl-propan-1-ol

A suspension of 3-quinolin-6-yl-prop-2-en-1-ol (575 mg, 3.108 mmol) and 10% wt Pd/C (165 mg, 0.155 mmol) in EtOH (10 mL) was stirred for 1.5 h under H₂ atmosphere. The reaction mixture was filtered over celite, and the filtrate was adsorbed on silica gel. Purification by flash chromatography on silica gel using a gradient of 30-100% EtOAc:Hexane afforded 311 mg of 3-quinolin-6-yl-propan-1-ol as a clear oil solidifying over time (53% yield): ¹H NMR (DMSO-d6) δ 1.82 (m, 2H), 2.81 (dd, 2H), 3.45 (q, 2H), 4.54 (t, 1H), 7.49 (dd, 1H), 7.64 (dd, 1H), 7.54 (d, 1H), 7.93 (d, 1H), 8.29 (dd, 1H), 8.83 (dd, 1H); MS (m/z) 188 [M+H⁺]⁺.

### Step 4: 3-Quinolin-6-yl-propionaldehyde

To a stirring solution of 3-quinolin-6-yl-propan-1-ol (310 mg, 1.66 mmol) in DCM (15 mL) was added Dess-Martin periodinane (630 mg, 1.48 mmol) in one portion. The reaction mixture was stirred at room temperature for 4 h, before adding 20 mL of 10% aqueous NaOH. The reaction mixture was stirred further for 15 min. The layers were separated. The aqueous layer was extracted with DCM. The organic layers were combined, washed with brine, dried over sodium sulfate, filtered, and adsorbed on silica gel. Purification by flash chromatography on silica gel using a gradient of 40-100% EtOAc:Hexane afforded 116 mg of 3-quinolin-6-yl-propionaldehyde as an oil (42% yield): ¹H NMR (DMSO-d6) δ 2.91 (t, 2H), 3.06 (t, 2H), 7.50 (dd, 1H), 7.66 (dd, 1H), 7.78 (d, 1H), 7.94 (d, 1H), 8.28 (dd, 1H), 8.84 (dd, 1H), 9.76 (s, 1H).

### Step 5: 2-Chloro-3-quinolin-6-yl-propionaldehyde

To a stirring solution of 3-quinolin-6-yl-propionaldehyde (50 mg, 0.27 mmol) in DCM (1 mL) at 0° C. was added DL-proline (3 mg, 0.027 mmol) followed by N-chlorosuccinimide (47 mg, 0.351 mmol). The reaction mixture was stirred at 0° C. for 1 h then at room temperature for 15 h. The reaction mixture was diluted with DCM, and the organic layer was washed with a saturated solution of sodium bicarbonate (2×) then brine, dried over sodium sulfate, and filtered. The filtrate was concentrated and dried in vacuo to provide 78 mg of crude 2-chloro-3-quinolin-6-yl-propionaldehyde as a yellow oil, that was used in the next step as such: MS (m/z) 252 [M+H⁺]⁺.

### Intermediate 4: 5-(1-methyl-1H-pyrazol-4-yl)-[1,3,4]thiadiazol-2-ylamine

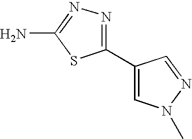

A mixture of 1-methyl-1H-pyrazole-4-carboxylic acid (300 mg, 2.38 mmol) and thiosemicarbazide (217 mg, 2.38 mmol) was treated with POCl₃ (0.85 mL). The reaction mixture was stirred at 100° C. for 1 h then cooled to room temperature. Water was added carefully. The reaction mixture was stirred at 100° C. for 2 h, and cooled to room temperature. Insolubles were filtered, and the filtrate was neutralized to pH 7-8 with 4 N aqueous NaOH. The aqueous layer was extracted with EtOAc (3×) and the organic layers were combined and adsorbed on silica gel. Purification by flash chromatography on silica gel using a gradient of 0-10% MeOH:DCM afforded 136 mg of 5-(1-methyl-1H-pyrazol-4-yl)-[1,3,4]thiadiazol-2-ylamine as off white crystals (31% yield): ¹H NMR (DMSO-d6) δ 3.86 (s, 3H), 7.19 (s, 2H), 7.77 (s, 1H), 8.16 (s, 1H); MS (m/z) 182 [M+H⁺]⁺.

### Intermediate 5: 4-Amino-5-quinolin-6-ylmethyl-4H-[1,2,4]triazole-3-thiol

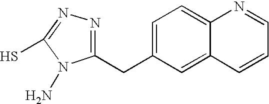

### Step 1: Quinolin-6-yl-acetic acid methyl ester

To a stirred solution of quinolin-6-yl-acetic acid (10 g, 53.0 mmol) in 100 ml of methanol was added 2.5 ml of concentrated H₂SO₄. The mixture was then heated to reflux for 3 hours. The reaction mixture was concentrated to give a brown residue, which was diluted with 100 ml of dichloromethane, washed with sat. aq. NaHCO₃ and brine, then the organic layer was dried over anhydrous Na₂SO₄ and concentrated to return title compound as a brown oil (7.9 g, 73.6%).

### Step 2: Quinolin-6-yl-acetic acid hydrazide

Quinolin-6-yl-acetic acid methyl ester (15 g, 74.5 mmol) was dissolved in 90 ml of ethanol, and hydrazine hydrate (16.6 mL, 342.9 mmol) was added dropwise to the solution while stirring. The resulting solution was heated to reflux for 1.5 h. Excess ethanol and hydrazine hydrate were distilled off and the contents allowed to cool. The precipitate was collected via filtration, washed with cold ethanol and dried in vacuo to return the title compound (15.4 g, 93.9%) as a white solid

### Step 3: N′-(2-Quinolin-6-yl-acetyl)-hydrazinecarbodithioic acid, potassium salt

Potassium hydroxide (3.9 g, 70 mmol) was dissolved in absolute ethanol (100 ml). To this solution, quinolin-6-yl-acetic acid hydrazide (15.4 g, 70 mmol) was added while the solution was cooled on ice. Carbon disulfide (5.32 g, 70 mmol) was then added in small portions with constant stirring. The reaction mixture was agitated continuously for a period of 15 h. The reaction mixture was then diluted with anhydrous diethyl ether (100 mL). The resulting solid was collected via filtration, washed with anhydrous diethyl ether (100 mL) and dried under vacuum to return title compound (22 g, 100%) as the potassium salt.

### Step 4: 4-Amino-5-quinolin-6-ylmethyl-4H-[1,2,4]triazole-3-thiol

A mixture of N′-(2-quinolin-6-yl-acetyl)-hydrazinecarbodithioic acid potassium salt (22 g, 70 mmol), water (3.5 mL) and hydrazine hydrate (10.5 mL, 210 mmol) was refluxed for 6 h. The color of the reaction mixture changed to green with the evolution of hydrogen sulfide gas. A homogenous reaction mixture was obtained during the reaction process. The reaction mixture was cooled to room temperature and diluted with water (100 mL). On acidification with concentrated hydrochloric acid the title compound precipitated from the solution (8 g, 44.4%) precipitated. The solid was collected via filtration, washed thoroughly with cold water and recrystallized from ethanol. To return the title compound as light yellow solid (8 g, 44% yield). ¹H NMR (300 MHz, DMSO-d6): 13.614 (s, 1H, SH), 8.87-8.89 (dd, J,=1.8 Hz, J₂=6 Hz, 1H), 8.33-8.36 (d, J=8.4 Hz, 1H), 7.97-8.00 (d, J=8.4 Hz, 1H), 7.85 (s, 1H), 7.69-7.72 (dd, J₁=1.8 Hz, J₂=10.8 Hz, 1H), 7.51-7.55 (m, 1H), 5.61 (s, 2H, NH₂), 4.26 (s, 2H, CH₂). ES-MS m/z: 258 (M+H⁺).

### Intermediate 6: (5-Phenyl-thiazol-2-yl)-hydrazine

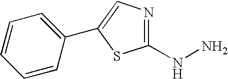

### Step 1: Bromo-Phenyl-Acetaldehyde

To a solution of phenyl-acetaldehyde (29 g, 0.24 mol) in CH₂Cl₂ (60 mL) was dropwise added a solution of Br₂ (38.7 g, 0.24 mol) in CH₂Cl₂ (25 mL) at −10° C. over 2 hours. The resulting solution was allowed to warm to room temperature and then heated to reflux overnight. Aqueous NaHCO₃ was added to the cooled mixture followed by extraction with CH₂Cl₂. The organic layer was dried over anhydrous Na₂SO₄ and concentrated in vacuo to return crude bromo-phenyl-acetaldehyde (47.5 g, 98.7%) as green liquid which was used directly in the next reaction.

### Step 2: 5-Phenyl-thiazol-2-ylamine

A mixture of bromo-phenyl-acetaldehyde (47.5 g, 0.239 mol), thiourea (36.7 g, 0.48 mol) and ethanol (170 mL) was heated to reflux overnight. The mixture was then cooled and the resulting precipitate filtered. The filtered precipitate was then washed with aqueous NaHCO₃. Recrystallization from methanol-water returned the title compound (15 g, 35.3%) as a yellow solid. ¹H NMR (300 MHz, DMSO-d6): 8.52 (s, 1H), 7.68 (s, 1H), 7.50 (m, 2H), 7.39 (m, 2H), 7.29 (m, 1H)

### Step 3: 2-Chloro-5-phenyl-thiazole

To a mixture of 5-phenyl-thiazol-2-ylamine (13 g, 74 mmol), CuCl₂ (20 g, 148 mmol) in CH₃CN (500 mL) was added isoamyl nitrite (17.3 g, 148 mmol) in a dropwise fashion at room temperature. The mixture was then stirred overnight at ambient temperature. The mixture was then concentrated in vacuo to remove CH₃CN and extracted with EtOAc. The organic layer was dried over anhydrous Na₂SO₄ and concentrated in vacuo to give crude title compound (8.5 g, 58.9%) as a brown solid. The crude product was purified via flash column chromatography eluting with petroleum ether:ethyl acetate 100:1 to give title compound (7.8 g, 54.0%) as a yellow solid. ¹H NMR (300 MHz, DMSO-d6): 8.13 (s, 1H), 7.65 (m, 2H), 7.43 (m, 3H); ES-MS: 196 (M+H⁺)

### Step 4: (5-Phenyl-thiazol-2-yl)-hydrazine

A mixture of 2-chloro-5-phenyl-thiazole (6.3 g, 32.2 mmol), hydrazine hydrate (8.05 g, 161 mmol) in pyridine (20 mL) was stirred at 60° C. overnight. The resulting mixture was concentrated in vacuo and the resulting solid stirred in diethyl ether, filtered and dried to give title compound (3.5 g, 55%) as a yellow solid. ¹H NMR (300 MHz, DMSO-d6): 8.13 (s, 1H), 7.65 (m, 2H), 7.43 (m, 3H); ES-MS: 192 (M+H⁺)

### Intermediate 7: 6-Phenyl-thiazolo[2,3-c][1,2,4]triazole-3-thiol

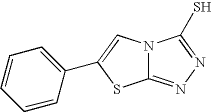

A mixture of (5-phenyl-thiazol-2-yl)-hydrazine (2.0 g, 10.5 mmol), thiocarbonyldiimidazole (2.79 g, 15.7 mmol) in DMF (15 mL) was stirred at 90° C. for 2 h. The mixture was concentrated in vacuo. The residue was dispersed in 0.6N aqueous NaOH (10 mL) and stirred for 1 h. The alkaline solution was treated with aqueous HCl to adjust pH=4˜5. The precipitate was collected by filtration, washed with Et₂O and dried to give title compound (1.01 g, 41.4%) as a green solid. ¹H NMR (300 MHz, DMSO-d6): 14.200 (m, 1H), 8.318 (s, 1H), 7.575 (m, 2H), 7.484 (m, 3H); ES-MS: 234 (M+H⁺); HPLC: 98.47% pure.

### Intermediate 8: [5-(1-Methyl-1H-pyrazol-4-yl)-[1,3,4]thiadiazol-2-yl]-hydrazine

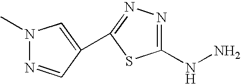

Step 1: 5-(1-Methyl-1H-pyrazol-4-yl)-[1,3,4]thiadiazol-2-ylamine

A mixture of 1-methyl-1H-pyrazole-4-carboxylic acid (3.46 g, 0.03 mol), and H₂NNHCSNH₂ (2.81 g, 0.03 mol) in POCl₃ (155 mL) was stirred at 60° C. for 1 h and heated to 90° C. for 2 h. After such time the mixture was concentrated in vacuo to remove POCl₃, and the title compound used in the next reaction without further purification. ES-MS m/z: 182 (M+H⁺).

### Step 2: 2-Chloro-5-(1-methyl-1H-pyrazol-4-yl)-[1,3,4]thiadiazole

To a mixture of 5-(1-methyl-1H-pyrazol-4-yl)-[1,3,4]thiadiazol-2-ylamine (5.55 g, 0.03 mol), Cu (0.6 g), in 37% aq. HCl (35 mL) and acetic acid (125 mL) was added aq. NaNO₂ (2.3 g, 0.033 mol) in H₂O (10 mL) in a dropwise fashion at 15° C. The mixture was then stirred at room temperature overnight, poured into water and extracted with CHCl₃, the aqueous layer was concentrated and basified with 50% NaOH and extracted with CHCl₃ and the combined the organic layers dried with Na₂SO₄ and concentrated in vacuum. The residue was purified via chromatography to return title compound (1.8 g, 29.9%) as a white solid. ES-MS m/z: 201 (M+H⁺).

### Step 3: 5-(1-Methyl-1H-pyrazol-4-yl)-[1,3,4]thiadiazol-2-yl]-hydrazine

A solution of 2-chloro-5-(1-methyl-1H-pyrazol-4-yl)-[1,3,4]thiadiazole (1.96 g, 9.79 mmol), hydrazine hydrate (1.47 g, 29.4 mmol) in ethanol (25 mL) was refluxed for 1 h. The resulting mixture was cooled, filtered and dried to give title compound (1.91 g, 100%) as a white solid. ES-MS m/z: 197 (M+H⁺).

### Intermediate 9: 6-(1-Methyl-1H-pyrazol-4-yl)-[1,2,4]triazolo[3,4-b][1,3,4]thiadiazole-3-thiol

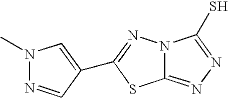

A mixture of 5-(1-methyl-1H-pyrazol-4-yl)-[1,3,4]thiadiazol-2-yl]-hydrazine (1.91 g, 9.79 mmol), thiocarbonyldiimidazole (2.71 g, 15.17 mmol) in dioxane (10.8 mL) and DMF (5.4 mL) was stirred at 90° C. for 2 h. The mixture was concentrated in vacuo and the residue dispersed in 0.6 mol/L of NaOH (40 mL) and stirred for 1 h. The alkaline solution was treated with charcoal, refluxed for 10 min and filtered, and the filtrate was adjusted to pH 4-5 with 37% aqueous HCl. The precipitate was collected by filtration to give crude product. This solid was dissolved in 5 mL of DMF at 80° C. and to which water (50 mL) was added to form a precipitate. The precipitate was filtered and dried to return title compound (11.0 g, 42.9%) as a white solid. ¹H NMR (300 MHz, DMSO-d6): 14.17 (s, 1H), 8.64 (s, 1H), 8.11 (d, 1H, J=0.6 Hz), 3.93 (s, 3H, CH₃). ES-MS m/z: 239 (M+H⁺).

### Intermediate 10: (7-Fluoro-quinolin-6-yl)-acetic acid hydrazide

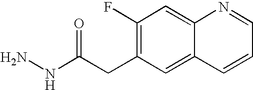

### Step 1: (7-Fluoro-quinolin-6-yl)-acetic acid methyl ester

A mixture of (7-fluoro-quinolin-6-yl)-acetic acid (2.6 g), 60 mL of methanol and 2.5 mL of concentrated sulfuric acid was stirred and heated to reflux for 3 h. Then the solution was concentrated and basified with NaHCO₃ followed by extraction with ethyl acetate. The organic layers were combined, dried and concentrated. 2.6 g of the title compound was obtained as a solid. ES-MS m/z: 220 (M⁺+1). Yield: 93.5%.

### Step 2: (7-Fluoro-quinolin-6-yl)-acetic acid hydrazide

To the refluxing solution of (7-fluoro-quinolin-6-yl)-acetic acid methyl ester (2.19 g, 10 mmol) and 5 mL of methanol was added hydrazine hydrate (500 mg). The mixture was refluxed for 3 h the solution was concentrated. The formed precipitate was collected by filtration and 2.3 g of the title compound was obtained after drying. The compound was used directly for the next step. ¹H NMR (DMSO-d6, 300 MHz): 9.30 (s, 1H), 8.88 (m, 1H), 8.37 (m, 1H), 7.96 (d, 1H), 7.70 (d, 1H), 7.52 (m, 1H), 4.27 (s, 2H), 3.63 (s, 2H)

### Intermediate 11: 4-Amino-5-(7-fluoro-quinolin-6-ylmethyl)-4H-[1,2,4]triazole-3-thiol

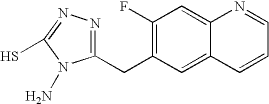

### Step 1: N′-[2-(7-Fluoro-quinolin-6-yl)-acetyl]-hydrazinecarbodithioic acid, potassium salt

Potassium hydroxide (840 mg, 15 mmol) was dissolved in ethanol (20 mL). To this solution (7-fluoro-quinolin-6-yl)-acetic acid hydrazide (intermediate 10) (2.19 g, 10 mmol) was added and cooled the solution in ice-bath. Carbon disulfide (1.5 mL, 15 mmol) was then added in small portions. The reaction mixture was stirred overnight, diluted with anhydrous ether, the title compound was collected by filtration after washing with anhydrous ether and dried to afford 3.5 g as yellow solid which was used directly for the next step.

### Step 2: 4-Amino-5-(7-fluoro-quinolin-6-ylmethyl)-4H-[1,2,4]triazole-3-thiol

A mixture of N′-[2-(7-fluoro-quinolin-6-yl)-acetyl]-hydrazinecarbodithioic acid potassium salt (3.5 g), 5 mL H₂O and hydrazine hydrate (7 mL) was heated to reflux for 2 h. Then the solution was cooled and water was added. On acidification with concentrated hydrochloric acid to pH 3-4, the title compound precipitated and was collected by filtration, washed with H₂O and further purified via flash column chromatography to return title compound (1.6 g, 58%). ¹H NMR (DMSO-d6, 300 MHz): 13.59 (s, 1H), 8.91 (m, 1H), 8.39 (m, 1H), 7.96 (d, 1H), 7.76 (d, 1H), 7.52 (m, 1H), 5.62 (s, 2H), 4.25 (s, 2H). ES-MS m/z: 276 (M⁺+H)

### Intermediate 12: 4-Amino-5-(5,7-difluoro-quinolin-6-ylmethyl)-4H-[1,2,4]triazole-3-thiol

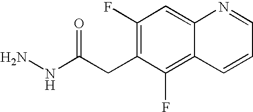

### Step 1: (5,7-Difluoro-quinolin-6-yl)-acetic acid methyl ester

A mixture of (5,7-difluoro-quinolin-6-yl)-acetic acid (2.6 g), 60 mL of methanol and 2.5 mL of concentrated sulfuric acid was stirred and heated to reflux for 3 h. Then the solution was concentrated and basified with NaHCO₃ followed by extraction with ethyl acetate. The organic layers were combined, dried and concentrated. 2.6 g of the title compound was obtained as a solid. ES-MS m/z: 238 (M⁺+1). Yield: 98%.

### Step 2: (5,7-Difluoro-quinolin-6-yl)-acetic acid hydrazide

To the refluxing solution of (5,7-difluoro-quinolin-6-yl)-acetic acid methyl ester (2.19 g, 10 mmol) and 5 mL of methanol was added hydrazine hydrate (500 mg). The mixture was refluxed for 3 h the solution was concentrated. The formed precipitate was collected by filtration and 2.3 g of the title compound was obtained after drying. The compound was used directly for the next step. ¹H NMR (DMSO-d6, 300 MHz): 9.34 (s, 1H), 8.96 (m, 1H), 8.48 (t, 1H), 7.76 (d, 1H), 7.62 (m, 1H), 4.27 (s, 2H), 3.66 (s, 2H). ES-MS m/z: 238 (M⁺+1).

### Intermediate 13: 4-Amino-5-(5,7-difluoro-quinolin-6-ylmethyl)-4H-[1,2,4]triazole-3-thiol

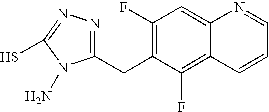

### Step 1: N′-[2-(5,7-Difluoro-quinolin-6-yl)-acetyl]-hydrazinecarbodithioic acid, potassium salt

Potassium hydroxide (840 mg, 15 mmol) was dissolved in ethanol (20 mL). To this solution (5,7-difluoro-quinolin-6-yl)-acetic acid hydrazide (2.19 g, 10 mmol) was added and cooled the solution in ice-bath. Carbon disulfide (1.5 mL, 15 mmol) was then added in small portions. The reaction mixture was stirred overnight, diluted with anhydrous ether, the title compound was collected by filtration after washing with anhydrous ether and dried to afford 3.5 g as a yellow solid which was used directly for the next step.

### Step 2: 4-Amino-5-(5,7-difluoro-quinolin-6-ylmethyl)-4H-[1,2,4]triazole-3-thiol

A mixture of N′-[2-(5,7-difluoro-quinolin-6-yl)-acetyl]-hydrazinecarbodithioic acid potassium salt (4.4 g), 30 mL H₂O and hydrazine hydrate (1 mL) was heated to reflux. 2 mL of hydrazine hydrate was added additionally and the solution was heated to reflux for 2 h, then the solution was cooled. The mixture was acidified with concentrated hydrochloric acid to pH 2, the formed precipitate was filtered, washed with H₂O, and then dissolved in aqueous sodium hydroxide and extracted with ethyl acetate. The aqueous phase was then acidified with conc. HCl and the solid was filtered and dried to return title compound (1.4 g, 56%). ¹H NMR (DMSO-d6, 300 MHz): 13.53 (s, 1H), 8.99 (r, 1H), 8.49 (t, 1H), 7.74 (d, 1H), 7.63 (m, 1H), 5.64 (s, 2H), 4.25 (s, 2H) ES-MS m/z: 294 (M⁺+H)

### Intermediate 14: (3-Bromo-quinolin-6-yl)-acetic acid hydrazide

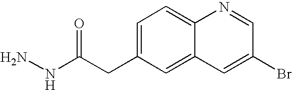

### Step 1: Quinolin-6-yl-acetic acid methyl ester

To a stirred solution of quinolin-6-yl-acetic acid (10 g, 53.0 mmol) in 100 ml of methanol was added 2.5 ml of concentrated H₂SO₄. The mixture was then heated to reflux for 3 hours. The reaction mixture was concentrated to give a brown residue, which was diluted with 100 ml of dichloromethane, washed with sat. aq. NaHCO₃ and brine, then the organic layer was dried over anhydrous Na₂SO₄ and concentrated to return title compound as a brown oil (7.9 g, 73.6%).

### Step 2: (3-Bromo-quinolin-6-yl)-acetic acid methyl ester

To a stirred solution of quinolin-6-yl-acetic acid methyl ester (21.6 g, 107 mmol) in 150 ml of carbon tetrachloride was treated with bromine (34.4 g, 215 mmol) and heated to reflux for 4 hours. The reaction mixture was treated with 17.0 g of pyridine, and further stirred for 2 hours under reflux. After cooling down to ambient temperature, the mixture was partitioned between dichloromethane and saturated aqueous sodium hydrogen carbonate, the organic layer was washed with water and brine, dried over magnesium sulfate then evaporated under reduced pressure to give a brown residue. The residue was purified by column chromatography, eluting with petroleum (60-90° C.) and then a 30:1 mixed solvent of petroleum and ethyl acetate to return title compound (13.6 g, 45.3%) as a white crystalline solid. (300 MHz, DMSO-d6): 8.89 (d, 1H), 8.27 (d, 1H), 8.06 (d, 1H), 7.67˜7.64 (m, 2H), 3.82 (s, 2H), 3.72 (s, 3H). ES-MS m/z: 280 (M+H⁺).

### Step 3: (3-bromo-quinolin-6-yl)-acetic acid

A mixture of (3-bromo-quinolin-6-yl)-acetic acid methyl ester (14.8 g, 52.8 mmol) and aqueous 2 N NaOH (80 mL, 160 mmol) was heated under reflux for 1.5 hours until reaction mixture became clear. After cooling down to room temperature, the reaction mixture was extracted with dichloromethane, the water layer was acidified with concentrated hydrochloric acid to pH 4, the white precipitate was filtered off dried to give the title compound as a white solid (10.3 g, 73.5%). ¹H NMR (300 MHz, DMSO-d6): 12.52 (b, 1H), 8.91 (d, 1H), 8.69 (d, 1H), 7.99 (d, 1H)), 7.82˜7.70 (m, 2H), 3.80 (s, 2H). ES-MS m/z: 266 (M+H⁺).

### Step 4: (3-Bromo-quinolin-6-yl)-acetic acid hydrazide

To a mixture of (3-bromo-quinolin-6-yl)-acetic acid (5 g, 18.9 mmol) in methanol was added concentrated sulfuric acid (1 mL). The mixture was heated to reflux for 18 hours. After such time sodium sulfate (20 g) was added to the cooled mixture then filtered. To the filtrate was added hydrazine hydrate (3.2 mL) and the mixture heated to reflux for 18 hours. After such time water (60 mL) was added and the mixture allowed to cool to room temperature. The formed precipitate was filtered and dried in vacuo to return title compound as a white solid (4.72 g, 16.9 mmol, 90%). ¹H NMR (500 MHz, DMSO-d6): 3.57 (2H, s), 4.26 (2H, d), 7.72 (1H, dd), 7.80 (1H, d), 7.97 (1H, d), 8.69 (1H, d), 8.90 (1H, d), 9.35 (1H, bs). ES-MS m/z: 280 (M+H⁺) 100%

### Intermediate 15: 5-(3-Bromo-quinolin-6-ylmethyl)-[1,2,4]triazole-3,4-diamine

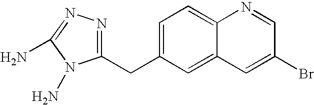

### Step 1: 5-(3-Bromo-quinolin-6-ylmethyl)-[1,3,4]oxadiazol-2-ylamine

A mixture of 3-bromo-quinolin-6-yl)-acetic acid hydrazide (4.12 g, 14.7 mmol), cyanogen bromide (1.1 eq, 16.2 mmol, 1.7 g), potassium hydrogen carbonate (1.25 eq, 18.3 mmol, 1.83 g) in methanol was stirred for 18 hours at ambient temperature. The mixture was then diluted with water (40 mL), the precipitate filtered and washed with cold methanol then dried in vacuo to return title compound as an off white solid (4.02 g, 13.2 mmol, 90%). ¹H NMR (500 MHz, DMSO-d6): 4.27 (2H, s), 6.94 (2H, s), 7.71 (1H, dd), 7.86 (1H, d), 8.02 (1H, d), 8.73 (1H, d), 8.93 (1H, d). ES-MS m/z: 305 (M+H⁺) 100%.

### Step 2: 5-(3-Bromo-quinolin-6-ylmethyl)-[1,2,4-]triazole-3,4-diamine

A mixture of 5-(3-bromo-quinolin-6-ylmethyl)-[1,3,4]oxadiazol-2-ylamine (3.53 g, 11.6 mmol) in water (15 ml) and hydrazine hydrate (30 mL) was heated in a microwave at 170° C., 2 bar for 1 hour. The cooled mixture was filtered and the solid washed with cold methanol and dried in vacuo to return title compound as a white solid (1.79 g, 5.6 mmol, 49%). ¹H NMR (500 MHz, DMSO-d6): 4.13 (2H, s), 5.46 (2H, s), 5.26 (2H, s), 7.23 (1H, d), 7.81 (1H, s), 7.98 (1H, d), 8.71 (1H, d), 8.91 (1H, d). ES-MS m/z: 319 (M+H⁺) 100%.

### Intermediate 16: 4-Amino-5-(3-bromo-5,7-difluoro-quinolin-6-ylmethyl)-4H-[1,2,4]triazole-3-thiol

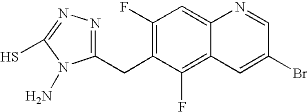

### Step 1: (3-Bromo-5,7-difluoro-quinolin-6-yl)-acetic acid methyl ester

A stirring solution of (5,7-difluoro-quinolin-6-yl)-acetic acid methyl ester (3.8 g, 15.8 mmol) in CCl₄ (25 mL) was treated with bromine (5.0 g, 31.7 mmol) and heated to reflux for 4 h. The reaction mixture was added with 2.5 g of pyridine, and further stirred for 2 h under reflux. After cooling down to ambient temperature, the mixture was partitioned between DCM and saturated aqueous NaHCO₃, the organic layer was washed with water and brine, dried over magnesium sulfate then evaporated under reduced pressure to give a brown residue. The residue was carefully purified by column chromatography with the elution being carried out using pure petroleum (60-90° C.), and then a 30/1 mixed solvent of petroleum and ethyl acetate to give product (3.0 g, 60%) as a white crystalline solid. MS m/z: 316, 318 (M+H⁺)

### Step 2: (3-Bromo-5,7-difluoro-quinolin-6-yl)-acetic acid hydrazide

(3-Bromo-5,7-difluoro-quinolin-6-yl)-acetic acid methyl ester (3.0, 9.5 mmol) was dissolved in 15 mL of ethanol, and hydrazine hydrate (2.8 mL, 55 mmol) was added drop wise to the solution while stirring. The resulting solution was allowed to reflux for 1.5 h. Excess ethanol and hydrazine hydrate were distilled off and the contents were allowed to cool. The precipitate was collected to give the product (2.8 g, 93%) which was used directly in the next step without further purification. MS m/z: 316, 318 (M+H⁺).

### Step 3: N′-[2-(3-Bromo-5,7-Difluoro-quinolin-6-yl)-acetyl]-hydrazinecarbodithioic acid, potassium salt

To an ice-cold solution of potassium hydroxide (0.74, 13 mmol) in absolute ethanol (20 mL) was added (3-bromo-5,7-difluoro-quinolin-6-yl)-acetic acid hydrazide (2.8 g, 8.8 mol. Carbon disulfide (4.15 g, 54.6 mmol) was then added in small portions with constant stirring. The reaction mixture was agitated continuously for 15 h. It was then diluted with anhydrous ether (50 mL). The precipitate was collected by filtration, further washed with anhydrous ether (50 mL) and dried under vacuum to afford the product (3 g, 79%) which was used in the next step without further purification.

### Step 4: 4-Amino-5-(3-bromo-5,7-difluoro-quinolin-6-ylmethyl)-4H-[1,2,4]triazole-3-thiol

A suspension of N′-[2-(3-bromo-5,7-Difluoro-quinolin-6-yl)-acetyl]-hydrazinecarbodithioic acid potassium salt (3 g, 6.9 mmol) in water (2 mL) and hydrazine hydrate (5 mL) was refluxed for 6 h. The color of the reaction mixture changed to green with the evolution of hydrogen sulfide gas. A homogenous reaction mixture was obtained during the reaction process. The reaction mixture was cooled to room temperature and diluted with water (5 mL). On acidification with concentrated hydrochloric acid a precipitate was formed, which was filtered, washed thoroughly with cold water and recrystallised from ethanol to give the product (2.2 g, 85%). ¹H NMR (300 MHz, DMSO-d6): 13.55 (s, 1H), 9.05 (d, 1H), 8.75 (d, 1H), 7.99 (d, 1H), 5.62 (s, 2H), 4.26 (s, 2H). MS m/z: 372, 374 (M+H⁺).

### Intermediate 17: 3-Bromo-6-[1,2,4]triazolo[4,3-b][1,2,4]triazin-3-ylmethyl-quinoline

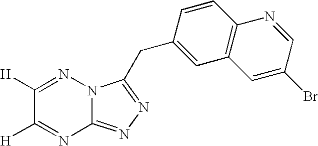

A mixture of 3-bromo-6-[1,2,4]triazolo[4,3-b][1,2,4]triazin-3-ylmethyl-quinoline (977 mg, 3 mmol), glyoxal (40% solution in water, 5 mL), acetic acid (10 mL) and water 2 mL) was stirred for 18 hours at ambient temperature. After such time the mixture was filtered, washed with methanol and the filtrate concentrated onto silica gel and purified via flash column chromatography (SiO₂, dichloromethane:methanol, 100:0 to 90:10) to return title compound as a white solid (298 mg. 0.9 mmol, 29%). ¹H NMR (500 MHz, DMSO-d6): 4.77 (2H, s), 7.79-7.85 (2H, m), 8.01 (1H, d), 8.67 (1H, s), 8.73-8.76 (2H, m), 8.92 (1H, d). ES-MS m/z: 341 (M+H⁺) 100%.

### Intermediate 18: 6-Methyl-[1,2,4]triazolo[3,4-b][1,3,4]thiadiazole-3-thiol

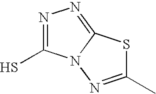

### Step 1: 1-methyl-1H-pyrazole-4-carboxaldehyde

POCl₃ (54.3 g, 0.35 mol) was added dropwise to a stirred solution of 1-methyl-1H-pyrazole (29.1 g, 0.35 mol) in dry DMF (90 mL) at 100° C. and the stirring was continued for 2 h. Then the reaction mixture was cooled and poured into ice water (350 mL), basified to pH 8 with 2 mol/L of NaOH, extracted with CHCl₃, dried over anhydrous Na₂SO₄ and concentrated in vacuum to give crude product. It was purified with chromatography to give compound 1-methyl-1H-pyrazole-4-carboxaldehyde (30 g, 77.7%) as a yellow oil. ES-MS m/z: 111 (M+H⁺).

### Step 2: 1-Methyl-1H-pyrazole-4-carboxylic acid

To a solution of 1-methyl-1H-pyrazole-4-carboxaldehyde (30 g, 0.27 mol) in acetone (150 mL) was added dropwise Jones reagent (260 mL, prepared by dissolving 69.0 g of CrO₃ in 59.8 mL of conc.H₂SO₄ and diluted to 260 mL with water) at 40° C. After reaction, the mixture was adjusted to pH 4 with 0.6 mol/L of NaOH. Then the precipitate was collected by filtration. The filtrate was extracted with EA and dried over anhydrous Na₂SO₄ and concentrated in vacuum to give 1-methyl-1H-pyrazole-4-carboxylic acid (10.7 g, 31%). ES-MS m/z: 127 (M+H⁺).

### Step 3: 5-(1-Methyl-1H-pyrazol-4-yl)-[1,3,4]thiadiazol-2-ylamine

A mixture of 1-methyl-1H-pyrazole-4-carboxylic acid (3.46 g, 0.03 mol), and H₂NNHCSNH₂ (2.81 g, 0.03 mol) in POCl₃ (15 mL) was stirred at 60° C. for 1 h and heated to 90° C. for 2 h. TLC showed the reaction was complete. The mixture was concentrated to remove POCl₃, which was used in the next reaction directly. ES-MS m/z: 182 (M+H⁺).

### Step 4: 2-Chloro-5-(1-methyl-1H-pyrazol-4-yl)-[1,3,4]thiadiazole

To a mixture of 5-(1-methyl-1H-pyrazol-4-yl)-[1,3,4]thiadiazol-2-ylamine (5.55 g, 0.03 mol), Cu (0.6 g), in 37% aq. HCl (35 mL) and HOAc (125 mL) was dropwise added aq. NaNO₂ (2.3 g, 0.033 mol) in H₂O (10 mL) at 15° C. Then the mixture was stirred at room temperature overnight, poured into water and extracted with CHCl₃, the aqueous layer was concentrated and basified with 50% NaOH and extracted with CHCl₃, combined the organic layer and dried with Na₂SO₄ and concentrated in vacuum to give crude 2-chloro-5-(1-methyl-1H-pyrazol-4-yl)-[1,3,4]thiadiazole. Then the crude product was purified with chromatography to give 2-chloro-5-(1-methyl-1H-pyrazol-4-yl)-[1,3,4]thiadiazole (1.8 g, 29.9%) as a white solid. ES-MS m/z: 201 (M+H⁺).

### Step 5: [5-(1-Methyl-1H-pyrazol-4-yl)-[1,3,4]thiadiazol-2-yl]-hydrazine

A solution of 2-chloro-5-(1-methyl-1H-pyrazol-4-yl)-[1,3,4]thiadiazole (1.96 g, 9.79 mmol), NH₂NH₂—H₂O (1.47 g, 29.4 mmol) in EtOH (25 mL) was refluxed for 1 h. TLC showed the reaction was complete. The resulting mixture was cooled, filtered and dried to give compound 5-(1-Methyl-1H-pyrazol-4-yl)-[1,3,4]thiadiazol-2-yl]-hydrazine (1.91 g, 100%) as a white solid. ES-MS m/z: 197 (M+H⁺).

### Step 6: 5-(1-Methyl-1H-pyrazol-4-yl)-3aH-pyrazolo[4,3-d]thiazole-3-thiol

5-(1-Methyl-1H-pyrazol-4-yl)-[1,3,4]thiadiazol-2-yl]-hydrazine (11.5 g, 88 mmol) was dissolved in 110 mL of EtOH and 29 mL of water, and then was added 5.3 g of KOH followed by 5.7 mL of CS₂. The mixture was stirred and heated to reflux for 2 hours under Nitrogen. Then, the mixture was cooled to room temperature and concentrated in vacuo. The residue was dissolved with 1N aqueous sodium hydroxide and insolubles were filtered off. The filtrate was acidified to pH 2-3 with 1N aqueous HCl. The resulting precipitate was collected, washed with water and dried in vacuo to provide 8.5 g of 5-(1-methyl-1H-pyrazol-4-yl)-3aH-pyrazolo[4,3-d]thiazole-3-thiol as a yellow solid (56%).

¹H NMR (DMSO-d6): 14.16 (s, 1H), 2.70 (s, 1H); MS (m/z): 173 [M+H]⁺.

### Intermediate 19: 6-Ethyl-[1,2,4]triazolo[3,4-b][1,3,4]thiadiazole-3-thiol

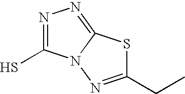

### Step 1: 2-chloro-5-(1-ethyl-1H-pyrazol-4-yl)-[1,3,4]thiadiazole

To a mixture of 5-(1-ethyl-1H-pyrazol-4-yl)-[1,3,4]thiadiazol-2-ylamine (5.0 g, 0.04 mol), Cu (0.39 g), in 37% aq. HCl (25 mL) and HOAc (75 mL) was dropwise added aq. NaNO₂ (2.3 g, 0.039 mol) in H₂O (10 mL) at 15° C. Then the mixture was stirred at room temperature overnight, poured into water and extracted with CHCl₃, the aqueous layer was concentrated and basified with 50% NaOH and extracted with CHCl₃, combined the organic layer and dried with Na₂SO₄ and concentrated in vacuum to give crude 2-chloro-5-(1-ethyl-1H-pyrazol-4-yl)-[1,3,4]thiadiazole. Then the crude product was purified with chromatography to give 2-chloro-5-(1-ethyl-1H-pyrazol-4-yl)-[1,3,4]thiadiazole (2.0 g, 35%) as a white solid. ES-MS m/z: 149 (M+H⁺).

### Step 2: [5-(1-ethyl-1H-pyrazol-4-yl)-[1,3,4]thiadiazol-2-yl]-hydrazine

To 2-chloro-5-(1-methyl-1H-pyrazol-4-yl)-[1,3,4]thiadiazole (1.95 g, 13.1 mmol) was added pyridine (20 mL) and the solution was cooled to 0 C. NH₂NH₂—H₂O (5.1 mL, 105 mmol) was then added and the solution heated to 65° C. for 2 h. The solvent was removed in vacuo and ethanol was added to obtain 5-(1-ethyl-1H-pyrazol-4-yl)-[1,3,4]thiadiazol-2-yl]-hydrazine (0.840 mg, 45%) as a pink solid. ES-MS m/z: 145 (M+H⁺).

### Step 3: 5-(1-ethyl-1H-pyrazol-4-yl)-3aH-pyrazolo[4,3-d]thiazole-3-thiol

5-(1-Ethyl-1H-pyrazol-4-yl)-[1,3,4]thiadiazol-2-yl]-hydrazine (830 mg, 5.76 mmol) was dissolved in 8 mL of EtOH and 2 mL of water, and then was added 0.36 g of KOH followed by 0.52 mL of CS₂. The mixture was stirred and heated to reflux for 2 hours under N2 (important!). Then, the mixture was cooled to room temperature and concentrated in vacuo. The residue was dissolved with 1N aqueous sodium hydroxide and insolubles were filtered off. The filtrate was acidified to pH 2-3 with 1N aqueous HCl. The resulting precipitate was collected, washed with water and dried in vacuo to provide 520 mg of 5-(1-ethyl-1H-pyrazol-4-yl)-3aH-pyrazolo[4,3-d]thiazole-3-thiol as a yellow solid (48%). MS (m/z): 187 [M+H]⁺.

### Intermediate 20: Trifluoromethanesulfonic Acid 3-(1-Methyl-1H-Pyrazol-4-yl)-Quinolin-6-yl Ester

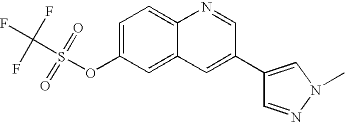

### Step 1: Acetic acid quinolin-6-yl ester

Quinolin-6-ol (135 g, 0.93 mol) was dissolved in pyridine (500 mL) and cooled to 0° C. in an ice-bath under a flow of nitrogen. Acetyl chloride (79 mL, 1.16 mol) was added to the reaction mixture slowly. Then it was stirred at room temperature for 3 hours. The mixture was partitioned between ethyl acetate (400 mL) and saturated aqueous NaHCO₃ (200 mL). The organic phase was separated and washed with brine (5×200 mL). The organic phase was dried over Na₂SO₄, filtered and concentrated in vacuo. The residue was purified by column chromatography to afford 120 g of acetic acid quinolin-6-yl ester as white solid (69% yield).

### Step 2: Acetic acid 3-bromo-quinolin-6-yl ester

To a mixture of acetic acid quinolin-6-yl ester (120 g, 0.642 mol) and pyridine (114 mL, 1.41 mol) in 6 L of CCl₄ was added Br₂ (66 mL, 1.28 mol) dropwise. The mixture was heated to reflux for 2 hours before being cooled to room temperature. The liquid in the flask was decanted and washed with saturated aqueous NaHCO₃ and water. The dark solid on the bottom of the flask was partitioned between aqueous NaHCO₃ and dichloromethane. The combined organic layers were washed with water again and dried before being evaporated to dryness in vacuo. The crude product was purified through flash column chromatography eluting with Petroleum Ether/ethyl acetate (10/1-1/1) to provide 108 g of acetic acid 3-bromo-quinolin-6-yl ester as a yellow solid (63% yield).

### Step 3: 3-(1-Methyl-1H-pyrazol-4-yl)-quinolin-6-ol

A mixture of acetic acid 3-bromo-quinolin-6-yl ester (108 g, 0.406 mol), 1-methyl-4-pyrazoleboronic acid pinacol ester (169 g, 0.752 mol), Na₂CO₃ (129 g, 1.28 mol), Pd(dppf)Cl₂ (32.8 g, 0.0406 mol), H₂O (607 mL) and 1,4-dioxane (1000 mL) was heated to 100° C. overnight. After cooling down to room temperature, most of the dioxane was removed under vacuo. The mixture was partitioned between ethyl acetate (500 mL) and brine (50 mL). The organic phase was separated and the aqueous phase was extracted with ethyl acetate (2×500 mL). The combined organic phases were dried over Na₂SO₄, filtered and concentrated in vacuo. The residue was purified by column chromatography to give 54 g of 3-(1-methyl-1H-pyrazol-4-yl)-quinolin-6-ol as a yellow solid (59% yield).

### Step 4: Trifluoro-methanesulfonic acid 3-(1-methyl-1H-pyrazol-4-yl)-quinolin-6-yl ester

A solution of 3-(1-methyl-1H-pyrazol-4-yl)-quinolin-6-ol (54 g, 0.24 mol) in Pyridine (400 mL) was cooled to 0° C. in an ice-bath under a flow of nitrogen. Triflic anhydride (48 mL, 0.28 mol) was added to the reaction mixture slowly and stirred at room temperature for 5 hours. The reaction mixture was partitioned between dichloromethane (300 mL) and saturated aqueous NaHCO₃ (200 mL). The organic phase was separated and washed by brine (5×300 mL). The organic phase was dried over Na₂SO₄, filtered and concentrated in vacuo. The residue was purified by column chromatography to give 58 g of trifluoro-methanesulfonic acid 3-(1-methyl-1H-pyrazol-4-yl)-quinolin-6-yl ester as white solid (70% yield): ¹HNMR (CDCl₃, 300 MHz): 9.30 (d, 1H), 8.62 (d, 1H), 8.43 (s, 1H), 8.16 (d, 1H), 8.11 (s, 1H), 8.10 (d, 1H), 7.76 (m, 1H), 3.92 (s, 3H); MS (m/z) 358 [M+H]⁺.

### Intermediate 21: 4-Amino-5-[3-(1-methyl-1H-pyrazol-4-yl)-quinolin-6-ylmethyl]-4H-[1,2,4]triazole-3-thiol

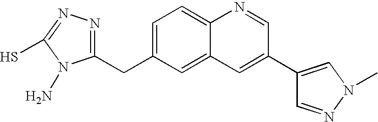

### Step 1: 13-(1-Methyl-1H-pyrazol-4-yl)-quinolin-6-yl]-acetic acid hydrazide

(3-Bromo-quinolin-6-yl)-acetic acid hydrazide (7 g, 25 mmol), 1-methyl-4-(4,4,5,5-tetramethyl-[1,3,2]dioxaborolan-2-yl)-1H-pyrazole (6.24 g. 30 mmol) and Pd(dppf)₂Cl₂ (915 mg, 1.25 mmol) were placed in a N₂ charged round bottom flask. Dimethoxyethane (100 mL), water (50 mL) and K₂CO₃ (10.3 g, 75 mmol) were added to the reaction vessel and the solution was bubbled with N₂ for 10 minutes. The mixture was heated under N₂ at 100° C. overnight. The solvent was removed and the precipitate formed. Crude [3-(1-methyl-1H-pyrazol-4-yl)-quinolin-6-yl]-acetic acid hydrazide (7.0 g) was used directly in the next step.

### Step 2: N′-{2-[3-(1-Methyl-1H-pyrazol-4-yl)-quinolin-6-yl]-acetyl}-hydrazinecarbodithioic acid, potassium salt

Potassium hydroxide (41 g, 533 mmol) was dissolved in absolute ethanol (325 ml). To this solution 3-(1-methyl-1H-pyrazol-4-yl)-quinolin-6-yl]-acetic acid hydrazide (10 g, 533 mmol) was added while the solution was cooled on ice. Carbon disulfide (24 mL, 70 mmol) was then added in small portions with constant stirring. The reaction mixture was refluxed, for a period of 15 h. The reaction mixture was then diluted with anhydrous diethyl ether (750 mL). The resulting solid was collected via filtration, washed with anhydrous diethyl ether (100 mL) and dried under vacuum to return title compound (14 g, 100%) as the potassium salt.

### Step 3: 4-Amino-5-[3-(1-methyl-1H-pyrazol-4-yl)-quinolin-6-ylmethyl]-4H-[1,2,4]triazole-3-thiol

N′-{2-[3-(1-Methyl-1H-pyrazol-4-yl)-quinolin-6-yl]-acetyl}-hydrazinecarbodithioic acid potassium salt (13.8 g, 35 mmol), hydrazine monohydrate (25 mL, 525 mmol) and water (100 mL) were heated at reflux overnight. The solution was then cooled and the mixture was acidified with concentrated hydrochloric acid to pH 2, the formed precipitate was filtered, washed with H₂O, and then dissolved in aqueous sodium hydroxide and extracted with ethyl acetate. The aqueous phase was then acidified with conc. HCl and the solid was filtered and dried to return title compound (9.1 g, 77% yield).

### Intermediate 22: 6-(6-Chloromethyl-[1,2,4]triazolo[3,4-b][1,3,4]thiadiazol-3-ylmethyl)-3-(1-methyl-1H-pyrazol-4-yl)-quinoline

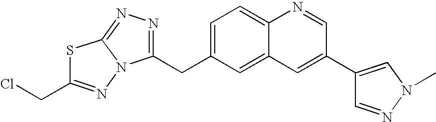

A mixture of 4-amino-5-[3-(1-methyl-1H-pyrazol-4-yl)-quinolin-6-ylmethyl]-4H-[1,2,4]triazole-3-thiol (2.0 g, 5.92 mmol), chloroacetic acid (1.68 g, 17.76 mmol) in phosphorous oxychloride (15 mL) was heated at 75° C. for 14 hours. The reaction mixture was cooled to room temperature and then the mixture was slowly added to isopropyl alcohol. The mixture was allowed to stand in an ice bath for 2 hours. The formed precipitate was filtered and washed with isopropyl alcohol. The resulting solid was dried to return title compound as a red-orange solid (1.14 g, 48.7%).

### Intermediate 23: 4-Amino-5-[5-fluoro-3-(1-methyl-1H-pyrazol-4-yl)-quinolin-6-ylmethyl]-4H-[1,2,4]triazole-3-thiol

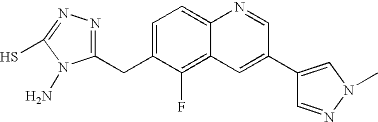

### Step 1: 5-Fluoro-6-Bromoquinoline

A mixture of 3-fluoro-4-bromoaniline (100 g, 526 mmole), 30 g of ferrous sulfate, 200 g of glycerol, 40 g of nitrobenzene and 100 ml of concentrated sulfuric acid was heated gently. After the first vigorous reaction, the mixture was boiled for five hours. Nitrobenzene was removed by distillation in vacuo. The aqueous solution was acidified with glacial acetic acid and dark brown precipitate separated, which was purified by flash chromatography (silica gel, petroleum/ethyl acetate=12/1) to give compound 5-fluoro-6-bromoquinoline and 7-fluoro-6-bromoquinoline as a white solid (80 g, 68%). The mixture was heated to reflux in PE. The solution was cooled to r.t. and filtered to collect 7-fluoro-6-bromoquinoline as a white solid. To the solution was added 2N HCl/MeOH, and the white solid precipitated from the solution. The solid was filtered and basified with aq. NaHCO3. The resulting precipitate was collected by filtration and dried to obtain 5-fluoro-6-bromoquinoline as a white solid. ¹H-NMR (DMSO-d₆, 300 MHz): 9.0 (d, 1H), 8.5 (d, 1H), 8.0 (1, H), 7.8 (d, 1H), 7.7 (m, 1H).

### Step 2: (5-Fluoro-quinolin-6-yl)-acetic acid tert-butyl ester

To a mixture of 5-fluoro-6-bromoquinoline (1 g, 4.5 mmol) and Pd(PPh₃)₄ (0.52 g, 0.45 mmol) was added a solution of tert-butylzincbromide acetate in THF (20 ml, 9 mmol). The mixture was heated in a microwave reactor for 30 min at 120° C. After cooling to r.t, the reaction mixture was quenched by sat. aq. NH₄Cl and extracted with EtOAc. The organic layer was dried over anhydrous Na₂SO₄ and concentrated. The residue was purified by column chromatography (silica gel, petroleum/ethyl acetate=10/1) to give the product (0.83 g, 71%). ¹H-NMR (CDCl₃, 300 MHz): 8.9 (d, 1H), 8.4 (d, 1H), 7.8 (d, 1H), 7.6 (m, 1H), 7.5 (m, 1H), 3.7 (d, 2H), 1.4 (s, 9H).

### Step 3: (5-Fluoro-quinolin-6-yl)-acetic acid methyl ester

5 g of (5-fluoro-quinolin-6-yl)-acetic acid tert-butyl ester (19 mmol) in 25 mL of aq. NaOH (4N) was heated to reflux for 4 h. The mixture was washed with EtOAc and the aqueous layer was added conc. HCl to pH=5 and the resulting precipitate was collected and washed with water to give 2.5 g of a white solid. It was then mixed with conc.H₂SO₄ (1.2 mL) and MeOH (20 mL) and the solution was heated to reflux for 6 h. After cooling the solvent was removed in vacuum. The residue was purified by column chromatography (silica gel, petroleum/ethyl acetate=10/1) to give the product (2.0 g, 48%). MS m/z: 220 (M+H⁺).

### Step 4: (3-Bromo-5-fluoro-quinolin-6-yl)-acetic acid methyl ester

To a solution of (5-fluoro-quinolin-6-yl)-acetic acid methyl ester (2.0 g, 9 mmol) in CCl₄ (20 mL) and pyridine (1.48 mL, 18 mmol) was added bromine (0.9 mL, 18 mmol) dropwise at 0-5° C. The solution was heated to reflux for 20 min. After cooling, the reaction was quenched by sat. aq. NaHCO₃ and the mixture was extracted with CH₂Cl₂ and concentrated. The residue was purified by column chromatography (silica gel, petroleum/ethyl acetate=1511) to give the product (1.8 g, 66%). ¹H-NMR (CDCl₃, 300 MHz): 8.9 (d, 1H), 8.5 (d, 1H), 7.8 (d, 1H), 7.6 (m, 1H), 3.9 (d, 2H), 3.7 (s, 3H). MS m/z: 298, 300 (M+H⁺).

### Step 5: (3-Bromo-5-fluoro-quinolin-6-yl)-acetic acid hydrazide

A solution of (3-bromo-5-fluoro-quinolin-6-yl)-acetic acid methyl ester (0.5 g, 1.68 mmol) and hydrazine hydrate (98%, 2 ml) in MeOH (15 ml) was heated to reflux for 1 h. The solvent was removed in vacuum and the resulting white solid was washed with MeOH to give the product (0.45 g, 89%). ¹H-NMR (DMSO-d6, 300 MHz): 9.34 (s, 1H), 8.99-9.00 (d, 1H), 8.72-8.73 (d, 1H), 7.85-7.88 (d, 1H), 7.73-7.79 (m, 1H), 4.26-4.27 (d, 2H), 3.64-3.66 (d, 2H). MS m/z: 298, 300 (M+H⁺).

### Step 6: [3-(1-Methyl-1H-pyrazol-4-yl)-5-fluoro-quinolin-6-yl]-acetic acid hydrazide

A mixture of (3-bromo-5-fluoro-quinolin-6-yl)-acetic acid hydrazide (0.45 g, 1.51 mmol), 1-methyl-1H-pyrazole-4-boronic acid pinacol ester (0.42 g, 2.01 mmol), K₂CO₃ (0.7 g, 5.04 mmol), Pd(dppf)Cl₂ (80 mg, 0.09 mmol), H₂O (2.5 mL) and dioxane (4.5 mL) was stirred at 100° C. overnight. After cooling down to r.t., most of the dioxane was removed in vacuo. The mixture was diluted with ethyl acetate (10 mL) and saturated brine (40 mL). The organic phase was separated and the aqueous phase was extracted with ethyl acetate (3*30 mL). The combined organic phases were dried over Na₂SO₄, filtered and concentrated. The residue was purified by column chromatography (silica gel, petroleum/ethyl acetate=8/1) to give the product (0.4 g, 89%) as a yellow solid. MS m/z: 300 (M+H⁺).

### Step 7: N′-[2-(3-(1-Methyl-1H-pyrazol-4-yl)-5-fluoro-quinolin-6-yl)-acetyl]-hydrazinecarbodithioic acid, potassium salt

Potassium hydroxide (0.11 g, 2.0 mmol) was dissolved in absolute ethanol (3 mL). To the above solution, [3-(1-methyl-1H-pyrazol-4-yl)-5-fluoro-quinolin-6-yl]-acetic acid hydrazide (0.4 g, 1.3 mmol) was added while the solution was cooled in an ice bath. Then carbon disulfide (0.31 g, 4 mmol) was added in small portions with constant stirring. The reaction mixture was agitated continuously for a period of 15 h. It was then diluted with anhydrous ether (10 mL). The resulting precipitate was filtered, washed with anhydrous ether (10 mL) and dried under vacuum to give the product (0.5 g, 93%), which was used in the next step without further purification.

### Step 8: 4-Amino-5-[5-fluoro-3-(1-methyl-1H-pyrazol-4-yl)-quinolin-6-ylmethyl]-4H-[1,2,4]triazole-3-thiol

A suspension of N′-[2-(3-(1-Methyl-1H-pyrazol-4-yl)-5-fluoro-quinolin-6-yl)-acetyl]-hydrazinecarbodithioic acid, potassium salt (0.5, 1.2 mmol) in water (1.0 mL) and hydrazine hydrate (3.8) was refluxed for 6 h. The color of the reaction mixture changed to green with the evolution of hydrogen sulfide gas. A homogenous reaction mixture was obtained during the reaction process. The reaction mixture was cooled to room temperature and diluted with water (10 mL). Upon acidification with concentrated hydrochloric acid a precipitate was formed. It was filtered, washed thoroughly with cold water and recrystallised from ethanol to give the product (220 mg, 51%). MS m/z: 356 (M+H⁺).

### Intermediate 24: 6-(6-Bromo-[1,2,4]triazolo[4,3-a]pyrimidin-3-ylmethyl)-quinoline

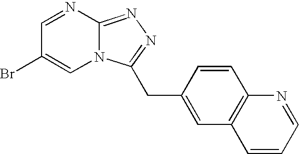

### Step 1: Quinolin-6-yl-acetic acid N′-(5-bromo-pyrimidin-2-yl)-hydrazide

5-Bromo-2-hydrazino-pyrimidine (1.02 g, 5.43 mmol) and 6-quinoline acetic acid (1.02 g, 5.43 mmol) were dissolved in dichloromethane (78 mL) and stirred at room temperature for 12 hours. The desired product was insoluble in dichloromethane and precipitated upon formation. The precipitate was filtered off and taken onto subsequent reactions as crude product, quinolin-6-yl-acetic acid N′-(5-bromo-pyrimidin-2-yl)-hydrazide (theoretical yield 1.94 g).

### Step 2: N′-(5-Bromopyridin-2-yl)-2-quinolin-6-yl)acetohydrazonoyl chloride

Quinolin-6-yl-acetic acid N′-(5-bromo-pyrimidin-2-yl)-hydrazide (1.06 g, 2.96 mmol) was suspended in Phosphorus oxychloride (30 ml). The reaction mixture was heated to 100° C. for 16 hours. The solution was concentrated in vacuo and taken onto the next reaction as crude product, N′-(5-Bromopyrimidin-2-yl)-2-quinolin-6-yl)acetohydrazonoyl chloride (theoretical yield 1.11 g).

### Step 3: 6-(6-Bromo-[1,2,4]triazolo[4,3-a]pyrimidin-3-ylmethyl)-quinoline

N′-(5-Bromopyrimidin-2-yl)-2-quinolin-6-yl)acetohydrazonoyl chloride (1.11 g, 0.56 mmol) was dissolved in Pyridine (50 ml) and stirred at room temperature for 3 hours. The desired product was insoluble in Pyridine and precipitated upon formation. The precipitate was filtered off to afford the desired product, 6-(6-Bromo-[1,2,4]triazolo[4,3-a]pyrimidin-3-ylmethyl)-quinoline (theoretical yield 1.00 g).

### Example 2

### General Method A

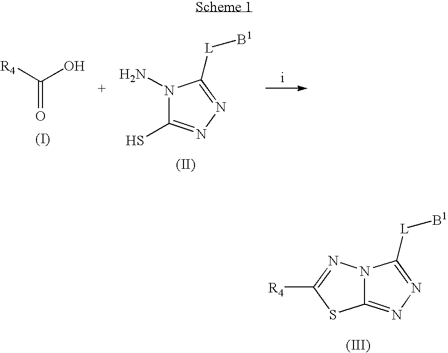

Compounds of formula (I) where R₄ is described herein are either available commercially or prepared using transformations known to those skilled in the art.

Compounds of general formula (II) where L and B¹ are described herein are either available commercially or prepared using methods described for the synthesis of intermediates 5 and transformations known to those skilled in the art.

Compounds of general formula (III) may be prepared from compounds of formula (I) and compounds of general formula (II) by process step (i), which comprises heating an amino thiol (II) and carboxylic acid (I) in the presence of POCl₃.

### Example 2A

### 6-[6-(1-Methyl-1H-pyrazol-4-yl)-[1,2,4]triazolo[3,4-b][1,3,4]thiadiazol-3-ylmethyl]-quinoline

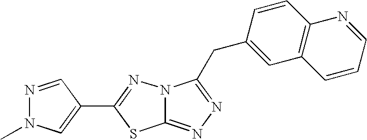

An equimolar mixture of 4-amino-5-quinolin-6-ylmethyl-4H-[1,2,4]triazole-3-thiol (0.78 g, 3.03 mmol), 1-methylpyrazol-4-carboxylic acid (0.39 g, 3.03 mmol) in phosphorous oxychloride (7.5 mL) was refluxed for 6 h. The reaction mixture was cooled to room temperature and then 30 g of crushed ice was added with stirring followed by addition of solid potassium hydroxide till the pH of the mixture was 8. The mixture was allowed to stand in an ice bath for 2 hours. The formed precipitate was filtered, washed with water, and placed into boiling ethanol and refluxed for 30 min, then allowed to cool. The resulting solid was filtered and dried to return title compound as an off white solid (195 mg, 18.5%).

### Example 3

### General Method B

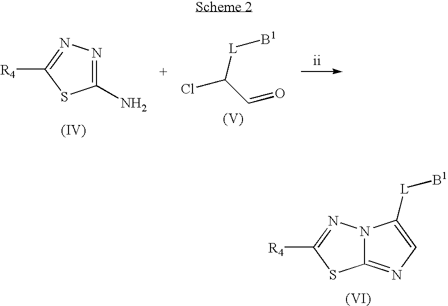

Compounds of formula (IV) where R₄ is described herein are either available commercially or prepared using methods described for the synthesis of intermediate 8 and transformations known to those skilled in the art.

Compounds of general formula (V) where L and B¹ are described herein are either available commercially or prepared using methods described for the synthesis of intermediate 3 and transformations known to those skilled in the art.

Compounds of general formula (VI) may be prepared from compounds of formula (IV) and compounds of general formula (V) by process step (ii), which comprises a cyclo-condensation reaction at elevated temperature, typically in the range of 70° C. to 180° C. in a suitable solvent, typically but not limited to ethanol or dimethylacetamide.

### Example 3A

### 6-[2-(1-methyl-1H-pyrazol-4-yl)-imidazo[2,1-b][1,3,4]thiadiazol-5-ylmethyl]-quinoline

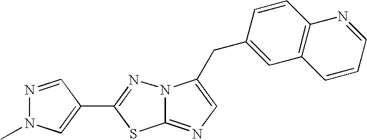

To a solution of crude 2-chloro-3-quinolin-6-yl-propionaldehyde (0.27 mmol) in EtOH (2 mL) was added 5-(1-methyl-1H-pyrazol-4-yl)-[1,3,4]thiadiazol-2-ylamine (40 mg, 0.225 mmol). The reaction mixture was stirred at 80° C. for 21 h. It was then transferred to a microwave vessel with an additional 3 mL of EtOH and the mixture was reacted in a microwave reactor at 150° C. for 3 h. The reaction mixture was concentrated in vacuo and the residue was treated with 10% aqueous NaOH. The aqueous layer was extracted with 10% MeOH/DCM (2×) and the organic layers were combined and adsorbed on silica gel. Purification by flash chromatography on silica gel using a gradient of 0-8% MeOH:DCM, followed by trituration with EtOAc and filtration, afforded 8 mg of 6-[2-(1-methyl-1H-pyrazol-4-yl)-imidazo[2,1-b][1,3,4]thiadiazol-5-ylmethyl]-quinoline as a white solid (10% yield): ¹H NMR (DMSO-d6) δ 3.90 (s, 3H), 4.43 (s, 2H), 7.10 (s, 1H), 7.50 (dd, 1H), 7.75 (dd, 1H), 7.86 (dd, 1H), 7.98 (d, 1H), 8.01 (s, 1H), 8.33 (dd, 1H), 8.49 (s, 1H), 8.85 (dd, 1H); MS (m/z) 347 [M+H⁺]⁺.

### Example 4

### General Method C

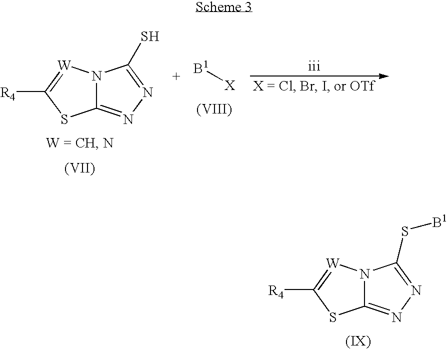

Compounds of general formula (IX) may be prepared from compounds of formula (VII) and compounds of general formula (VIII) by process step (iii), which comprises a S-substitution reaction in a suitable solvent, in the presence of a base, a metal catalyst, and a ligand. Compounds of formula (VIII) are either available commercially or prepared from commercial compounds using standard chemical reactions and transformations known to those skilled in the art. The S-substitution reaction can be carried out as described in the literature: Itoh, T. et al Org. Lett. 2004, 6, 4587; Schopfer, U. et al Tetrahedron 2001, 57, 3069; Buchwald, S. L. et al Org. Lett. 2002, 4, 3517; Cheng, C.-H. et al Org. Lett. 2006, 8, 5613. Typical conditions comprise 1 equivalent of thiol (VII), 1 equivalent of aryl halide or triflate (VIII), 2 equivalents of diisopropylethylamine, 0.05 equivalents of tris(dibenzylideneacetone)di-palladium (0), and 0.1 equivalent of 4,5-bis(diphenylphosphino)-9,9-dimethylxanthene (Xantphos) in DMF at 100° C. for several hours. Alternatively, in case of an activated aryl or heteroaryl halide (VIII), process step (iii) can proceed via a nucleophilic substitution reaction in presence of a base in a suitable solvent. Typical conditions comprise 1 equivalent of thiol (VII), 1.1 equivalent of activated aryl or heteroaryl halide (VIII), and 1.2 equivalent of potassium hydroxide in ethanol at 70° C. for several hours.

### Example 4A

### 6-[6-(1-Methyl-1H-pyrazol-4-yl)-[1,2,4]triazolo[3,4-b][1,3,4]thiadiazol-3-ylsulfanyl]-quinoline

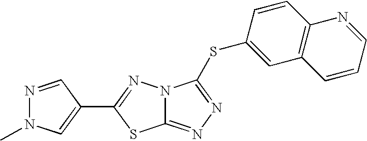

A solution of trifluoro-methanesulfonic acid quinolin-6-yl ester (119 mg, 0.429 mmol), diisopropylethylamine (0.224 mL, 1.29 mmol) in DMF (2 mL) under nitrogen was degassed by bubbling in nitrogen for 5 min. Tris(dibenzylideneacetone)dipalladium (20 mg, 0.021 mmol), Xantphos (25 mg, 0.043 mmol), 6-(1-Methyl-1H-pyrazol-4-yl)-[1,2,4]triazolo[3,4-b][1,3,4]thiadiazole-3-thiol (intermediate 9) (102 mg, 0.429 mmol) were added, and the mixture was degassed for another 5 min. The reaction mixture was stirred at 100° C. overnight. The reaction mixture was cooled to room temperature and the DMF was removed by rotary evaporation under reduced pressure. Purification by flash chromatography on silica gel using a gradient of 0-15% MeOH:DCM afforded a yellowish solid. The yellowish solid was treated with EtOH, stirred and filtered to obtain title compound (22.0 mg, 14% yield) as a white solid.

### Example 5

### Suzuki Coupling (General Method D)

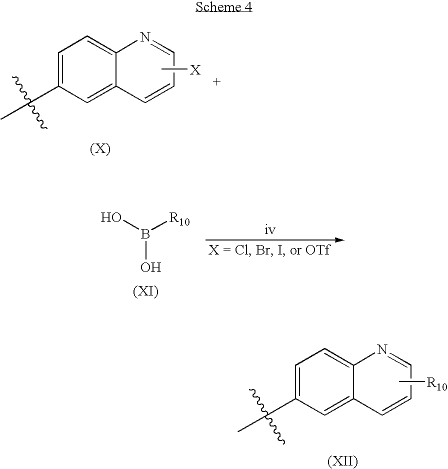

Compounds of general formula (XII) where R₁₀ is as described herein may be prepared according to general reaction scheme 4. Compounds of formula (XI) are either available commercially or prepared from commercial compounds using standard chemical reactions and transformations. Compounds of formula (X) can be prepared according to methods described herein. Compounds of general formula (XII) may be prepared from compounds of formula (X) and compounds of general formula (XI) by process step (iv), which comprises a Suzuki coupling reaction in a suitable solvent, in the presence of a base and a palladium catalyst. The Suzuki coupling reaction can be carried out as described in the literature: Suzuki, A. Pure & Appli. Chem. 1985, 57, 1749 and reference contained within; Angew. Chem. Int. Ed, 2002, 41, 4176-4211. Typical conditions comprise heating 1 equivalent of aryl halide or triflate (X), 1.1 equivalents of boronic acid (XI) or its boronate ester equivalent, 2 equivalents of potassium carbonate, 0.05-0.1 equivalents of palladium catalyst (dichloro[1,1′-bis(diphenylphosphino)ferrocene]palladium(II) dichloromethane adduct or dichlorobis(triphenylphosphine) palladium (II)) in a mixture of 1,4-dioxane and water under microwave heating at 130° C. for 20 minutes.

### Example 5A

### 3-(1-Methyl-1H-pyrazol-4-yl)-6-[1,2,4]triazolo[4,3-b][1,2,4]triazin-3-ylmethyl-quinoline

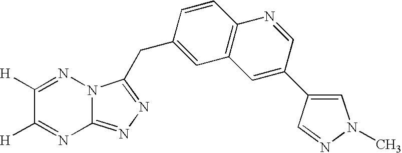

To a degassed (bubbled nitrogen for 15 mins) mixture of water (1 mL) and 1,4-dioxane (21 mL) was added 3-bromo-6-[1,2,4]triazolo[4,3-b][1,2,4]triazin-3-ylmethyl-quinoline (69 mg, 0.2 mmol, 1.0 equiv.), N-methylpyrazole pinacolboronic ester (51 mg, 0.25 mmol, 1.2 equiv.), potassium carbonate (2 equiv., 0.41 mmol, 56 mg) and dichlorobis(triphenylphosphine) palladium (II) (8 mg, 0.07 equiv). The microwave tube was capped and heated in a microwave reactor at 130° C. for 20 minutes. After such time the mixture was concentrated onto silica gel and purified via flash column chromatography eluting with dichloromethane:methanol 100:0 to 90:10 to return title compound as a white solid (11 mg, 29% yield)

### Example 5B

### 6-(6-Phenyl-[1,2,4]triazolo[4,3-a]pyrimidin-3-ylmethyl)-quinoline

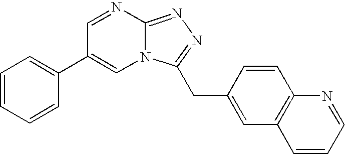

6-(6-Bromo-[1,2,4]triazolo[4,3-a]pyrimidin-3-ylmethyl)-quinoline (36.1 mg, 0.106 mmol) and Phenyl boronic acid (13 mg, 0.106 mmol) were placed in a microwave vial, suspended in dimethoxyethane (850 uL) and purged with Nitrogen gas. Next, sodium carbonate (34 mg, 0.318 mmol) in water (425 uL) was added the reaction vessel and purged with nitrogen gas again. Finally, bis(triphenylphosphine)palladium (II) chloride (3.7 mg, 0.005 mmol) was added to the reaction mixture. The vial was sealed and heated to 150° C. for 10 minutes in the microwave. The reaction mixture was diluted with Ethyl acetate (2 ml) and filtered through celite. The celite was rinsed with 5 ml of Methanol. The filtrate was concentrated down in vacuo and purified by flash chromatography (0-10% MeOH/DCM gradient) to afford the desired product, 6-(6-Phenyl-[1,2,4]triazolo[4,3-a]pyrimidin-3-ylmethyl)-quinoline (1.6 mg, 4.5% yield).

### Example 6

### General Method E

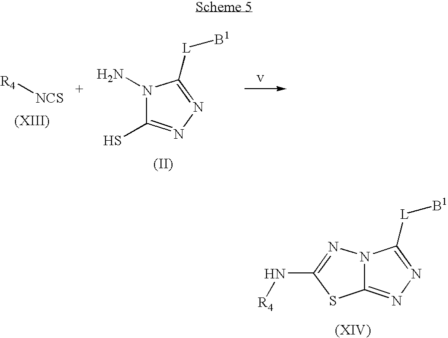

Compounds of formula (XIII) where R4 is described herein are either available commercially or prepared using transformations known to those skilled in the art.

Compounds of general formula (II) where L and B¹ are described herein are either available commercially or prepared using methods described for the synthesis of intermediate 5 and transformations known to those skilled in the art.

Compounds of general formula (XIV) may be prepared from compounds of formula (XIII) and compounds of general formula (II) by process step (v), which comprises heating an amino thiol (II) and an isothiocyanate (XIII) in a suitable solvent such as N,N-dimethylacetamide (DMA).

### Example 6A

### cyclopropyl-(3-quinolin-6-ylmethyl-[1,2,4]triazolo[3,4-b][1,3,4]thiadiazol-6-yl)-amine

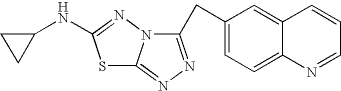

4-Amino-5-quinolin-6-ylmethyl-4H-[1,2,4]triazole-3-thiol (50 mg, 0.195 mmol) and cyclopropyl isothiocyanate (23 uL, 0.234 mmol) were heated at 160° C., overnight in DMA (1 mL). The desired product was purified by preparative HPLC to give 3 mg of cyclopropyl-(3-quinolin-6-ylmethyl-[1,2,4]triazolo[3,4-b][1,3,4]thiadiazol-6-yl)-amine (3 mg, 5% yield). (DMSO-d6) δ 0.56 (m, 2H), 0.75 (m, 2H), 2.65 (m, 1H), 4.47 (s, 2H), 7.52 (dd, 1H), 7.72 (dd, 1H), 7.86 (d, 1H), 7.98 (d, 1H), 8.32 (dd, 1H), 8.62 (br s, 1H), 8.87 (dd, 1H). MS (m/z) M+H=323.

### Example 7

### General Method F

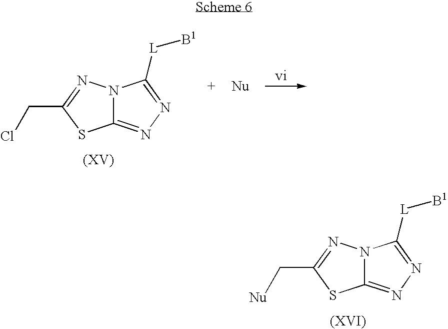

Compounds of general formula (XVI) may be prepared from compounds of formula (XV) and an appropriate nucleophile Nu by process step (vi), which comprises a nucleophilic substitution reaction in a suitable solvent in the presence of a base. Typically, compound (XV) and the nucleophile are mixed in a polar aprotic solvent, such as DMSO, at room temperature or elevated temperature.

### Example 7A

### (2-Fluoro-ethyl)-{3-[3-(1-methyl-1H-pyrazol-4-yl)-quinolin-6-ylmethyl]-1,2,4]triazolo[3,4-b][1,3,4]thiadiazol-6-ylmethyl}-amine

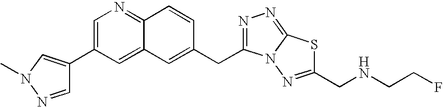

To a solution of 6-(2-chloromethyl-[1,2,4]triazolo[5,1-b][1,3,4]thiadiazol-6-ylmethyl)-3-(1-methyl-1H-pyrazol-4-yl)-quinoline (100 mg, 0.253 mmol) in DMSO (1 mL) was added 2-fluoroethylamine hydrochloride (125 mg, 1.26 mmol) and potassium carbonate (280 mg, 2.024 mmol). The reaction mixture was stirred at room temperature for 2 h. It was then filtered and purified by preparative HPLC to give the title compound as an off white solid (7.9 mg, 6.4%).

### Example 8

### Salt Formation (General Method G)

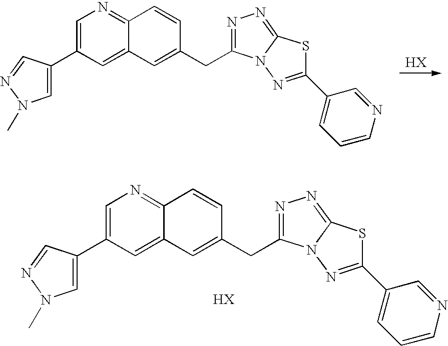

3-(1-Methyl-1H-pyrazol-4-yl)-6-[6-(1-methyl-1H-pyrazol-4-yl)-[1,2,4]triazolo[4,3-b]pyridazin-3-ylmethyl]-quinoline (50 mg, 0.118 mmol) was suspended in MeOH (2 mL) in a vial. A 2M solution of the corresponding acid in water (0.124 mmol, 1.05 equiv.) was added. The vial was heated at 80° C. and more MeOH was added until a clear solution was obtained. The solution was cooled to r.t. and concentrated in vacuo. The residue was triturated with diethyl ether, filtered, washed with diethyl ether, and dried in vacuo to provide the corresponding salt of 3-(1-methyl-1H-pyrazol-4-yl)-6-[6-(1-methyl-1H-pyrazol-4-yl)-[1,2,4]triazolo[4,3-b]pyridazin-3-ylmethyl]-quinoline.

### Example 9

### General Method H

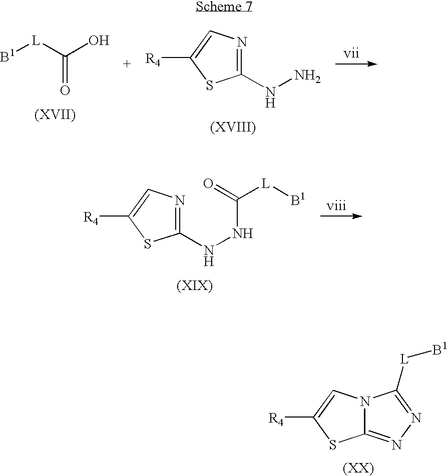

Compounds of formula (XVIII) where R₄ is described herein are either available commercially or prepared using methods described for the synthesis of intermediate 6 and transformations known to those skilled in the art.

Compounds of general formula (XVII) where L and B¹ are described herein are either available commercially or prepared using methods described for the synthesis of intermediate 1 and transformations known to those skilled in the art.

Compounds of general formula (XIX) may be prepared from compounds of formula (XVII) and compounds of general formula (XVIII) by process step (vii), where an amide coupling reagent, such as DCC, EDC, HATU, HBTU, or PyBOP, is used in the presence or absence of a catalytic amount of DMAP, HOBT, or HOAT, and in the presence of absence of a base, in a suitable solvent at temperatures ranging from 0° C. to 200° C. Typical conditions include 1 equivalent of carboxylic acid (XVII), 1 equivalent of hydrazine (XVIII), 1.5 equivalent of EDC, 1 equivalent of HOBT in DMF at room temperature for several hours.

Compounds of general formula (XX) may be prepared from compounds of formula (XIX) by process step (viii), which comprises heating hydrazide (XIX) in the presence of POCl₃.

### Example 9A

### 6-(6-Phenyl-thiazolo[2,3-c][1,2,4]triazol-3-ylmethyl)-quinoline

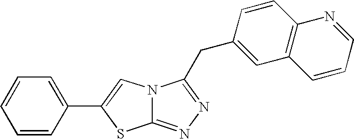

### Step 1: Quinolin-6-yl-acetic acid N′-(5-phenyl-thiazol-2-yl)-hydrazide

A mixture of (5-phenyl-thiazol-2-yl)-hydrazine (1.3 g, 6.8 mmol), quinolin-6-yl-acetic acid (1.27, 6.8 mmol), EDC (1.95, 10.3 mmol), HOBt (0.92, 6.8 mmol) and K₂CO₃ (4.70 g, 34.0 mmol) in CH₂Cl₂ (30 mL) was stirred at room temperature overnight. TLC showed the reaction was complete. The mixture was filtered and washed with water (20 ml×3) and EtOAc (20 ml×3) to give the product (1.2 g, 49.0%) as a pale solid. ¹H NMR (300 MHz, DMSO-d6): 8.875 (m, 1H), 8.355 (m, 1H), 8.985 (m, 2H), 7.90 (d, 1H), 7.720 (m, 1H), 7.600 (m, 2H), 7.530 (m, 1H), 7.400 (m, 2H), 7.320 (m, 1H), 5.400 (m, 2H), 4.437 (s, 2H). ES-MS: 361 (M+H⁺)

### Step 2: 6-(6-Phenyl-thiazolo[2,3-c][1,2,4]triazol-3-ylmethyl)-quinoline

A mixture of quinolin-6-yl-acetic acid N′-(5-phenyl-thiazol-2-yl)-hydrazide (700 mg, 1.94 mmol) and POCl₃ (10 ml) was stirred at 100° C. for 8 hours. TLC showed the reaction was complete. The mixture was concentrated to remove POCl₃ and added to aqueous Na₂CO₃ to adjust pH 68. Then the mixture was extracted with EtOAc (20 ml×3). The resulting mixture was purified by chromatography (EtOAc:CH₂Cl₂=1:9) to give the product (210 mg, 31.0%) as a pale solid.

The structure, name, physical and biological data are further described in tabular form below in Table I.

TABLE 1

| Enzyme | | | | | |
|---|---|---|---|---|---|
| Assay | XTT | | | | |
| c-MET | Assay | | | | MS |
| IC₅₀ | (GTL16) | | | | (m/z) |
| (nM) | IC₅₀ | Method | Structure | ¹H NMR (500 MHz) | [M + H⁺]⁺ |
| I | II | A | 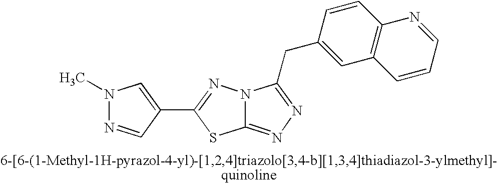 | (DMSO-d6) δ 3.91 (s, 3H), 4.63 (s, 2H), 7.51 (dd, 1H), 7.75 (d, 1H), 7.91 (s, 1H), 7.99 )d, 1H), 8.07 (s, 1H), 8.34 (d, 1H), 8.58 (s, 1H), 8.87 (d, 1H) | 348 |
| I | III | A | 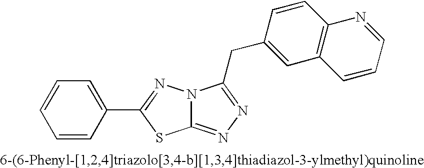 | (DMSO-d6) δ 4.72 (s, 2H), 7.54 (dd, 1H), ), 7.60-7.69 (m, 3H), 7.83 (dd, 1H), ), 7.94-8.05 (m, 3H), 8.38 (d, 1H), 8.90 (dd, 1H) | 344 |
| I | I | A | 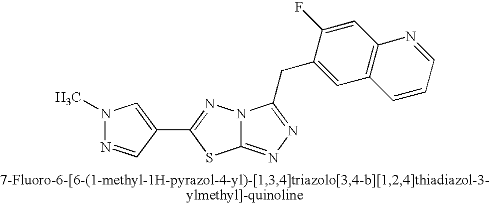 | (DMSO-d6) δ 3.91 (s, 3H), 4.64 (s, 2H), 7.52 (dd, 1H), 7.82 (d, 1H), 8.02-8.07 (m, 2H), 8.41 (d, 1H), 8.59 (s, 1H), 8.91 (d, 1H) | 366 |
| I | I | A | 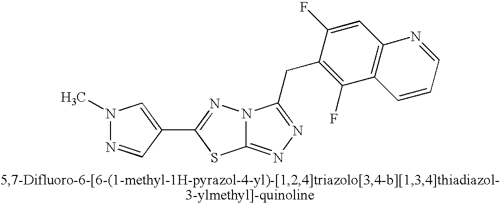 | (DMSO-d6) δ 3.92 (s, 3H), 4.62 (s, 2H), 7.62 (dd, 1H), 7.77 (d, 1H), 8.02 (s, 1H), 8.50-8.57 (m, 2H), 8.99 (d, 1H) | 384 |
| I | II | B | 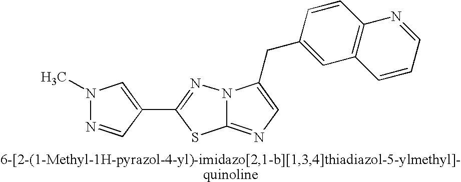 | (DMSO-d6) δ 3.90 (s, 3H), 4.43 (s, 2H), 7.10 (s, 1H), 7.50 (dd, 1H), 7.75 (dd, 1H), 7.86 (dd, 1H), 7.98 (d, 1H), 8.01 (s, 1H), 8.33 (dd, 1H), 8.49 (s, 1H), 8.85 (dd, 1H) | 347 |
| III | — | A | 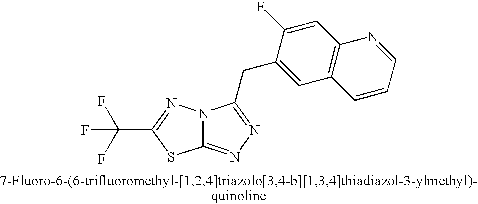 | (DMSO-d6) δ 4.71 (s, 2H), 7.53 (dd, 1H), 7.81 (d, 1H), 8.05 (d, 1H), 8.39 (dd, 1H), 8.92 (dd, 1H) | 354 |
| II | III | C | 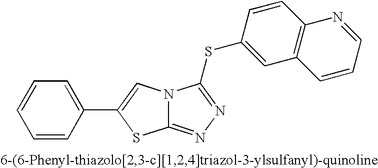 | (DMSO-d6) δ: 7.36-7.94 (m, 3H), 7.48 (dd, 1H), 7.63 (dd, 1H), 7.66-7.67 (m, 2H), 7.93 (s, 1H), 7.94 (d, 1H), 8.27 (dd, 1H), 8.51 (s, 1H), 8.83 (dd, 1H) | 361 |
| I | II | C | 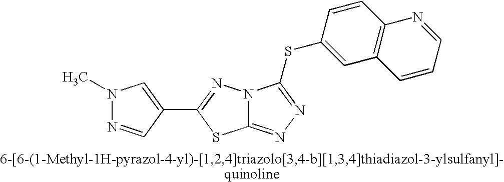 | (DMSO-d6) δ: 7.55 (dd, 1H), 7.72 (dd, 1H), 8.00 (d, 1H), 8.05 (d, 1H), 8.80 (s, 1H), 8.34 (dd, 1H), 8.61 (s, 1H), 8.90 (dd, 1H) | 366 |
| I | I | D | 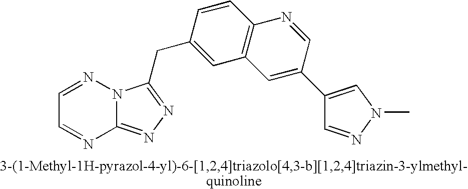 | (DMSO-d6) δ: 3.90 (3H, s), 4.73 (2H, s), 7.65 (1H, d), 7.76 (1H, s), 7.92 (1H, d), 8.07 (1H, s), 8.37 (1H, s), 8.40 (1H, s), 8.72 (1H, d), 8.73 (1H, d), 9.13 (1H, s) | 343 |
| II | II | A | 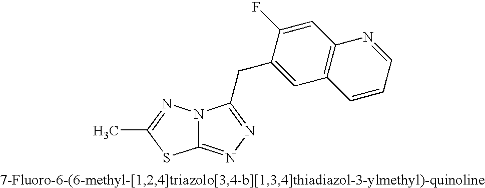 | (DMSO-d6) δ 2.71 (s, 3H), 4.62 (s, 2H), 7.52 (dd, 1H), 7.80 (d, 1H), 7.99 (d, 1H), 8.39 (d, 1H), 8.91 (d, 1H) | 300 |
| II | III | A | 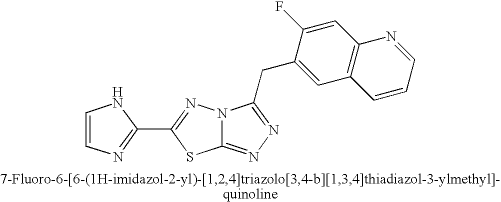 | (DMSO-d6) δ 4.66 (s, 2H), 7.30-7.49 (2H, bm), 7.52 (1H, dd), 7.82 (1H, d), 8.01 (1H, d), 8.38 (1H, dd), 8.90 (1H, dd), 13.80 (1H, bs) | 352 |
| I | I | A | 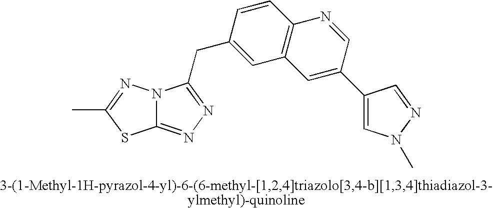 | (DMSO-d6) δ 2.69 (s, 3H), 3.91 (s, 3H), 4.95 (s, 2H), 7.61 (d, 1H), 7.76 (s, 1H), 7.92 (d, 1H), 8.07 (s, 1H), 3.83 (s, 1H), 8.42 (s, 1H), 9.14 (s, 1H) | 362 |
| II | — | C | 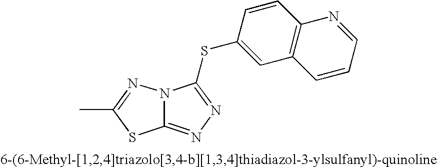 | (DMSO-d6) δ 2.70 (s, 3H), 7.53 (d, 1H), 7.64 (d, 1H), 7.97 (d, 1H), 8.07 (s, 1H), 8.32 (d, 1H), 8.88 (s, 1H) | 300 |
| I | I | A | 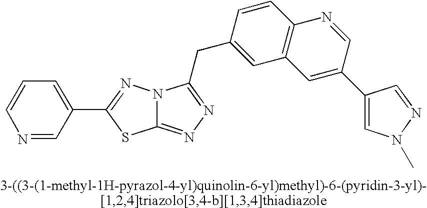 | (DMSO-d6) δ 3.90 (s, 3H), 4.70 (s, 2H), 7.64 (m, 1H), 7.73 (dd, 1H), 7.87 (s, 1H), 7.96 (d, 1H), 8.08 (s, 1H), 8.32 (m, 1H), 8.38 (s, 1H), 8.47 (s, 1H), 8.81 (dd, 1H), 9.12 (s, 1H), 9.15 (d, 1H) | 425 |
| I | I | F | 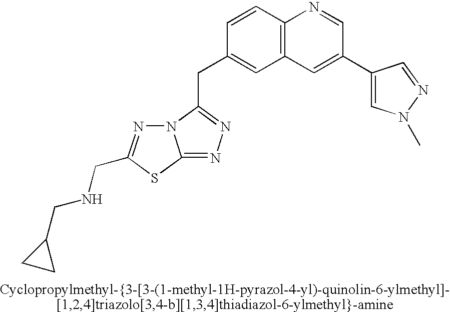 | (DMSO-d6) δ .02 (m, 2H), 0.23 (m, 2H), 0.83 (m, 1H), 2.89 (m, 2H), 3.57 (s, 2H), 3.77 (s, 3H), 4.16 (s, 2H), 7.46 (m, 2H), 7.58 (s, 1H), 7.75 (d, 1H), 7.94 (s, 1H), 8.23 (s, 1H), 8.25 (s, 1H), 8.98 (s, 1H) | 431 |
| I | I | A | 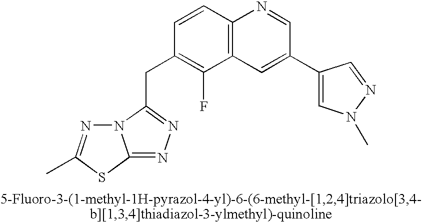 | (DMSO-d6) δ 2.70 (s, 3H), 3.91 (3H, s), 4.62 (s, 2H), 7.66 (t, 1H), 7.83 (d, 1H), 8.20 (s, 1H), 8.50 (s, 1H), 8.56 (1H, s), 9.26 (s, 1H) | 379 |
| II | II | A | 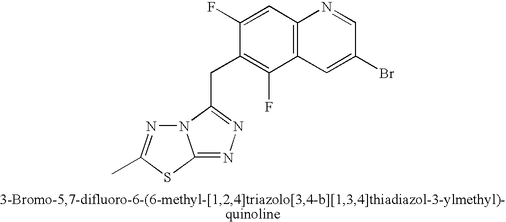 | (DMSO-d6) δ 2.71 (3H, s), 4.62 (2H, s), 7.78 (1H, d), 8.78 (1H, m), 9.06 (1H, d) | 396 |
| I | I | C | 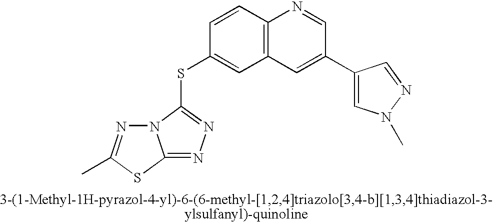 | (DMSO-d6) δ 2.69 (s, 3H), 3.90 (3H, s), 7.59 (1H, dd), 7.84 (1H, d), 7.93 (1H, d), 8.06 (1H, d), 8.37 (1H, s), 8.42 (1H, d), 9.17 (1H, d) | 380 |
| II | — | A | 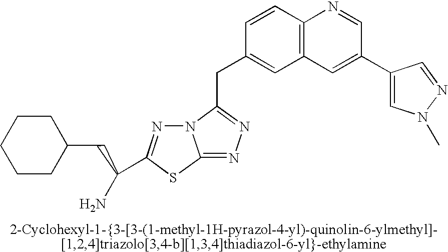 | (DMSO-d6) δ 0.75-1.80 (m, 13H), 3.91 (s, 3H), 4.15 (t, 1H), 4.60 (s, 2H), 7.61 (dd, 1H), 7.76 (d, 1H), 7.93 (d, 1H), 8.08 (s, 1H), 8.38 (s, 1H), 8.43 (d, 1H), 9.14 (d, 1H) | 473 |
| II | — | A | 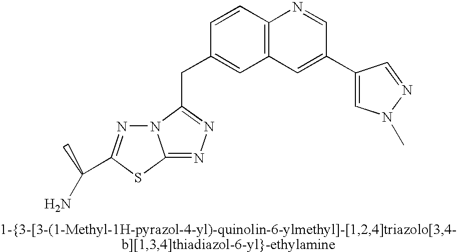 | (DMSO-d6) δ 1.35 (d, 3H), 3.86 (s, 3H), 4.19 (q, 1H), 4.53 (s, 2H), 7.58 (dd, 1H), 7.73 (d, 1H), 7.89 (d, 1H), 8.04 (d, 1H), 8.33 (s, 1H), 8.39 (d, 1H), 9.09 (d, 1H) | 391 |
| I | I | A | 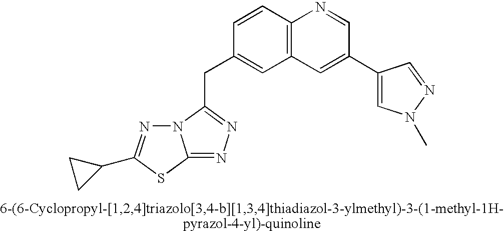 | (DMSO-d6) δ 1.10 (m, 2H), 1.20 (m, 2H), 2.40 (m, 1H), 3.91 (s, 3H), 4.57 (s, 2H), 7.64 (dd, 1H), 7.78 (d, 1H), 7.94 (d, 1H), 8.10 (s, 1H), 8.40 (s, 1H), 8.44 (d, 1H), 9.15 (d, 1H) | 388 |
| I | I | E | 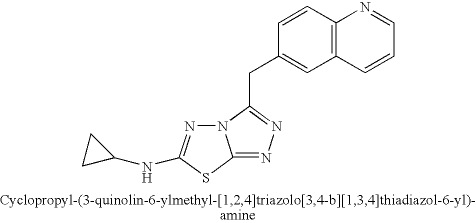 | (DMSO-d6) δ 0.56 (m, 2H), 0.75 (m, 2H), 2.65 (m, 1H), 4.47 (s, 2H), 7.52 (dd, 1H), 7.72 (dd, 1H), 7.86 (d, 1H), 7.98 (d, 1H), 8.32 (dd, 1H), 8.62 (br s, 1H), 8.87 (dd, 1H) | 323 |
| II | — | A | 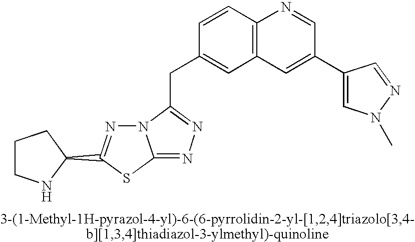 | (DMSO-d6) δ 1.67 (m, 2H), 1.83 (m, 1H), 2.15 (m, 1H), 2.81 (m, 1H), 2.90 (m, 1H), 3.86 (s, 3H), 4.46 (m, 1H), 4.53 (s, 2H) | 417 |
| I | I | F | 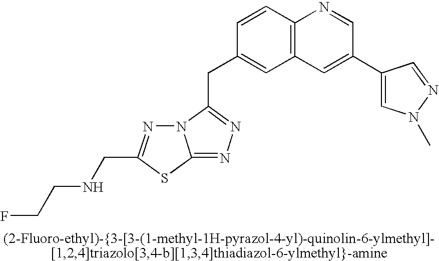 | (DMSO-d6) δ 3.42 (q, 1H), 3.47 (q, 1H), 3.84 (s, 3H), 4.23 (s, 2H), 4.41 (t, 1H), 4.51 (t, 1H), 7.53 (d, 1H), 7.66 (s, 1H), 7.75 (t, 1H), 7.84 (d, 1H), 8.01 (s, 1H), 8.31 (s, 1H), 8.34 (d, 1H), 9.06 (d, 1H) | 424 |
| I | II | F | 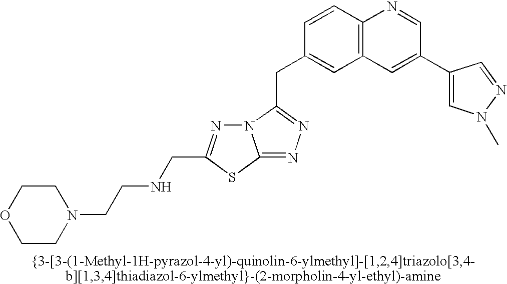 | (DMSO-d6) δ 2.17 (s, 4H), 2.30 (m, 4H), 3.21 (m, 2H), 3.48 (m, 2H), 3.64 (s, 2H), 3.84 (s, 3H), 4.25 (s, 2H), 7.40 (t, 1H), 7.52 (dd, 1H), 7.63 (s, 1H), 7.83 (d, 1H), 8.01 (s, 1H), 8.30 (s, 1H), 8.34 (d, 1H), 9.06 (d, 1H) | 491 |
| I | I | A | 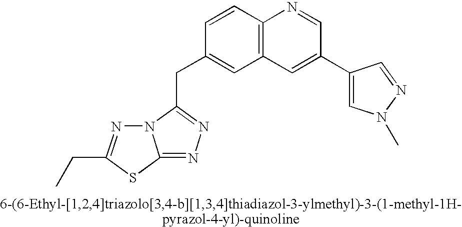 | (DMSO-d6) δ 1.30 (t, 3H), 3.05 (q, 2H), 3.92 (s, 3H), 4.61 (s, 2H), 7.66 (dd, 1H), 7.80 (d, 1H), 7.95 (dd, 1H), 8.10 (d, 1H) 8.40 (s, 1H), 8.45 (d, 1H), 9.16 (d, 1H) | 376 |
| I | I | F | 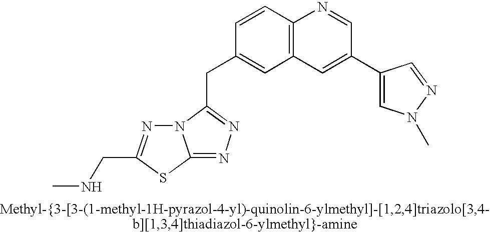 | (DMSO-d6) δ 2.76 (d, 3H), 3.69 (s, 2H), 3.90 (s, 3H), 4.31 (s, 2H), 7.52 (m, 1H), 7.63 (dd, 1H), 7.74 (s, 1H), 7.90 (d, 1H), 8.08 (s, 1H), 8.38 (s, 1H), 8.41 (d, 1H), 9.12 (d, 1H) | 391 |
| II | — | F | 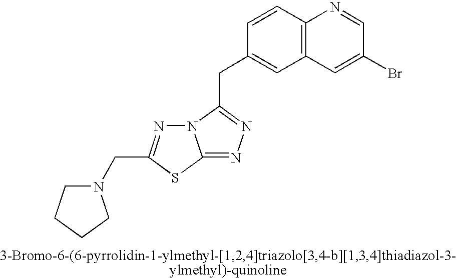 | (DMSO-d6) δ 1.89 (m, 6H), 3.37 (m, 2H), 3.50 (m, 2H), 3.91 (s, 2H), 4.30 (s, 2H), 7.75 (dd, 1H), 7.80 (s, 1H), 7.96 (d, 1H), 8.68 (d, 1H), 8.88 (d, 1H) | 429 |
| I | II | C | 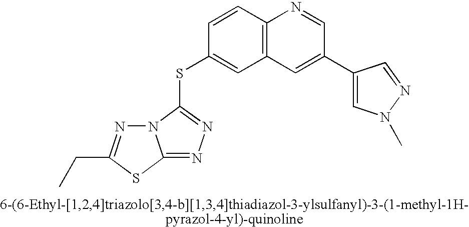 | (DMSO-d6) δ 1.26 (t, 3H), 3.06 (q, 2H), 3.90 (s, 3H), 7.60 (dd, 1H), 7.87 (d, 1H), 7.94 (d, 1H), 8.07 (s, 1H), 8.43 (d, 1H), 9.17 (d, 1H) | 394 |
| I | I | F | 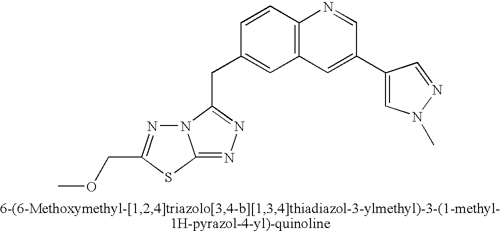 | (DMSO-d6) δ 3.91 (s, 3H), 3.98 (s, 2H), 4.36 (s, 2H), 7.63 (d, 1H), 7.79 (s, 3H), 7.92 (d, 1H), 8.09 (s, 1H) 8.38 (s, 1H), 8.43 (s, 1H), 9.14 (s, 1H) | 392 |
| I | I | F | 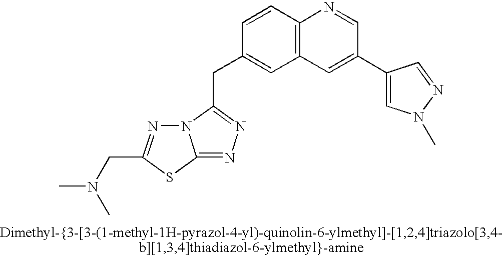 | (DMSO-d6) δ 2.96 (s, 6H), 3.84 (s, 3H), 3.89 (s, 2H), 7.55 (d, 1H), 7.67 (s, 1H), 7.83 (d, 1H), 8.02 (s, 1H), 8.31 (s, 1H), 8.34 (s, 1H), 9.06 (s, 1H) | 405 |
| I | I | F | 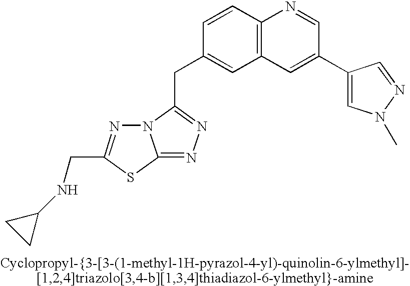 | (DMSO-d6) δ 0.37 (m, 2H), 0.64 (m, 2H), 2.61 (m, 1H), 3.56 (s, 2H), 3.84 (s, 3H), 4.26 (s, 2H), 7.59 (d, 1H), 7.7 (s, 1H), 7.83 (d, 1H), 8.01 (s, 1H), 8.31 (s, 1H), 8.34 (d, 1H), 9.06 (s, 1H) | 417 |
| I | I | F | 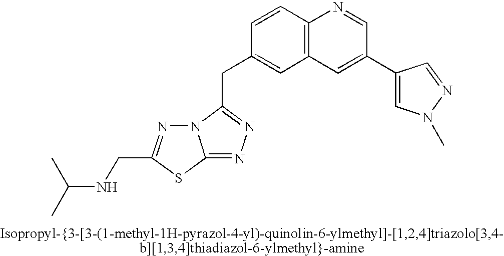 | (DMSO-d6) δ 1.04 (d, 6H), 2.90 (br s, 1H), 3.59 (s, 2H), 3.80 (m, 1H), 3.84 (s, 3H), 4.23 (s, 2H) | 419 |
| I | II | F | 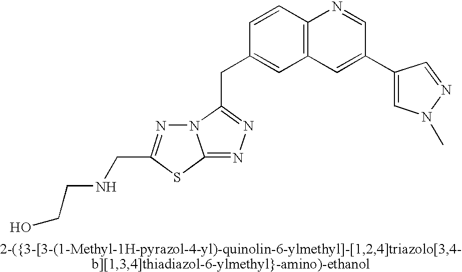 | (DMSO-d6) δ 3.22 (m, 2H), 3.48 (t, 2H), 3.66 (s, 2H), 3.84 (s, 3H), 4.23 (s, 2H), 7.54 (m, 1H), 7,68 (s, 1H), 7.83 (d, 1H), 8.01 (s, 1H), 8.31 (s, 1H), 8.35 (s, 1H), 9.06 (s, 1H) | 421 |
| III | — | A | 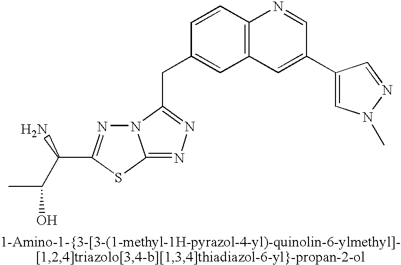 | (DMSO-d6) δ 1.00 (d, 3H), 3.84 (m, 4H), 3.96 (d, 1H), 4.53 (s, 2H), 7.59 (dd, 1H), 7.73 (s, 1H), 7.86 (d, 1H), 8.02 (s, 1H), 8.32 (1H, s), 8.36 (d, 1H), 9.08 (d, 1H) | 422 |
| I | II | A | 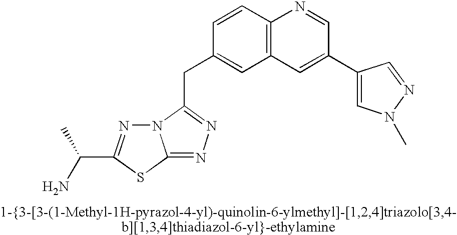 | (DMSO-d6) δ 1.34 (d, 3H), 3.84 (s, 3H), 4.17 (q, 1H), 4.52 (s, 2H), 7.57 (dd, 1H), 7.72 (br s, 1H), 7.87 (d, 1H) 8.03 (s, 1H), 8.32 (s, 1H), 8.37 (d, 1H), 9.08 (d, 1H) | 391 |
| II | — | A | 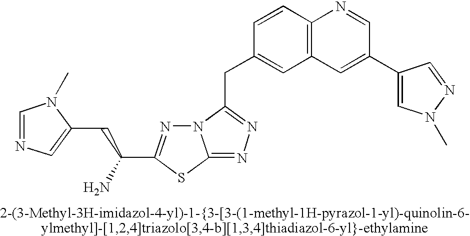 | (DMSO-d6) δ 2.90 (m, 1H), 3.03 (m, 1H), 3.84 (s, 3H), 4.32 (t, 1H), 4.51 (s, 2H), 6.66 (s, 1H), 7.39 (s, 1H), 7.55 (dd, 1H), 7.70 (s, 1H), 7.88 (d, 1H), 8.04 (s, 1H), 8.40 (d, 1H), 9.08 (d, 1H) | 471 |
| II | II | A | 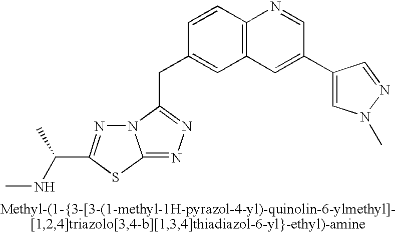 | (DMSO-d6) δ 1.31 (d, 3H), 2.24 (d, 3H), 3.85 (d, 3H), 3.88 (m, 1H), 4.52 (s, 2H), 7.58 (dd, 1H), 7.73 (s, 1H), 7.87 (d, 1H), 8.03 (s, 1H), 8.32 (s, 1H), 8.37 (d, 1H), 9.08 (d, 1H) | 406 |
| I | II | A | 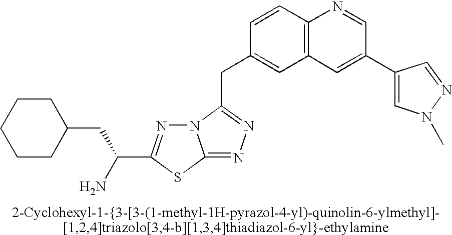 | (DMSO-d6) δ 0.70-1.53 (m, 13H), 3.84 (s, 3H), 4.08 (t, 1H), 4.53 (s, 2H), 7.55 (dd, 1H), 7.69 (s, 1H), 7.86 (d, 1H), 8.01 (s, 1H), 8.31 (s, 1H), 8.36 (s, 1H), 9.07 (d, 1H) | 473 |
| I | I | A | 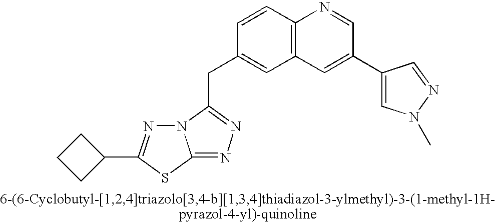 | (DMSO-d6) δ 1.86 (m, 1H), 2.00 (m, 1H), 2.23 (m, 2H), 2.32 (m, 2H), 3.32 (m, 4H), 4.54 (s, 2H), 7.59 (dd, 1H), 7.73 (s, 1H), 7.87 (d, 1H), 8.02 (s, 1H), 8.32 (s, 1H), 8.37 (s, 1H), 9.08 (d, 1H) | 402 |
| I | I | F | 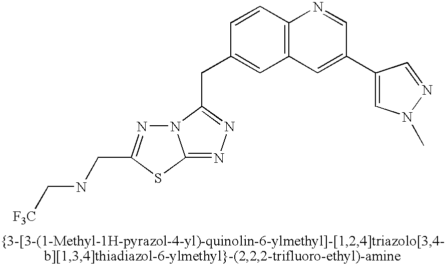 | (DMSO-d6) δ 3.78 (s, 2H), 3.87 (s, 3H), 4.10 (m, 2H), 4.28 (s, 2H), 7.57 (dd, 1H), 7.70 (d, 1H), 7.85 (d, 1H), 8.03 (s, 1H), 8.09 (t, 1H), 8.33 (s, 1H), 9.09 (d, 1H) | 459 |
| I | II | F | 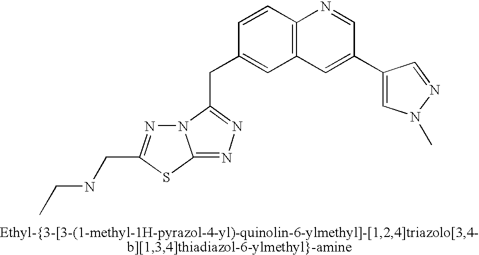 | (DMSO-d6) δ 1.08 (t, 3H), 3.13 (d, 2H), 3.65 (s, 1H), 3.88 (s, 3H), 4.27 (s, 2H), 7.46 (br t, 1H), 7.58 (dd, 1H), 7.70 (d, 1H), 7.87 (d, 1H), 8.05 (s, 1H), 8.34 (s, 1H), 8.37 (d, 1H), 9.10 (d, 1H) | 405 |
| I | − | A | 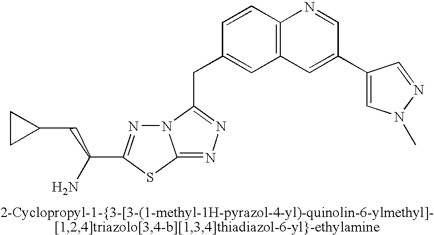 | (DMSO-d6) δ 0.10 (m, 1H), 0.30 (m, 1H), 0.83 (m, 1H), 1.68 (t, 1H), 4.00 (s, 1H), 4.28 (t, 1H), 4.68 (s, 1H), 7.70 (dd, 1H), 7.83 (s, 1H), 8.01 (d, 1H), 8.18 (s, 1H), 8.47 (s, 1H), 8.50 (d, 1H), 9.23 (d, 1H) | 431 |
| III | — | H | 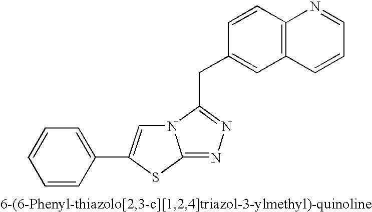 | (DMSO-d6) δ 4.64 (2H, s), 7.47 (4H, m), 7.53 (2H, d), 7.67 (1H, d), 7.98 (2H, t), 8.34 (1H, d), 8.65 (1H, s), 8.87 (1H, m). | 343 |
| I | — | G | 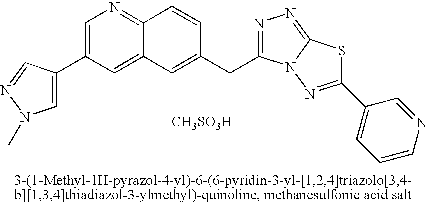 | ¹H NMR (DMSO-d6, 500 MHz) δ 2.33 (s, 3H), 3.93 (s, 3H), 4.77 (s, 2H), 7.67 (dd, 1H), 7.92 (d, 1H), 8.03 (s, 1H), 8.08 (d, 1H), 8.15 (s, 1H), 8.35 (dt, 1H), 8.45 (s, 1H), 8.83 (dd, 1H), 8.87 (bs, 1H), 9.14 (d, 1H), 9.34 (d, 1H) | 425 |
| I | — | G | 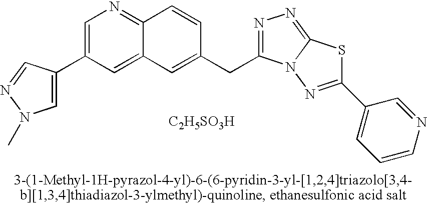 | ¹H NMR (DMSO-d6, 500 MHz) δ 1.06 (t, 3H), 2.39 (q, 2H), 3.92 (s, 3H), 4.75 (s, 2H), 7.66 (dd, 1H), 7.87 (dd, 1H), 7.99 (s, 1H), 8.06 (d, 1H), 8.14 (s, 1H), 8.35 (dt, 1H), 8.43 (s, 1H), 8.77 (bs, 1H), 8.83 (dt, 1H), 9.14 (d, 1H), 9.30 (d, 1H) | 425 |
| I | — | G | 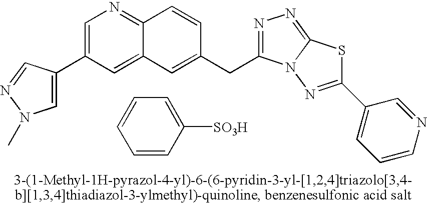 | ¹H NMR (DMSO-d6, 500 MHz) δ 3.92 (s, 3H), 4.76 (s, 2H), 7.30 (m, 3H), 7.59 (m, 2H), 7.66 (dd, 1H), 7.87 (dd, 1H), 8.00 (s, 1H), 8.06 (d, 1H), 8.14 (s, 1H), 8.36 (dt, 1H), 8.43 (s, 1H), 8.79 (bs, 1H), 8.83 (dd, 1H), 9.14 (d, 1H), 9.31 (d, 1H) | 425 |
| I | — | G | 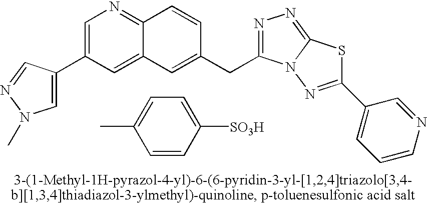 | ¹H NMR (DMSO-d6, 500 MHz) δ 2.28 (s, 3H), 3.92 (s, 3H), 4.75 (s, 2H), 7.11 (d, 2H), 7.47 (d, 2H), 7.67 (dd, 1H), 7.88 (dd, 1H), 8.00 (s, 1H), 8.05 (d, 1H), 8.14 (s, 1H), 8.35(dt, 1H), 8.43 (s, 1H), 8.79 (bs, 1H), 8.83 (dd, 1H), 9.14 (d, 1H), 9.31 (d, 1H) | 425 |
| I | — | G | 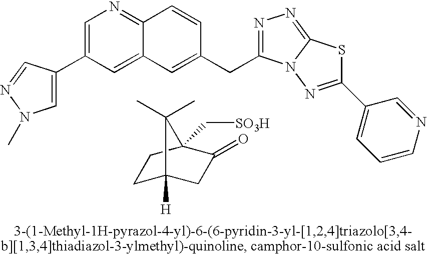 | ¹H NMR (DMSO-d6, 500 MHz) δ 0.74 (s, 3H), 1.06 (s, 3H), 1.28 (d, 2H), 1.79 (d, 1H), 1.84 (m, 1H), 1.93 (t, 1H), 2.23 (dt, 1H), 2.37 (d, 1H), 2.69 (t, 1H), 2.86 (d, 1H), 3.92 (s, 3H), 4.76 (s, 2H), 7.67 (dd, 1H), 7.88 (dd, 1H), 8.00 (s, 1H), 8.06 (d, 1H), 8.14 (s, 1H), 8.35 (dt, 1H), 8.44 (s, 1H), 8.80 (bs, 1H), 8.83 (dd, 1H), 9.14 (d, 1H), 9.31 (d, 1H) | 425 |
| I | — | G | 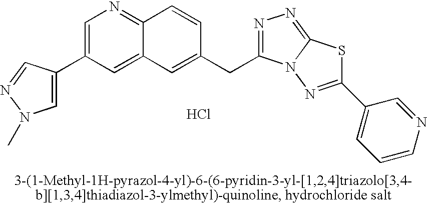 | ¹H NMR (DMSO-d6, 500 MHz) δ 3.92 (s, 3H), 4.75 (s, 2H), 7.67 (dd, 1H), 7.88 (dd, 1H), 7.99 (s, 1H), 8.09 (d, 1H), 8.14 (s, 1H), 8.35 (dt, 1H), 8.44 (s, 1H), 8.78 (bs, 1H), 8.83 (dd, 1H), 9.14 (d, 1H), 9.31 (d, 1H) | 425 |
| I | — | G | 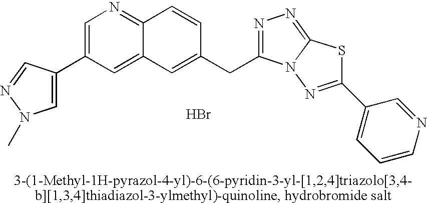 | ¹H NMR (DMSO-d6, 500 MHz) δ 3.92 (s, 3H), 4.76 (s, 2H), 7.67 (dd, 1H), 7.89 (dd, 1H), 8.01 (s, 1H), 8.06 (d, 1H), 8.15 (s, 1H), 8.35 (dt, 1H), 8.44 (s, 1H), 8.81 (bs, 1H), 8.83 (dd, 1H), 9.14 (d, 1H), 9.32 (d, 1H) | 425 |
| IV | — | D | 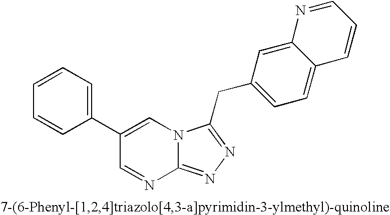 | ¹H NMR (DMSO-d6, 500 MHz) δ 4.47 (s, 2H), 7.55 (m, 4H), 7.81 (dd, 1H), 7.86 (m, 2H), 7.94 (d, 1H), 7.99 (d, 1H), 8.34 (dd, 1H), 8.87 (dd, 1H), 9.22 (d, 1H), 9.70 (d, 1H) | 338 |
| IV | — | D | 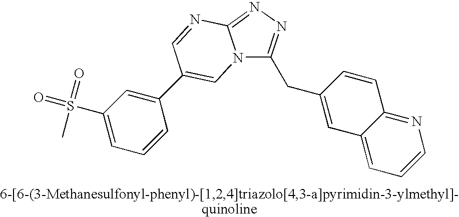 | ¹H NMR (DMSO-d6, 500 MHz) δ 2.54 (s, 3H), 4.48 (s, 2H), 7.53 (dd, 1H), 7.83 (m, 2H), 7.95 (d, 1H), 8.00 (d, 2H), 8.21 (d, 1H), 8.34 (d, 1H), 8.38 (s, 1H), 8.87 (dd, 1H), 9.30 (d, 1H), 9.87 (d, 1H) | 416 |
| IV | — | D | 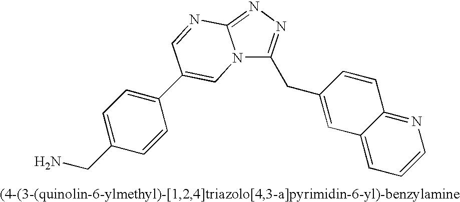 | ¹H NMR (DMSO-d6, 500 MHz) δ 3.76 (s, 2H), 4.38 (s, 2H), 7.42 (m, 3H), 7.62 (m, 2H), 7.73 (dd, 1H), 7.83 (d, 1H), 7.88 (d, 1H), 8.22 (d, 1H), 8.70 (dd, 1H), 9.02 (d, 1H), 9.26 (d, 1H) | 367 |
| I | I | D | 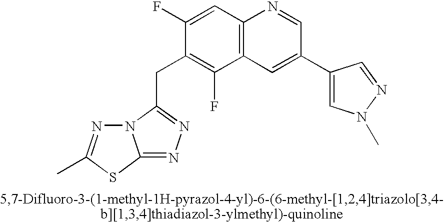 | ¹H NMR (DMSO-d6, 300 MHz) δ 2.71 (s, 3H), 3.91 (s, 3H), 4.61 (s, 2H), 7.7 (d, 1H), 8.19 (s, 1H), 8.49 (s, 1H), 8.55 (d, 1H), 9.23 (d, 1H) | 398 |
| I | I | C | 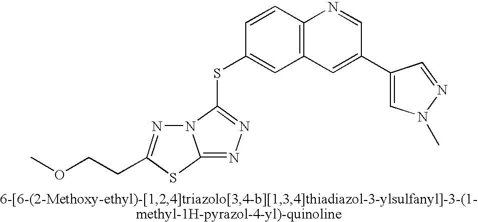 | ¹H NMR (DMSO-d6, 500 MHz) δ 3.26 (m, 5H), 3.65 (t, 2H), 3.91 (s, 3H), 7.60 (dd, 1H), 7.87 (d, 1H), 7.96 (d, 1H), 8.80 (s, 1H), 8.38 (s, 1H), 8.44 (d, 1H), 9.18 (d, 1H) | 424 |
| III | — | A | 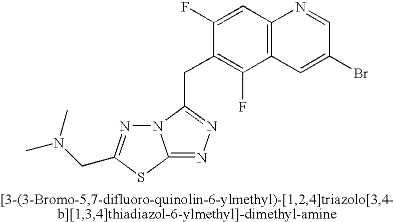 | ¹H NMR (DMSO-d6, 500 MHz) δ 2.30 (s, 6H), 3.80 (s, 2H), 4.60 (s, 2H), 7.80 (d, 1H), 8.80 (d, 1H), 9.10 (s, 1H) | 439 |
| IV | — | A | 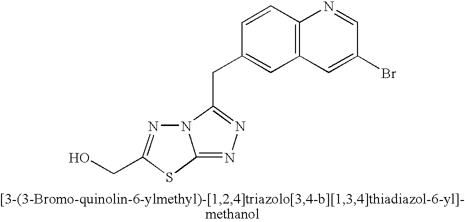 | ¹H NMR (DMSO-d6, 500 MHz) δ 3.10 (s, 2H), 4.15 (s, 2H), 7.66 (s, 1H), 7.69 (dd, 1H), 7.88 (d, 1H), 8.61 (d, 1H), 8.81 (d, 1H) | 376 |

wherein:
I c-MET IC₅₀ or GLT16 IC₅₀ ≦ 100 nM;
II 100 nM < c-MET IC₅₀ or GLT16 IC₅₀ ≦ 1 μM;
III 1 μM < c-MET IC₅₀ or GLT16 IC₅₀ ≦ 10 μM; and
IV c-MET IC₅₀ or GLT16 IC₅₀ > 10 μM.

### Example 10

### General Method I

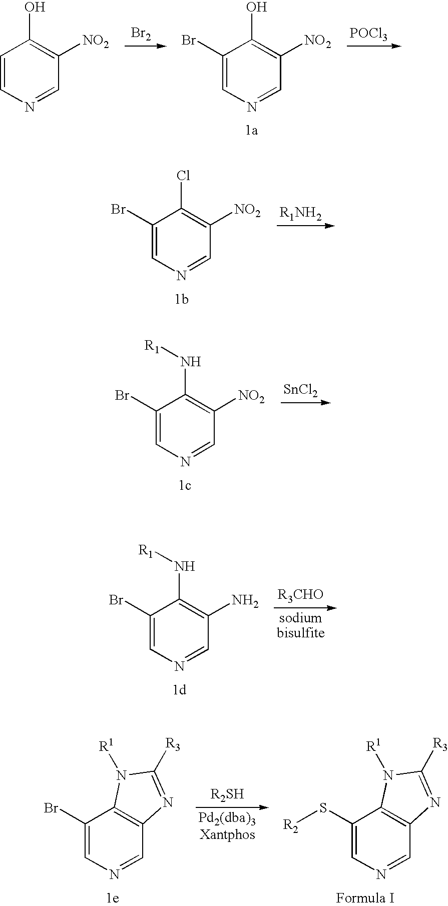

Compounds of general formula I can be made following general route 1. Starting from 3-nitro-4-hydroxypyridine, bromination followed by chlorination with POCl₃ provides compound 1b. Nucleophilic substitution with an amine followed by a reduction of the nitro with tin chloride gives diamine id. Cyclization with an aldehyde in the presence of sodium bisulfite provides compound 1e, which undergoes a palladium-catalyzed S-arylation to give compounds of formula I.

### Example 11

### General Method J

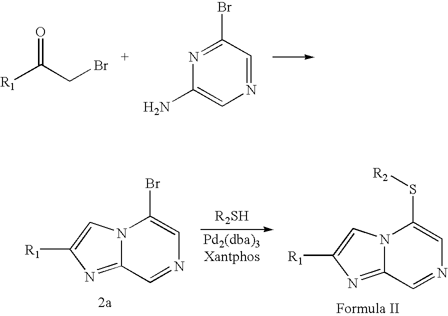

Compounds of general formula II can be made following general route 2. Readily available acyl bromides are condensed with 2-amino-6-bromopyrazine to give compound 2a. Compound 2a then undergoes a palladium-catalyzed S-arylation to give compounds of formula II.

### Example 12

### General Method K

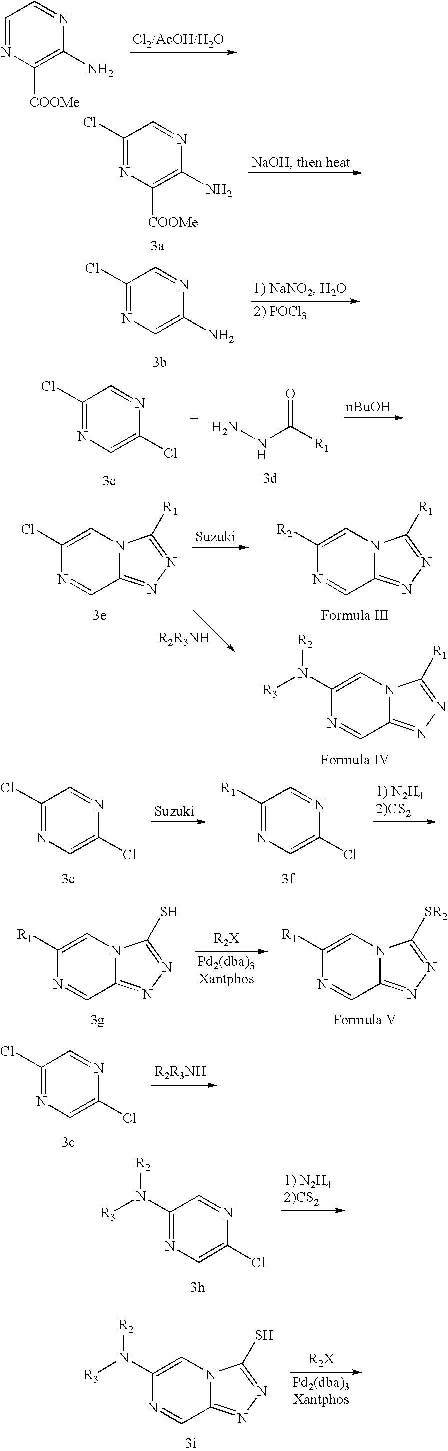

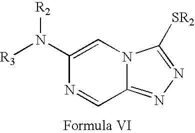

Compounds of general formula III, IV, V and VI can be made following general route 3. Commercially available 3-amino-pyrazine-2-carboxylic acid methyl ester is chlorinated with chlorine in acetic/water mixtures to give compound 3a. Saponification followed by decarboxylation under thermal conditions provides aminopyrazine 3b. The amino group is converted to the hydroxyl group under Sandmeier conditions, followed by chlorination in the presence of POCl₃ to give dichloropyrazine 3c. Condensation of 3c with readily available hydrazide 3d under thermal conditions provides 3e, which undergoes Suzuki coupling to deliver compounds of general formula III. Alternatively, nucleophilic substitution or palladium-catalyzed amination reactions of compound 3e with an appropriate amine provide compounds of general formula IV. Suzuki coupling conditions on compound 3c provide compound 3f. Reaction with hydrazine, followed by cyclization in the presence of carbon disulfide gives thiol 3 g, which undergoes a palladium-catalyzed S-arylation with an appropriate halide or triflate to give compounds of formula V. Alternatively, nucleophilic substitution or palladium-catalyzed amination reactions of compound 3c with an appropriate amine provide compound 3 h. Reaction with hydrazine, followed by cyclization in the presence of carbon disulfide gives thiol 31, which undergoes a palladium-catalyzed S-arylation with an appropriate halide or triflate to give compounds of formula VI.

### Example 13

### General Method L

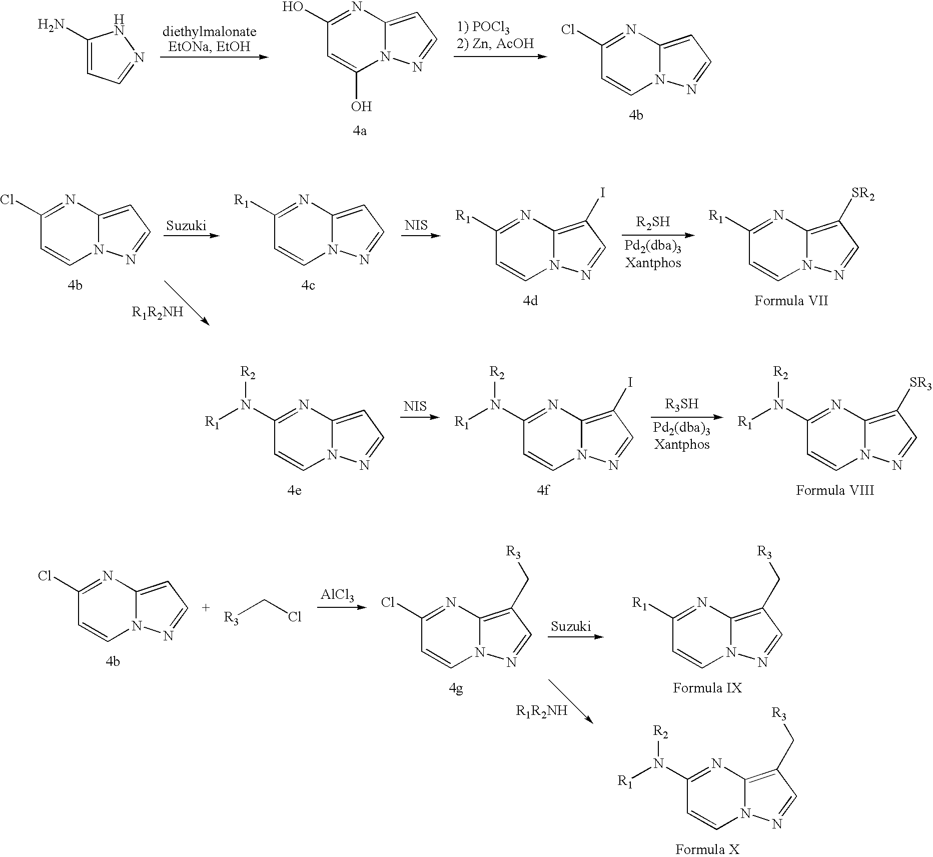

Compounds of general formula VII, VIII, IX and X can be made following general route 4. Commercially available 3-aminopyrazole is condensed with diethylmalonate to give compound 4a. Double chlorination is achieved with POCl₃ and selective dechlorination in the presence of zinc in acetic acid affords compound 4b. Suzuki coupling conditions provide compound 4c, which is further iodinated with NIS to give compound 4d. Subsequent palladium-catalyzed S-arylation reaction delivers compounds of general formula VII. Alternatively, nucleophilic substitution or palladium-catalyzed amination reactions of compound 4b with an appropriate amine provide compound 4e. Subsequent iodination with NIS provide compound 4f, which undergoes palladium-catalyzed S-arylation reaction to provide compounds of general formula VIII. In another route, compound 4b is subjected to Friedel-Crafts conditions in the presence of an alkyl chloride to give compound 4 g, which undergoes subsequent Suzuki reaction to provide compounds of general formula IX. Alternatively, compound 4 g undergoes nucleophilic substitution or palladium-catalyzed amination reactions with an appropriate amine to give compounds of general formula X.

### Example 14

### General Method M

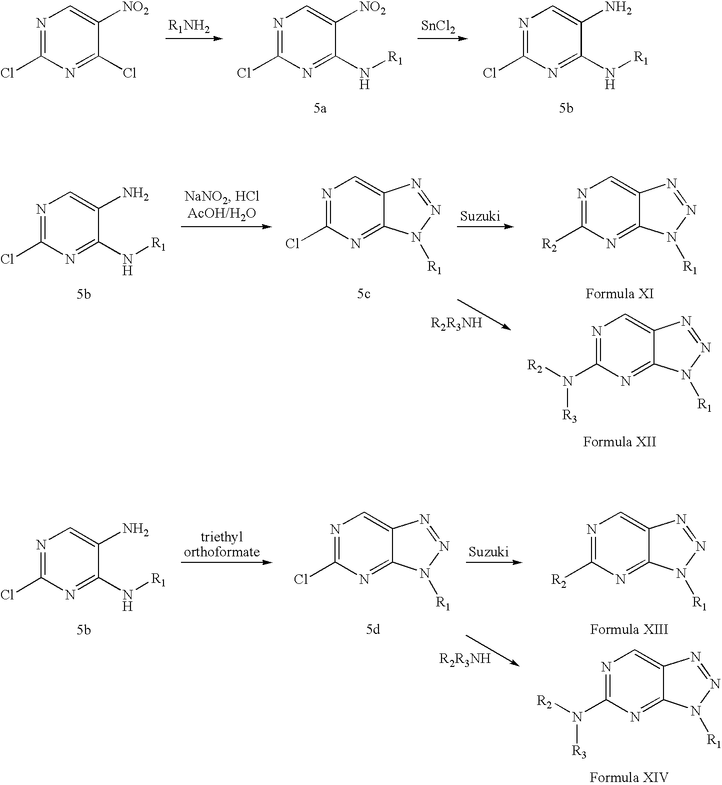

Compounds of general formula XI, XII, XIII and XIV can be made following general route 5. Commercially available 2,4-dichloro-5-nitro-pyrimidine undergoes nucleophilic substitution with an amine to give compound 5a, which is then reduced to compound 5b in the presence of tin chloride. Compound 5b undergoes ring cyclization to diazabenzotriazole 5c, which is then subjected to Suzuki coupling conditions to give compounds of general formula XI. Alternatively, nucleophilic substitution or palladium-catalyzed amination reactions of compound 5c with an appropriate amine provide compounds of general formula XII. In another route, compound 5b undergoes ring cyclization in the presence of triethyl orthoformate to give diazabenzimidazole 5d, which is then subjected to Suzuki coupling conditions to give compounds of general formula XIII. Alternatively, nucleophilic substitution or palladium-catalyzed amination reactions of compound 5d with an appropriate amine provide compounds of general formula XIV.

### Example 15

The following compounds are made according to the general methods described above.

A-B-C-D

| A | A | A | |
|---|---|---|---|
| 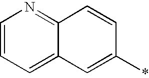 | 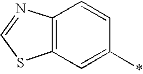 | 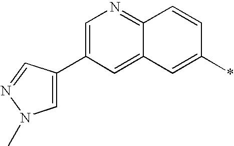 | 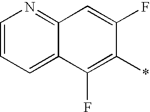 |
| 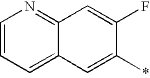 | 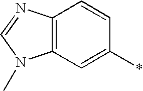 | 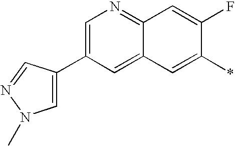 | 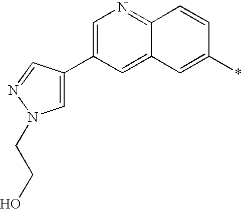 |
| 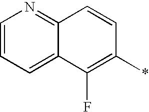 | 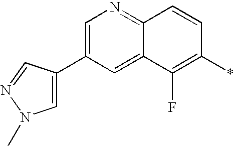 | 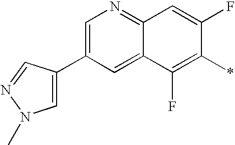 | |
| B = S | B = CH₂ | B = CF₂ | B = CH(CH₃) |

| C | C | C |
|---|---|---|
| 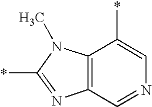 | 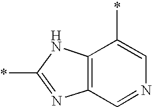 | 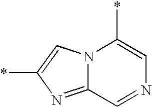 |
| 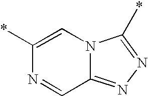 | 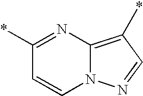 | 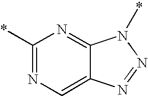 |
| 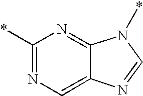 | | |

| | | | | |
|---|---|---|---|---|
| D = CH₃ | D = CH₂CH₃ | D = NHCH₃ | D = NHCH(CH₃) | D = CH₂NHCH₃ |
| 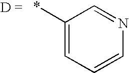 | 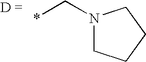 | 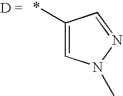 | 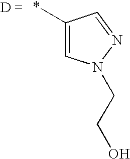 | |

### Example 16

### In Vitro Assays

Kinase assays known to those of skill in the art may be used to assay the inhibitory activities of the compounds and compositions of the present disclosure. Kinase assays include, but are not limited to, the following examples.

Screening data was evaluated using the equation: Z′=1-[3*(σ₊+σ₋)/|μ₊−μ₋|] (Zhang, et al., 1999 J Biomol Screening 4(2) 67-73), where pi denotes the mean and ca the standard deviation. The subscript designates positive or negative controls. The Z′ score for a robust screening assay should be ≧0.50. The typical threshold=μ₊−3*σ₊. Any value that falls below the threshold was designated a “hit”. Dose response was analyzed using the equation: y=min+{(max−min)/(1+10^{[compound]-logIC50})}, where y is the observed initial slope, max=the slope in the absence of inhibitor, min=the slope at infinite inhibitor, and the IC₅₀ is the concentration of compound that corresponds to ½ the total observed amplitude (Amplitude=max−min).

### MET Luminescence-Based Enzyme Assay

Materials: Poly Glu-Tyr (4:1) substrate (Sigma Cat# P-0275), ATP (Sigma Cat#A-3377, FW=551), HEPES buffer, pH 7.5, Bovine serum albumin (BSA) (Roche 92423420), MgCl₂, Staurosporine (Streptomyces sp. Sigma Cat#85660-1MG), white Costar 384-well flat-bottom plate (VWR Cat#29444-088). MET kinase (see below), Kinase-Glom (Promega Cat#V6712).

Stock Solutions: 10 mg/ml poly Glu-Tyr in water, stored at −20° C.; 100 mM HEPES buffer, pH 7.5 (5 ml 1M stock+45 ml miliQH₂O); 10 mM ATP (5.51 mg/ml in dH₂O) stored at −20° C. (diluted 50 μl into total of 10 ml miliQH₂O daily=50 μM ATP working stock); 1% BSA (1 g BSA in 100 ml 0.1M HEPES, pH 7.5, stored at −20° C.), 100 mM MgCl₂; 200 μM Staurosporine, 2× Kinase-Glo™ reagent (made fresh or stored at −20° C.).

Standard Assay Setup for 384-well format (20 μl kinase reaction, 40 μl detection reaction): 10 mM MgCl₂; 0.3 mg/mil poly Glu-Tyr; 0.1% BSA; 1 μl test compound (in DMSO); 0.4 μg/ml MET kinase; 10 μM ATP; 100 mM HEPES buffer. Positive controls contained DMSO with no test compound. Negative controls contained 10M staurosporine. The kinase reactions were initiated at time t=0 by the addition of ATP. Kinase reactions were incubated at 21° C. for 60 min, then 20 μl of Kinase-Glo™ reagent were added to each well to quench the kinase reaction and initiate the luminescence reaction. After a 20 min incubation at 21° C., the luminescence was detected in a plate-reading luminometer.

### Purification of Met:

The cell pellets produced from half of a 12 L Sf9 insect cell culture expressing the kinase domain of human Met were resuspended in a buffer containing 50 mM Tris-HCl pH 7.7 and 250 mM NaCl, in a volume of approximately 40 ml per 1 L of original culture. One tablet of Roche Complete, EDTA-free protease inhibitor cocktail (Cat# 1873580) was added per 1 L of original culture. The suspension was stirred for 1 hour at 4° C. Debris was removed by centrifugation for 30 minutes at 39,800×g at 4° C. The supernatant was decanted into a 500 ml beaker and 10 ml of 50% slurry of Qiagen Ni-NTA Agarose (Cat# 30250) that had been pre-equilibrated in 50 mM Tris-HCl pH 7.8, 50 mM NaCl, 10% Glycerol, 10 mM Imidazole, and 10 mM Methionine, were added and stirred for 30 minutes at 4° C. The sample was then poured into a drip column at 4° C. and washed with 10 column volumes of 50 mM Tris-HCl pH 7.8, 500 mM NaCl, 10% Glycerol, 100 mM Imidazole, and 10 mM Methionine. The protein was eluted using a step gradient with two column volumes each of the same buffer containing 50 mM, 200 mM, and 500 mM Imidazole, sequentially. The 6× Histidine tag was cleaved overnight using 40 units of TEV protease (Invitrogen Cat# 10127017) per 1 mg of protein while dialyzing in 50 mM Tris-HCl pH 7.8, 500 mM NaCl, 10% Glycerol, 10 mM Imidazole, and 10 mM Methionine at 4° C. The 6× Histidine tag was removed by passing the sample over a Pharmacia 5 ml IMAC column (Cat# 17-0409-01) charged with Nickel and equilibrated in 50 mM Tris-HCl pH 7.8, 500 mM NaCl, 10% Glycerol, 10 mM Imidazole, and 10 mM Methionine. The cleaved protein bound to the Nickel column at a low affinity and was eluted with a step gradient. The step gradient was run with 15% and then 80% of the B-side (A-side=50 mM Tris-HCl pH 7.8, 500 mM NaCl, 10% Glycerol, 10 mM Imidazole, and 10 mM Methionine; B-side=50 mM Tris-HCl pH 7.8, 500 nM NaCl, 10% Glycerol, 500 mM Imidazole, and 10 mM Methionine) for 4 column volumes each. The Met protein eluted in the first step (15%), whereas the non-cleaved Met and the cleaved Histidine tag eluted in the 80% fractions. The 15% fractions were pooled after SDS-PAGE gel analysis confirmed the presence of cleaved Met; further purification was done by gel filtration chromatography on an Amersham Biosciences HiLoad 16/60 Superdex 200 prep grade (Cat# 17-1069-01) equilibrated in 500 nM Tris-HCl pH 8.5, 15 mM NaCl, 10% Glycerol and 5 mM DTT. The cleanest fractions were combined and concentrated to 10.4 mg/ml by centrifugation in an Amicon Ultra-1510,000 Da MWCO centrifugal filter unit (Cat# UFC901024).

### Cell Assays

GTL16 cells were maintained in DMEM Medium supplemented with 10% fetal bovine serum (FBS) 2 mM L-Glutamine and 100 units penicillin/100 μg streptomycin, at 37° C. in 5% CO₂.

TPR-MET Ba/F3 cells were created by stably transducing the human TPR-MET gene into Ba/F3 cells using a retroviral system. All cell lines were grown in RPMI-1640 supplemented with 1× penicillin/streptomycin and 10% fetal bovine (Invitrogen, Carlsbad, Calif.). The cells were maintained in a 5% CO₂ humidified incubator at 37° C.

### Cell Survival Assays

Compounds were Tested in the Following Assays in Duplicate.

96-well XTT assay (GTL16 cells): One day prior to assay the growth media was aspirated off and assay media was added to cells. On the day of the assay, the cells were grown in assay media containing various concentrations of compounds (duplicates) on a 96-well flat bottom plate for 72 hours at 37° C. in 5% CO₂. The starting cell number was 5000 cells per well and volume was 120 μl. At the end of the 72-hour incubation, 40 μl of XTT labeling mixture (50:1 solution of sodium 3′-[1-(phenylaminocarbonyl)-3,4-tetrazolium]-bis(4-methoxy-6-nitro) benzene sulfonic acid hydrate and Electron-coupling reagent: PMS (N-methyl dibenzopyrazine methyl sulfate) were added to each well of the plate. After an additional 5 hours of incubation at 37° C., the absorbance reading at 450 nm with a background correction of 650 nm was measured with a spectrophotometer.

96-well XTT assay (Ba/F3 cells): Cells were grown in growth media containing various concentrations of compounds (duplicates) on a 96-well plate for 72 hours at 37° C. The starting cell number was 5000-8000 cells per well and volume was 120 μl. At the end of the 72-hour incubation, 40 μl of XTT labeling mixture (50:1 solution of sodium 3′-[1-(phenylamino-carbonyl)-3,4-tetrazolium]-bis(4-methoxy-6-nitro) benzene sulfonic acid hydrate and Electron-coupling reagent: PMS (N-methyl dibenzopyrazine methyl sulfate) were added to each well of the plate. After an additional 2-6 hours of incubation at 37° C., the absorbance reading at 405 nm with background correction at 650 nm was measured with a spectrophotometer.

### Phosphorylation Assays

Met phosphorylation assay: GTL16 cells were plated out at 1×10̂6 cells per 60×15 mm dish (Falcon) in 3 mL of assay media. The following day compound at various concentrations were added in assay media and incubated for 1 hour at 37° C. 5% CO2. After 1 hour the media was aspirated, and the cells were washed once with 1×PBS. The PBS was aspirated and the cells were harvested in 100 μL of modified RIPA lysis buffer (Tris.Cl pH 7.4, 1% NP-40, 5 mM EDTA, 5 mM NaPP, 5 mM NaF, 150 mM NaCl, Protease inhibitor cocktail (Sigma), 1 mM PMSF, 2 mM NaVO₄) and transferred to a 1.7 mL eppendorf tube and incubated on ice for 15 minutes. After lysis, the tubes were centrifuged (10 minutes, 14,000 g, 4° C.). Lysates were then transferred to a fresh eppendorf tube. The samples were diluted 1:2 (250,000 cells/tube) with 2×SDS PAGE loading buffer and heated for 5 minutes at 98° C. The lysates were separated on a NuPage 4-12% Bis-Tris Gel 1.0 mm×12 well (Invitrogen), at 200V, 400 mA for approximately 40 minutes. The samples were then transferred to a 0.45 micron Nitrocellulose membrane Filter Paper Sandwich (Invitrogen) for 1 hour at 75V, 400 mA. After transferring, the membranes were placed in blocking buffer for 1 hour at room temperature with gentle rocking. The blocking buffer was removed and a 1:500 dilution of anti-Phospho-Met (Tyr1234/1235) antibody (Cell Signaling Technologies Cat. # 3126L) in 5% BSA, 0.05% Tween®20 in 1×PBS was added and the blots were incubated overnight at room temperature. The following day the blots were washed three times with 1×PBS, 0.1% Tween®20. A 1:3000 dilution of HRP conjugated goat anti-rabbit antibody (Jackson ImmunoResearch Laboratories Cat. # 111-035-003) in blocking buffer, was added and incubated for 1 hr at room temperature with gentle rocking. The blot was wash 3 times in PBS, 0.1% Tween®20 and visualized by chemiluminescence with SuperSignal West Pico Chemiluminescent Substrate (Pierce #34078).

What is claimed is:

1. A compound having the structure of Formulas (I1), (I2), (I3) or (I4): 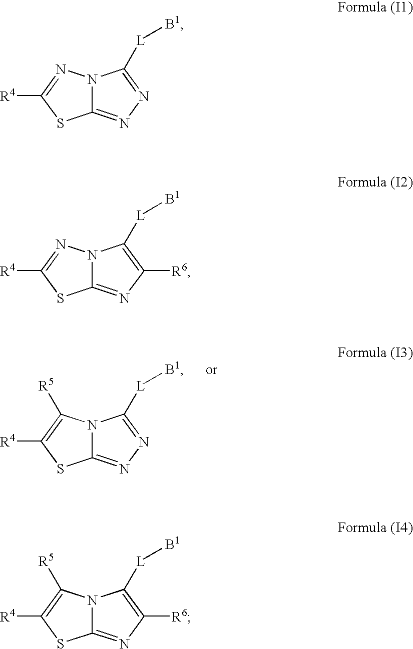
wherein:
L is 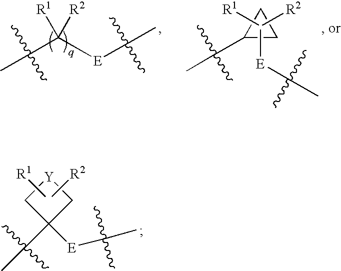
E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;
R⁴ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, (CH₂)ⱼNR²⁰C(O)NR¹⁸R¹⁹, (CH₂)ⱼS(O)ₘR²¹, —(CH₂)ⱼNR²⁰S(O)₂R²¹;
R⁵ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹;
R⁴ and R⁵ optionally form substituted or unsubstituted cycloalkyl, substituted or unsubstituted heterocycloalkyl, substituted or unsubstituted aryl, or substituted or unsubstituted heteroaryl,
R⁶ is hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoheterocycloalkyl, substituted or unsubstituted aminocycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)NR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, —(CH₂)ⱼNR²⁰C(O)NR¹⁸R¹⁹, —(CH₂)ⱼS(O)²¹, —(CH₂)ⱼNR²⁰S(O)₂R²¹, —(CH₂)ⱼS(O)₂NR¹⁸R¹⁹;
R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶;
R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;
B¹ is 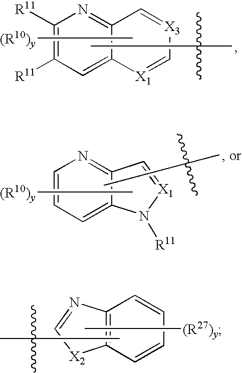
wherein:
X₁ is independently N or CR¹¹;
X₂ is NR¹¹, O, or S; and
X₃ is CR¹⁰ or N;
R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein y is independently an integer from 0 to 4;
R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴; wherein each j is independently an integer from 0 to 6, and m is independently an integer from 0 to 2;
with the proviso that when R¹¹ is independently a direct bond, then R¹⁰ or R²⁷ cannot all be H;
R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

2. The compound of claim 1 having the structure of Formulas (I1a), (I1b), (I2a), (I2b), (I3a), (I3b), (I4a) or (I4b): 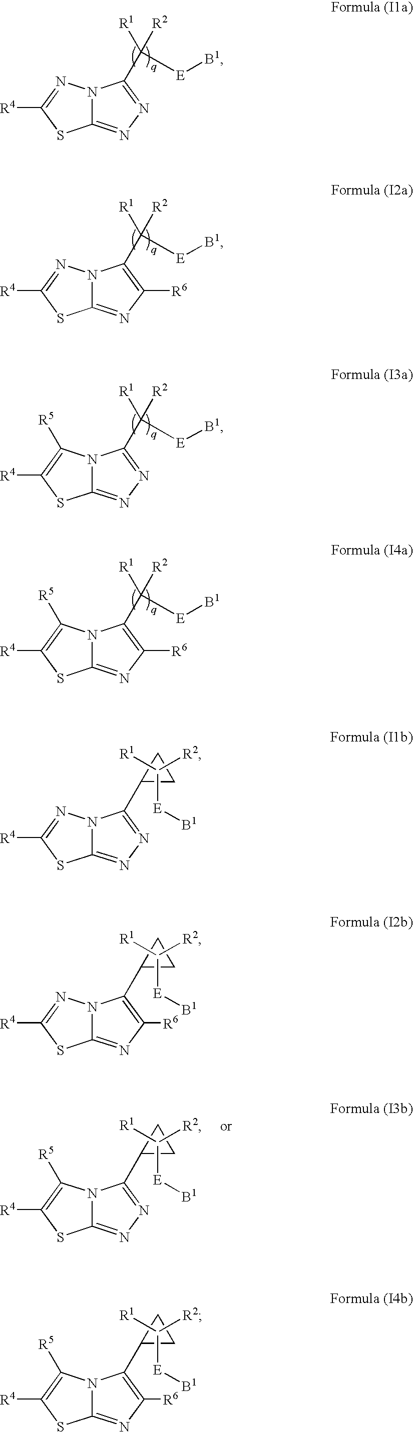
or an enantiomer, diastereomer, racemate, or pharmaceutically acceptable salt, or solvate thereof.

3. The compound of claim 2, wherein B¹ is

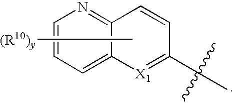

4. The compound of claim 3, wherein X₁ is CR¹¹; and wherein R¹¹ and each R¹⁰ are independently hydrogen, halogen, nitro, cyano, hydroxyl, substituted or unsubstituted alkyl, substituted or unsubstituted aryl, substituted or unsubstituted heteroaryl, and y is an integer from 0 to 5.

5. The compound of claim 4, having the structure of Formulas (I1a), (I2a), (I3a) or (I4a): 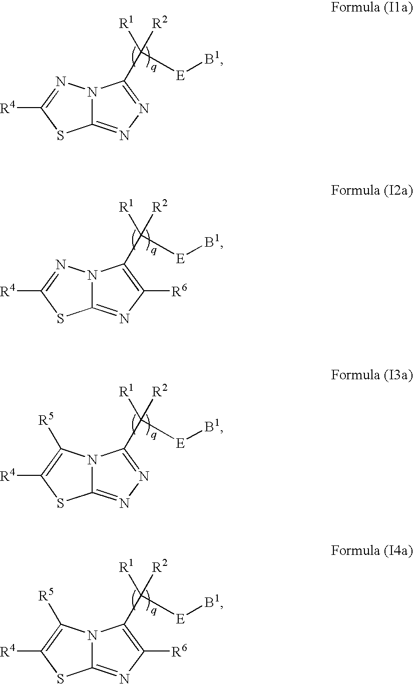
wherein: y is 1 or 2; q is 0-2; and E is a direct bond or S.

6. The compound of claim 3, wherein R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, and y is independently an integer from 0 to 3.

7. The compound of claim 5, wherein R¹⁰ is independently hydrogen, halogen or substituted or unsubstituted heteroaryl, wherein the optional heteroaryl substituents are selected from halogen, C₁-C₃ alkyl, and C₁-C₃ haloalkyl.

8. The compound of claim 3, wherein R⁴ is selected from a group consisting of a substituted or unsubstituted alkyl, perfluoroalkyl, substituted or unsubstituted alkylaminoalkyl, substituted or unsubstituted aminocycloalkyl, substituted or unsubstituted aminoalkylenecycloalkyl, substituted or unsubstituted heterocycloalkyl, substituted or unsubstituted alkylheterocycloalkyl, substituted or unsubstituted heteroalkyl, substituted or unsubstituted heteroaryl, substituted or unsubstituted aryl, and (CH₂)ⱼNR¹⁸R¹⁹.

9. The compound of claim 3, wherein R¹⁰ is independently a substituted or unsubstituted 2H-pyrrolyl, substituted or unsubstituted 2-pyrrolinyl, substituted or unsubstituted 3-pyrrolinyl, substituted or unsubstituted pyrrolidinyl, substituted or unsubstituted dioxolanyl, substituted or unsubstituted 2-imidazolinyl, substituted or unsubstituted imidazolidinyl, substituted or unsubstituted 2-pyrazolinyl, substituted or unsubstituted pyrazolidinyl, substituted or unsubstituted piperidinyl, substituted or unsubstituted morpholinyl, substituted or unsubstituted thiomorpholinyl, substituted or unsubstituted piperazinyl, substituted or unsubstituted phenyl, substituted or unsubstituted phenoxy, substituted or unsubstituted naphthyl, substituted or unsubstituted biphenyl, substituted or unsubstituted thiophenyl, substituted or unsubstituted pyrrolyl, substituted or unsubstituted pyrazolyl, substituted or unsubstituted imidazolyl, substituted or unsubstituted pyrazinyl, substituted or unsubstituted oxazolyl, substituted or unsubstituted isoxazolyl, substituted or unsubstituted thiazolyl, substituted or unsubstituted furyl, substituted or unsubstituted thienyl, substituted or unsubstituted pyridinyl, substituted or unsubstituted O-pyridinyl, substituted or unsubstituted pyrimidyl, substituted or unsubstituted benzothiazolyl, substituted or unsubstituted purinyl, substituted or unsubstituted benzimidazolyl, substituted or unsubstituted indolyl, substituted or unsubstituted isoquinolinyl, substituted or unsubstituted quinoxalinyl, substituted or unsubstituted quinolinyl, substituted or unsubstituted benzooxazolyl, substituted or unsubstituted [1,5]naphthyridinyl, substituted or unsubstituted pyrido[3,2-d]pyrimidinyl, substituted or unsubstituted [1,7]naphthyridinyl, substituted or unsubstituted 1H-pyrrolo[2,3-b]pyridinyl, substituted or unsubstituted pyrazolo[4,3-b]pyridinyl, substituted or unsubstituted pyrrolo[2,3-b]pyridinyl, substituted or unsubstituted thieno[2,3-b]pyridinyl, substituted or unsubstituted thiazolo[5,4-b]pyridinyl, substituted or unsubstituted pyridinyl-2-one, substituted or unsubstituted imidazo[1,2-b]pyridazinyl, substituted or unsubstituted pyrazolo[1,5-a]pyrimidinyl, substituted or unsubstituted pyridazinyl-3-one, substituted or unsubstituted imidazo[2,1-b][1,3,4]thiaciazolyl, substituted or unsubstituted imidazo[2,1-b]thiazolyl, substituted or unsubstituted isothiazolyl, substituted or unsubstituted triazolyl, or substituted or unsubstituted imidazo[4,5-b]pyridinyl.

10. The compound of claim 9, wherein:
R¹⁰ is substituted with 1 to 3 R²⁹ groups, wherein:
R²⁹ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR³⁰, —(CH₂)ⱼC(O)R³⁰, —(CH₂)ⱼC(O)OR³⁰, —(CH₂)ⱼNR³¹R³², —(CH₂)ⱼC(O)NR³¹R³², —(CH₂)ⱼOC(O)NR³¹R³², —(CH₂)ⱼNR³³C(O)R³⁰, —(CH₂)ⱼNR³³C(O)OR³⁰, —(CH₂)ⱼNR³³C(O)NR³¹R³², —(CH₂)ⱼS(O)ₘR³⁴, —(CH₂)ⱼNR³³S(O)₂ R³⁴, —(CH₂)ⱼS(O)₂NR³¹R³², wherein each j is independently an integer from 0 to 6; and m is independently an integer from 0 to 2;
R³⁰ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;
R³¹, R³², R³³, and R³⁴ are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or
R³¹ and R³² together with the N atom to which they are attached, independently form substituted or unsubstituted heterocycloalkyl, or substituted or unsubstituted heteroaryl, or
R³⁰ and R³³ together with the N atom to which they are attached, independently form substituted or unsubstituted heterocycloalkyl, or substituted or unsubstituted heteroaryl, or
R³³ and R³¹ or R³³ and R³² together with the N atom to which they are attached, each independently form substituted or unsubstituted heterocycloalkyl, or substituted or unsubstituted heteroaryl, or
R³³ and R³⁴ together with the N atom to which they are attached, independently form substituted or unsubstituted heterocycloalkyl, or substituted or unsubstituted heteroaryl;
wherein any of the R³⁰, R³¹, R³², R³³, and R³⁴ groups are each optionally independently substituted with 1 to 3 groups, each group independently selected from hydrogen, halogen, hydroxyl, amino, aminomonoalkyl, aminodialkyl, cyano, nitro, difluoromethyl, trifluoromethyl, oxo, alkyl, —O-alkyl, and —S-alkyl.

11. The compound of claim 3, wherein R¹⁰ is independently a substituted or unsubstituted pyrazolyl.

12. The compound of claim 11, wherein R⁴ is selected from a group consisting of a substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted heterocycloalkyl, substituted or unsubstituted alkylheterocycloalkyl, substituted or unsubstituted heteroalkyl, substituted or unsubstituted heteroaryl, substituted or unsubstituted aryl, substituted or unsubstituted alkylaryl, and —(CH₂)ⱼNR¹⁸R¹⁹.

13. The compound of claim 1 selected from:

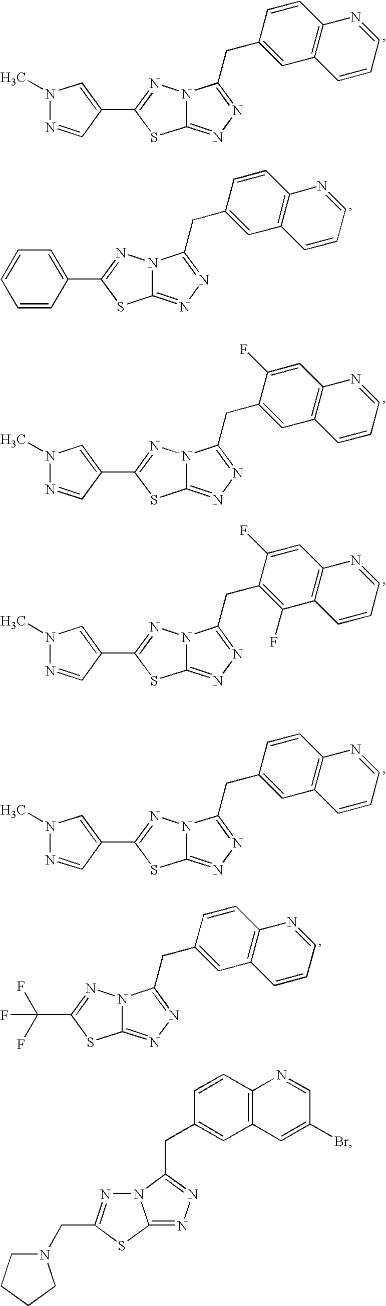

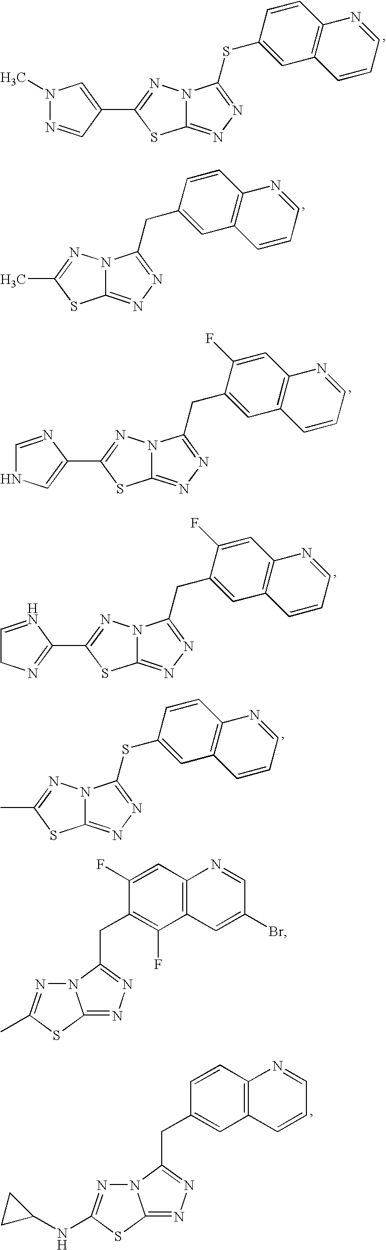

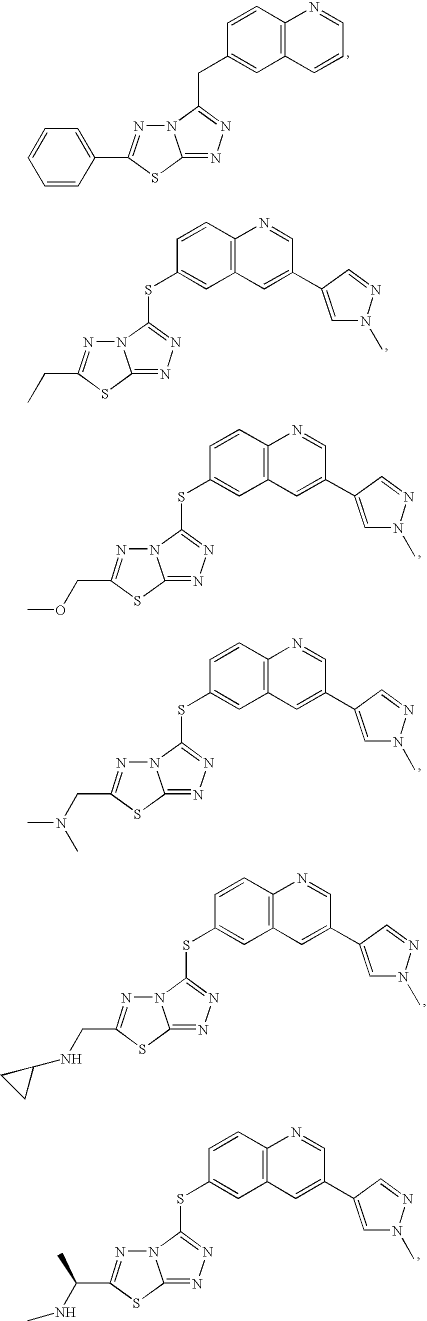

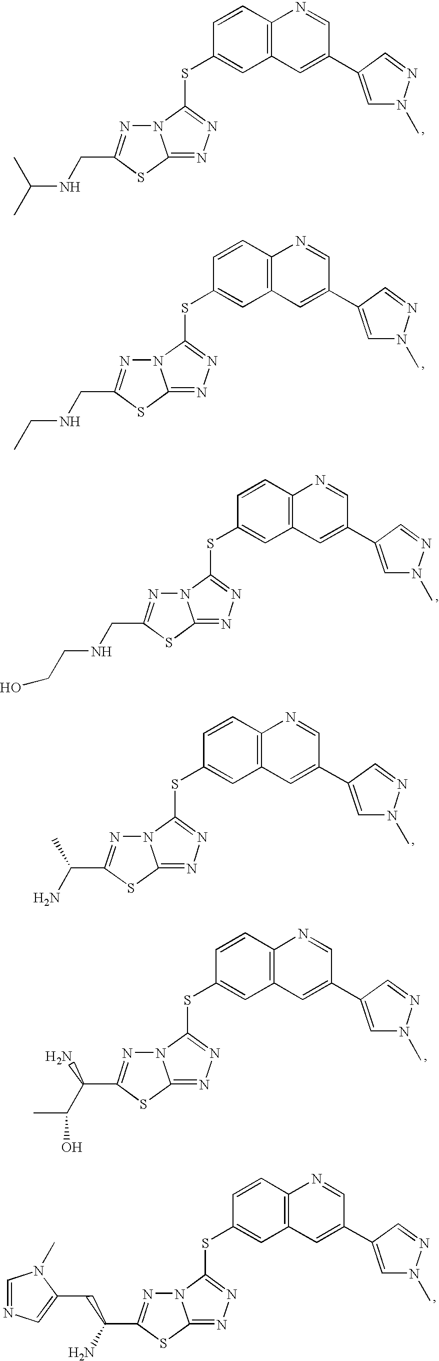

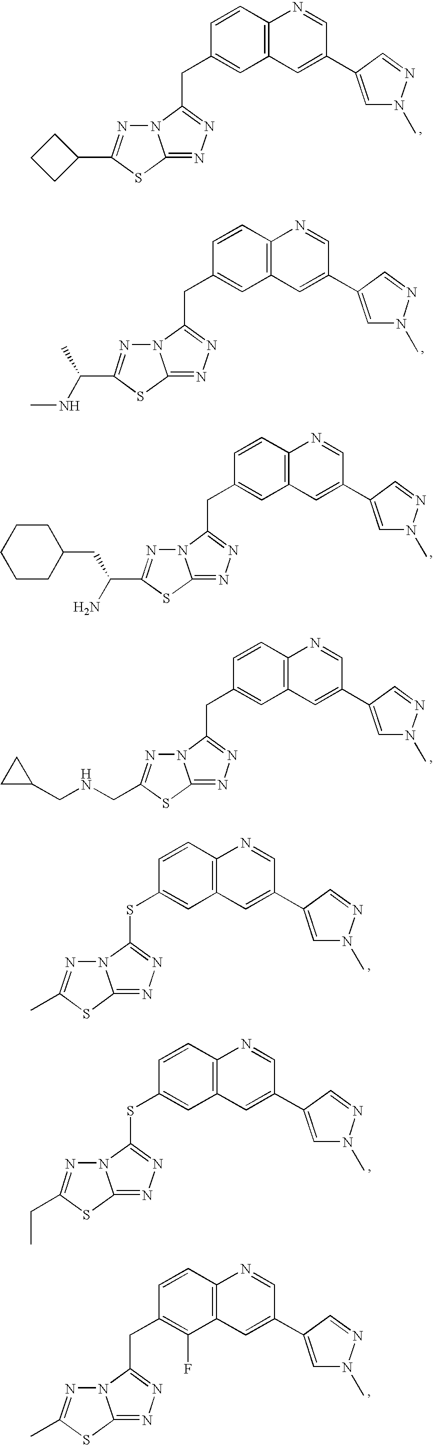

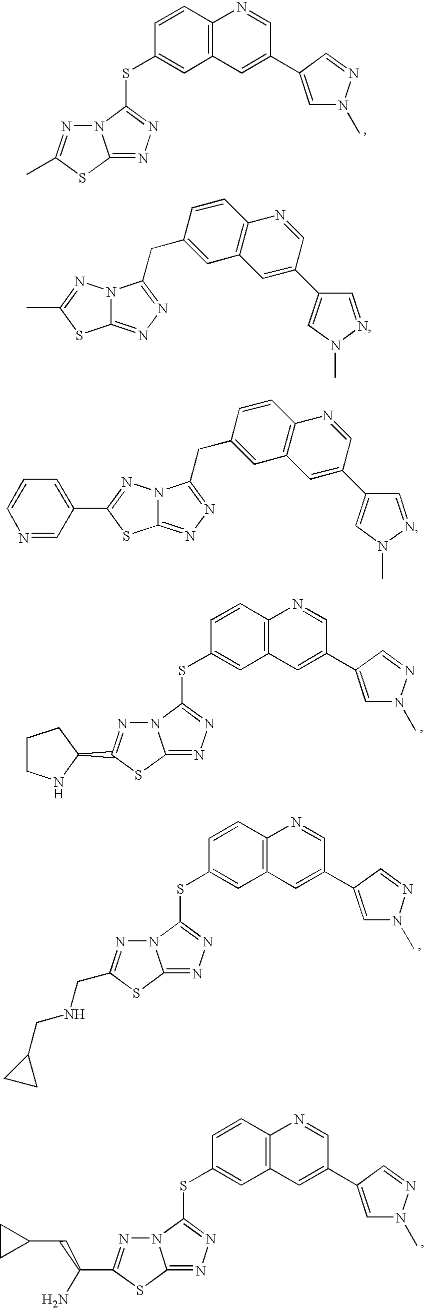

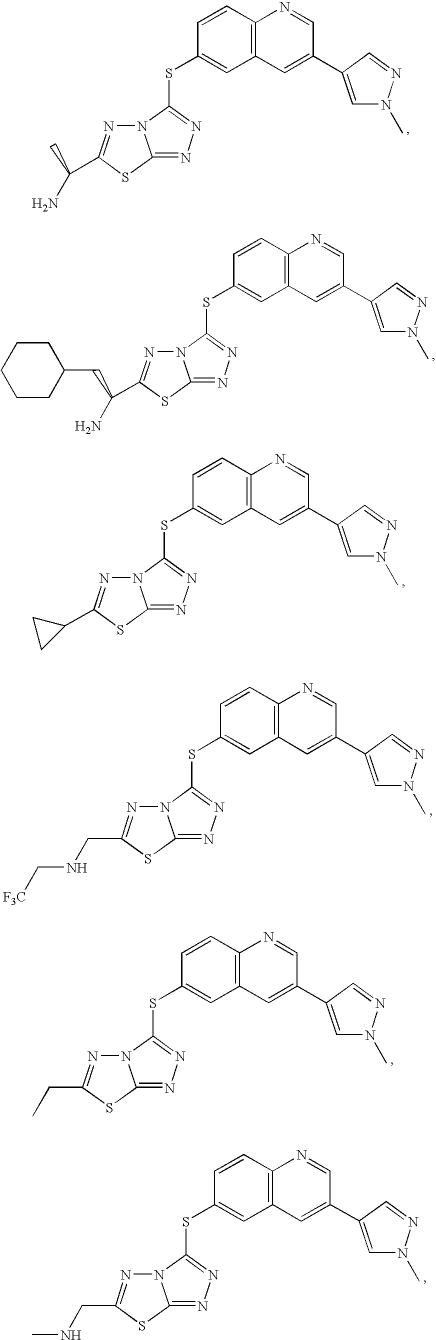

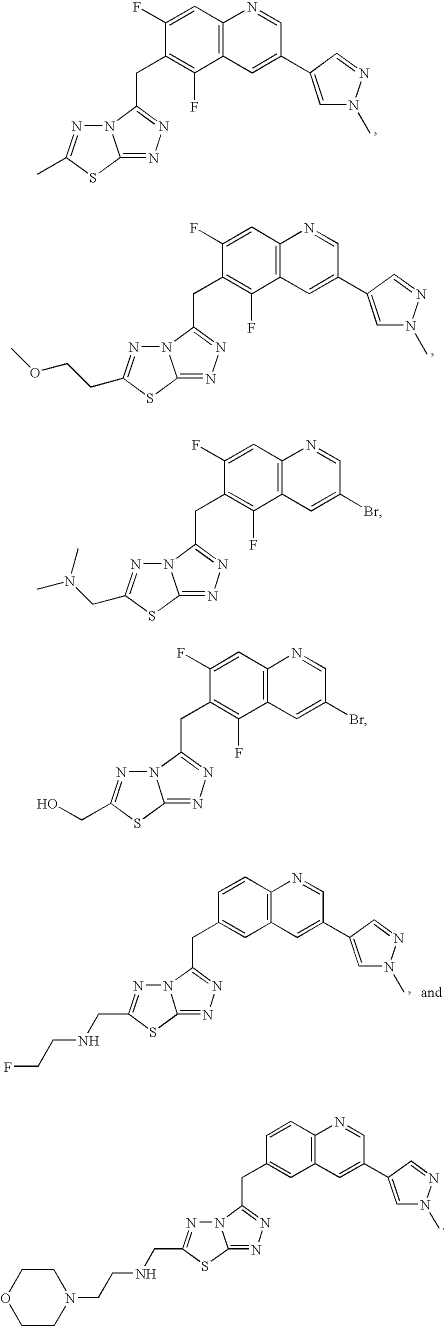

14. A compound having the structure of Formulas (I5), (I6), (I7) or (I8): 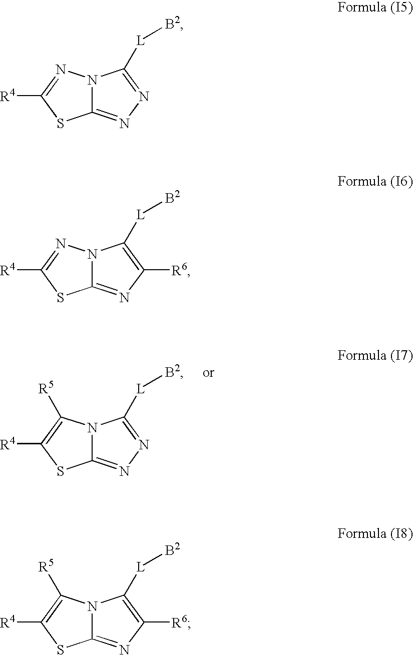
wherein:
L is 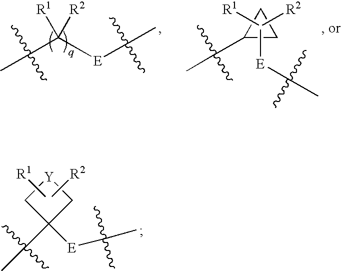
E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;
R⁴, R⁵, and R⁶ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷—(CH₂)ⱼNR¹⁸R¹⁹, (CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ⱼNR²⁰C(O)OR¹⁷, (CH₂)ⱼNR²⁰C(O)NR¹⁸R¹⁹, (CH₂)S(O)ₘR²¹, —(CH₂)ⱼNR²⁰S(O)₂R²¹, —(CH₂)ⱼS(O)₂NR¹⁸R¹⁹;
wherein each j is independently an integer from 0 to 6; and m is independently an integer from 0 to 2;
R⁴ and R⁵ optionally form substituted or unsubstituted cycloalkyl, substituted or unsubstituted heterocycloalkyl, substituted or unsubstituted aryl, or substituted or unsubstituted heteroaryl;
R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶, wherein each j is independently an integer from 0 to 6, and m is independently an integer from 0 to 2;
R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;
B² is: 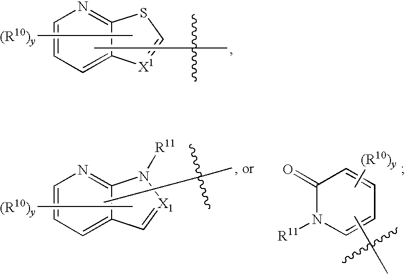
wherein:
X₁ is independently N or CR¹¹;
R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;
y is independently an integer from 0 to 4;
R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)NR²⁵C(O)OR²², —(CH₂)ⱼNR²⁵C(O)NR²³R²⁴, (CH₂)ⱼS(O)R²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;
R¹², R¹³, R¹⁴, R¹⁵, R¹⁶, R¹⁷, R¹⁸, R¹⁹, R²⁰, R²¹, R²², R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl; or an enantiomer, diastereomer, racemate, or pharmaceutically acceptable salt, or solvate thereof.

15. The compound of claim 14 having the structure of Formulas (I5a), (I5b), (I6a), (I6b), (I7a), (I7b), (I8a), or (I8b): 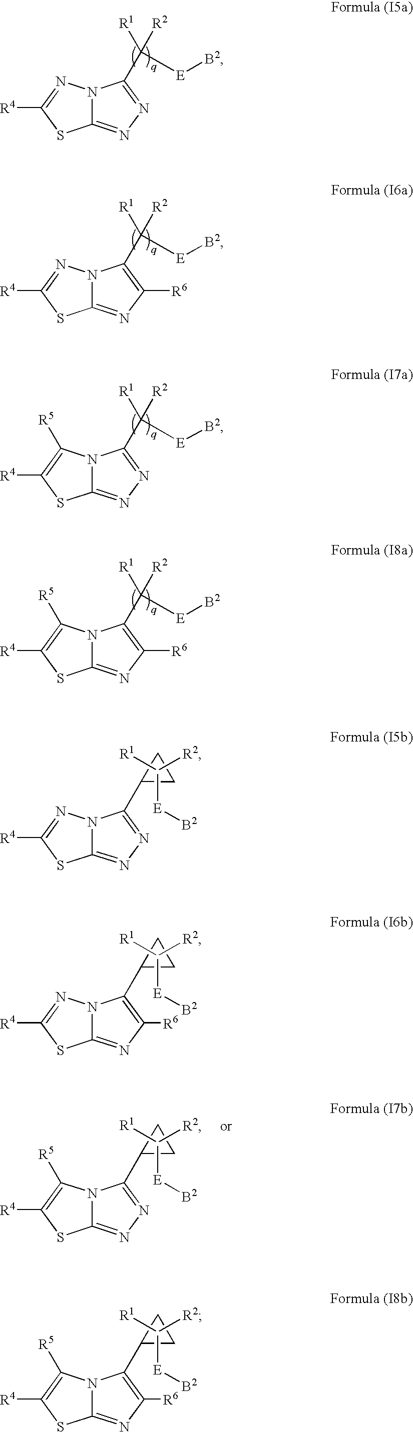
or an enantiomer, diastereomer, racemate, or pharmaceutically acceptable salt, or solvate thereof.

16. A compound having the structure of Formulas (I9), (I10), (I11), (I12), (I13), (I14), or (I15): 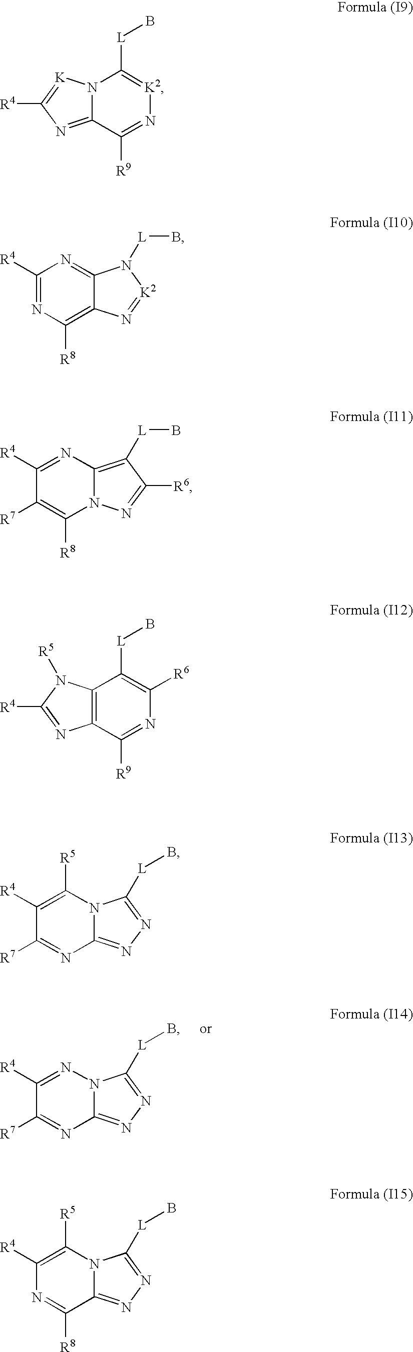
wherein:
K is N or CR⁵;
K² is N or CR⁶; 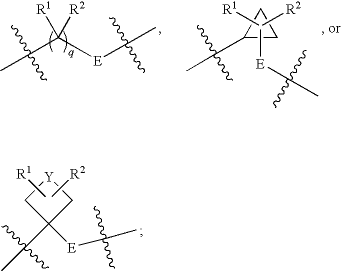
L is
wherein:
E is independently a direct bond, O, C═O, S(O)ᵤ, or NR³;
Y is CH₂, CF₂, O, C(O)—, OC(O)—, NR³, or S(O)ᵤ;
q is an integer from 0 to 4;
u is an integer from 0 to 2;
R⁴, R⁵, R⁶, R⁷, R⁸, and R⁹ are each independently hydrogen, halogen, nitro, cyano, hydroxyl, substituted or unsubstituted alkyl, substituted or unsubstituted cycloalkyl, perfluoroalkyl, substituted or unsubstituted alkylaminoalkyl, substituted or unsubstituted alkylaminocycloalkyl, substituted or unsubstituted alkylaminoalkylenecycloalkyl, substituted or unsubstituted alkylaminoheterocycloalkyl, substituted or unsubstituted aminocycloalkyl, substituted or unsubstituted aminoalkylenecycloalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted alkylaryl, substituted or unsubstituted alkylheterocycloalkyl, substituted or unsubstituted heteroarylalkyl, —(CH₂)ⱼOR¹⁷, —(CH₂)ⱼC(O)R¹⁷, —(CH₂)ⱼC(O)OR¹⁷, —(CH₂)ⱼNR¹⁸R¹⁹, —(CH₂)ⱼC(O)NR¹⁸R¹⁹, —(CH₂)ⱼOC(O)NR¹⁸R¹⁹, —(CH₂)ⱼNR²⁰C(O)R¹⁷, —(CH₂)ᵢNR²⁰C(O)OR¹⁷, —(CH₂)ⱼNR²⁰C(O)NR¹⁸R¹⁹, —(CH₂)ⱼS(O)ₘR²¹, —(CH₂)ⱼNR²⁰S(O)₂R²¹, —(CH₂)ⱼS(O)₂NR¹⁸R¹⁹, wherein each j is independently an integer from 0 to 6; and m is independently an integer from 0 to 2;
R⁴ and R⁵ optionally form substituted or unsubstituted cycloalkyl, substituted or unsubstituted heterocycloalkyl, substituted or unsubstituted aryl, or substituted or unsubstituted heteroaryl, or
R⁴ and R⁷ optionally form substituted or unsubstituted cycloalkyl, substituted or unsubstituted heterocycloalkyl, substituted or unsubstituted aryl, or substituted or unsubstituted heteroaryl, or
R⁷ and R⁸ optionally form substituted or unsubstituted cycloalkyl, substituted or unsubstituted heterocycloalkyl, substituted or unsubstituted aryl, or substituted or unsubstituted heteroaryl;
R¹ and R² are each independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR¹², —(CH₂)ⱼC(O)R¹², —(CH₂)ⱼC(O)OR¹², —(CH₂)ⱼNR¹³R¹⁴, —(CH₂)ⱼC(O)NR¹³R¹⁴, —(CH₂)ⱼOC(O)NR¹³R¹⁴, —(CH₂)ⱼNR¹⁵C(O)R¹², —(CH₂)ⱼNR¹⁵C(O)OR¹², —(CH₂)ⱼNR¹⁵C(O)NR¹³R¹⁴, —(CH₂)ⱼS(O)ₘR¹⁶, —(CH₂)ⱼS(O)₂NR¹³R¹⁴, or —(CH₂)ⱼNR¹⁵S(O)₂R¹⁶, wherein each j is independently an integer from 0 to 6, and m is independently an integer from 0 to 2;
R³ is independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, or substituted or unsubstituted heteroarylalkyl;
B is a substituted or unsubstituted heteroaryl selected from: 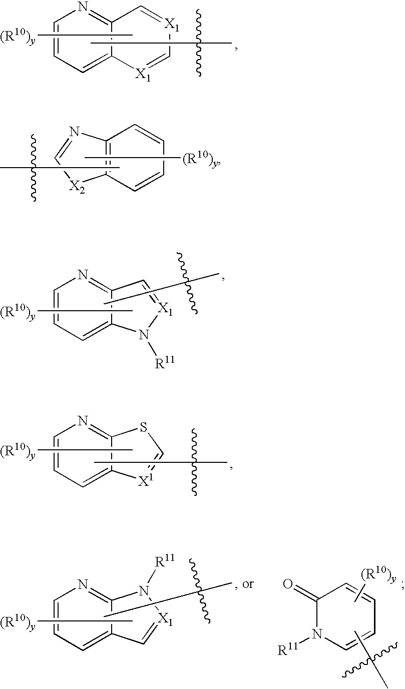
wherein:
X₁ is independently N or C; and
X₂ is N(R¹¹), O, or S;
R¹⁰ is independently hydrogen, halogen, nitro, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)ⱼC(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, (CH₂)ⱼNR²⁵S(O)₂ (O)₂NR²³R²⁴ wherein each j is independently an integer from 0 to 6; and m is independently an integer from 0 to 2;
y is independently an integer from 0 to 5;
R¹¹ is independently a direct bond, hydrogen, cyano, hydroxyl, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted arylalkyl, substituted or unsubstituted heteroaryl, substituted or unsubstituted heteroarylalkyl, —(CH₂)ⱼOR²², —(CH₂)ⱼC(O)R²², —(CH₂)ⱼC(O)OR²², —(CH₂)ⱼNR²³R²⁴, —(CH₂)C(O)NR²³R²⁴, —(CH₂)ⱼOC(O)NR²³R²⁴, —(CH₂)ⱼNR²⁵C(O)R²², —(CH₂)ⱼNR²⁵C(O)OR²², —(CH₂)ⱼNR²⁵C(O)NR²³R²⁴, —(CH₂)ⱼS(O)ₘR²⁶, —(CH₂)ⱼNR²⁵S(O)₂R²⁶, —(CH₂)ⱼS(O)₂NR²³R²⁴, wherein each j is independently an integer from 0 to 6; and m is independently an integer from 0 to 2;
R¹², R¹⁷ and R²² are each independently hydrogen, substituted or unsubstituted alkyl, substituted or unsubstituted cycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl;
R¹³, R¹⁴, R¹⁵, R¹⁶, R¹⁸, R¹⁹, R²⁰, R²¹R²³, R²⁴, R²⁵, and R²⁶ are each independently hydrogen, substituted or unsubstituted alkyl, substituted or unsubstituted cycloalkyl, substituted or unsubstituted alkylcycloalkyl, perfluoroalkyl, substituted or unsubstituted heteroalkyl, substituted or unsubstituted heterocycloalkyl, substituted or unsubstituted aryl, substituted or unsubstituted —O-aryl, substituted or unsubstituted arylalkyl, substituted or unsubstituted heteroaryl, substituted or unsubstituted —O-heteroaryl, or substituted or unsubstituted heteroarylalkyl, or
R¹³ and R¹⁴, R¹⁸ and R¹⁹, and R²³ and R²⁴ together with the N atom to which they are attached, each independently form substituted or unsubstituted heterocycloalkyl, or substituted or unsubstituted heteroaryl, or
R¹² and R¹⁵, R¹⁷ and R²⁰, and R²² and R²⁵ together with the N atom to which they are attached, each independently form substituted or unsubstituted heterocycloalkyl, or substituted or unsubstituted heteroaryl, or
R¹³ and R¹⁵ or R¹⁴ and R¹⁵, R¹⁵ and R²⁰ or R¹⁹ and R²⁰, and R²³ and R²⁵ or R²⁴ and R²⁵ together with the N atom to which they are attached, each independently form substituted or unsubstituted heterocycloalkyl, or substituted or unsubstituted heteroaryl, or
R¹⁵ and R¹⁶, R²⁰ and R²¹, and R²⁵ and R²⁶ together with the N atom to which they are attached, each independently form substituted or unsubstituted heterocycloalkyl, or substituted or unsubstituted heteroaryl;
wherein any of the R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, and R²⁴ groups are each optionally independently substituted with 1 to 3 groups, each group independently selected from hydrogen, halogen, hydroxyl, amino, aminomonoalkyl, aminodialkyl, cyano, morpholine, nitro, difluoromethyl, trifluoromethyl, oxo, alkyl, —O-alkyl, and —S-alkyl;
with the proviso that when the core structure of the compound having a structure of Formula (I14) is [1,2,4]triazolo-[4,3-b][1,2,4]triazine, then R¹⁰ is not hydrogen, halogen, nitro, cyano, hydroxyl, substituted or unsubstituted alkyl, perfluoroalkyl, —(CH₂)ⱼOR²², —(CH₂)ⱼC(O)R²², —(CH₂)ⱼC(O)OR²², —(CH₂)ⱼNR²³R²⁴, —(CH₂)ⱼS(O)ₘR²⁶(CH₂)ⱼC(O)NR²³R²⁴, —(CH₂)ⱼS(O)₂NR²³R²⁴; or when the core structure of the compound having a structure of Formula (I13) is [1,2,4]triazolo[4,3-a]pyrimidine then R¹⁰ is not H;
or an enantiomer, diastereomer, racemate, or pharmaceutically acceptable salt, or solvate thereof.

17. The compound of claim 16 having the structure of Formulas (I9a), (I9b), (I10a), (I10b), (I11a), (I11b), (I12a), (I12b), (I13a), (I13b), (I14a), (I14b), (I15a) or (I15b): 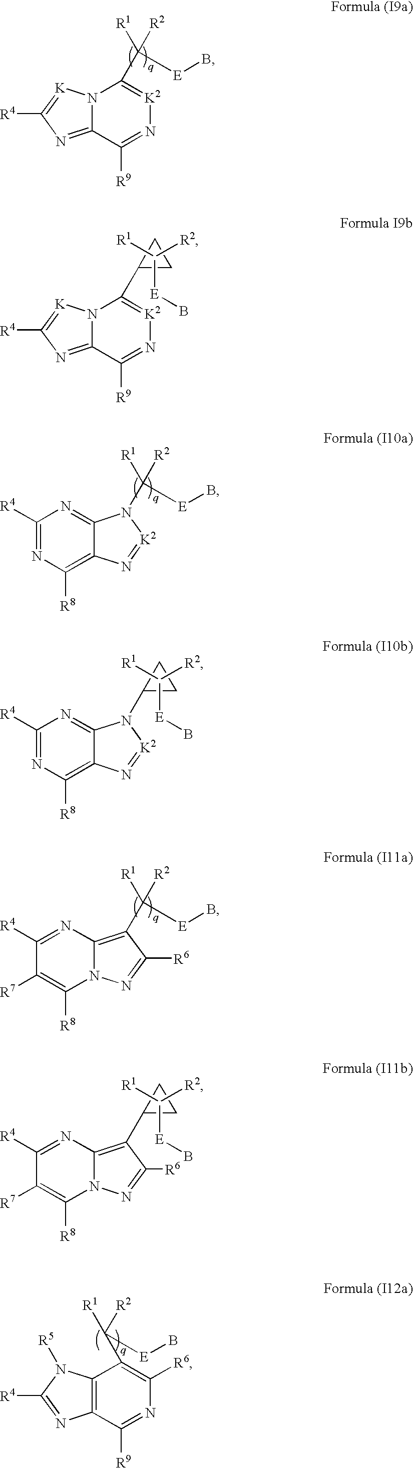 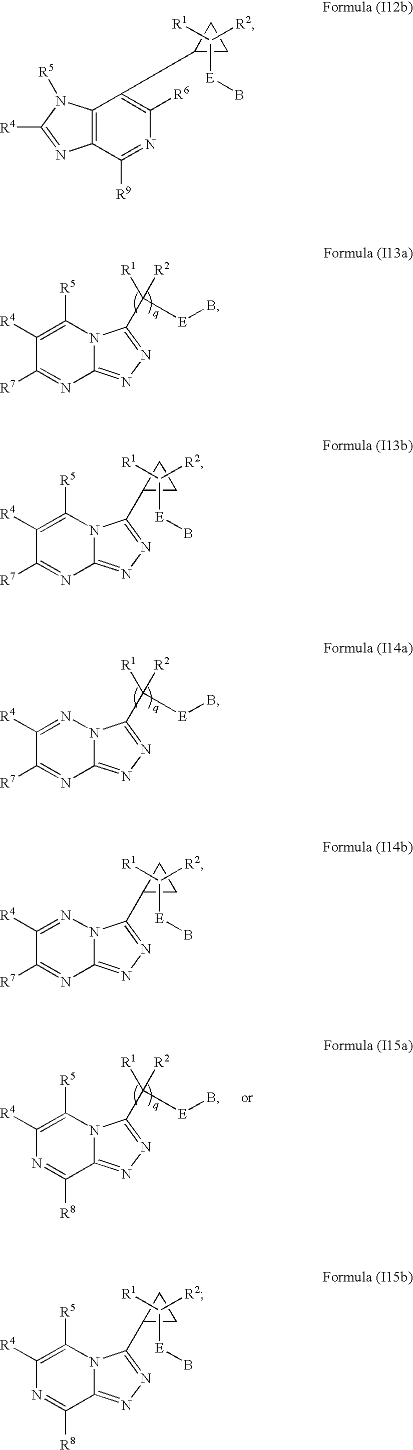
or an enantiomer, diastereomer, racemate, or pharmaceutically acceptable salt, or solvate thereof.

18. The compound of claim 17 wherein B is 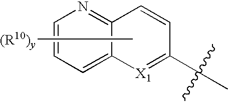
and R¹⁰ is independently a substituted or unsubstituted pyrazolyl.

19. A method of modulating the activity of a protein tyrosine kinase comprising contacting the protein tyrosine kinase with a compound of claim 1; or an enantiomer, diastereomer, racemate, or pharmaceutically acceptable salt, or solvate thereof.

20. The method of claim 19, wherein the protein kinase is Met receptor tyrosine kinase.

21. A method for treating cancer in a subject in need of treatment, comprising administering to the patient a therapeutically effective amount of a compound of claim 1.

22. The method of claim 21, wherein the cancer is bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

23. The method of claim 22, further comprising administering at least one of radiation and one or more chemotherapeutic agents.
